SEQUENCE LISTING (11 sequences)

SEQ ID NO: 1 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide
gcagtatgttgatgga

SEQ ID NO: 2 - RNA, 16 nt, synthetic construct
  source 1..16: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 14: mod_base = OTHER; note = cytidine phosphorothioate
  modified_base 15: mod_base = OTHER; note = 2'-O-methyl modified nucleotide phosphorothioate
  modified_base 16: mod_base = OTHER; note = 2'-O-methyl modified nucleotide
tgtgacttccagacca

SEQ ID NO: 3 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-O-methyl modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-deoxy-fluoro modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-O-methyl modified nucleotide
gtatcctaatggtgta

SEQ ID NO: 4 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
taatcctcactctaaa

SEQ ID NO: 5 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide phosphorothioate
gcagtatgttgatgga

SEQ ID NO: 6 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide phosphorodithioate
gcagtatgttgatgga

SEQ ID NO: 7 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide
tcatctcaagtcttaa

SEQ ID NO: 8 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide phosphorothioate
tcatctcaagtcttaa

SEQ ID NO: 9 - RNA, 16 nt, synthetic construct
  source 1..16: mol_type = other RNA
  modified_base 1: mod_base = OTHER; note = 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-deoxy-fluoro modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-deoxy-fluoro modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide
  modified_base 16: mod_base = OTHER; note = 2'-deoxy-fluoro modified nucleotide phosphorodithioate
tcatctcaagtcttaa

SEQ ID NO: 10 - RNA, 19 nt, synthetic construct
  source 1..19: mol_type = other RNA
  modified_base 1: mod_base = OTHER; note = phosphate 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-O-methyl modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide phosphorothioate
  modified_base 16: mod_base = OTHER; note = 2'-deoxy-fluoro modified nucleotide phosphorothioate
  modified_base 17: mod_base = OTHER; note = 2'-O-methyl modified nucleotide phosphorothioate
  modified_base 18: mod_base = OTHER; note = 2'-deoxy-fluoro modified nucleotide phosphorothioate
  modified_base 19: mod_base = OTHER; note = 2'-O-methyl modified nucleotide
tccatcaacatactgcttc

SEQ ID NO: 11 - RNA, 19 nt, synthetic construct
  source 1..19: mol_type = other RNA
  modified_base 1: mod_base = OTHER; note = phosphate 2'-O-methyl modified nucleotide phosphorothioate
  modified_base 2: mod_base = OTHER; note = 2'-deoxy-fluoro modified nucleotide phosphorothioate
  modified_base 3: mod_base = OTHER; note = 2'-O-methyl modified nucleotide
  modified_base 4: mod_base = OTHER; note = 2'-deoxy-fluoro modified nucleotide
  modified_base 5: mod_base = OTHER; note = 2'-O-methyl modified nucleotide
  modified_base 6: mod_base = OTHER; note = 2'-deoxy-fluoro modified nucleotide
  modified_base 7: mod_base = OTHER; note = 2'-O-methyl modified nucleotide
  modified_base 8: mod_base = OTHER; note = 2'-deoxy-fluoro modified nucleotide
  modified_base 9: mod_base = OTHER; note = 2'-O-methyl modified nucleotide
  modified_base 10: mod_base = OTHER; note = 2'-deoxy-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-deoxy-fluoro modified nucleotide
  modified_base 15: mod_base = OTHER; note = 2'-O-methyl modified nucleotide phosphorothioate
  modified_base 16: mod_base = OTHER; note = 2'-deoxy-fluoro modified nucleotide phosphorothioate
  modified_base 17: mod_base = OTHER; note = 2'-O-methyl modified nucleotide phosphorothioate
  modified_base 18: mod_base = OTHER; note = 2'-deoxy-fluoro modified nucleotide phosphorothioate
  modified_base 19: mod_base = OTHER; note = 2'-O-methyl modified nucleotide
ttaagacttgagatgatcc

(19) United States
(12) Patent Application Publication
(10) Pub. No.: US 2023/0372508 A1
(43) Pub. Date: Nov. 23, 2023

(54) LINKERS COUPLING FUNCTIONAL LIGANDS TO MACROMOLECULES

(71) Applicant: OliX US, Inc., San Diego, CA (US)

(72) Inventors: Dongwon Shin, San Diego, CA (US); Namho Kim, San Diego, CA (US); Raymond Emehiser, Santee, CA (US)

(21) Appl. No.: 18/320,753

(22) Filed: May 19, 2023

Related U.S. Application Data
(60) Provisional application No. 63/343,737, filed on May 19, 2022.

(51) Int. Cl. A61K 47/64

(52) U.S. Cl. CPC A61K 47/6455

(57) ABSTRACT

Provided herein are functional moieties including at least one β-amino-acid within an amino-acid cluster for conjugation with one or more ligands. The functional moieties are useful for linking a macromolecule with the one or more functional ligands, which may be used to facilitate delivery of the macromolecule to a site within a subject.

### RELATED APPLICATIONS

This application claims priority of U.S. Provisional Patent Application No. 63/343,737, filed May 19, 2022, the entire content of which is incorporated herein by reference.

### SEQUENCE LISTING

This application contains a sequence listing having the filename 0817444_00020_SL.xml, which is 107,276 bytes in size, created on May 10, 2023, the entire content of which is incorporated herein by reference.

### BACKGROUND

It is important to maximize the pharmaceutical potency and reduce or avoid off-target effects of therapeutics, including siRNA. For example, due to the limitation of siRNA application such as nuclease degradation, short-lived circulation, immune recognition in blood circulation, accumulation in undesired tissue, effective transmembrane trafficking, and endosomal and lysosomal escape to the cytoplasm, many research groups have pursued the investigation of various chemical conjugates and developed the delivery systems. The introduction of chemical modifications into oligonucleotides have been able to overcome the above-mentioned limitations in some areas. Particularly in the blood circulation, the ligand-siRNA conjugates exhibited proper transport of siRNA to desired tissues and cells by specific recognition and interactions between the ligands and the surface receptor. This active targeting strategy achieves robust gene silencing at low doses as well as reducing or avoiding unwanted side effects and toxicity by reducing siRNA accumulation in unintended tissues. There have been several well-known ligands, including: N-acetylgalactosamine (GalNAc) to hepatocytes through asialoglycoprotein receptor (ASGPR) and Mannose/N-acetylglucosamine (GlcNAc) to macrophages through mannose receptor. On the other hand, conjugation of lipophilic molecules such as cholesterol, bile acids and fatty acids increased the binding affinity of siRNA to plasma proteins, thereby improving siRNA delivery through passive targeting and/or through active targeting that intercepts the endogenous lipid transport pathway. In addition, multi-conjugated siRNA has been well-proven as the effective strategy for delivering siRNA to desired tissues and cells. Representatively, tri-GalNAc is one of the well-known conjugation strategy for delivering siRNA to hepatocytes.

Bile acid conjugation has long been investigated as absorption enhancers due to its efficient recycling pathway in the human body. To introduce the chemical conjugates into siRNA, there have been two main approaches, particularly for tri-GalNAc motif. The first strategy, ‘cluster-based approach’, follows the design principle of trivalent structure, and the second strategy, ‘monomer-based approach’, constructs GalNAc cluster structures by multiple couplings of phosphoramidite derived from GalNAc. Whatever selected, there have been two practical methods to introduce the chemical conjugates; firstly to using a solid support containing cluster or monomer and secondly to utilize the phosphoramidite of cluster or monomer. These strategies can also be applied to most chemical conjugations to macromolecules, including siRNA.

Typically, oligonucleotide synthesizers are used to perform each cycle, which may include a number of chemical steps, in order to improve overall yield of a final desired oligonucleotide. Solid support is a useful tool for preparing macromolecules, including siRNA, by sequentially iterating the coupling cycles. For example, the introduction of chemical conjugation can be initiated at the 3′-position utilizing a solid support containing the conjugate cluster. On the other hand, phosphoramidite chemistry has been well-established since it was first described in the 1980s. Sequential addition of monomeric conjugate phosphoramidite can change the number of conjugates by automation. It is also possible to combine the synthetic methods using solid support and phosphoramidite of chemical conjugation.

Preparation of oligomers, including oligonucleotides, carbohydrates, peptides, or the like, may be performed via iterations of synthetic cycles. For example, deoxyribonucleic acid (DNA) synthesis may comprise a first monomer bound to a solid support on which an oligomer of DNA is prepared by cycling through steps including deblocking the first monomer, and coupling of a second monomer to the first monomer. Optional steps include capping of uncoupled first monomers, and oxidation. Iterative cycling of these steps may generate the desired length and sequence of molecule, which cycle is then ended upon final processing of the oligomer including a final deprotection sequence and deblocking of, typically, a chromophoric protecting moiety, e.g., a trityl (including for use with nucleic acids) or a fluorenylmethyloxycarbonyl (Fmoc) moiety (including for use with amide backbone molecules, or chimeras), and purification. Similar cycles are utilized for synthesizing peptides, carbohydrates, or other molecules amenable to preparation by iterative synthesis cycling. Many commercial entities provide services to prepare molecules in this way, including Glen Research, Integrated DNA Technologies, Panagene, GlycoUniverse, CSBio, as well as many others. A variety of benchtop machines are available for researchers to build their own molecules, including Kilobaser, Biolytic's Dr. Oligo series, Biolytic's ABI series, the MerMade series, the Expedite series, the Glyconeer, the Biotage series, and many other synthesizers.

To date, many research groups have developed various methods for introducing the chemical conjugates into siRNA, and peptide-based approaches have shown great promise for structural variation and modification. Multivalent conjugates can be easily introduced using functional groups of amino acids such as lysine, aspartic acid, glutamic acid. However, the fragility of the peptide backbone to proteinases also limited its use in physiological condition through blood circulation. Accordingly, there is an increasing demand for a more stable peptide backbone structure.

Thus, provided herein are pharmaceutically stability improved functional moieties and their uses and synthetic preparation for chemical conjugates.

### SUMMARY

Provided herein are pharmacological stability-improving functional moieties (e.g., amino-acid clusters, which may be functionalized with one or more ligands), which are useful in preparing functionalized compounds and oligonucleotides, their preparation, and uses thereof.

In some embodiments, provided herein are compounds comprising one or more of the following formula:

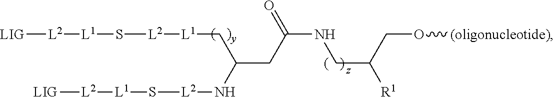

or a stereoisomer or a salt thereof.

In some embodiments, provided herein are compounds comprising one or more of the following formula:

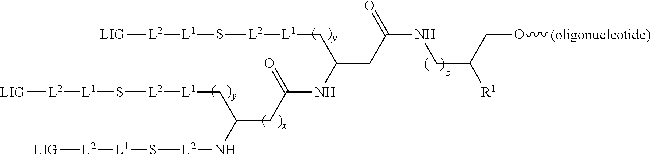

or a stereoisomer or a salt thereof.

In some embodiments, provided herein are compounds comprising one or more of the following formula:

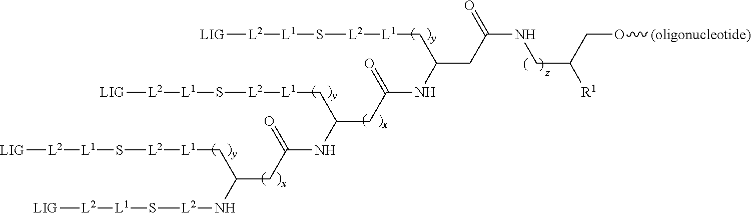

or a stereoisomer or a salt thereof.

In some embodiments, provided herein are compounds comprising one or more of the following formula:

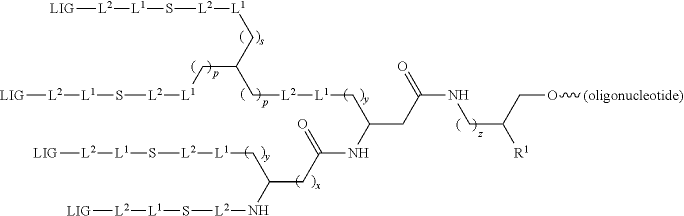

or a stereoisomer or a salt thereof.

In some embodiments, provided herein are compounds comprising one or more of the following formula:

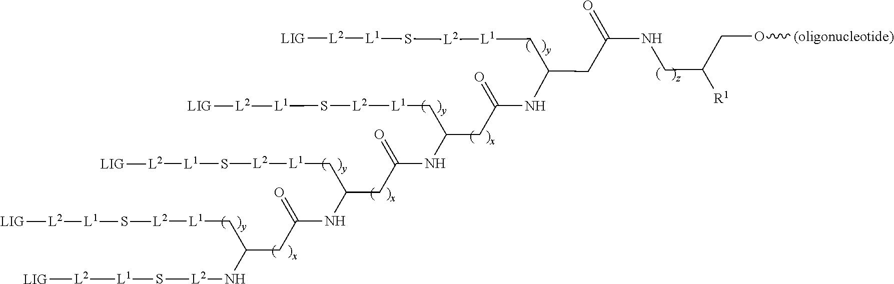

or a stereoisomer or a salt thereof.

In some embodiments, provided herein are compounds comprising one or more of the following formula:

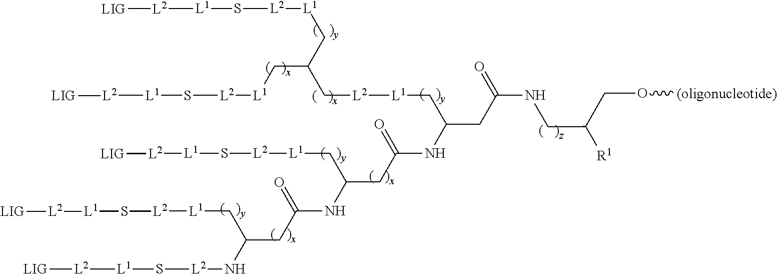

or a stereoisomer or a salt thereof.

In some embodiments, provided herein are compounds comprising one or more of the following formula:

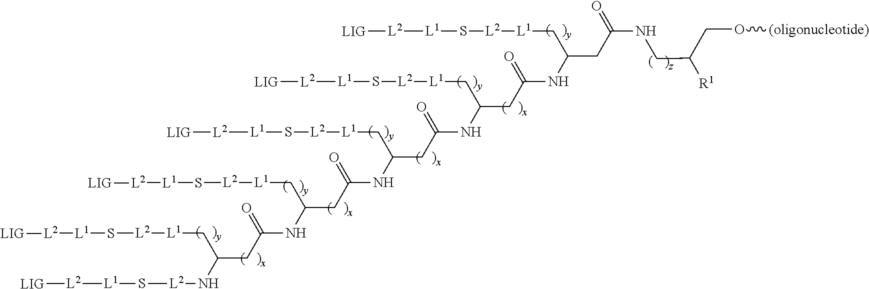

or a stereoisomer or a salt thereof.

In some embodiments, provided herein are compounds comprising one or more of the following formula:

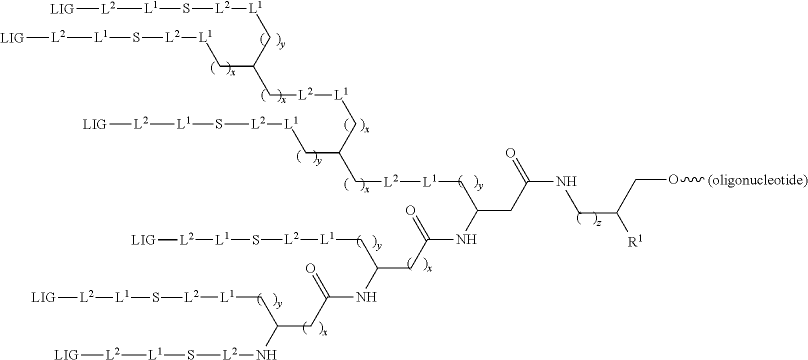

or a stereoisomer or a salt thereof.

In some embodiments, provided herein are compounds comprising one or more of the following formula:

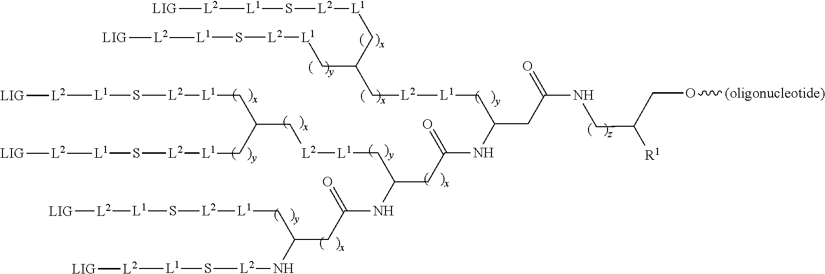

or a stereoisomer or a salt thereof.

In some embodiments of the formulae provided herein, “oligonucleotide” may be replaced with a phosphoramidite or “macromolecule,” wherein the macromolecule comprises one or more of a solid support or an oligomer, including those selected, independently, from oligonucleotides, carbohydrates, peptides, or the like. Thus, in some embodiments of the formulae provided herein, the moiety

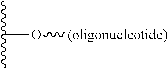

may be replaced by

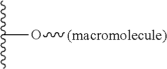

### or —OP(N(iPr)₂)(OEtCN).

### BRIEF DESCRIPTION OF THE DRAWINGS

FIG. 1 shows proteinase K stability of oligonucleotide-amino-acid cluster ligand conjugates provided herein for about 5 days of exposure as described in the examples herein.

FIG. 2 shows proteinase K stability of oligonucleotide-amino-acid cluster ligand conjugates provided herein for about 7 days of exposure as described in the examples herein.

FIG. 3 shows physiological stability of comparative oligonucleotide-amino-acid cluster ligand conjugates, without a β-amino acid in the cluster, as described in the examples herein.

FIG. 4 shows physiological stability of comparative oligonucleotide-amino-acid cluster ligand conjugates, without a β-amino acid in the cluster, and oligonucleotide-amino-acid cluster ligand conjugates provided herein as described in the examples herein.

FIG. 5 shows physiological stability of comparative oligonucleotide-amino-acid cluster ligand conjugates, without a β-amino acid in the cluster, as described in the examples herein where the oligonucleotide includes a duplex.

FIG. 6 shows mouse liver homogenate stability of oligonucleotide-amino-acid cluster ligand conjugates provided herein as described in the examples herein.

FIG. 7 shows in vivo efficacy test 1 results for tri-GalNAc amino-acid cluster conjugated oligonucleotide duplexes as described in the examples herein.

FIG. 8 shows in vivo efficacy test 2 results for tri-GalNAc amino-acid cluster conjugated oligonucleotide duplexes as described in the examples herein.

### DETAILED DESCRIPTION

Nucleic acid-based therapeutics modulating gene expression have been developed for clinical use at a steady pace for decades. Several products based on antisense oligonucleotides (ASOs), aptamers and small interfering RNAs (siRNAs) have recently been launched and many candidates are in pipelines in academia and pharmaceutical industries. Among them, small interfering RNAs (siRNAs), also called short interfering RNA or silencing RNA, are a class of double stranded RNAs that are non-coding RNA molecules, usually 20-24 base pairs in their natural length, and function within the RNA interference (RNAi) pathway. After transcription, it interferes with the translation of mRNA by breaking down the expression of a specific gene with a complementary nucleotide sequence. Naturally occurring siRNAs have a well-defined structure, which is a short double-stranded RNA (dsRNA) with a phosphorylated 5′-end and hydroxylated 3′-end with two overhanging nucleotides. Since in principle any gene can be knocked down by synthetic siRNA with complementary sequences, siRNA is an important tool to validate gene function and drug targeting in the post-genomic era. Patisiran (Onpattro, Alnylam Pharmaceuticals, FDA approval in 2018) was the first marketed siRNA-based drug for the cure of polyneuropathy caused by hereditary TTR-mediated amyloidosis. Recently, another siRNA drug, Givosiran (Givlaari, Alnylam Pharmaceuticals) received FDA approval in 2019 for the treatment of acute hepatic porphyria.

Targeted delivery is a major hurdle for effective RNA therapeutics. To maximize therapeutic efficacy and reduce or avoid off-target effects of siRNAs, a series of chemical conjugation patterns have been developed and evaluated preclinically and clinically with respect to their effects on activity, stability, specificity and biological safety. Chemical conjugation of molecules to therapeutic oligonucleotides is an attractive strategy for improving their physicochemical and pharmaceutical properties. There are many candidates developed to enhance pharmaceutical efficacy, such as receptor ligands (N-acetylgalactosamine, mannose, N-acetylglucosamine), lipids (cholesterol, bile acid derivatives, and fatty acids), specific small molecules, polymers (polyethylene glycol; PEG), peptides (cell-penetrating peptides; CPPs), aptamers and antibodies.

Active tissue-specific targeting can be achieved through conjugation of oligonucleotides to receptor ligands that promote specific binding of target cells and mediate tissue-specific delivery. Following the discovery of N-acetylgalactosamine (GalNAc) conjugates that bind to the asialoglycoprotein receptor (ASGPR), the targeted delivery of oligonucleotides to hepatocytes has become a groundbreaking approach in the field of oligonucleotide therapeutics. Alnylam pharmaceutical developed the well-known proline-based tri-antennary GalNAc conjugation linkers. Arrowhead pharmaceuticals also developed its own multivalent GalNAc conjugation linkers using peptidyl backbone structures. Dicerna Pharmaceuticals has introduced the GalNAc sugars attached to the extended region of oligonucleotides tetraloop (namely, GalXC compound).

On the other hand, the mannose receptor is known as C-type lectin dominantly present on the surface of macrophages, immature dendritic cells, and liver sinusoidal endothelial cells, but is also expressed on the surface of skin cells such as human dermal fibroblasts and keratinocytes. The receptor recognizes terminal mannose, N-acetylglucosamine and fucose residues on glycans attached to proteins found on the surface of some microorganisms. This discovery led to the development of mannose-based chemical conjugation on oligonucleotides. Conjugation of hydrophobic lipids such as cholesterol, bile acids and fatty acids has been developed to improve delivery of oligonucleotides by promoting endosomal release and longer plasma half-life and accumulation in the liver upon systemic administration. Such modifications may enhance the delivery to the liver but also to peripheral tissues such as muscle via passive targeting by increasing the binding affinity of oligonucleotides to plasma proteins and/or via active targeting by hijacking endogenous lipid transport pathways. Bile acids are steroid molecules that derive from the catabolism of cholesterol and are essential for the digestion and absorption of lipids and fat-soluble vitamins, and cross multiple cellular membranes through active and passive transport processes during enterohepatic circulation. This specific behavior of bile acids has led to various studies of oligonucleotide delivery. Many small molecules have been screened to find the effective delivery modalities. M. Zirial group reported bisphenol A diglycidyl ether and 50 chemical compounds enhanced the siRNA delivery using two well-established siRNA delivery systems, lipid nanoparticles (LNPs) and cholesterol-conjugated siRNAs in two different endocytic mechanisms (Nucleic Acids Research 2015, 7984). R. L. Juliano group reported a series of 3-eazapteridine analogs (Nucleic Acids Research, 2015, 1987) and 3-deazapteridine derivatives as enhanced delivery modalities (Nucleic Acids Research 2018, 1601). D. Lee group reported a series of L-type calcium channel blockers (CCBs) and amlodipine that increase the efficacy of a cell penetrating asymmetric siRNAs (cp-asiRNAs), e.g., a lipophilic moiety-conjugated RNAi. cp-asiRNAs can be efficiently internalized into cells and can knock down the target gene without any transfection reagent (J. Invest. Dermatol. 2016, 2305). Polymers such as PEG is usually introduced to improve stability, avoid rapid degradation and enhance the cellular uptake. CPPs are short peptide sequences posing the ability to cross a cellular membrane by endocytosis and facilitating endosomal escape by destabilizing the endosomes compartments. Aptamers have been shown to mediate the delivery of therapeutic oligonucleotides as aptamer-on conjugates, or within nanoparticle formulations. Further development of aptamer-oligonucleotides has shown evidence of oligonucleotide protected from nuclease degradation and have increased plasma half-life. Another promising delivery modality is antibody-RNA conjugates (ARCs), which typically include monoclonal antibodies or antibody fragments with functional oligonucleotides.

Chemical conjugation to oligonucleotides can be categorized in two approaches: monomer-based approach and cluster-based approach. Early research in the field of chemical conjugation was initiated by monomer-based approach using solid support or phosphoramidite with single conjugation linker. For example, 3′-Cholesteryl-TEG CPG or cholesteryl-TEG phosphoramidite is a commercial product that utilizes a monomer-based approach to introduce cholesterol into nucleotides. This strategy is more efficient for introducing multiple heterogeneous chemical conjugates into oligonucleotides by solid phase oligonucleotide synthesis. Recent research has shifted towards performing many cluster-based approach after the successful launch of tri-antennary GalNAc cluster by Alnylam Pharmaceuticals Inc. Although the structure is fixed during the synthetic process, it has the advantage of being able to introduce a complex chemical conjugate at once by o using appropriate coupling method to the oligonucleotide.

Chemical conjugation can be performed to any position of oligonucleotide in siRNA. Because antisense strand usually contains 5′-phosphate, chemical modification is more focused on 3′-position of sense strands, which can be achieved by 1) solid phase oligonucleotide synthesis using chemical conjugate containing solid support or phosphoramidite with cluster or monomer, and/or 2) reverse phase oligonucleotide synthesis followed by post-modification at 3′-position.

Alnylam Pharmaceuticals introduced the tri-antennary GalNAc structure into oligonucleotides using tri-antennary GalNAc cluster containing solid support or phosphoramidite. AM Chemicals suggested to introduce the monomer-based GalNAc conjugation using its monomer containing solid support or phosphoramidite.

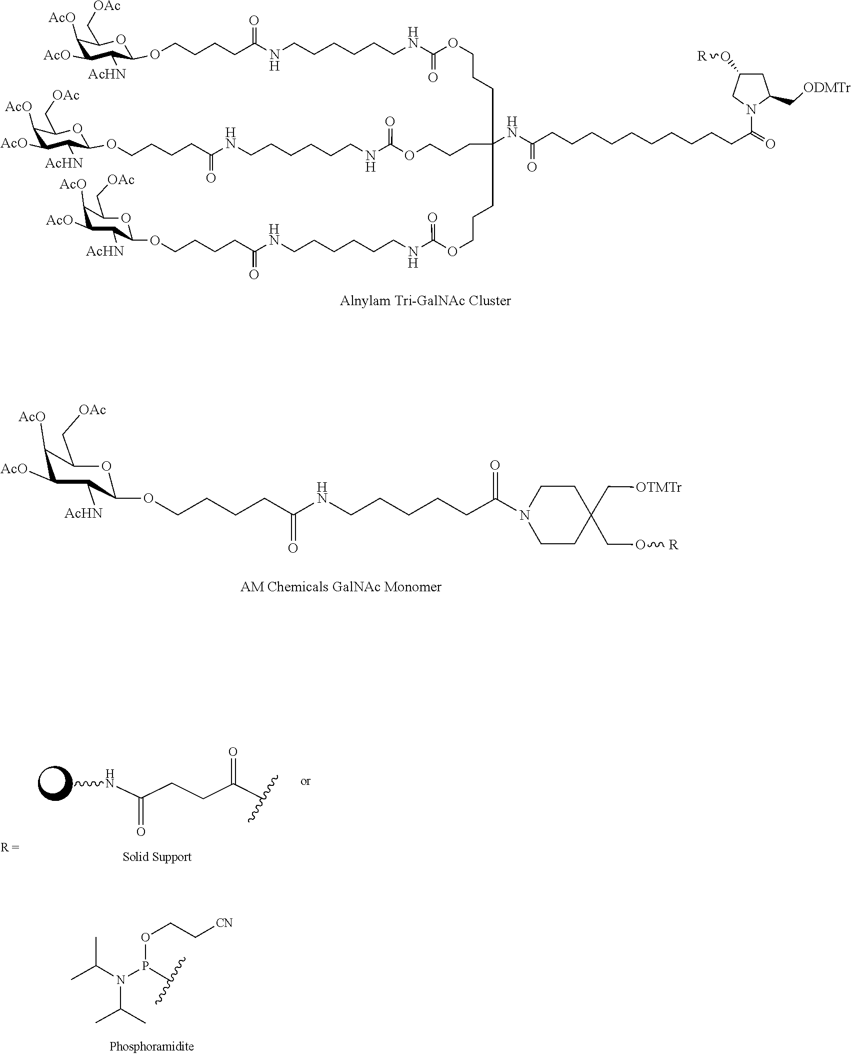

Amino acid-based functional moieties comprising various chemical conjugates such as GalNAc and Mannose have been previously described. Oligonucleotides containing tri-GalNAc cluster using L-lysine backbone showed an initial mRNA knockdown effect but rapidly reduced the activity of siRNA due to its low stability under physiological conditions. Oligonucleotides containing a D-lysine-based tri-GalNAc cluster were also evaluated and found to exhibit similar initial mRNA knockdown efficiencies as in the case of L-lysine backbone. However, the durability was still not enough to extend its effect by a month. Therefore, the need for chemical conjugates having a more stable structure, a long-lasting effect or stability, remains.

Thus, provided herein are pharmaceutically or pharmacologically stable moieties that may impart such characteristics to the macromolecule they can be attached too.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “composition” refers to a mixture of at least two or more components.

The terms “effective amount” and “therapeutically effective amount” refer to an amount of therapeutic compound, combination of compounds, or composition, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “pharmaceutical composition” refers to a mixture of at least one compound described herein with a pharmaceutically acceptable carrier. The pharmaceutical composition facilitates administration of the compound, or combination thereof, to a patient or subject. Multiple techniques of administering a compound, combination, or composition, exist including, but not limited to, intravenous, oral, aerosol, parenteral, ophthalmic, pulmonary, and topical administration. For example, administration of therapeutic proteins, peptides, oligosaccharides, or oligonucleotides is, in some instances, via oral, inhalational, or injected routes of administration.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. Accordingly, for the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

Compounds

It has been discovered that clusters of amino acids that include at least one beta-amino acid, such as beta-lysine or beta-glutamate, are more stable than L- or D-amino acid clusters. It was discovered that when conjugated to a macromolecule, such as one comprising an oligonucleotide, the amino acid cluster imparts markedly improved stability, at least or up to 60 days, to conditions mimicking one or more environments inside a subject (e.g., proteinase K), such as a lumen, such as the physiological environment of blood circulation. The observed improvement in stability imparted to the macromolecule to which the described amino acid clusters renders such complexes suitable for in vivo delivery with sustained therapeutic efficacy by virtue of its pharmacological stability. Further improvement of stability was observed when the oligonucleotide included one or more modifications, including phosphorothioate linkages in the backbone replacing standard phosphate backbone linkages between nucleosides.

Thus, in some embodiments, the compounds provided herein comprise the following formulae:

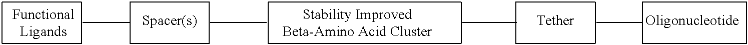

or a salt thereof, which may be written as (J¹-J²)ₓₓ-J³-J⁴-J⁵, or a salt thereof, wherein J¹ is the one or more Functional Ligand, J² is the one or more Spacer, J³ is the Stability Improved Beta-Amino Acid Cluster (SIBAAC), J⁴ is the Tether, xx is 2, 3, 4, 5, or 6, and J⁵ is the macromolecule (e.g., phosphoramidite, solid support, oligomer, e.g., peptide or protein, oligosacharride, or oligonucleotide).

In some embodiments, the oligonucleotide comprises ribonucleic acid, deoxyribonucleic acid, or both. In some embodiments, the oligonucleotide comprises an RNAi, mRNA, miRNA, siRNA, snoRNA, saRNA, or piRNA oligonucleotide. In some embodiments, the oligonucleotide comprises single-stranded oligonucleotide. In some embodiments, the oligonucleotide is 50 nucleotides (“nt”) in length or less, whether single-stranded or double-stranded. In some embodiments, the oligonucleotide is about 5-50 nt, 5-40 nt, 5-30 nt, 5-25 nt, 5-20 nt, 5-15 nt, 5-10 nt, 10-30 nt, 10-25 nt, 10-20 nt, 10-15 nt, 15-30 nt, 15-25 nt, 15-20 nt, 20-30 nt, 20-25 nt, about 5 nt, 10 nt, 15 nt, 20 nt, 25 nt, 30 nt, 40 nt, or 50 nt in length.

In some embodiments, the oligonucleotide is about 14, 15, 16, 17, 18, 19, 20, 21, or 22 nt in length. In some embodiments, the recited oligonucleotide length or range refers to the recited length or range value±2 nt.

In some embodiments, oligonucleotide is, independently, selected from, but not limited to, natural (naked) RNAs, partially or fully modified RNAs, which is connected to tether through phosphate, phosphorothioate, or phosphorodithioate linkage.

In some embodiments, oligonucleotide is connected to the tether at the 5′-end or 3′-end of oligonucleotide. In some embodiments, oligonucleotide is connected to the tether at the 5′-end and 3′-end of oligonucleotide.

In some embodiments, Tether is a divalent or trivalent alkyl linker. In some embodiments, Tether comprises a linker to Stability Improved Beta-Amino Acid Cluster, Spacer(s), and Functional Ligands. In some embodiments, Tether comprises a linker to oligonucleotide. In some embodiments, Tether comprises two linkers for one triphenylmethyl derivative and one solid support. In some embodiments, Tether comprises two linkers for one triphenylmethyl derivative and one phosphoramidite.

In some embodiments, Tether is, independently, selected from, but not limited to, divalent linker or trivalent linker between Oligonucleotide and Stability Improved Beta-Amino Acid Cluster.

In some embodiments, Stability Improved Beta-Amino Acid Cluster comprises one or more beta-amino acids. In some embodiments, Stability Improved Beta-Amino Acid Cluster comprises one or more amino acids. In some embodiments, the beta-amino acids comprise a beta-homolysine, beta-lysine, beta-homoglutamic acid, beta-glutamic acid. In some embodiments, the amino acids comprise a lysine or glutamic acid. In some embodiments, beta-amino acid and amino acid is D-isomer or L-isomer. In some embodiments, Stability Improved Beta-Amino Acid Cluster comprises a combination of beta-amino acids and amino acids. In some embodiments, Stability Improved Beta-Amino Acid Cluster comprises a combination of D-beta-amino acids and D-amino acids. In some embodiments, Stability Improved Beta-Amino Acid Cluster comprises a combination of D-beta-amino acids and L-amino acids. In some embodiments, Stability Improved Beta-Amino Acid Cluster comprises a combination of L-beta-amino acids and D-amino acids. In some embodiments, Stability Improved Beta-Amino Acid Cluster comprises a combination of L-beta-amino acids and L-amino acids.

In some embodiments, Stability Improved Beta-Amino Acid Cluster is, independently, selected from, but not limited to, divalent cluster, trivalent cluster, linear or 2-prong (2+2) tetravalent clusters, linear or 2-prong (3+2) pentavalent clusters, or linear or 2-prong (3+3) or 3-prong (2+2+2) hexavalent cluster containing beta-amino acid resistant to decomposition in physiological conditions between Spacer(s) and Tether.

In some embodiments, Spacer(s) is, independently, selected from, but not limited to, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n is 1 to about 6 between Stability Improve Beta-Amino Acid Cluster and Functional Ligands. In some embodiments, Spacer(s) is a combination of —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n is 1 to about 6.

In some embodiments, Functional Ligands is, independently, selected from, but not limited to, carbohydrate receptor ligands such as N-acetylgalactosamine, N-acetylglucosamine, and mannose, lipids such as cholesterol, bile acid derivatives, and fatty acids, retinoic acid, cell penetrating peptides (CPPs), specific small molecules showing cell-targeting effects, polymers such as poly glycols, aptamers and antibodies, connected to Spacer(s).

In some embodiments, Functional Ligands includes carbohydrate receptor ligands. In some embodiments, carbohydrate receptor ligands are, independently, selected from, but not limited to, N-acetylgalactosamine and its acetate derivates, N-acetylglucosamine and its acetyl derivatives, mannose and its acetate derivatives.

In some embodiments, Functional Ligands includes lipids. In some embodiments, lipids are, independently, selected from, but not limited to, cholesterol and its derivatives. In some embodiments, lipids are, independently, selected from, but not limited to, bile acid derivatives such as cholic acid, chenodeoxycholic acid, lithocholic acid, ursodeoxycholic acid, 3p-hydroxy 5-cholenoic acid and their derivatives. In some embodiments, lipids are, independently, selected from, but not limited to, C₆₋₃₀ saturated fatty acids such as caproic acid (hexanoic acid; C6:0), enathic acid (heptanoic acid; C7:0), caprylic acid (octanoic acid; C8:0), pelargoic acid (nonanoic acid; C9:0), capric acid (n-decanoic acid; C10:0), Undecylic acid (n-undecanoic acid, C11:0), lauric acid (n-dodecanoic acid; C12:0), Tridecylic acid (n-tridecanoic acid, C13:0), myristic acid (n-tetradecanoic acid; C14:0), pentadecylic acid (n-pentadecanoic acid; C15:0), palmitic acid (n-hexadecanoic acid; C16:0), margaric acid (n-heptadecanoic acid; C17:0), stearic acid (n-octadecanoic acid; C18:0), nonadecylic acid (n-nonadecanoic acid; C19:0), arachidic acid (n-eicosanoic acid; C20:0), heneicosylic acid (n-heneicosanoic acid; C21:0), behenic acid (n-docosanoic acid; C22:0), tricosylic acid (n-tricosanoic acid; C23:0), lignoceric acid (n-tetracosanoic acid; C24:0), pentacosylic acid (n-pentacosanoic acid; C25:0), cerotic acid (n-hexacosanoic acid; C26:0), carboceric acid (n-heptacosanoic acid; C27:0), montanic acid (octacosanoic acid; C28:0), nonacosylic acid (n-nonacosanoic acid; C29:0), or melissic acid (n-triacontanoic acid; C30:0). In some embodiments, lipids are, independently, selected from, but not limited to, saturated fatty acid derivatives containing one or more alcohol at certain position such as 12-hydroxydodecanoic acid, 2-hydroxyoctadecanoic acid, 12-hydroxyoctadecanoic acid, 18-hydroxyoctadecanoic acid. In some embodiments, lipids are, independently, selected from, but not limited to, C₁₀₋₃₀ unsaturated fatty acids such as oleic acid (C18:1, 9-cis), elaidic acid (C18:1, 9-trans), linoleic acid (C18:2, 9,12-cis), alpha-linolenic acid (C18:3, 9,12,15-cis), gamma-linolenic acid (C18:3, 6,9,12-cis), arachidonic acid (C20:4, 5,8,11,14-cis), eicosapentaenoic acid (C20:5, 5,8,11,14,17-cis), or docosahexaenoic acid (C22:6, 4,7,10,13,16,19-cis).

In some embodiments, Functional Ligands is retinoic acid (all-trans-3,7-Dimethyl-9-(2,6,6-trimethylcyclohex-1-en-1-yl)nona-2,4,6,8-tetraenoic acid).

In some embodiments, Functional Ligands includes cell penetrating peptides (CPPs) such as penetratin, Tat fragment (48-60), signal sequence-based peptide, PVEC, transportan, amphiphilic model peptide, Arg9, Bacterial cell wall permeating protein, LL-37, cecropin P1, alpha-defensin, beta-defensin, bactenecin, RR-39, and indolicidin (recited from Patent No. WO2009/073809).

In some embodiments, Functional Ligands includes specific small molecules showing cell-targeting effects such as biotin. In some embodiments, Functional Ligands is specific small molecules showing fluorescence such as Cy³ or Cy⁵ dyes.

In some embodiments, Functional Ligands includes cell penetrating polymers. In some embodiments, cell penetrating polymers is, independently, selected from, but not limited to, poly ethylene glycol (PEG; —(CH₂CH₂O)ₙ—, n=2˜20), poly propylene glycol (PPG; —(CH₂CH₂CH₂O)ₙ—, n=2˜20), poly isopropylene glycol (PiPG; —(CH(CH₃)CH₂O)ₙ—, n=2˜20), or poly tetrahydrofuran glycol (PTHFG; —(CH₂CH₂CH₂CH₂O)ₙ—, n=2˜20).

In some embodiments, Functional Ligands includes aptamers.

In some embodiments, Functional Ligands includes antibodies such as Brentuximabvedotin or Gemtuzumab ozogamicin).

In some embodiments, the functional ligand (e.g., LIG) is, independently, a C₆₋₃₀ fatty acid or hydroxy fatty acid, a partially unsaturated fatty acid, including DHA (Docosahexaenoyl), or retinoic acid (retinoyl). In some embodiments, the ligand (e.g., LIG) is, independently, 2-(acetylamino)-2-deoxy-D-galactosyl, β-D-(acetylamino)-2-deoxy-D-glycopyranosyl, 4-aminobutanoyl, 2-(2-aminoethoxy)acetyl, 2-(2-(2-Aminoethoxy)ethoxy)acetyl, 3-(2-(2-Aminoethoxy)ethoxy)propanoyl, Aminoacetyl, (S)-3,7-Diaminoheptanoyl, (S)-3-Aminohexanedioyl, (2S)-2,6-Diaminohexanoyl, (2R)-2,6-Diaminohexanoyl, Nanoanoyl, Decanoyl, Undecanoyl, Dodecanoyl, 12-Hydroxydodecanoyl, Tridecanoyl, Tetradecanoyl, Pentadecanoyl, Hexadecanoyl, Heptadecanoyl, Octadecanoyl, 18-Hydroxystearyl, 12-Hydroxystearyl, 2-Hydroxystearyl, Icosanoyl, Docosanoyl, (4Z,7Z,10Z,13Z,16Z,19Z)-Docosa-4,7,10,13,16,19-hexaenoyl, (5Z,8Z,11Z,14Z)-Eicosa-5,8,11,14-tetraenoyl, (5Z,8Z,11Z,14Z,17Z)-eicosa-5,8,11,14-pentaenoyl, (9Z,12Z,15Z)-octadeca-9,12,15-trienoyl, (6Z,9Z,12Z)-octadeca-6,9,12-trienoyl, (2E,4E,6E,8E)-3,7-Dimethyl-9-(2,6,6-trimethylcyclohex-1-en-1-yl)nona-2,4,6,8-tetraenoyl, (9Z)-Octadec-9-enoyl, (E)-Octadec-9-enoyl, or (9Z,12Z)-octadeca-9,12-dienoyl.

In some embodiments, each component is connected to the other component through one or more bonds, independently, selected from, but not limited to C₁₋₂₀ alkyl, C₂₋₂₀ alkenyl, C2-20 alkynyl, C₃₋₂₀ cycloalkyl, C₄₋₂₀ cycloalkenyl, C₅₋₂₀ cycloalkynyl, C₁₋₂₀ heterocycloalkyl, C₂₋₂₀ heterocycloalkenyl, C₂₋₂₀ heterocycloalkynyl, C₁₋₂₀ aralkyl, C₁₋₂₀ aralkenyl, C₁₋₂₀ aralkynyl, C₁₋₂₀ heteroaralkyl, C₁₋₂₀ heteroaralkenyl, C₁₋₂₀ heteroaralkynyl, —O—, —C(O)—, —N(H)—, —N(C₁₋₅ alkyl)-, —S—, —S(O)—, —SO₂—, —SO₂NH—, —NHSO₂—, —CₙH₂ₙ₊₂—, —CₙH₂ₙ—, —CₙH₂ₙ₋₂—, —S—S—, —RC═N—, —N═CR—, —O═N═C—, —C═N—O—, —O—C(O)—O—, —C(O)—NR—, —NR—C(O)—, —O—C(O)—N(C₁₋₅ alkyl)-, —N(C₁₋₅ alkyl)-C(O)—O—, —N(C₁₋₅ alkyl)-C(O)—N(C₁₋₅ alkyl)-, —N(C₁₋₅ alkyl)-C(S)—N(C₁₋₅ alkyl)-, —N(C₁₋₅ alkyl)SO₂N(C₁₋₅ alkyl)-, phosphate, phosphorothioate, phosphorodithioate and/or combination thereof.

In some embodiments, the solid support is selected from, but not limited to, a silica gel, a controlled pore glass (CPG), or a resin, for example, a polystyrene resin (PS).

In some embodiments, pharmaceutically stability improved moieties are composed with a solid support and triphenylmethyl derivative for oligonucleotide synthesis. In some embodiments, pharmaceutically stability improved moieties are composed with a phosphoramidite and triphenylmethyl derivative for oligonucleotide synthesis.

In some embodiments, provided herein are synthetic processes of pharmaceutically stability improved functional moieties. In some embodiments, provide herein are synthetic processes of oligonucleotides containing pharmaceutically stability improved functional moieties using solid support or phosphoramidite by normal or reverse oligonucleotide synthetic method.

In some embodiments, the oligonucleotide referred to herein includes at least one selected from those of Table 1.

TABLE 1
Selected oligonucleotide sequences.

| Seq ID Number | Sequence ID (5′ → 3′) |
|---|---|
| Seq ID No: 1 | mG*fC*mA fG fU fA mU fG mU fU |
| Sense | mG fA mU fG mG fA |
| Seq ID No: 10 | [Phos]-mU*fC*mC fA mU fC mA fA |
| Antisense | mC fA mU mA mC fU mG*fC*mU*fU* |
| | mC |
| Seq ID No: 5 | mG*fC*mA fG fU fA mU fG mU fU |
| Sense | mG fA mU fG mG fA* |
| Seq ID No: 6 | mG*fC*mA fG fU fA mU fG mU fU |
| Sense | mG fA mU fG mG fA** |
| Seq ID No: 7 | mU*fC*mA fU fC fU mC fA mA fG |
| Sense | mU fC mU fU mA fA |
| Seq ID No: 8 | mU*fC*mA fU fC fU mC fA mA fG |
| Sense | mU fC mU fU mA fA* |
| Seq ID No: 9 | mU*fC*mA fU fC fU mC fA mA fG |
| Sense | mU fC mU fU mA fA** |
| Seq ID No: 11 | [Phos]-mU*fU*mA fA mG fA mC fU |
| Antisense | mU fG mA mG mA fU mG*fA*mU*fC* |
| | mC |
| Seq ID No: 2 | mu mG U G A C U U C C A G A C* |
| Sense | mC*mA |
| Seq ID No: 3 | mG*mU*mA fU fC fC mU mA mA mU |
| Semse | fG fG mU fG mU mA |
| Seq ID No: 4 | mU*fA*mA fU fC fC mU mC mA mC |
| Semse | mU mC mU mA mA mA |

Blank between nucleosides and/or conjugation linker: Phosphate backbone
*between nucleosides and/or conjugation linker: phosphorothioate backbone
** between nucleosides and/or conjugation linker: phosphorodithioate backbone

Tables 2-10 describe certain compounds provided herein having an amino acid cluster with a β-amino acid covalently linked to a macromolecule. The compounds in these tables include α-lysine and α-glutamic acid amino acids, which are (D)-amino acids for compounds 1-56, 192-198, 201, 204, 207, 210, 213, and 216-286, and (L)-amino acids for compounds 200, 203, 206, 209, 212, and 215. The compounds in these tables include a β³-lysine or β³ glutamic acid moiety. In some embodiments, the β³-lysine or β³-glutamic acid moiety may be replaced by the corresponding β²-lysine or β²-glutamic acid moiety, or by the corresponding β^{2,3}-lysine or β^{2,3}-glutamic acid moiety. The structure of such amino acids is shown below for convenience, where R represents the amino acid side chain.

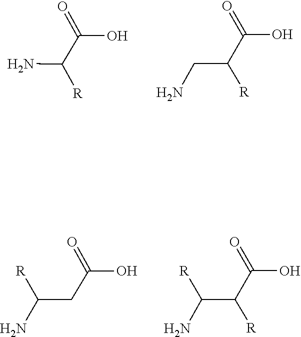

α-amino acid β²-amino acid β³-amino acid β^{2,3}-amino acid Thus, in some embodiments, provided herein are compounds, comprising a formula:

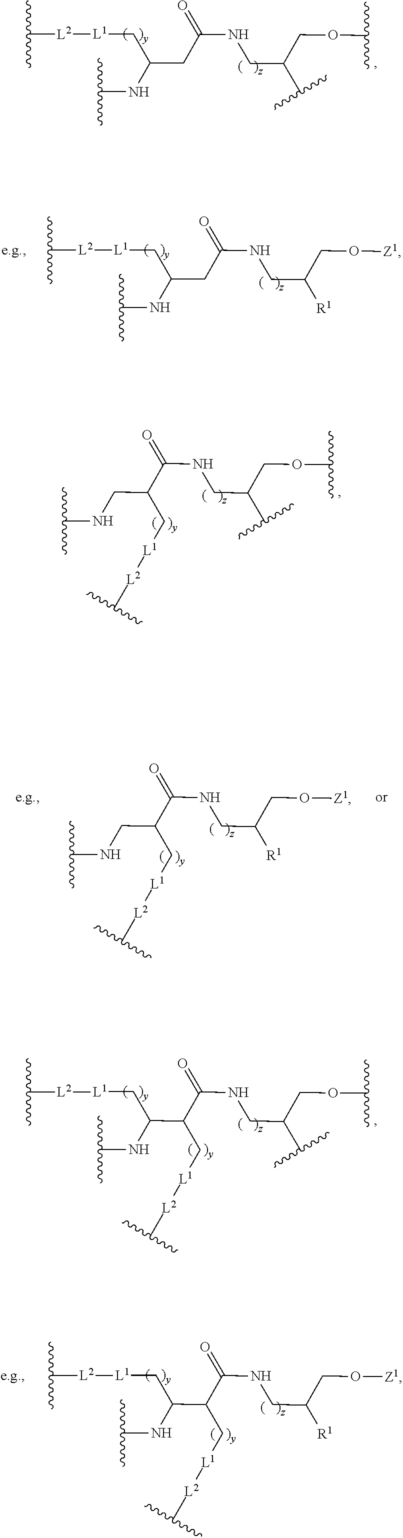

wherein:
y is 0, 1, 2, 3, 4, 5, or 6;
z is 0, 1, 2, 3, 4, 5, or 6;
L¹ is N(H) and L² is C(O), or L¹ is C(O) and L² is N(H);
R¹ is H, CH₂OH, CH₂O-trityl (CH₂O-Tr), CH₂O-monomethoxytrityl (CH₂O-MMTr), CH₂O-dimethoxytrityl (CH₂O-DMTr), or CH₂O-trimethoxytrityl (CH₂O-TMTr); and
Z¹ comprises a macromolecule (e.g., including, but not limited to, oligonucleotide, peptide, or solid support); or
R¹ is H, CH₂O-Tr, CH₂O-MMTr, CH₂O-DMTr, or CH₂O-TMTr or, another CH₂O-trityl moiety referred to herein, and Z¹ is a phosphoramidite, e.g.,

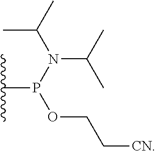 [SMILES: *P(OCCC#N)N(C(C)C)C(C)C]

TABLE 2
Examples of 2 + 2 compounds, which may be in salt form.

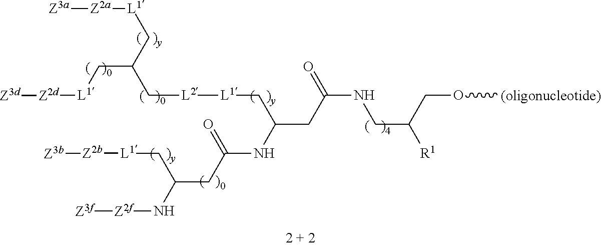

| | | | | | | | | Oligo |
|---|---|---|---|---|---|---|---|---|
| | | | | | | | | SEQ |
| # | Z^{3a}, Z^{3b}, Z^{3d}, Z^{3f} | Z^{2a}, Z^{2b} | Z^{2d}, Z^{2f} | L^{2′} | L^{1′} | y | R¹ | ID NO |
| 1 | GalNAc | C5-AEEA | C5-AEEA-GABA | C(O) | N(H) | 4 | CH₂OH | 1 |
| 2 | GalNAc | C5-AEEA | C5-AEEA-GABA | C(O) | N(H) | 4 | H | 1 |
| 3 | GalNAc | C5-AEEA | C5-AEEA-GLY | N(H) | C(O) | 2 | CH₂OH | 1 |
| 4 | GalNAc | C5-AEEA | C5-AEEA-GLY | N(H) | C(O) | 2 | H | 1 |
| 5 | GluNAc | C5-AEEA | C5-AEEA-GABA | C(O) | N(H) | 4 | CH₂OH | 2 |
| 6 | GluNAc | C5-AEEA | C5-AEEA-GABA | C(O) | N(H) | 4 | H | 2 |
| 7 | GluNAc | C5-AEEA | C5-AEEA-GLY | N(H) | C(O) | 2 | CH₂OH | 2 |
| 8 | GluNAc | C5-AEEA | C5-AEEA-GLY | N(H) | C(O) | 2 | H | 2 |
| 9 | Mannose | C5-AEEA | C5-AEEA-GABA | C(O) | N(H) | 4 | CH₂OH | 2 |
| 10 | Mannose | C5-AEEA | C5-AEEA-GABA | C(O) | N(H) | 4 | H | 2 |
| 11 | Mannose | C5-AEEA | C5-AEEA-GLY | N(H) | C(O) | 2 | CH₂OH | 2 |
| 12 | Mannose | C5-AEEA | C5-AEEA-GLY | N(H) | C(O) | 2 | H | 2 |
| 13 | PA | AEEA | AEEA-GABA | C(O) | N(H) | 4 | CH₂OH | 4 |
| 14 | PA | AEEA | AEEA-GABA | C(O) | N(H) | 4 | H | 4 |

TABLE 3
Examples of 2 + 2 + 2 compounds, which may be in salt form.

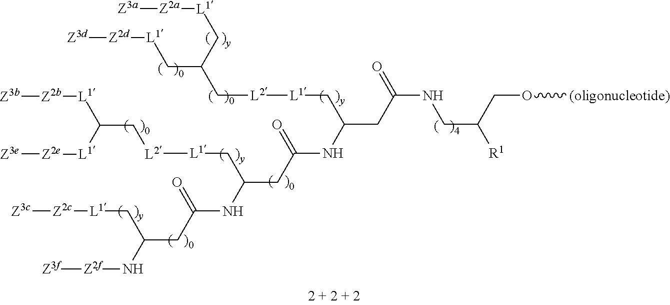

| | | | | | | | | Oligo |
|---|---|---|---|---|---|---|---|---|
| | Z^{3a}, Z^{3b}, Z^{3c}, Z^{3d}, | | | | | | | SEQ |
| # | Z^{3e}, Z^{3f} | Z^{2a}, Z^{2b}, Z^{2c} | Z^{2d}, Z^{2e}, Z^{2f} | L^{2′} | L^{1′} | y | R¹ | ID NO |
| 15 | GalNAc | C5-AEEA | C5-AEEA-GABA | C(O) | N(H) | 4 | CH₂OH | 1 |
| 16 | GalNAc | C5-AEEA | C5-AEEA-GABA | C(O) | N(H) | 4 | H | 1 |
| 17 | GalNAc | C5-AEEA | C5-AEEA-GLY | N(H) | C(O) | 2 | CH₂OH | 1 |
| 18 | GalNAc | C5-AEEA | C5-AEEA-GLY | N(H) | C(O) | 2 | H | 1 |
| 19 | GluNAc | C5-AEEA | C5-AEEA-GABA | C(O) | N(H) | 4 | CH₂OH | 2 |
| 20 | GluNAc | C5-AEEA | C5-AEEA-GABA | C(O) | N(H) | 4 | H | 2 |
| 21 | GluNAc | C5-AEEA | C5-AEEA-GLY | N(H) | C(O) | 2 | CH₂OH | 2 |
| 22 | GluNAc | C5-AEEA | C5-AEEA-GLY | N(H) | C(O) | 2 | H | 2 |
| 23 | Mannose | C5-AEEA | C5-AEEA-GABA | C(O) | N(H) | 4 | CH₂OH | 2 |
| 24 | Mannose | C5-AEEA | C5-AEEA-GABA | C(O) | N(H) | 4 | H | 2 |
| 25 | Mannose | C5-AEEA | C5-AEEA-GLY | N(H) | C(O) | 2 | CH₂OH | 2 |
| 26 | Mannose | C5-AEEA | C5-AEEA-GLY | N(H) | C(O) | 2 | H | 2 |
| 27 | PA | AEEA | AEEA-GABA | C(O) | N(H) | 4 | CH₂OH | 4 |
| 28 | PA | AEEA | AEEA-GABA | C(O) | N(H) | 4 | H | 4 |

TABLE 4
Examples of 3 + 2 compounds, which may be in salt form.

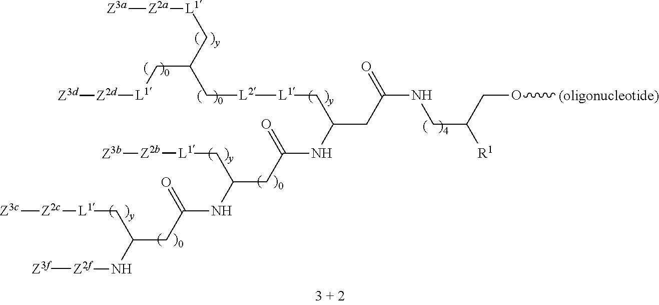

| | | | | | | | | Oligo |
|---|---|---|---|---|---|---|---|---|
| | | | | | | | | SEQ |
| # | Z^{3a}, Z^{3b}, Z^{3c}, Z^{3d}, Z^{3f} | Z^{2a}, Z^{2b}, Z^{2c} | Z^{2d}, Z^{2f} | L^{2′} | L^{1′} | y | R¹ | ID NO |
| 29 | GalNAc | C5-AEEA | C5-AEEA-Gly | N(H) | C(O) | 2 | CH₂OH | 1 |
| 30 | GalNAc | C5-AEEA | C5-AEEA-Gly | N(H) | C(O) | 2 | H | 1 |
| 31 | GalNAc | C5-AEEA | C5-AEEA-GABA | C(O) | N(H) | 4 | CH₂OH | 1 |
| 32 | GalNAc | C5-AEEA | C5-AEEA-GABA | C(O) | N(H) | 4 | H | 1 |
| 33 | GluNAc | C5-AEEA | C5-AEEA-Gly | N(H) | C(O) | 2 | CH₂OH | 2 |
| 34 | GluNAc | C5-AEEA | C5-AEEA-Gly | N(H) | C(O) | 2 | H | 2 |
| 35 | GluNAc | C5-AEEA | C5-AEEA-GABA | C(O) | N(H) | 4 | CH₂OH | 2 |
| 36 | GluNAc | C5-AEEA | C5-AEEA-GABA | C(O) | N(H) | 4 | H | 2 |
| 37 | Mannose | C5-AEEA | C5-AEEA-Gly | N(H) | C(O) | 2 | CH₂OH | 2 |
| 38 | Mannose | C5-AEEA | C5-AEEA-Gly | N(H) | C(O) | 2 | H | 2 |
| 39 | Mannose | C5-AEEA | C5-AEEA-GABA | C(O) | N(H) | 4 | CH₂OH | 2 |
| 40 | Mannose | C5-AEEA | C5-AEEA-GABA | C(O) | N(H) | 4 | H | 2 |
| 41 | PA | C5-AEEA | AEEA-GABA | C(O) | N(H) | 4 | CH₂OH | 4 |
| 42 | PA | C5-AEEA | AEEA-GABA | C(O) | N(H) | 4 | H | 4 |

TABLE 5
Examples of 3 + 3 compounds, which may be in salt form.

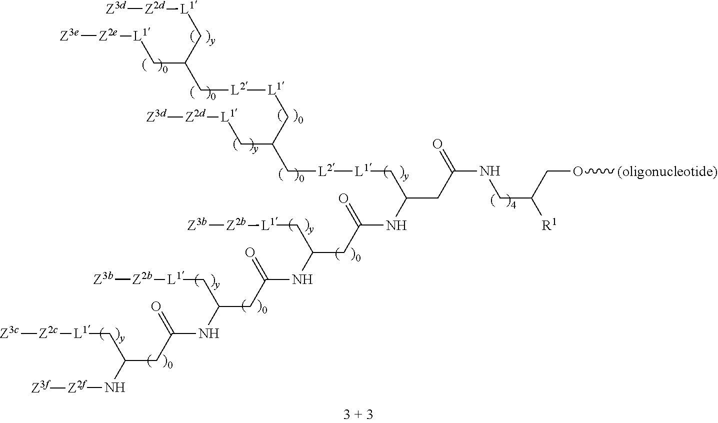

| | | | | | | | | Oligo |
|---|---|---|---|---|---|---|---|---|
| | Z^{3a}, Z^{3b}, Z^{3c}, Z^{3d}, | | | | | | | SEQ |
| # | Z^{3e}, Z^{3f} | Z^{3a}, Z^{3b}, Z^{3c}, Z^{3d} | Z^{3e}, Z^{3f} | L^{2′} | L^{1′} | y | R¹ | ID NO |
| 43 | GalNAc | C5-AEEA | C5-AEEA-GABA | C(O) | N(H) | 4 | CH₂OH | 1 |
| 44 | GalNAc | C5-AEEA | C5-AEEA-GABA | C(O) | N(H) | 4 | H | 1 |
| 45 | GalNAc | C5-AEEA | C5-AEEA-GLY | N(H) | C(O) | 2 | CH₂OH | 1 |
| 46 | GalNAc | C5-AEEA | C5-AEEA-GLY | N(H) | C(O) | 2 | H | 1 |
| 47 | GluNAc | C5-AEEA | C5-AEEA-GABA | C(O) | N(H) | 4 | CH₂OH | 2 |
| 48 | GluNAc | C5-AEEA | C5-AEEA-GABA | C(O) | N(H) | 4 | H | 2 |
| 49 | GluNAc | C5-AEEA | C5-AEEA-GLY | N(H) | C(O) | 2 | CH₂OH | 2 |
| 50 | GluNAc | C5-AEEA | C5-AEEA-GLY | N(H) | C(O) | 2 | H | 2 |
| 51 | Mannose | C5-AEEA | C5-AEEA-GABA | C(O) | N(H) | 4 | CH₂OH | 2 |
| 52 | Mannose | C5-AEEA | C5-AEEA-GABA | C(O) | N(H) | 4 | H | 2 |
| 53 | Mannose | C5-AEEA | C5-AEEA-GLY | N(H) | C(O) | 2 | CH₂OH | 2 |
| 54 | Mannose | C5-AEEA | C5-AEEA-GLY | N(H) | C(O) | 2 | H | 2 |
| 55 | PA | C5-AEEA | AEEA-GABA | C(O) | N(H) | 4 | CH₂OH | 4 |
| 56 | PA | C5-AEEA | AEEA-GABA | C(O) | N(H) | 4 | H | 4 |

TABLE 6
Examples of Linear-2 compounds, which may be in salt form.

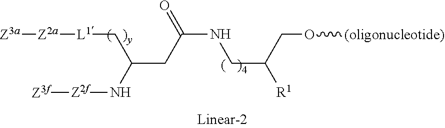

| | | | | | | | Oligo |
|---|---|---|---|---|---|---|---|
| | | | | | | | SEQ |
| # | Z^{3a}, Z^{3f} | Z^{2a} | Z^{2f} | L^{1′} | y | R¹ | ID NO |
| 57 | 3β-hydroxy 5-cholenoic acid | AEEA | AEEA-GABA | N(H) | 4 | CH₂OH | 3 |
| 58 | 3β-hydroxy 5-cholenoic acid | AEEA | AEEA-GABA | N(H) | 4 | H | 3 |
| 59 | ACA | — | GABA | N(H) | 4 | CH₂OH | 4 |
| 60 | ALA | AEEA | AEEA-GABA | N(H) | 4 | CH₂OH | 4 |
| 61 | ALA | AEEA | AEEA-GABA | N(H) | 4 | H | 4 |
| 62 | ARA | AEEA | AEEA-GABA | N(H) | 4 | CH₂OH | 4 |
| 63 | ARA | AEEA | AEEA-GABA | N(H) | 4 | H | 4 |
| 64 | BA | — | GABA | N(H) | 4 | CH₂OH | 4 |
| 65 | BA | — | GABA | N(H) | 4 | H | 4 |
| 66 | BA | AEA | AEA-GABA | N(H) | 4 | CH₂OH | 4 |
| 67 | BA | AEA | AEA-GABA | N(H) | 4 | H | 4 |
| 68 | BA | AEEA | AEEA-GABA | N(H) | 4 | CH₂OH | 4 |
| 69 | BA | AEEA | AEEA-GABA | N(H) | 4 | H | 4 |
| 70 | BA | AEEP | AEEP-GABA | N(H) | 4 | CH₂OH | 4 |
| 71 | BA | AEEP | AEEP-GABA | N(H) | 4 | H | 4 |
| 72 | BA | — | — | N(H) | 4 | CH₂OH | 4 |
| 73 | BA | — | — | N(H) | 4 | H | 4 |
| 74 | CA | — | GABA | N(H) | 4 | CH₂OH | 4 |
| 75 | Chenocholic acid | AEEA | AEEA-GABA | N(H) | 4 | CH₂OH | 3 |
| 76 | Chenocholic acid | AEEA | AEEA-GABA | N(H) | 4 | H | 3 |
| 77 | Cholesterol | AEEA | AEEA-GABA | N(H) | 4 | CH₂OH | 4 |
| 78 | Cholesterol | AEEA | AEEA-GABA | N(H) | 4 | H | 4 |
| 79 | Cholic acid | AEEA | AEEA-GABA | N(H) | 4 | CH₂OH | 3 |
| 80 | Cholic acid | AEEA | AEEA-GABA | N(H) | 4 | H | 3 |
| 81 | DDA | — | GABA | N(H) | 4 | CH₂OH | 4 |
| 82 | DDA 12-OH | — | GABA | N(H) | 4 | CH₂OH | 4 |
| 83 | DHA | AEEA | AEEA-GABA | N(H) | 4 | CH₂OH | 4 |
| 84 | DHA | AEEA | AEEA-GABA | N(H) | 4 | H | 4 |
| 85 | EA | AEEA | AEEA-GABA | N(H) | 4 | CH₂OH | 4 |
| 86 | EA | AEEA | AEEA-GABA | N(H) | 4 | H | 4 |
| 87 | EPA | AEEA | AEEA-GABA | N(H) | 4 | CH₂OH | 4 |
| 88 | EPA | AEEA | AEEA-GABA | N(H) | 4 | H | 4 |
| 89 | GalNAc | C5 | C5 | N(H) | 4 | CH₂OH | 1 |
| 90 | GalNAc | C5 | C5 | N(H) | 4 | H | 1 |
| 91 | GalNAc | C5-AEA | C5-AEA-GABA | N(H) | 4 | CH₂OH | 1 |
| 92 | GalNAc | C5-AEA | C5-AEA-GABA | N(H) | 4 | H | 1 |
| 93 | GalNAc | C5-AEEA | C5-AEEA-GABA | N(H) | 4 | CH₂OH | 1 |
| 94 | GalNAc | C5-AEEA | C5-AEEA-GABA | N(H) | 4 | H | 1 |
| 95 | GalNAc | C5-AEEP | C5-AEEP-GABA | N(H) | 4 | CH₂OH | 1 |
| 96 | GalNAc | C5-AEEP | C5-AEEP-GABA | N(H) | 4 | H | 1 |
| 97 | GalNAc | C5 | C5-GABA | N(H) | 4 | CH₂OH | 1 |
| 98 | GalNAc | C5 | C5-GABA | N(H) | 4 | H | 1 |
| 99 | GalNAc | C5 | C5-Gly | C(O) | 2 | CH₂OH | 1 |
| 100 | GalNAc | C5 | C5-Gly | C(O) | 2 | H | 1 |
| 101 | GLA | AEEA | AEEA-GABA | N(H) | 4 | CH₂OH | 4 |
| 102 | GLA | AEEA | AEEA-GABA | N(H) | 4 | H | 4 |
| 103 | GluNAc | C5 | C5 | N(H) | 4 | CH₂OH | 2 |
| 104 | GluNAc | C5 | C5 | N(H) | 4 | H | 2 |
| 105 | GluNAc | C5-AEA | C5-AEA-GABA | N(H) | 4 | CH₂OH | 2 |
| 106 | GluNAc | C5-AEA | C5-AEA-GABA | N(H) | 4 | H | 2 |
| 107 | GluNAc | C5-AEEA | C5-AEEA-GABA | N(H) | 4 | CH₂OH | 2 |
| 108 | GluNAc | C5-AEEA | C5-AEEA-GABA | N(H) | 4 | H | 2 |
| 109 | GluNAc | C5-AEEP | C5-AEEP-GABA | N(H) | 4 | CH₂OH | 2 |
| 110 | GluNAc | C5-AEEP | C5-AEEP-GABA | N(H) | 4 | H | 2 |
| 111 | GluNAc | C5 | C5-GABA | N(H) | 4 | CH₂OH | 2 |
| 112 | GluNAc | C5 | C5-GABA | N(H) | 4 | H | 2 |
| 113 | GluNAc | C5 | C5-Gly | C(O) | 2 | CH₂OH | 2 |
| 114 | GluNAc | C5 | C5-Gly | C(O) | 2 | H | 2 |
| 115 | HDA | — | GABA | N(H) | 4 | CH₂OH | 4 |
| 116 | LA | AEEA | AEEA-GABA | N(H) | 4 | CH₂OH | 4 |
| 117 | LA | AEEA | AEEA-GABA | N(H) | 4 | H | 4 |
| 118 | Lithocholic acid | AEEA | AEEA-GABA | N(H) | 4 | CH₂OH | 3 |
| 119 | Lithocholic acid | AEEA | AEEA-GABA | N(H) | 4 | H | 3 |
| 120 | MA | — | GABA | N(H) | 4 | CH₂OH | 4 |
| 121 | Mannose | C5 | C5 | N(H) | 4 | CH₂OH | 2 |
| 122 | Mannose | C5 | C5 | N(H) | 4 | H | 2 |
| 123 | Mannose | C5-AEA | C5-AEA-GABA | N(H) | 4 | CH₂OH | 2 |
| 124 | Mannose | C5-AEA | C5-AEA-GABA | N(H) | 4 | H | 2 |
| 125 | Mannose | C5-AEEA | C5-AEEA-GABA | N(H) | 4 | CH₂OH | 2 |
| 126 | Mannose | C5-AEEA | C5-AEEA-GABA | N(H) | 4 | H | 2 |
| 127 | Mannose | C5-AEEP | C5-AEEP-GABA | N(H) | 4 | CH₂OH | 2 |
| 128 | Mannose | C5-AEEP | C5-AEEP-GABA | N(H) | 4 | H | 2 |
| 129 | Mannose | C5 | C5-GABA | N(H) | 4 | CH₂OH | 2 |
| 130 | Mannose | C5 | C5-GABA | N(H) | 4 | H | 2 |
| 131 | Mannose | C5 | C5-Gly | C(O) | 2 | CH₂OH | 2 |
| 132 | Mannose | C5 | C5-Gly | C(O) | 2 | H | 2 |
| 133 | OA | AEEA | AEEA-GABA | N(H) | 4 | CH₂OH | 4 |
| 134 | OA | AEEA | AEEA-GABA | N(H) | 4 | H | 4 |
| 135 | PA | — | GABA | N(H) | 4 | CH₂OH | 4 |
| 136 | PA | — | GABA | N(H) | 4 | H | 4 |
| 137 | PA | C5-AEA | AEA-GABA | N(H) | 4 | CH₂OH | 4 |
| 138 | PA | C5-AEA | AEA-GABA | N(H) | 4 | H | 4 |
| 139 | PA | C5-AEEA | AEEA-GABA | N(H) | 4 | CH₂OH | 4 |
| 140 | PA | C5-AEEA | AEEA-GABA | N(H) | 4 | H | 4 |
| 141 | PA | C5-AEEP | AEEP-GABA | N(H) | 4 | CH₂OH | 4 |
| 142 | PA | C5-AEEP | AEEP-GABA | N(H) | 4 | H | 4 |
| 143 | PA | — | — | N(H) | 4 | CH₂OH | 4 |
| 144 | PA | — | — | N(H) | 4 | H | 4 |
| 145 | PA/CA | — | GABA | N(H) | 4 | CH₂OH | 4 |
| 146 | PA/CA | AEA | AEA-GABA | N(H) | 4 | CH₂OH | 4 |
| 147 | PA/CA | AEEA | AEEA-GABA | N(H) | 4 | CH₂OH | 4 |
| 148 | PA/CA | AEEP | AEEP-GABA | N(H) | 4 | CH₂OH | 4 |
| 149 | PA/CA | — | — | N(H) | 4 | CH₂OH | 4 |
| 150 | PA/DDA | — | GABA | N(H) | 4 | CH₂OH | 4 |
| 151 | PA/DDA | AEA | AEA-GABA | N(H) | 4 | CH₂OH | 4 |
| 152 | PA/DDA | AEEA | AEEA-GABA | N(H) | 4 | CH₂OH | 4 |
| 153 | PA/DDA | AEEP | AEEP-GABA | N(H) | 4 | CH₂OH | 4 |
| 154 | PA/DDA | — | — | N(H) | 4 | CH₂OH | 4 |
| 155 | PA/MA | — | GABA | N(H) | 4 | CH₂OH | 4 |
| 156 | PA/MA | AEA | AEA-GABA | N(H) | 4 | CH₂OH | 4 |
| 157 | PA/MA | AEEA | AEEA-GABA | N(H) | 4 | CH₂OH | 4 |
| 158 | PA/MA | AEEP | AEEP-GABA | N(H) | 4 | CH₂OH | 4 |
| 159 | PA/MA | — | — | N(H) | 4 | CH₂OH | 4 |
| 160 | PA/PDA | — | GABA | N(H) | 4 | CH₂OH | 4 |
| 161 | PA/PDA | AEA | AEA-GABA | N(H) | 4 | CH₂OH | 4 |
| 162 | PA/PDA | AEEA | AEEA-GABA | N(H) | 4 | CH₂OH | 4 |
| 163 | PA/PDA | AEEP | AEEP-GABA | N(H) | 4 | CH₂OH | 4 |
| 164 | PA/PDA | — | — | N(H) | 4 | CH₂OH | 4 |
| 165 | PA/PGA | — | GABA | N(H) | 4 | CH₂OH | 4 |
| 166 | PA/PGA | AEA | AEA-GABA | N(H) | 4 | CH₂OH | 4 |
| 167 | PA/PGA | AEEA | AEEA-GABA | N(H) | 4 | CH₂OH | 4 |
| 168 | PA/PGA | AEEP | AEEP-GABA | N(H) | 4 | CH₂OH | 4 |
| 169 | PA/PGA | — | — | N(H) | 4 | CH₂OH | 4 |
| 170 | PA/TDA | — | GABA | N(H) | 4 | CH₂OH | 4 |
| 171 | PA/TDA | AEA | AEA-GABA | N(H) | 4 | CH₂OH | 4 |
| 172 | PA/TDA | AEEA | AEEA-GABA | N(H) | 4 | CH₂OH | 4 |
| 173 | PA/TDA | AEEP | AEEP-GABA | N(H) | 4 | CH₂OH | 4 |
| 174 | PA/TDA | — | — | N(H) | 4 | CH₂OH | 4 |
| 175 | PA/UDA | — | GABA | N(H) | 4 | CH₂OH | 4 |
| 176 | PA/UDA | AEA | AEA-GABA | N(H) | 4 | CH₂OH | 4 |
| 177 | PA/UDA | AEEA | AEEA-GABA | N(H) | 4 | CH₂OH | 4 |
| 178 | PA/UDA | AEEP | AEEP-GABA | N(H) | 4 | CH₂OH | 4 |
| 179 | PA/UDA | — | — | N(H) | 4 | CH₂OH | 4 |
| 180 | PDA | — | GABA | N(H) | 4 | CH₂OH | 4 |
| 181 | PGA | — | GABA | N(H) | 4 | CH₂OH | 4 |
| 182 | RA | AEEA | AEEA-GABA | N(H) | 4 | CH₂OH | 4 |
| 183 | RA | AEEA | AEEA-GABA | N(H) | 4 | H | 4 |
| 184 | SA | — | GABA | N(H) | 4 | CH₂OH | 4 |
| 185 | SA 12-OH | — | GABA | N(H) | 4 | CH₂OH | 4 |
| 186 | SA 18-OH | — | GABA | N(H) | 4 | CH₂OH | 4 |
| 187 | SA 2-OH | — | GABA | N(H) | 4 | CH₂OH | 4 |
| 188 | TDA | — | GABA | N(H) | 4 | CH₂OH | 4 |
| 189 | UDA | — | GABA | N(H) | 4 | CH₂OH | 4 |
| 190 | Ursodeoxycholic acid | AEEA | AEEA-GABA | N(H) | 4 | CH₂OH | 3 |
| 191 | Ursodeoxycholic acid | AEEA | AEEA-GABA | N(H) | 4 | H | 3 |

TABLE 7
Examples of Linear-3 compounds, which may be in salt form.

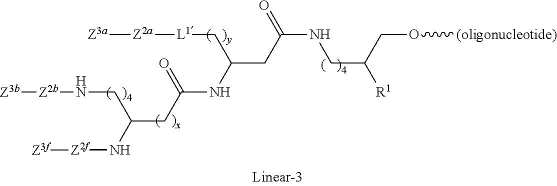

| | | | | | | | | Oligo |
|---|---|---|---|---|---|---|---|---|
| | Z^{3a}, Z^{3b}, | | | | | | | SEQ |
| # | Z^{3f} | Z^{2a}, Z^{2b} | Z2^{f} | L^{1′} | y | X | R¹ | ID NO |
| 192 | BA | — | GABA | N(H) | 4 | 0 | CH₂OH | 4 |
| 193 | BA | AEA | AEA-GABA | N(H) | 4 | 0 | CH₂OH | 4 |
| 194 | BA | AEEA | AEEA-GABA | N(H) | 4 | 0 | CH₂OH | 4 |
| 195 | BA | AEEP | AEEP-GABA | N(H) | 4 | 0 | CH₂OH | 4 |
| 196 | BA | — | — | N(H) | 4 | 0 | CH₂OH | 4 |
| 197 | GalNAc | C5-AEEA | C5-AEEA-GABA | N(H) | 4 | 0 | CH₂OH | 1 |
| 198 | GalNAc | C5-AEEA | C5-AEEA-GABA | N(H) | 4 | 0 | H | 1 |
| 199 | GalNAc | C5 | C5-GABA | N(H) | 4 | 1 | CH₂OH | 1 |
| 200 | GalNAc | C5 | C5-GABA | N(H) | 4 | 0 | CH₂OH | 1 |
| 201 | GalNAc | C5 | C5-GABA | N(H) | 4 | 0 | CH₂OH | 1 |
| 202 | GalNAc | C5 | C5-GABA | N(H) | 4 | 1 | CH₂OH | 5 |
| 203 | GalNAc | C5 | C5-GABA | N(H) | 4 | 0 | CH₂OH | 5 |
| 204 | GalNAc | C5 | C5-GABA | N(H) | 4 | 0 | CH₂OH | 5 |
| 205 | GalNAc | C5 | C5-GABA | N(H) | 4 | 1 | CH₂OH | 6 |
| 206 | GalNAc | C5 | C5-GABA | N(H) | 4 | 0 | CH₂OH | 6 |
| 207 | GalNAc | C5 | C5-GABA | N(H) | 4 | 0 | CH₂OH | 6 |
| 208 | GalNAc | C5 | C5-GABA | N(H) | 4 | 1 | CH₂OH | 7 |
| 209 | GalNAc | C5 | C5-GABA | N(H) | 4 | 0 | CH₂OH | 7 |
| 210 | GalNAc | C5 | C5-GABA | N(H) | 4 | 0 | CH₂OH | 7 |
| 211 | GalNAc | C5 | C5-GABA | N(H) | 4 | 1 | CH₂OH | 8 |
| 212 | GalNAc | C5 | C5-GABA | N(H) | 4 | 0 | CH₂OH | 8 |
| 213 | GalNAc | C5 | C5-GABA | N(H) | 4 | 0 | CH₂OH | 8 |
| 214 | GalNAc | C5 | C5-GABA | N(H) | 4 | 1 | CH₂OH | 9 |
| 215 | GalNAc | C5 | C5-GABA | N(H) | 4 | 0 | CH₂OH | 9 |
| 216 | GalNAc | C5 | C5-GABA | N(H) | 4 | 0 | CH₂OH | 9 |
| 217 | GalNAc | C5 | C5-GABA | N(H) | 4 | 0 | H | 1 |
| 218 | GalNAc | C5-AEEA | C5-AEEA-GLY | C(O) | 2 | 0 | CH₂OH | 1 |
| 219 | GalNAc | C5-AEEA | C5-AEEA-GLY | C(O) | 2 | 0 | H | 1 |
| 220 | GalNAc | C5 | C5-GLY | C(O) | 2 | 0 | CH₂OH | 1 |
| 221 | GalNAc | C5 | C5-GLY | C(O) | 2 | 0 | H | 1 |
| 222 | GluNAc | C5-AEEA | C5-AEEA-GABA | N(H) | 4 | 0 | CH₂OH | 2 |
| 223 | GluNAc | C5-AEEA | C5-AEEA-GABA | N(H) | 4 | 0 | H | 2 |
| 224 | GluNAc | C5 | C5-GABA | N(H) | 4 | 0 | CH₂OH | 2 |
| 225 | GluNAc | C5 | C5-GABA | N(H) | 4 | 0 | H | 2 |
| 226 | GluNAc | C5-AEEA | C5-AEEA-GLY | C(O) | 2 | 0 | CH₂OH | 2 |
| 227 | GluNAc | C5-AEEA | C5-AEEA-GLY | C(O) | 2 | 0 | H | 2 |
| 228 | GluNAc | C5 | C5-GLY | C(O) | 2 | 0 | CH₂OH | 2 |
| 229 | GluNAc | C5 | C5-GLY | C(O) | 2 | 0 | H | 2 |
| 230 | Mannose | C5-AEEA | C5-AEEA-GABA | N(H) | 4 | 0 | CH₂OH | 2 |
| 231 | Mannose | C5-AEEA | C5-AEEA-GABA | N(H) | 4 | 0 | H | 2 |
| 232 | Mannose | C5 | C5-GABA | N(H) | 4 | 0 | CH₂OH | 2 |
| 233 | Mannose | C5 | C5-GABA | N(H) | 4 | 0 | H | 2 |
| 234 | Mannose | C5-AEEA | C5-AEEA-GLY | C(O) | 2 | 0 | CH₂OH | 2 |
| 235 | Mannose | C5-AEEA | C5-AEEA-GLY | C(O) | 2 | 0 | H | 2 |
| 236 | Mannose | C5 | C5-GLY | C(O) | 2 | 0 | CH₂OH | 2 |
| 237 | Mannose | C5 | C5-GLY | C(O) | 2 | 0 | H | 2 |
| 238 | PA | — | GABA | N(H) | 4 | 0 | CH₂OH | 4 |
| 239 | PA | — | GABA | N(H) | 4 | 0 | H | 4 |
| 240 | PA | AEA | AEA-GABA | N(H) | 4 | 0 | CH₂OH | 4 |
| 241 | PA | AEA | AEA-GABA | N(H) | 4 | 0 | H | 4 |
| 242 | PA | AEEA | AEEA-GABA | N(H) | 4 | 0 | CH₂OH | 4 |
| 243 | PA | AEEA | AEEA-GABA | N(H) | 4 | 0 | H | 4 |
| 244 | PA | AEEA | AEEP-GABA | N(H) | 4 | 0 | CH₂OH | 4 |
| 245 | PA | AEEP | AEEP-GABA | N(H) | 4 | 0 | H | 4 |
| 246 | PA | — | — | N(H) | 4 | 0 | CH₂OH | 4 |
| 247 | PA | — | — | N(H) | 4 | 0 | H | 4 |

TABLE 8
Examples of Linear-4 compounds, which may be in salt form.

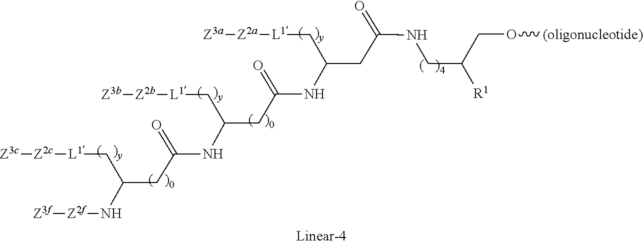

| | | | | | | | Oligo |
|---|---|---|---|---|---|---|---|
| | Z^{3a}, Z^{3b}, | | | | | | SEQ |
| # | Z^{3c}, Z^{3f} | Z^{2a}, Z^{2b}, Z^{2c} | Z^{2f} | L^{1′} | y | R¹ | ID NO |
| 248 | GalNAc | C5-AEEA | C5-AEEA-GABA | N(H) | 4 | CH₂OH | 1 |
| 249 | GalNAc | C5-AEEA | C5-AEEA-GABA | N(H) | 4 | H | 1 |
| 250 | GalNAc | C5-AEEA | C5-AEEA-GLY | C(O) | 2 | CH₂OH | 1 |
| 251 | GalNAc | C5-AEEA | C5-AEEA-GLY | C(O) | 2 | H | 1 |
| 252 | GluNAc | C5-AEEA | C5-AEEA-GABA | N(H) | 4 | CH₂OH | 2 |
| 253 | GluNAc | C5-AEEA | C5-AEEA-GABA | N(H) | 4 | H | 2 |
| 254 | GluNAc | C5-AEEA | C5-AEEA-GLY | C(O) | 2 | CH₂OH | 2 |
| 255 | GluNAc | C5-AEEA | C5-AEEA-GLY | C(O) | 2 | H | 2 |
| 256 | Mannose | C5-AEEA | C5-AEEA-GABA | N(H) | 4 | CH₂OH | 2 |
| 257 | Mannose | C5-AEEA | C5-AEEA-GABA | N(H) | 4 | H | 2 |
| 258 | Mannose | C5-AEEA | C5-AEEA-GLY | C(O) | 2 | CH₂OH | 2 |
| 259 | Mannose | C5-AEEA | C5-AEEA-GLY | C(O) | 2 | H | 2 |
| 260 | PA | AEEA | AEEA-GABA | N(H) | 4 | CH₂OH | 4 |
| 261 | PA | AEEA | AEEA-GABA | N(H) | 4 | H | 4 |

TABLE 9
Examples of Linear-5 compounds, which may be in salt form.

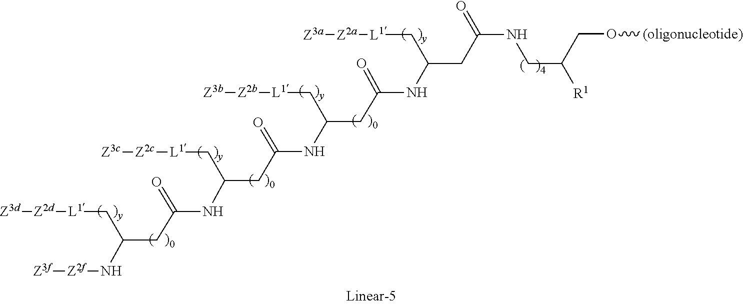

| | Z^{3a}, Z^{3b}, | | | | | | Oligo |
|---|---|---|---|---|---|---|---|
| | Z^{3c}, Z^{3d} | Z^{2a}, Z^{2b}, | | | | | SEQ |
| # | Z^{3f} | Z^{2c}, Z^{2d} | Z^{2f} | L^{1′} | y | R¹ | ID NO |
| 262 | GalNAc | C5 | C5 | C(O) | 2 | CH₂OH | 1 |
| 263 | GalNAc | C5 | C5 | C(O) | 2 | H | 1 |
| 264 | GalNAc | C5-AEEA | C5-AEEA-GABA | N(H) | 4 | CH₂OH | 1 |
| 265 | GalNAc | C5-AEEA | C5-AEEA-GABA | N(H) | 4 | H | 1 |
| 266 | GluNAc | C5 | C5 | C(O) | 2 | CH₂OH | 2 |
| 267 | GluNAc | C5 | C5 | C(O) | 2 | H | 2 |
| 268 | GluNAc | C5-AEEA | C5-AEEA-GABA | N(H) | 4 | CH₂OH | 2 |
| 269 | GluNAc | C5-AEEA | C5-AEEA-GABA | N(H) | 4 | H | 2 |
| 270 | Mannose | C5 | C5 | C(O) | 2 | CH₂OH | 2 |
| 271 | Mannose | C5 | C5 | C(O) | 2 | H | 2 |
| 272 | Mannose | C5-AEEA | C5-AEEA-GABA | N(H) | 4 | CH₂OH | 2 |
| 273 | Mannose | C5-AEEA | C5-AEEA-GABA | N(H) | 4 | H | 2 |
| 274 | PA | AEEA | AEEA-GABA | N(H) | 4 | CH₂OH | 4 |
| 275 | PA | AEEA | AEEA-GABA | N(H) | 4 | H | 4 |

TABLE 10
Examples of Linear-6 compounds, which may be in salt form.

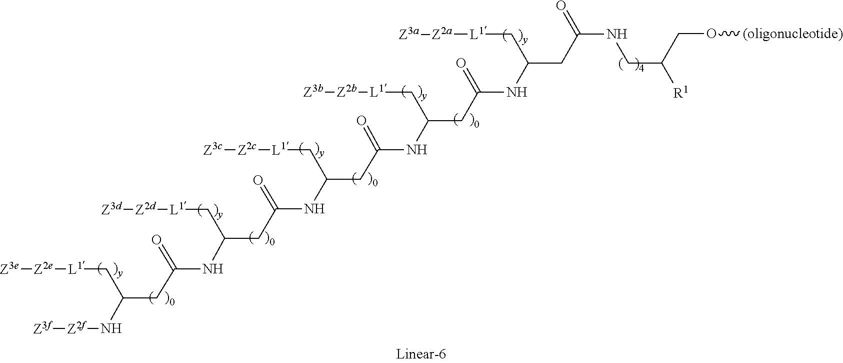

| | | | | | | | Oligo |
|---|---|---|---|---|---|---|---|
| | Z³ª, Z^{3b}, Z^{3c}, | Z^{2a}, Z^{2b}, Z^{2c}, Z^{2d}, | | | | | SEQ |
| # | Z^{3d}, Z^{3e}, Z^{3f} | Z^{2e} | Z^{2f} | L^{1′} | y | R¹ | ID NO |
| 276 | GalNAc | C5-AEEA | C5-AEEA-GABA | N(H) | 4 | CH₂OH | 1 |
| 277 | GalNAc | C5-AEEA | C5-AEEA-GABA | N(H) | 4 | H | 1 |
| 278 | GalNAc | C5-AEEA | C5-AEEA-GLY | C(O) | 2 | CH₂OH | 1 |
| 279 | GalNAc | C5-AEEA | C5-AEEA-GLY | C(O) | 2 | H | 1 |
| 280 | GluNAc | C5-AEEA | C5-AEEA-GABA | N(H) | 4 | CH₂OH | 2 |
| 281 | GluNAc | C5-AEEA | C5-AEEA-GABA | N(H) | 4 | H | 2 |
| 282 | GluNAc | C5-AEEA | C5-AEEA-GLY | C(O) | 2 | CH₂OH | 2 |
| 283 | GluNAc | C5-AEEA | C5-AEEA-GLY | C(O) | 2 | H | 2 |
| 284 | Mannose | C5-AEEA | C5-AEEA-GABA | N(H) | 4 | CH₂OH | 2 |
| 285 | Mannose | C5-AEEA | C5-AEEA-GABA | N(H) | 4 | H | 2 |
| 286 | Mannose | C5-AEEA | C5-AEEA-GLY | C(O) | 2 | CH₂OH | 2 |
| 287 | Mannose | C5-AEEA | C5-AEEA-GLY | C(O) | 2 | H | 2 |
| 288 | PA | AEEA | AEEA-GABA | N(H) | 4 | CH₂OH | 4 |
| 289 | PA | AEEA | AEEA-GABA | N(H) | 4 | H | 4 |

In some embodiments of Tables 2-10, “(oligonucleotide)” in the formulae may be replaced with a phosphoramidite moiety, e.g., P(N(iPr)₂)(OEtCN), and in such case R¹ as CH₂OH is instead CH₂O Z² where Z² includes an acid labile trityl moiety described herein, including, without limitation, Tr, MMTr, DMTr, or TMTr.

In some embodiments, provided herein are compounds including a formula:

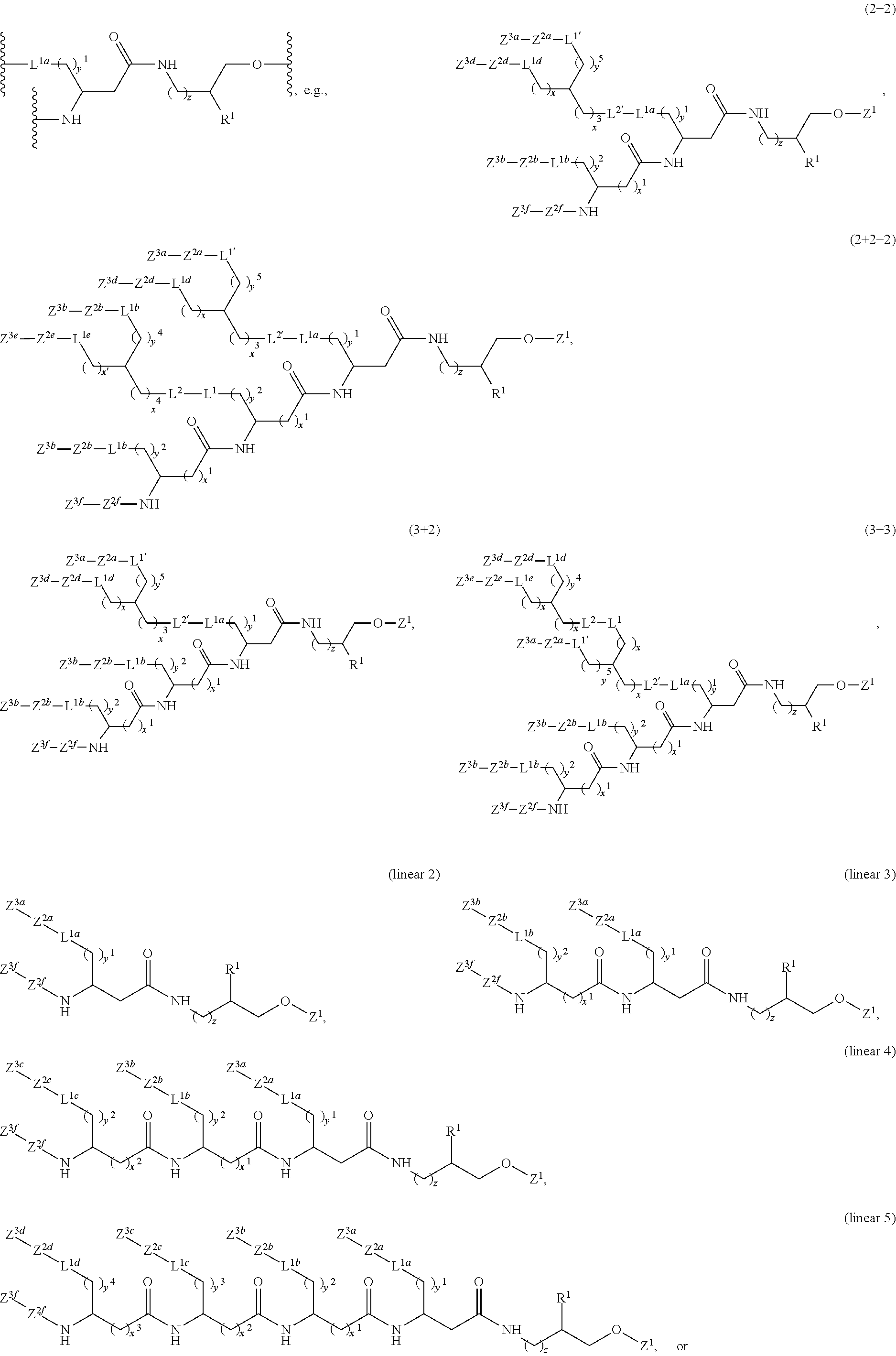

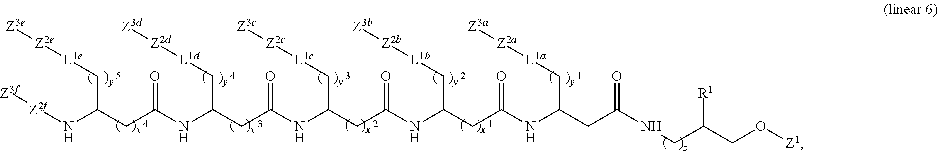

or a salt thereof,
wherein
Z¹ is H, a phosphoramidite, a solid support, or a macromolecule;
R¹ is H, CH₂OH, or CH₂OZ²;
Z² is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
z is 0, 1, 2, 3, 4, 5, or 6;
x, x′, x¹, x², x³, and x⁴ are each, independently, 0, 1, 2, 3, 4, 5, or 6;
y, y′, y¹, y², y³, y⁴, and y⁵, are each, independently, 0, 1, 2, 3, 4, 5, or 6;
Z^{2a}, Z^{2b}, Z^{2c}, Z^{2d}, Z^{2e}, and Z^{2f} are each, independently, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n is 1 to about 6;
Z^{3a}, Z^{3b}, Z^{3c}, Z^{3d}, Z^{3e}, and Z^{3f} are each, independently, selected from carbohydrate receptor ligands, such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies; and
L¹, L^{1′}, L^{1a}, L^{1b}, L^{1c}, L^{1d}, L^{1e}, L², and L^{2′}, are each, independently, N(H) or C(O).

In some embodiments, z is 0, 1, 2, 3, 4, 5, or 6;

In some embodiments, x, x′, x¹, x², x³, and x⁴ are each, independently, 0 or 1. In some embodiments, x, x′, x², x³, and x⁴ are 0, and x¹ is 1. In some embodiments, x, x′, x¹, x², x³, and x⁴ are 0.

In some embodiments, y, y′, y¹, y², y³, y⁴, and y⁵, are each, independently, 2, 3, 4, or 5. In some embodiments, y, y′, y¹, y², y³, y⁴, and y⁵, are each, independently, 2 or 4. In some embodiments, y, y′, y¹, y², y³, y⁴, and y⁵, are 2. In some embodiments, y, y′, y¹, y², y³, y⁴, and y⁵, are 4.

In some embodiments, Z^{2a}, Z^{2b}, Z^{2c}, Z^{2d}, Z^{2e}, and Z^{2f} are each, independently, selected from a structure of Table 16. (e.g., AEA-GABA, AEEA-GABA, AEEP-GABA, C5, C5-AEA-GABA, C5-AEEA-GABA, C5-AEEA-GLY, C5-AEEP-GABA, C5-GABA, C5-Gly, or GABA). In some embodiments, 2, 3, 4, 5, or all of Z^{2a}, Z^{2b}, Z^{2c}, Z^{2d}, Z^{2e}, and Z^{2f} are the same.

In some embodiments, Z^{3a}, Z^{3b}, Z^{3c}, Z^{3d}, Z^{3e}, and Z^{3f} are each, independently, selected from a ligand of Table 15 (e.g., GalNAc, GluNAc, PGA, CA, UDA, DDA, DDA 12-OH, TDA, MA, PDA, PA, HDA, SA, SA 18-OH, SA 12-OH, SA 2-OH, ACA, BA, DHA, ARA, EPA, ALA, GLA, RA, OA, EA, LA, or C5), a mannose, a cholesterol, a bile acid, a fatty acid, a cell penetrating peptide, a cell-targeting molecule having a molecular weight of about 30 to about 500 Da, a polyglycol, an aptamer, or an antibody. In some embodiments, Z^{3a}, Z^{3b}, Z^{3c}, Z^{3d}, Z^{3e}, and Z^{3f} are each, independently, selected from GalNAc, GluNAc, PGA, CA, UDA, DDA, DDA 12-OH, TDA, MA, PDA, PA, HAD, SA, SA 18-OH, SA 12-OH, SA 2-OH, ACA, BA, DHA, ARA, EPA, ALA, GLA, RA, OA, EA, LA, C5, a mannose, a cholesterol, a bile acid, a fatty acid, or a polyglycol.

In some embodiments, Z^{3a}, Z^{3b}, Z^{3c}, Z^{3d}, Z^{3e}, and Z^{3f} are 3p-hydroxy 5-cholenoic acid, ACA, ALA, ARA, BA, CA, Chenocholic acid, Cholesterol, Cholic acid, DDA, DDA 12-OH, DHA, EA, EPA, GalNAc, GLA, GluNAc, HDA, LA, Lithocholic acid, MA, Mannose, OA, PA, each independently selected from PA or CA, each independently selected from PA or DDA, each independently selected from PA or MA, each independently selected from PA or PDA, each independently selected from PA or PGA, each independently selected from PA or TDA, each independently selected from PA or UDA, PDA, PGA, RA, SA, SA 12-OH, SA 18-OH, SA 2-OH, TDA, UDA, or Ursodeoxycholic acid. In some embodiments, 2, 3, 4, 5, or all of Z^{3a}, Z^{3b}, Z^{3c}, Z^{3d}, Z^{3e}, and Z^{3f} are the same.

In some embodiments of the formulae herein:
R¹ is H or CH₂OZ², and Z¹ is H, a solid support, an oligomer, or

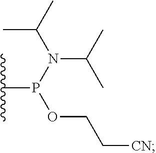 [SMILES: *P(OCCC#N)N(C(C)C)C(C)C]

or R¹ is H, CH₂OH, or CH₂OZ², and Z¹ is H, a solid support, or an oligomer;
Z² is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
z is 4;
x, x′, x¹, x², x³, and x⁴ are each, independently, 0 or 1 (e.g., x, x′, x², x³, and x⁴ are 0 and x¹ is 1, or x, x′, x¹, x², x³, and x⁴ are 0);
y, y′, y¹, y², y³, y⁴, and y⁵, are each, independently, 0, 1, 2, 3, 4, 5, or 6 (e.g., y, y′, y¹, y², y³, y⁴, and y⁵, are each, independently, 2 or 4, e.g., y, y′, y¹, y², y³, y⁴, and y⁵, are 2, or y, y′, y¹, y², y³, y⁴, and y⁵, are 4);
Z^{2a}, Z^{2b}, Z^{2c}, Z^{2d}, Z^{2e}, and Z^{2f} are each, independently, selected from AEA-GABA, AEEA-GABA, AEEP-GABA, C5, C5-AEA-GABA, C5-AEEA-GABA, C5-AEEA-GLY, C5-AEEP-GABA, C5-GABA, C5-Gly, or GABA (e.g., Z^{2a}, Z^{2b}, Z^{2c}, Z^{2d}, Z^{2e}, and Z^{2f} are each selected from two of AEA-GABA, AEEA-GABA, AEEP-GABA, C5, C5-AEA-GABA, C5-AEEA-GABA, C5-AEEA-GLY, C5-AEEP-GABA, C5-GABA, C5-Gly, or GABA, or Z^{2a}, Z^{2b}, Z^{2c}, Z^{2d}, Z^{2e}, and Z^{2f} are the same); and
Z^{3a}, Z^{3b}, Z^{3c}, Z^{3d}, Z^{3e}, and Z^{3f} are 3p-hydroxy 5-cholenoic acid, ACA, ALA, ARA, BA, CA, Chenocholic acid, Cholesterol, Cholic acid, DDA, DDA 12-OH, DHA, EA, EPA, GalNAc, GLA, GluNAc, HDA, LA, Lithocholic acid, MA, Mannose, OA, PA, each independently selected from PA or CA, each independently selected from PA or DDA, each independently selected from PA or MA, each independently selected from PA or PDA, each independently selected from PA or PGA, each independently selected from PA or TDA, each independently selected from PA or UDA, PDA, PGA, RA, SA, SA 12-OH, SA 18-OH, SA 2-OH, TDA, UDA, or Ursodeoxycholic acid.

In some embodiments, the compounds provided herein comprise one or more of following formulae: divalent oligonucleotide

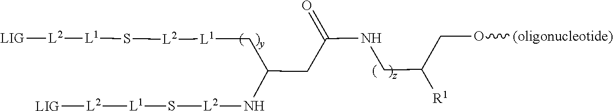

or a stereoisomer or a salt thereof,
wherein
each y is, independently, selected from the number of 1, 2, 3, 4, 5 or 6;
each z is, independently, selected from the number of 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: trivalent oligonucleotide

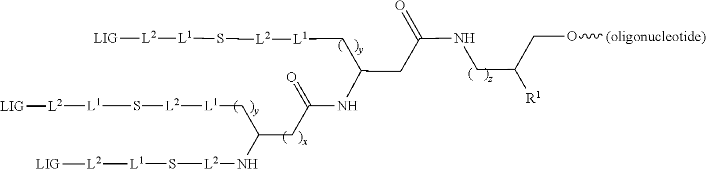

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: tetravalent linear oligonucleotide

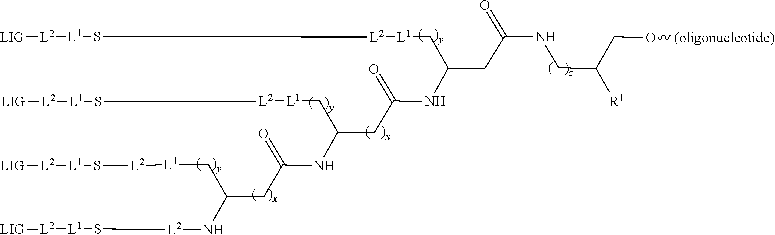

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: tetravalent 2+2 oligonucleotide

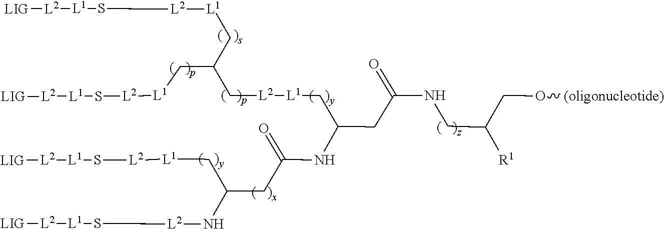

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: pentavalent linear oligonucleotide

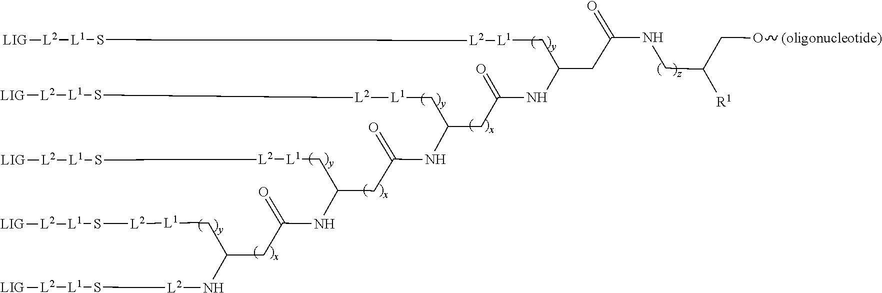

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: pentavalent 3+2 oligonucleotide

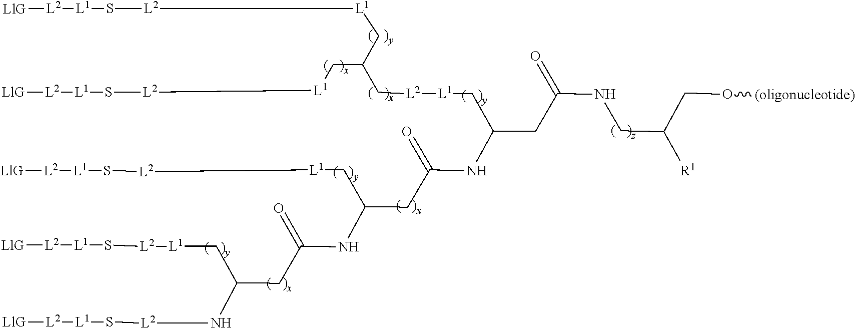

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: hexavalent linear oligonucleotide

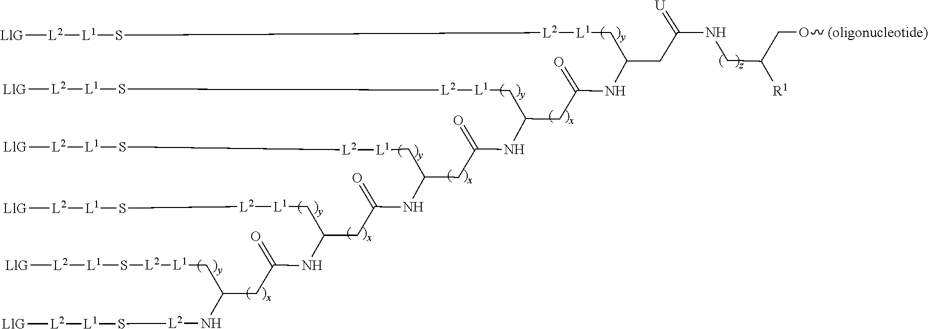

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: hexavalent 3+3 oligonucleotide

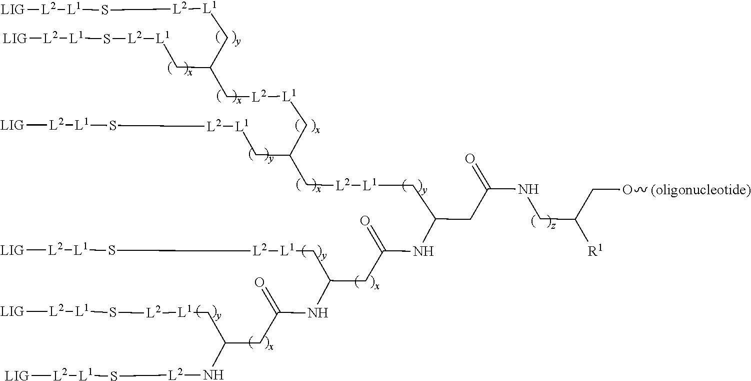

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: hexavalent 2+2+2 oligonucleotide

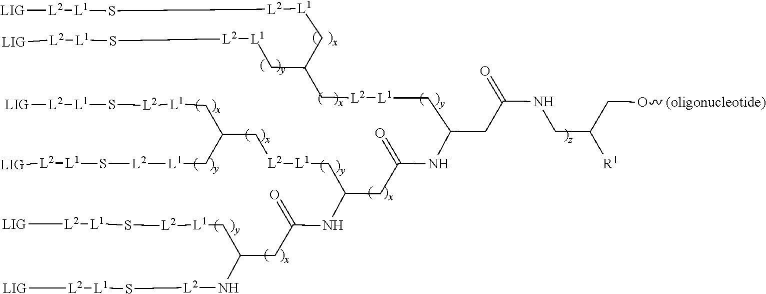

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
each R¹ is, independently, selected from hydrogen (—H) or methylene alcohol (—CH₂OH);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae: divalent solid support

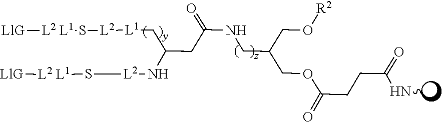

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene resin (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: trivalent solid support

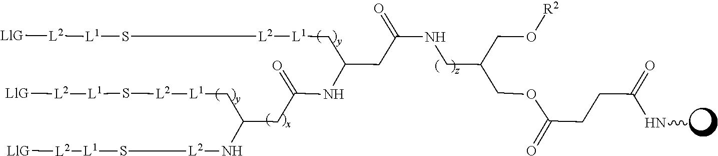

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent linear solid support

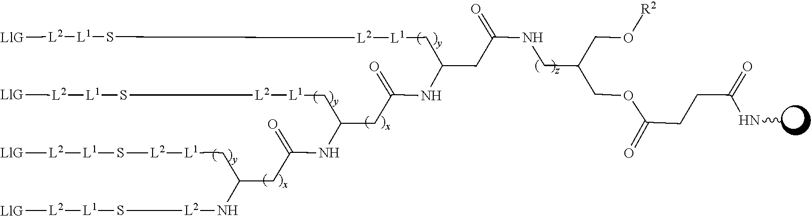

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent 2+2 solid support

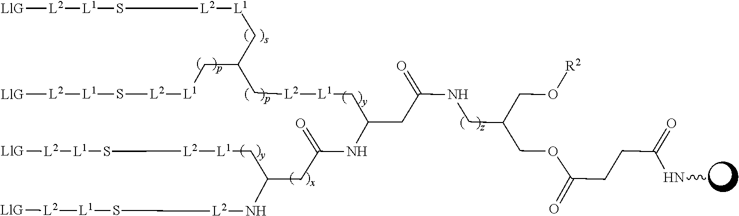

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent linear solid support

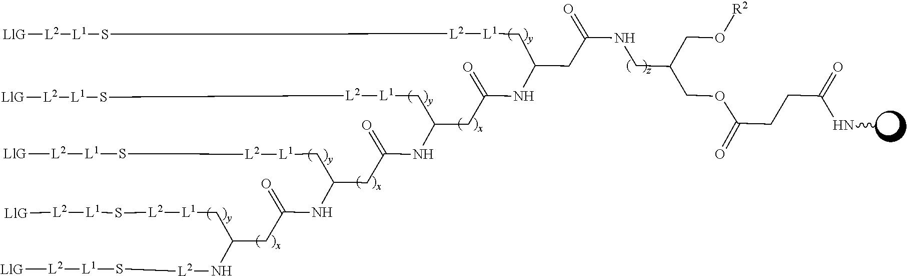

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent 3+2 solid support

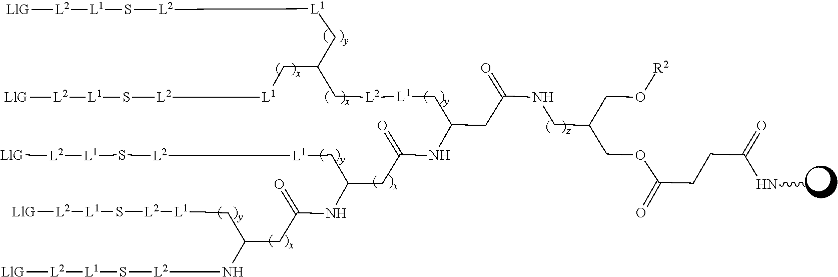

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent linear solid support

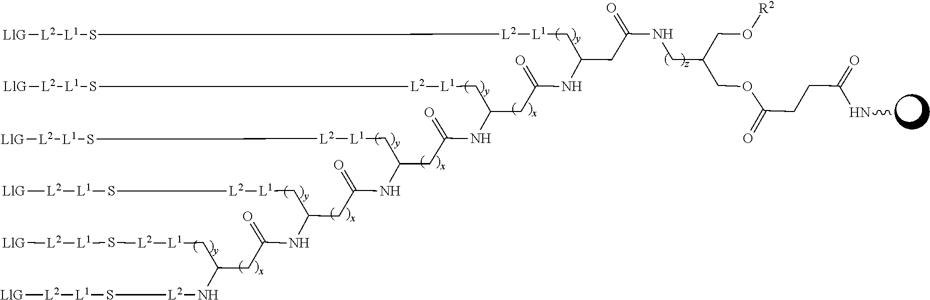

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 3+3 solid support

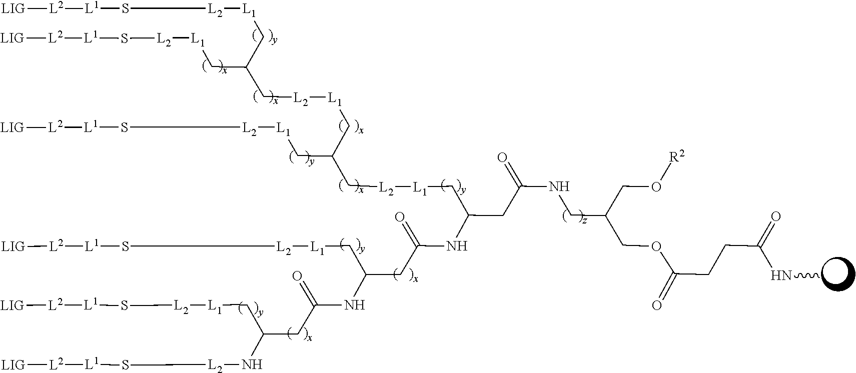

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 2+2+2 solid support

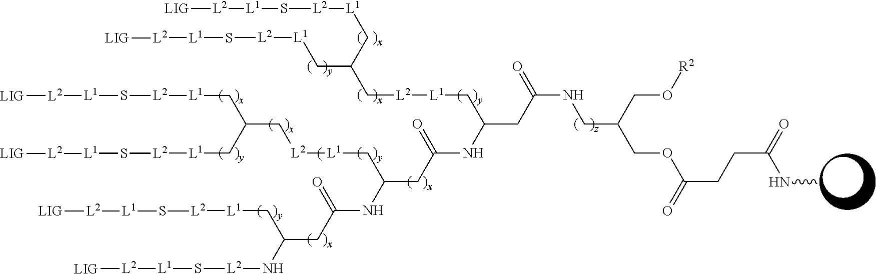

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
black circle is solid support, selected from silica gel, controlled pore glass (CPG), or polystyrene (PS);
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: divalent amidite

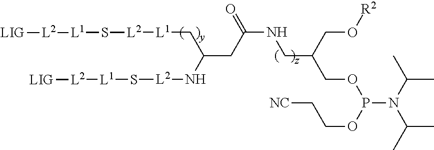

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: trivalent amidite

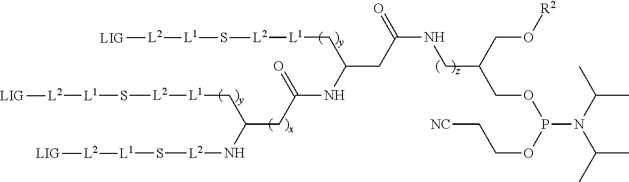

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent linear amidite

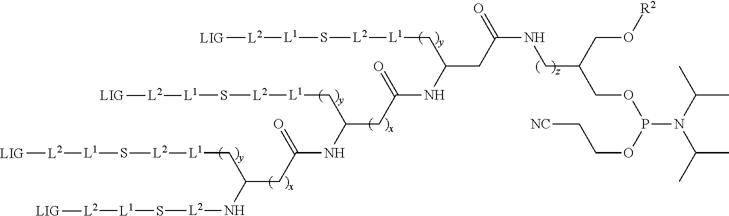

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent 2+2 amidite

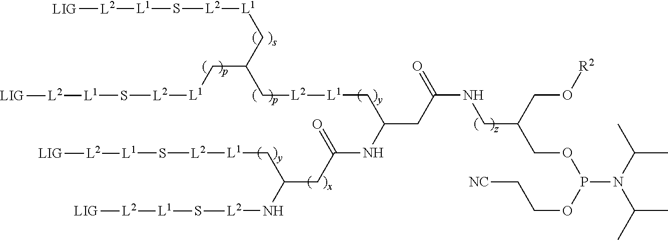

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent linear amidite

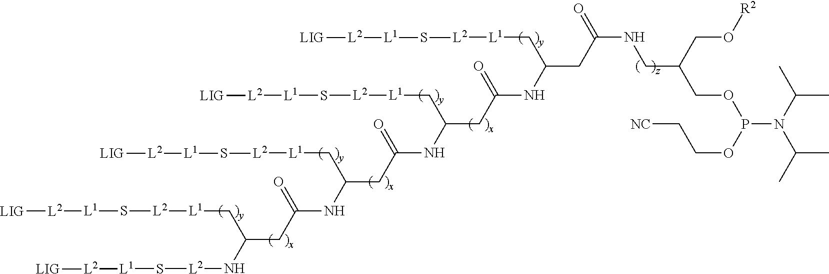

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent 3+2 amidite

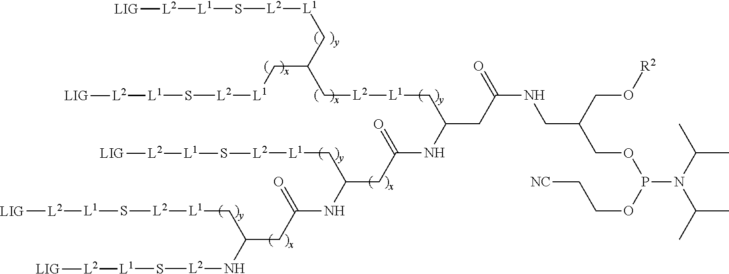

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent linear amidite

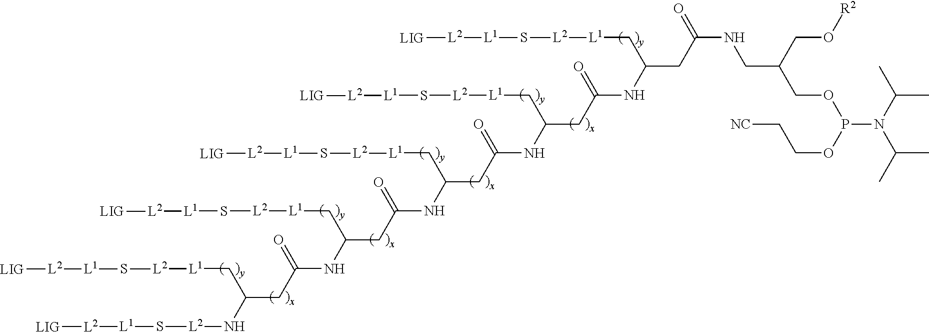

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 3+3 amidite

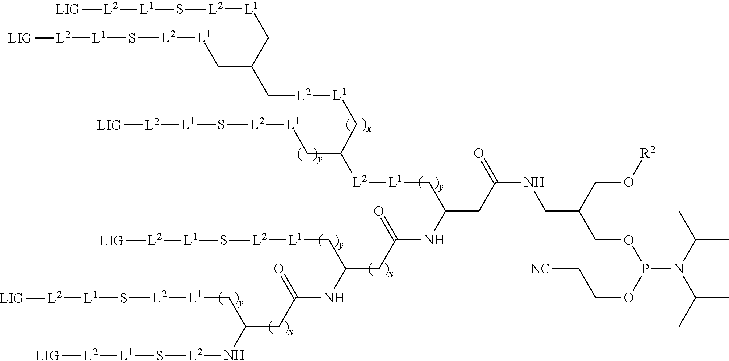

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 2+2+2 amidite

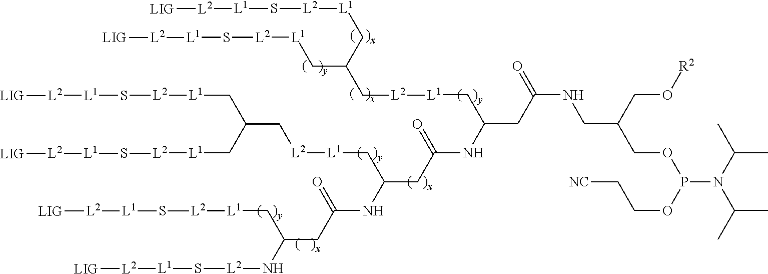

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each z is, independently, selected from the number of 0, 1, 2, 3, 4, 5 or 6;
R² is, independently, selected from, but not limited to, triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments of the formulae provided herein having a first β-amino acid, which is conjugated to a non-amino acid moiety at the carboxy-terminus of the first β-amino acid, e.g., a conjugating moiety to the macromolecule, phosphoramidite, or Z¹, or the like, a second β-amino acid is attached at the amino-terminus of the first β-amino acid such that a β-amino acid dipeptide is formed from the first and second β-amino acids, optionally wherein all other amino-acids in the formulae (e.g., the amino-acid cluster) are standard (e.g., α) D-amino-acids. Thus, generically, in some embodiments, x in the formulae herein is independently 0 or 1, wherein at least one x is 1. In some embodiments, x in the formulae herein is independently 0 or 1, wherein the x closest to z, or z's corresponding position in the formulae, is 1, optionally wherein all other “x” moieties are 0 (e.g., D-α-amino-acid).

In some embodiments, the compounds provided herein comprise one or more of following formulae: divalent acid cluster

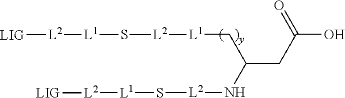

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: trivalent acid cluster

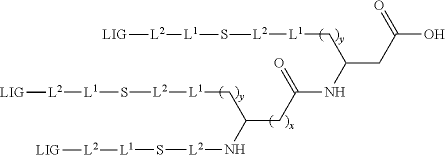

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent acid cluster

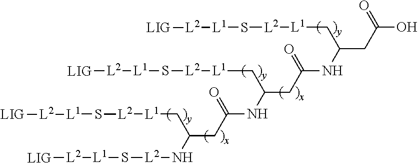

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: tetravalent 2+2 acid cluster

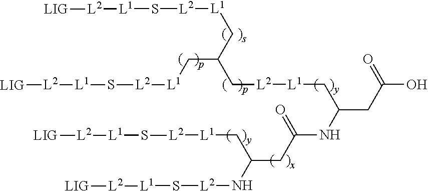

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent linear acid cluster

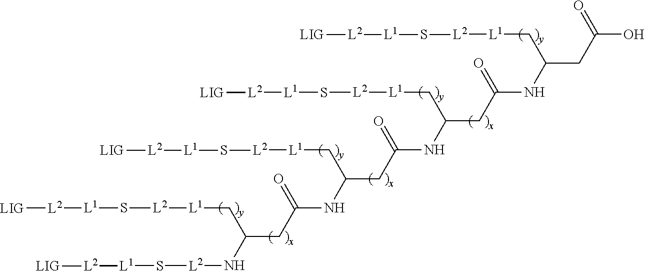

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: pentavalent 3+2 acid cluster

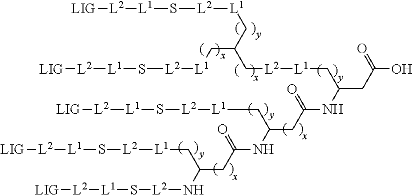

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent linear acid cluster

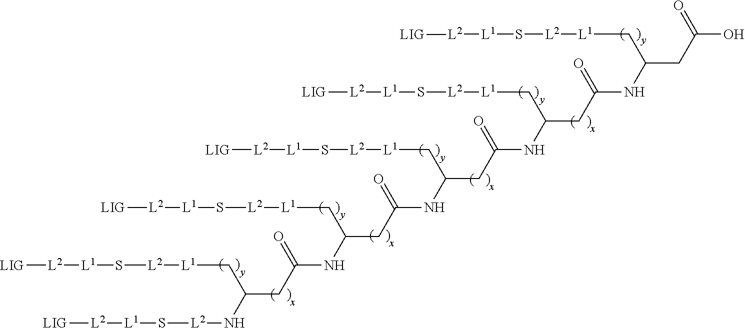

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 3+3 acid cluster

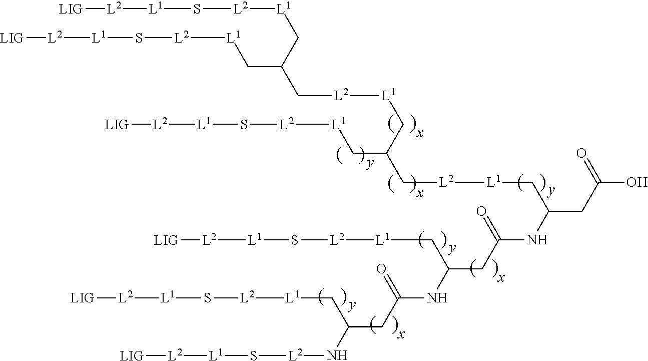

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments, the compounds provided herein comprise one or more of following formulae: hexavalent 2+2+2 acid cluster

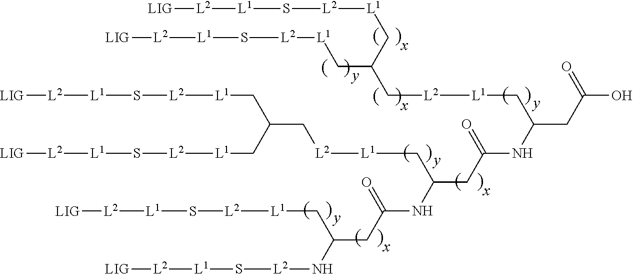

or a stereoisomer or a salt thereof,
wherein
each x is, independently, selected from the number of 0, 1, 2, 3, 4, 5 or 6;
each y is, independently, selected from the number of 0, 1, 2, 3, 4, 5 or 6;
each L¹ is, independently, selected from —N(H)— or —C(O)—;
each L² is, independently, selected from —N(H)— or —C(O)—;
each S is, independently, selected from null, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n=1˜6; and
each LIG is, independently, selected from carbohydrate receptor ligands such as N-acetylgalactosamine derivatives, N-acetylglucosamine derivatives, and mannose derivatives, lipids such as cholesterol, bile acid derivatives, and fatty acid derivatives, cell penetrating peptides (CPPs), specific small molecules showing cell-targeting effects, polymers such as poly glycols, aptamers and antibodies.

In some embodiments of the formulae herein, reference to C_{x-y}, e.g., C₁₋₂₀, C₂₋₂₀, C₃₋₂₀, C₄₋₂₀, or C₅₋₂₀ each, independently, may be replaced with C₉₋₂₂. For example, C₁₋₂₀ alkyl may be replaced in the formulae with C₉₋₂₂ alkyl.

In some embodiments, the compounds provided herein comprise one or more of following formulae: divalent

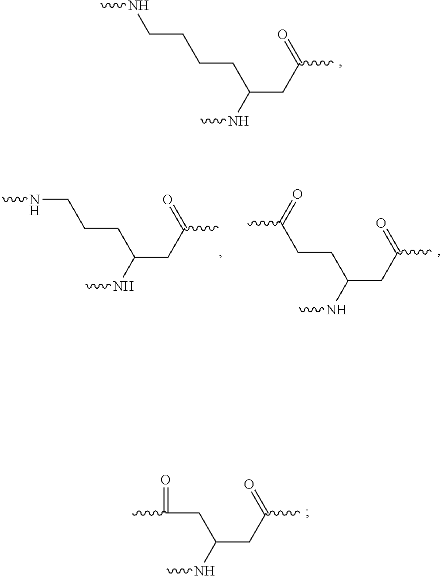

or a stereoisomer or a salt thereof.

In some embodiments, the compounds provided herein comprise one or more of following formulae: trivalent

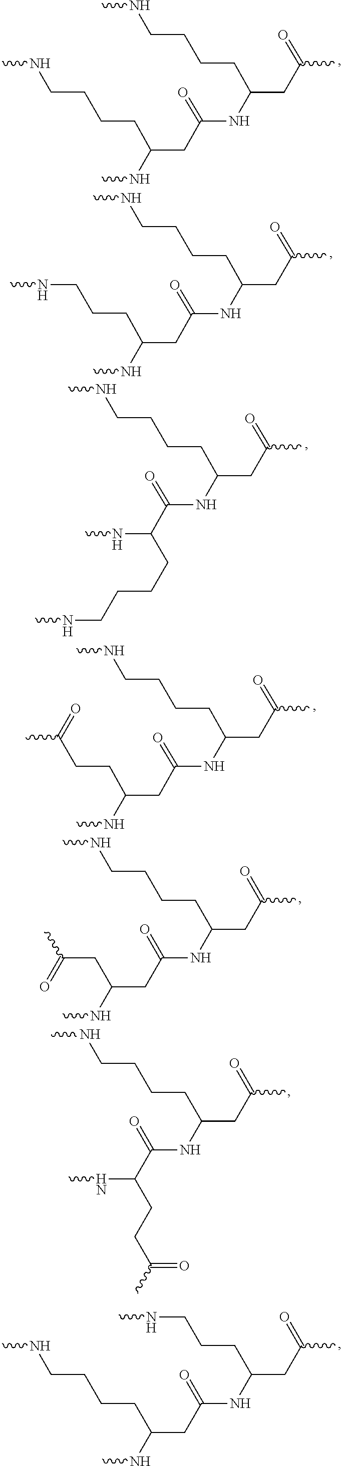

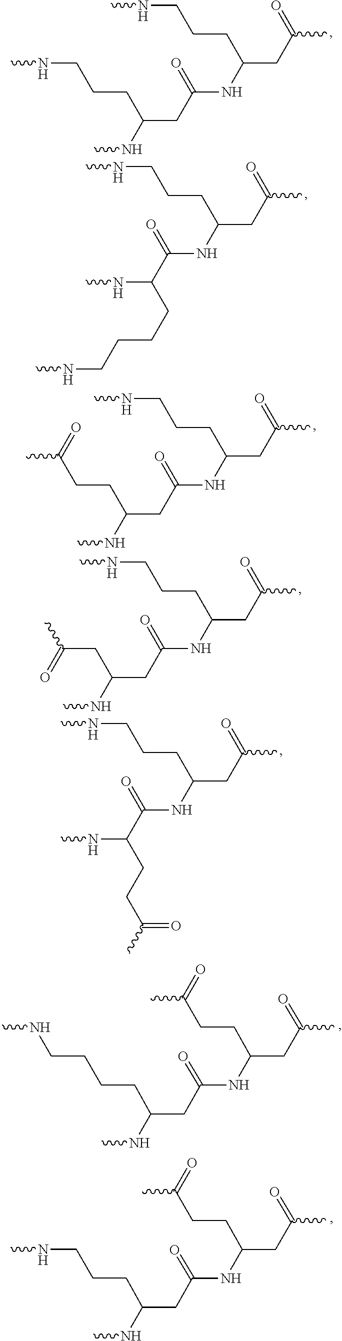

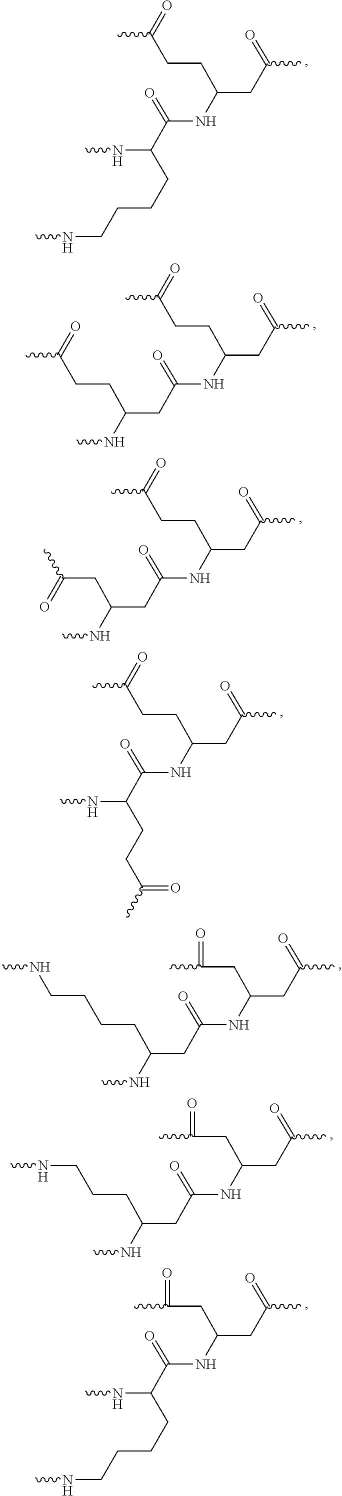

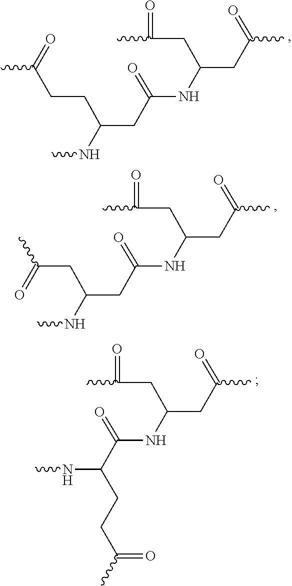

or a stereoisomer or a salt thereof.

In some embodiments, the compounds provided herein comprise one or more of following formulae: tetravalent linear

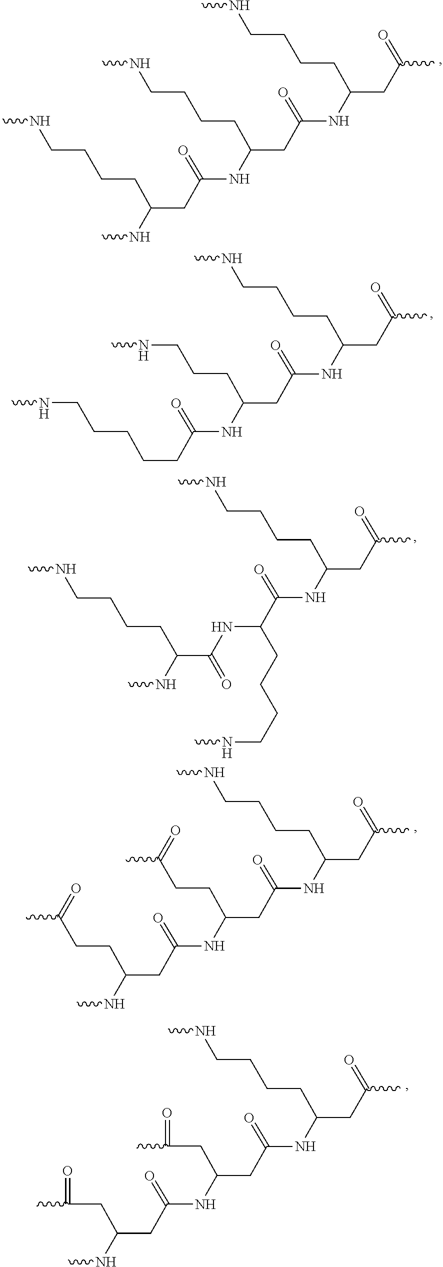

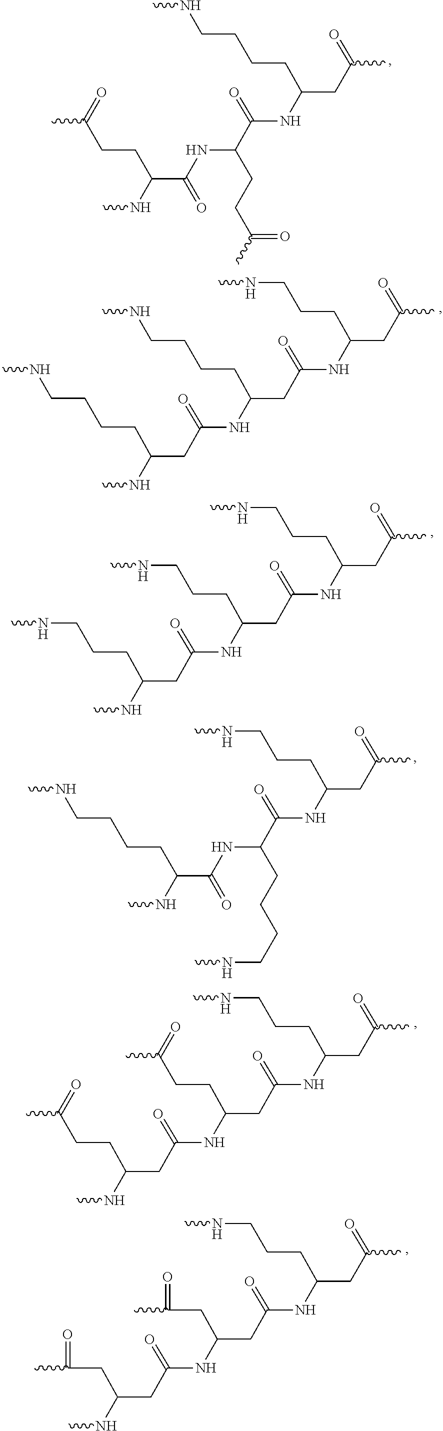

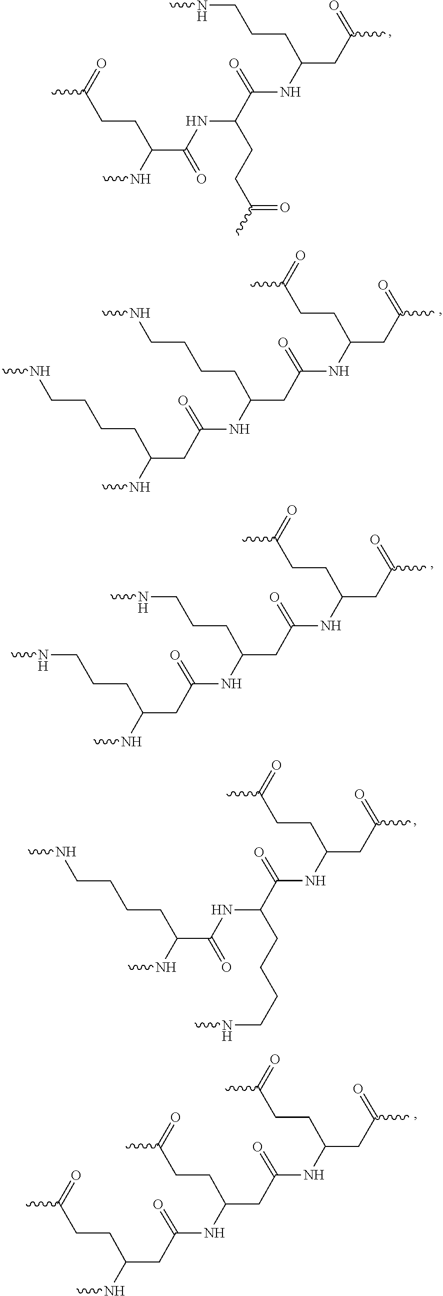

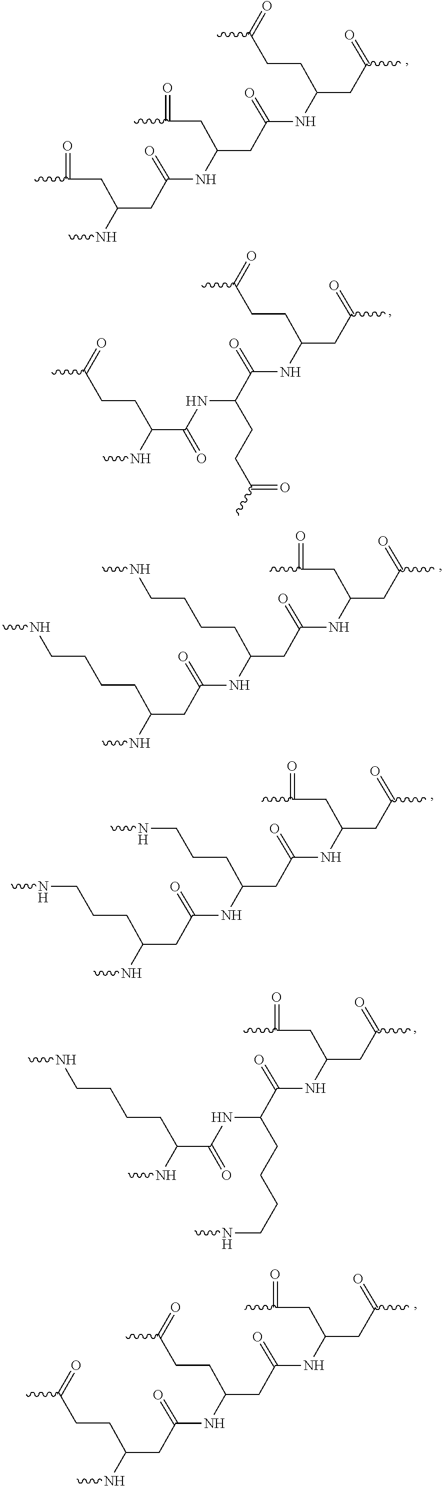

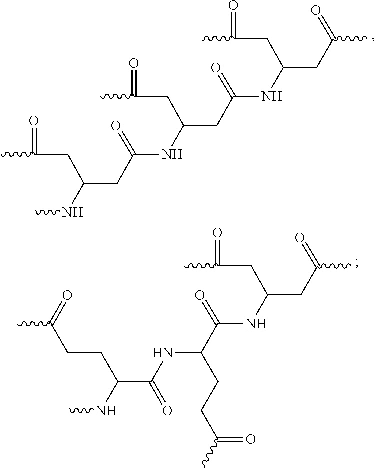

or a stereoisomer or a salt thereof.
In some embodiments, the compounds provided herein comprise one or more of following formulae: tetravalent 2+2

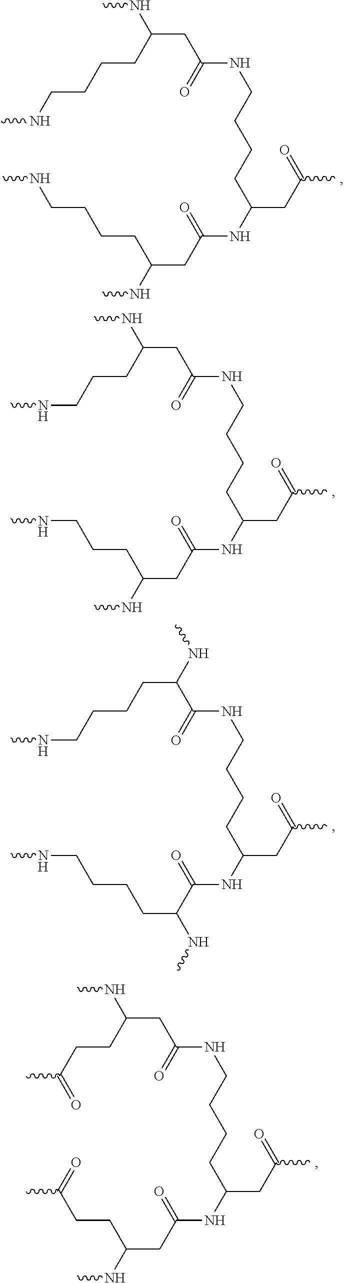

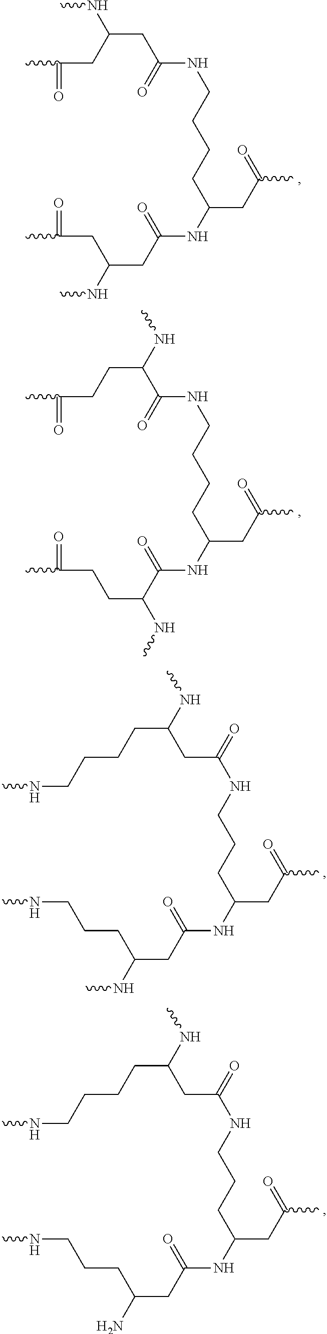

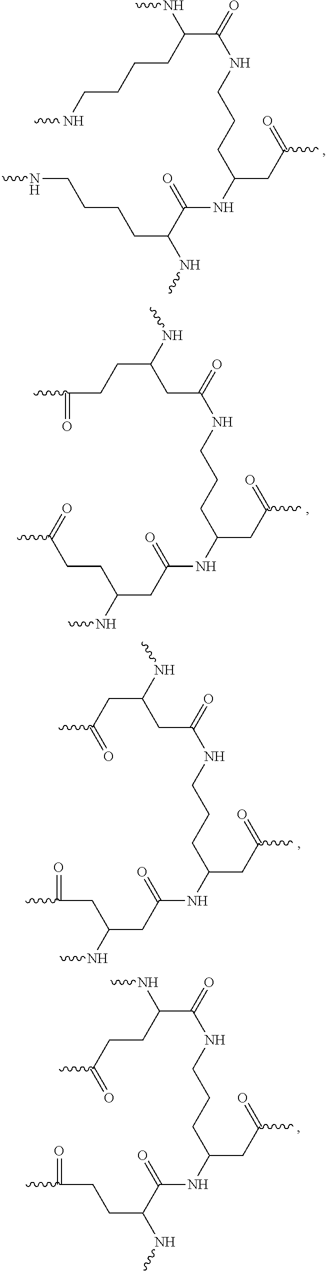

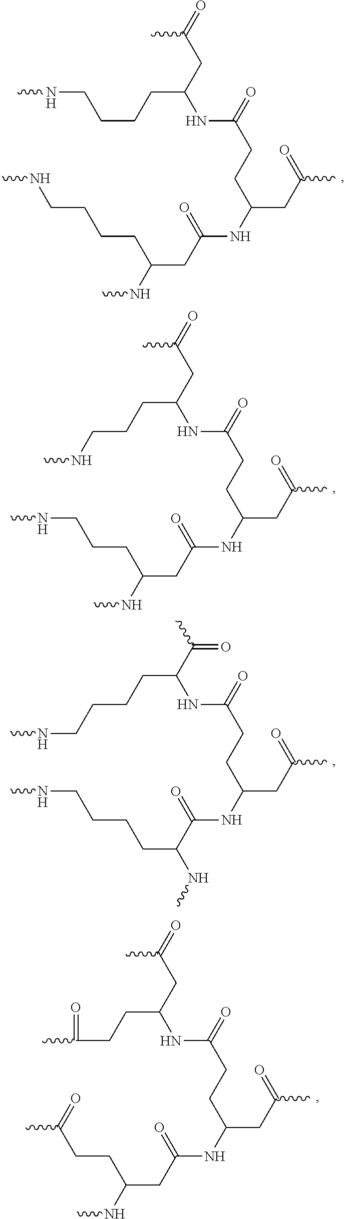

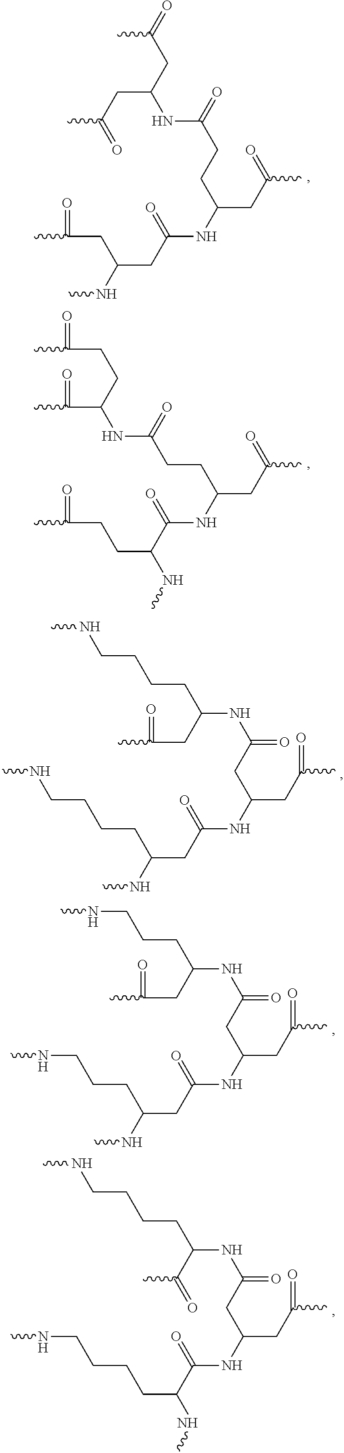

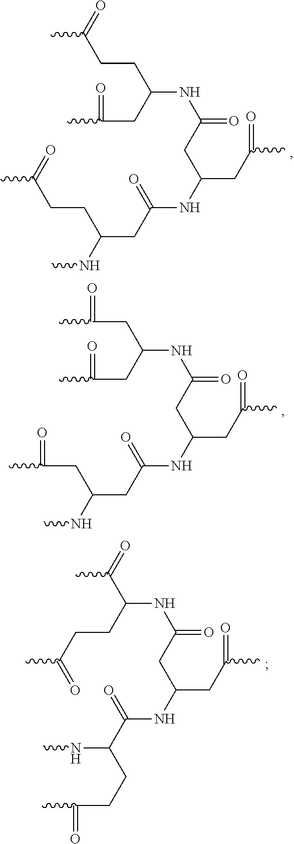

or a stereoisomer or a salt thereof.

In some embodiments, the compounds provided herein comprise one or more of following formulae: pentavalent linear

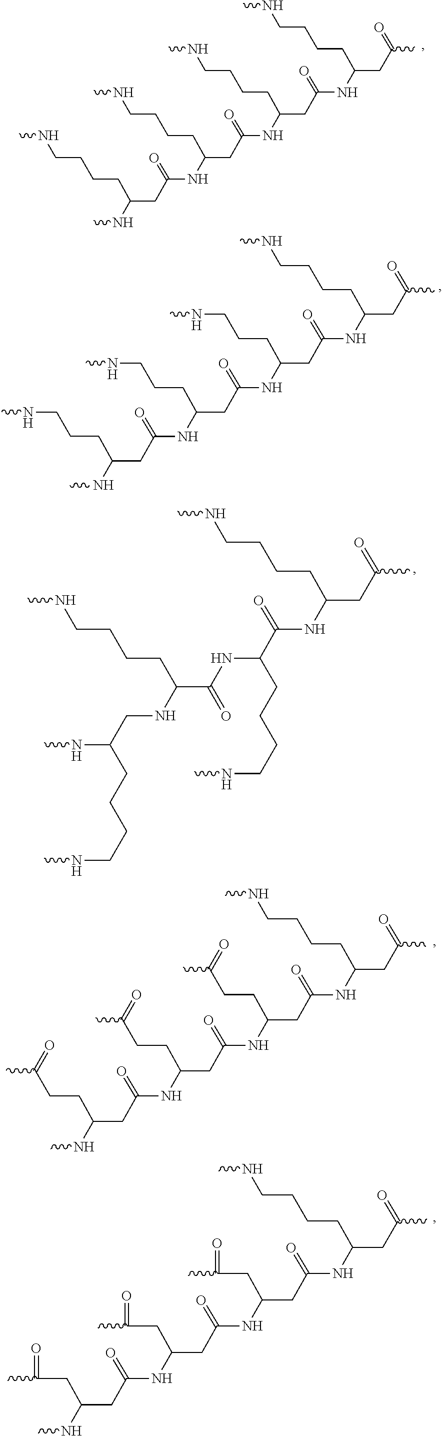

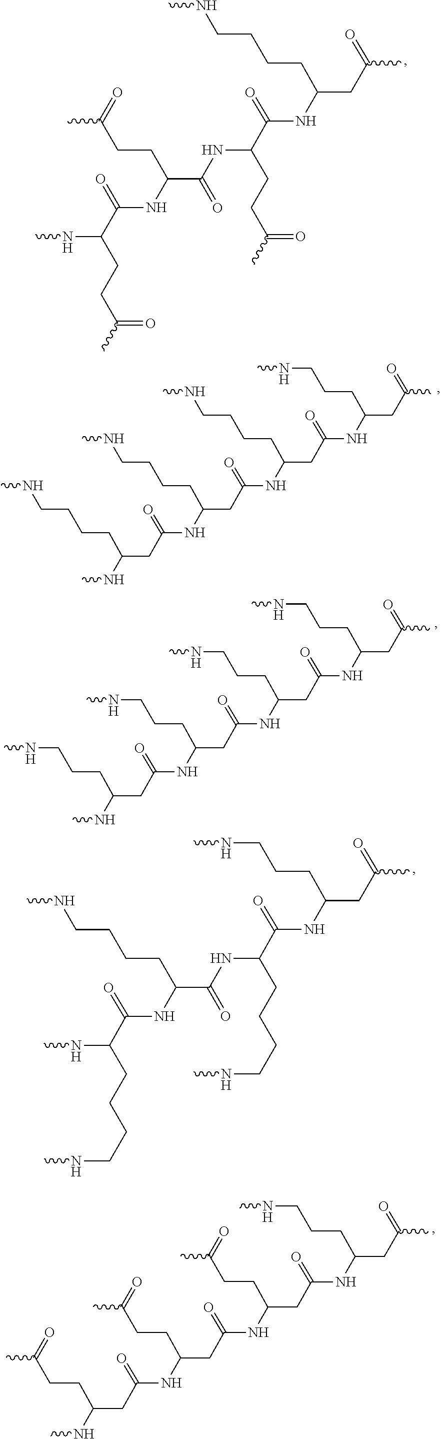

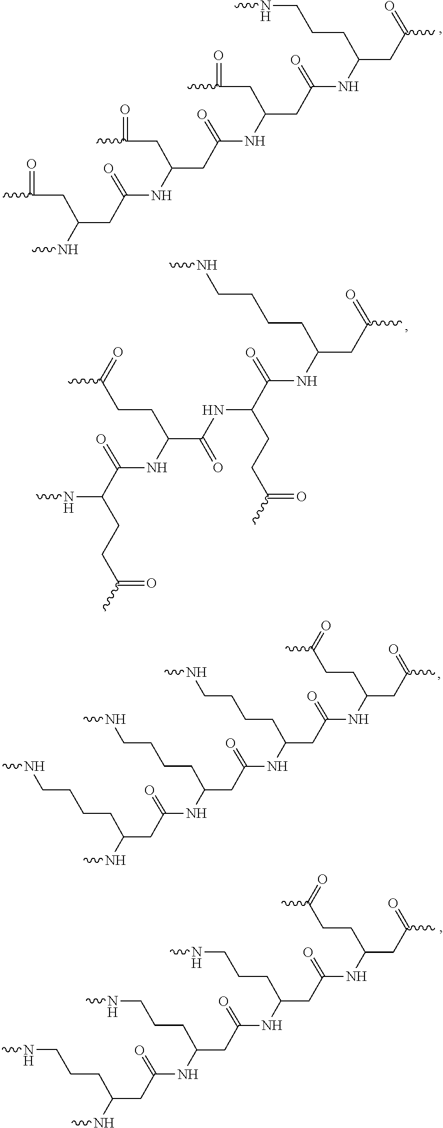

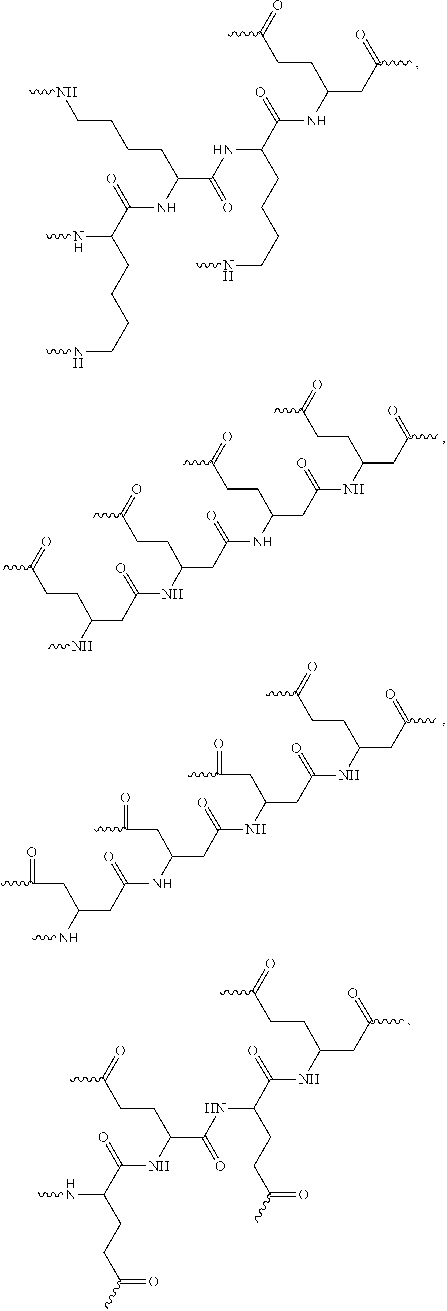

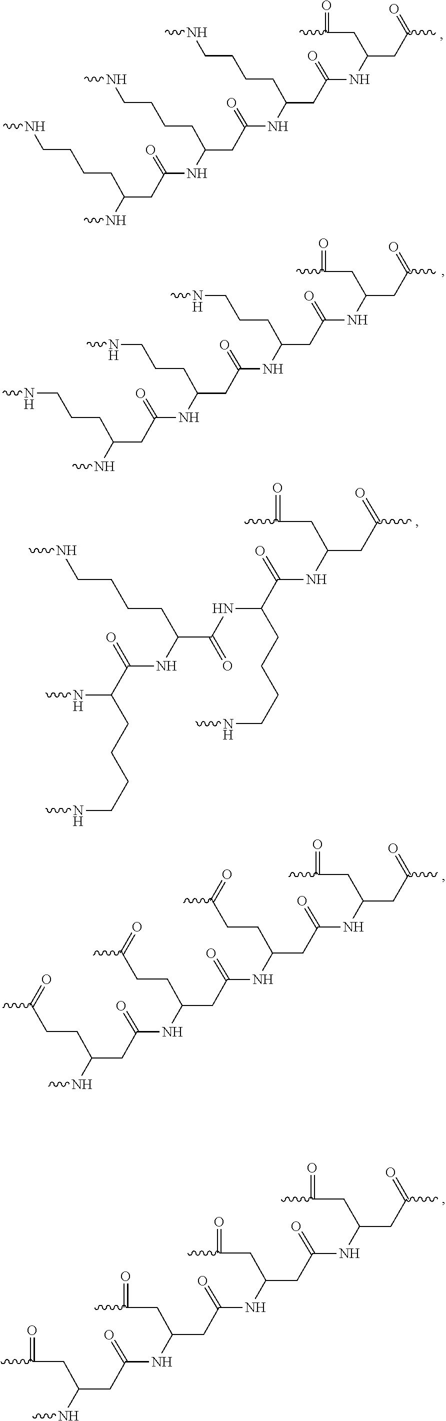

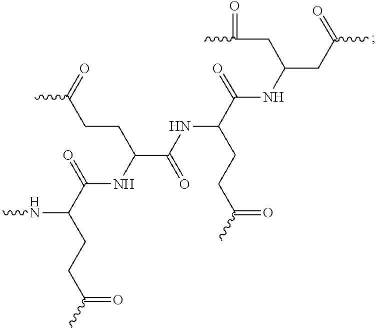

or a stereoisomer or a salt thereof.

In some embodiments, the compounds provided herein comprise one or more of following formulae: pentavalent 3+2

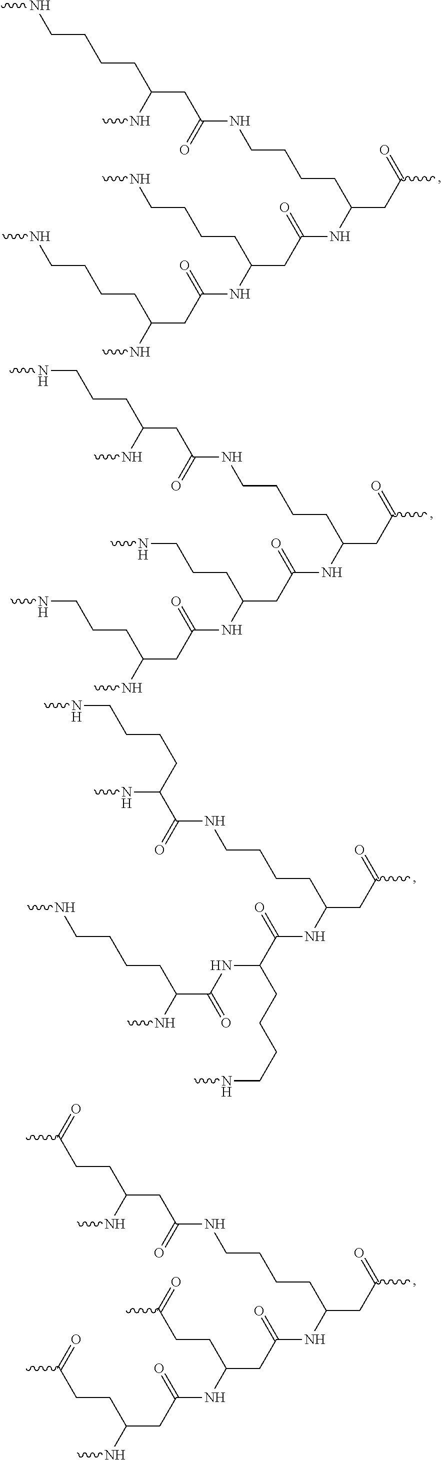

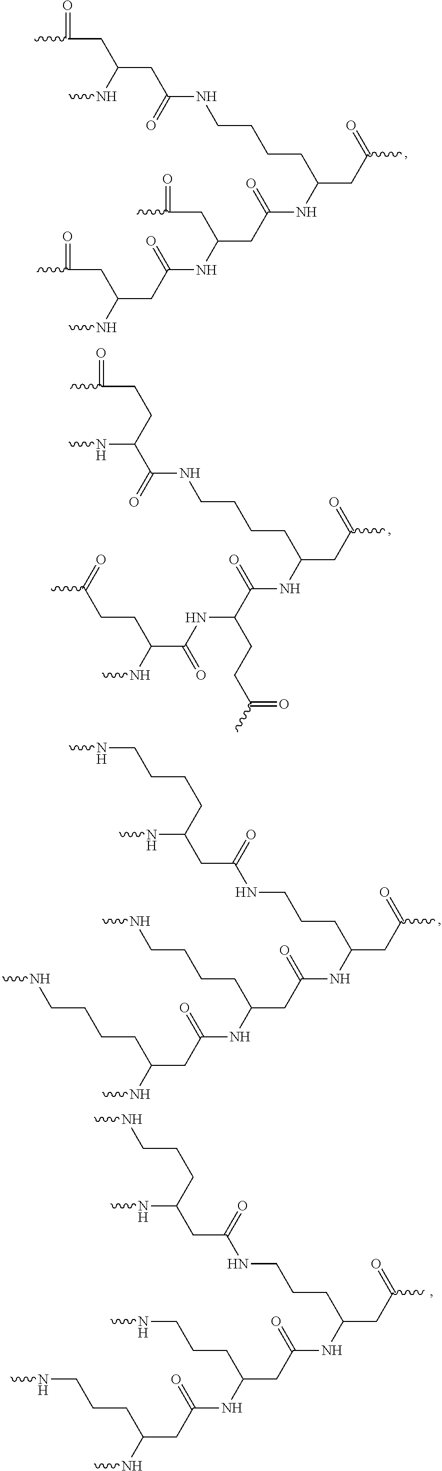

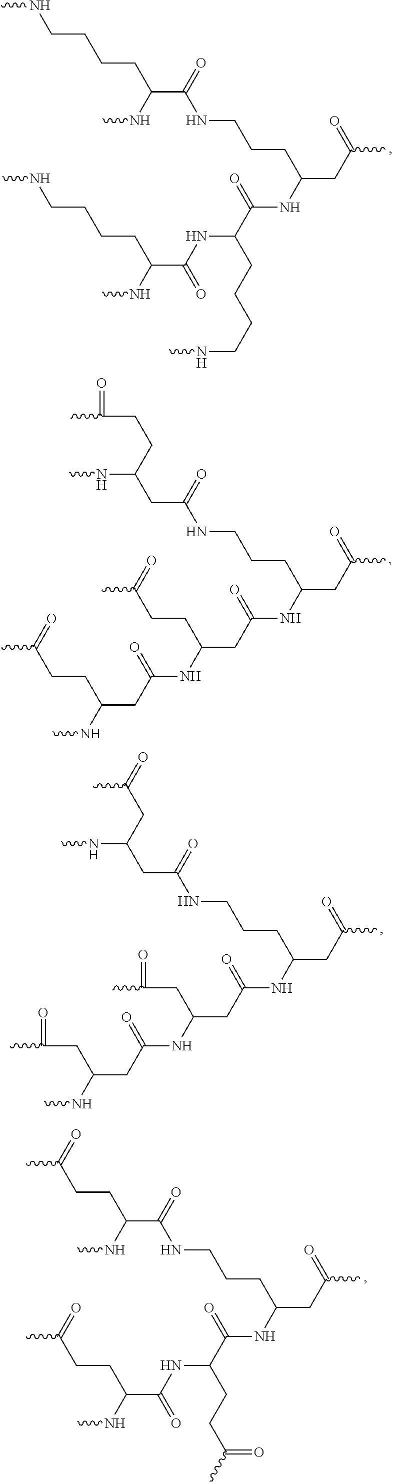

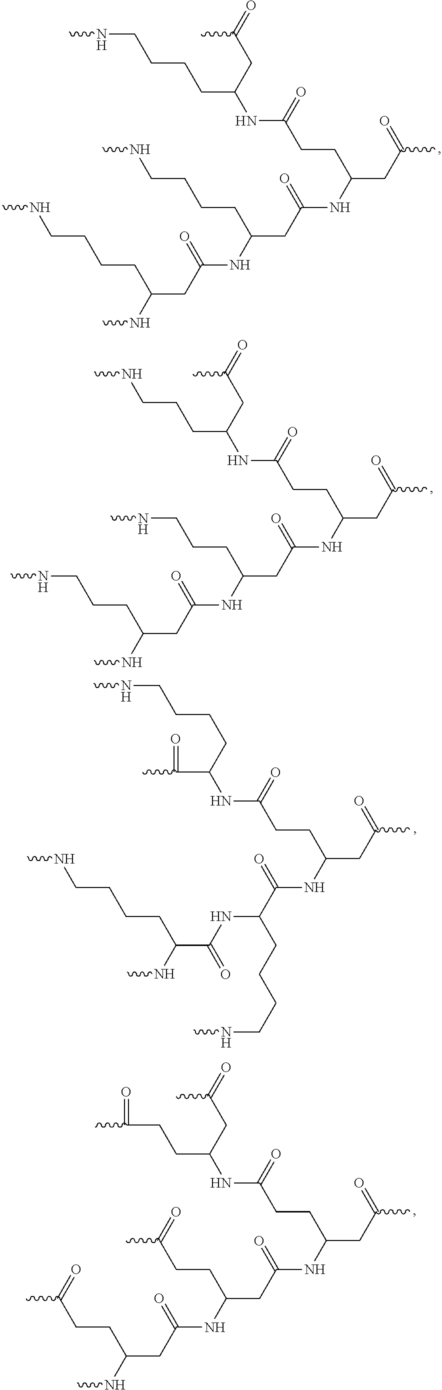

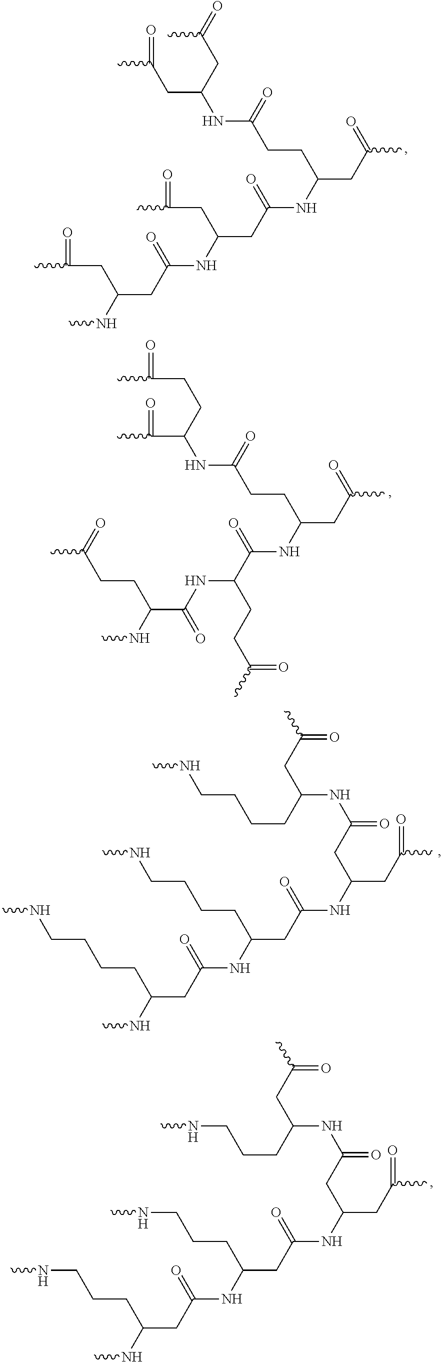

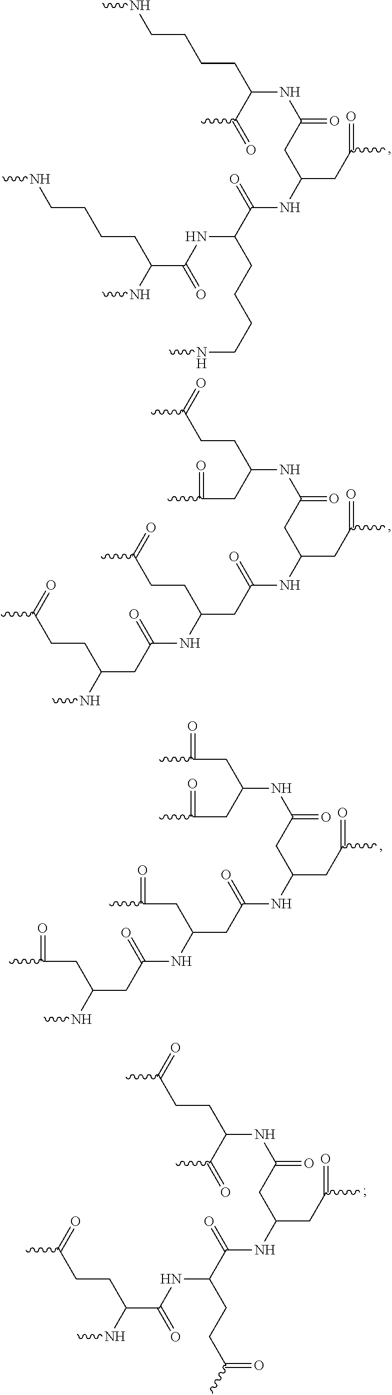

or a stereoisomer or a salt thereof.

In some embodiments, the compounds provided herein comprise one or more of following formulae: hexavalent linear

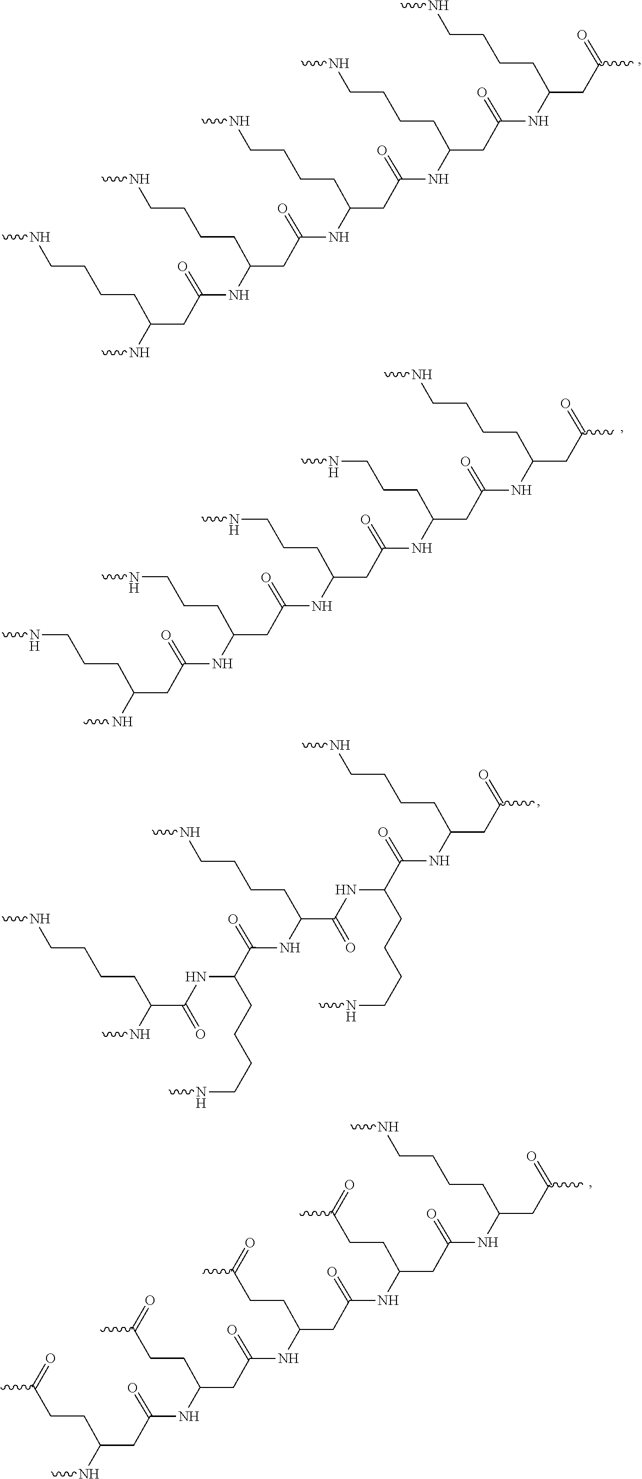

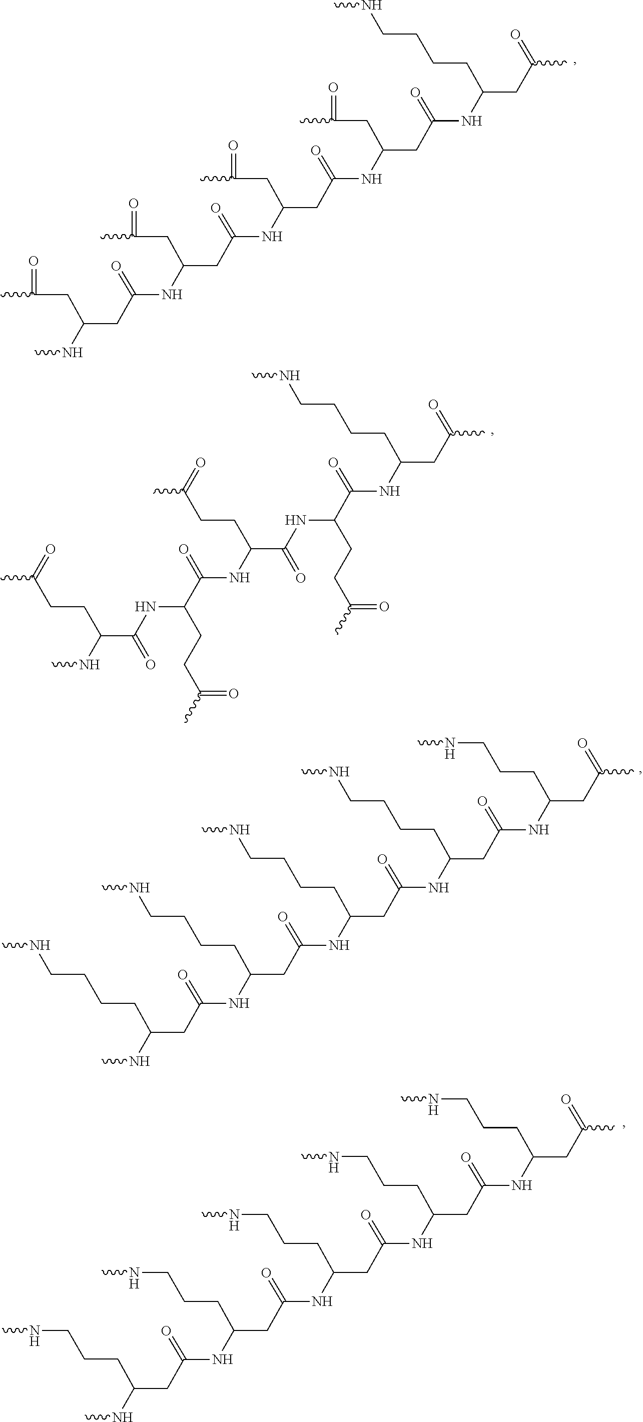

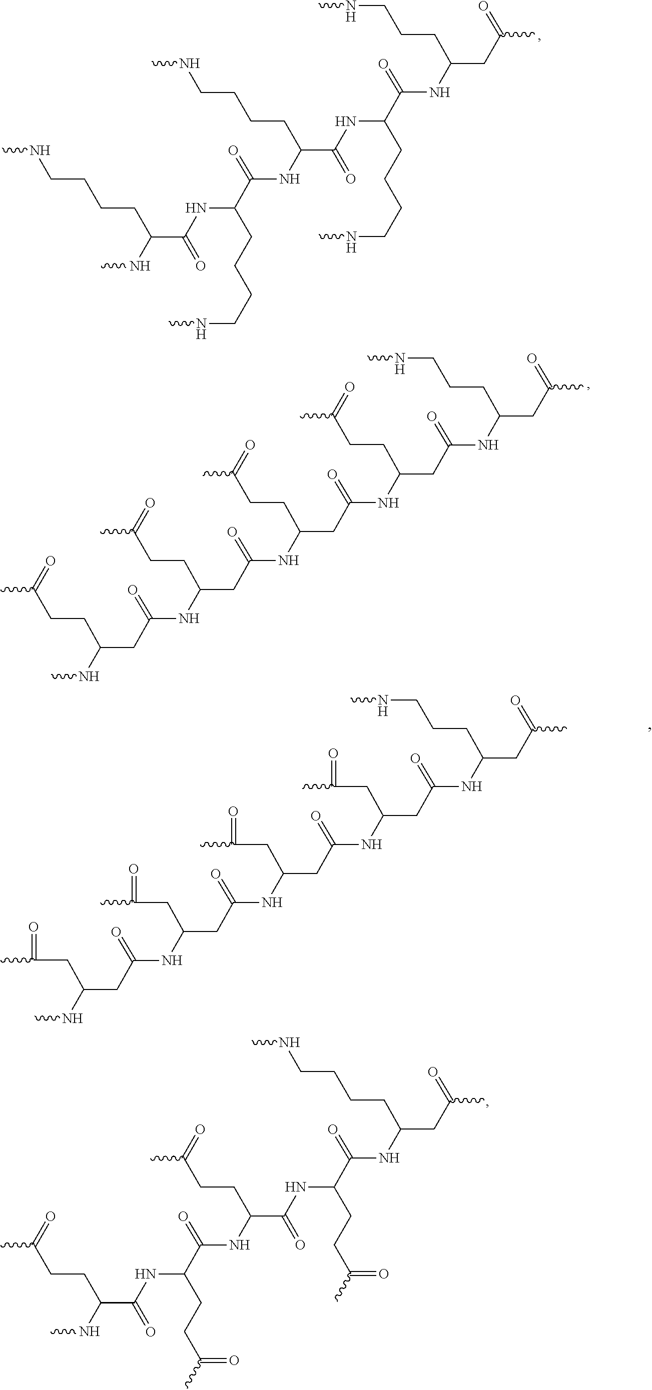

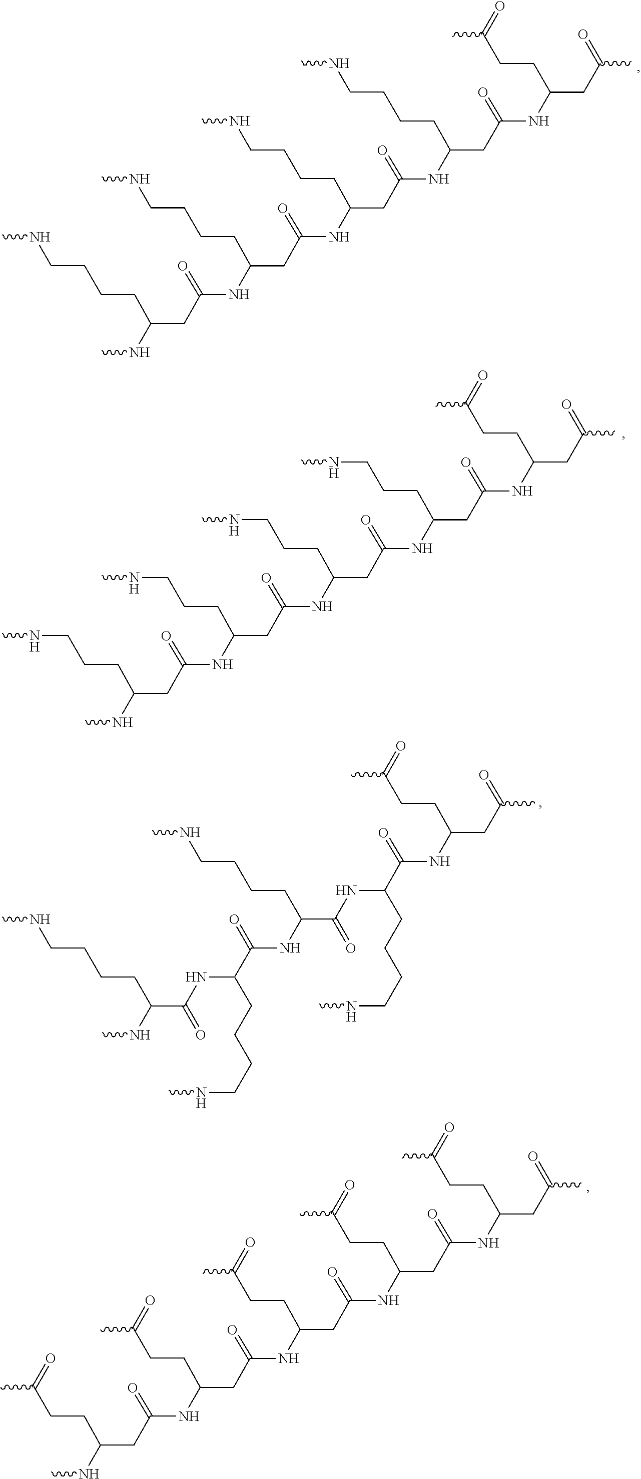

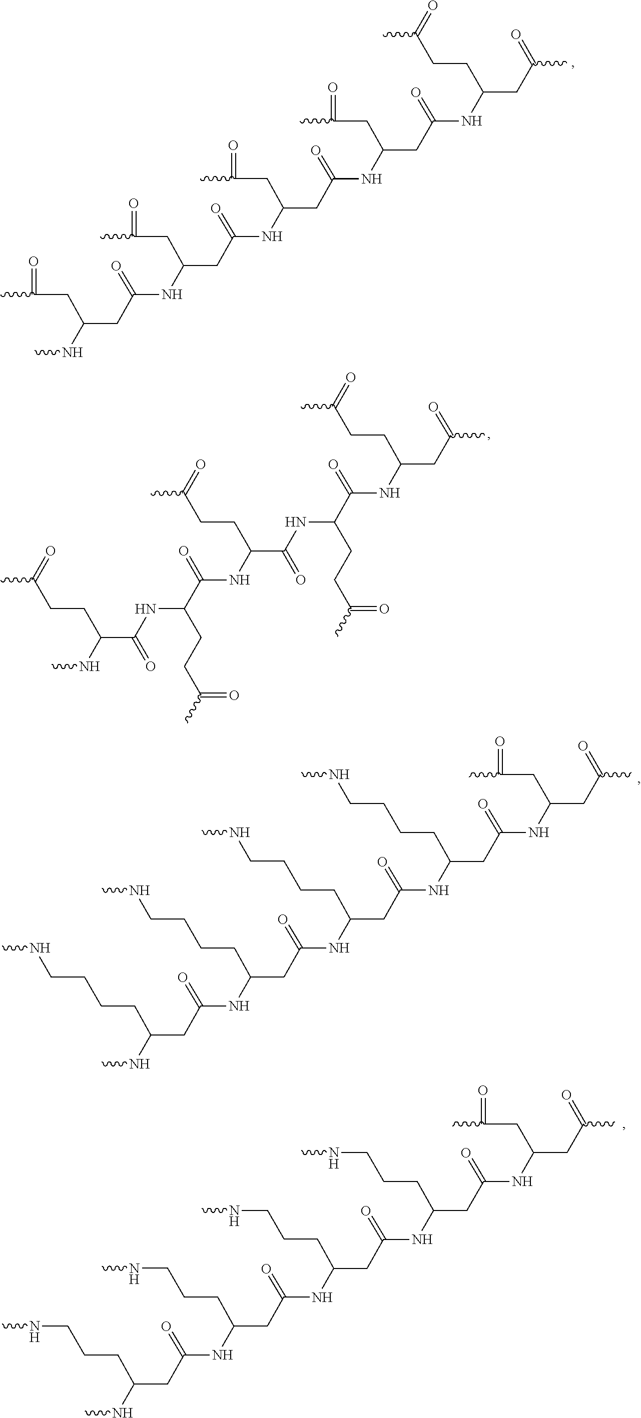

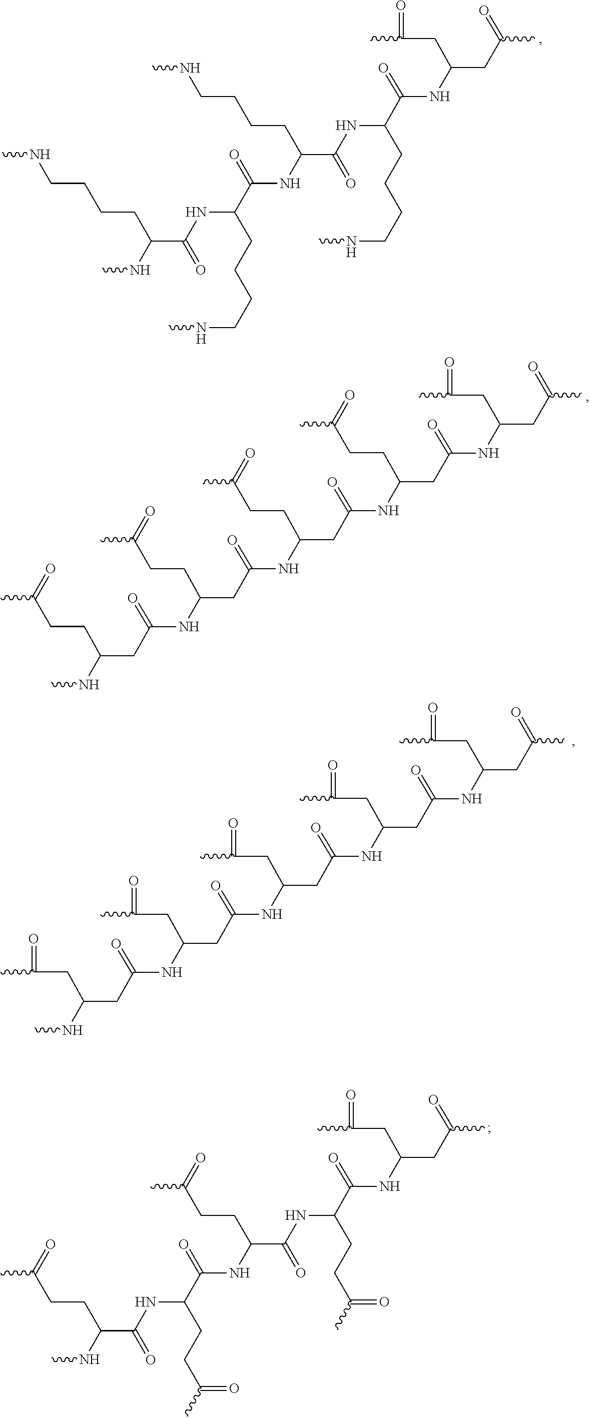

or a stereoisomer or a salt thereof.

In some embodiments, the compounds provided herein comprise one or more of following formulae: hexavalent 3+3

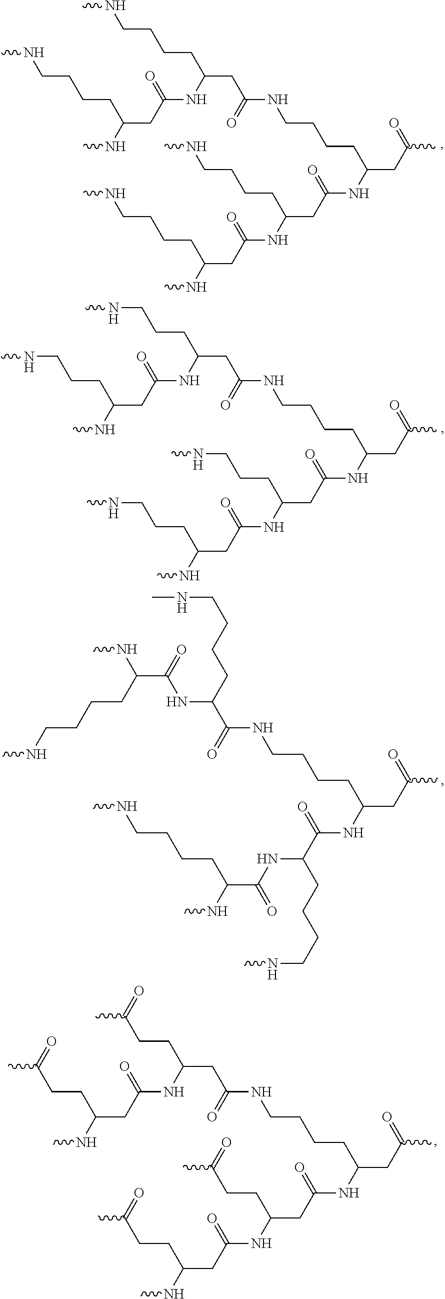

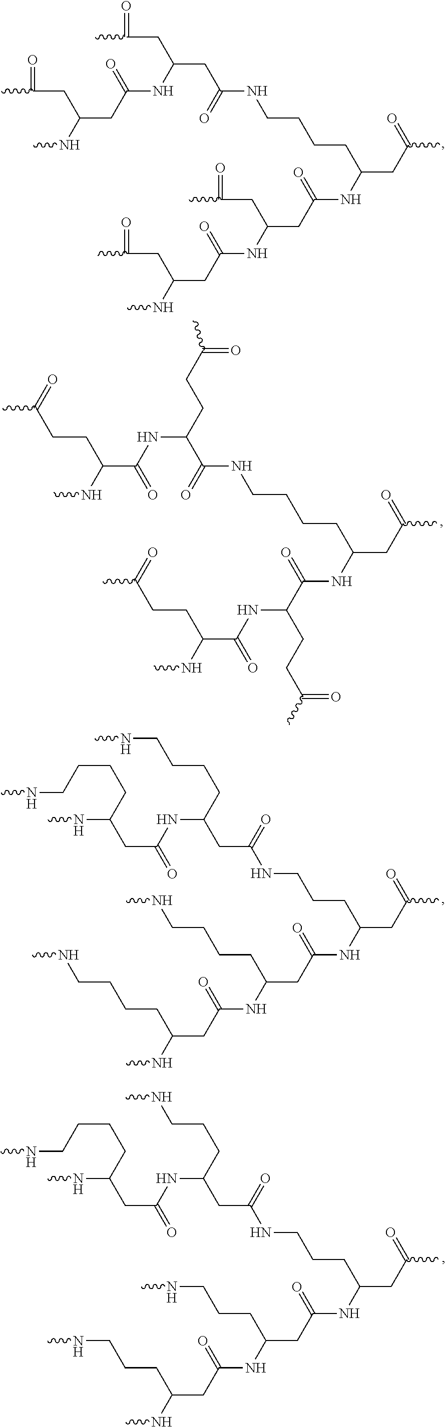

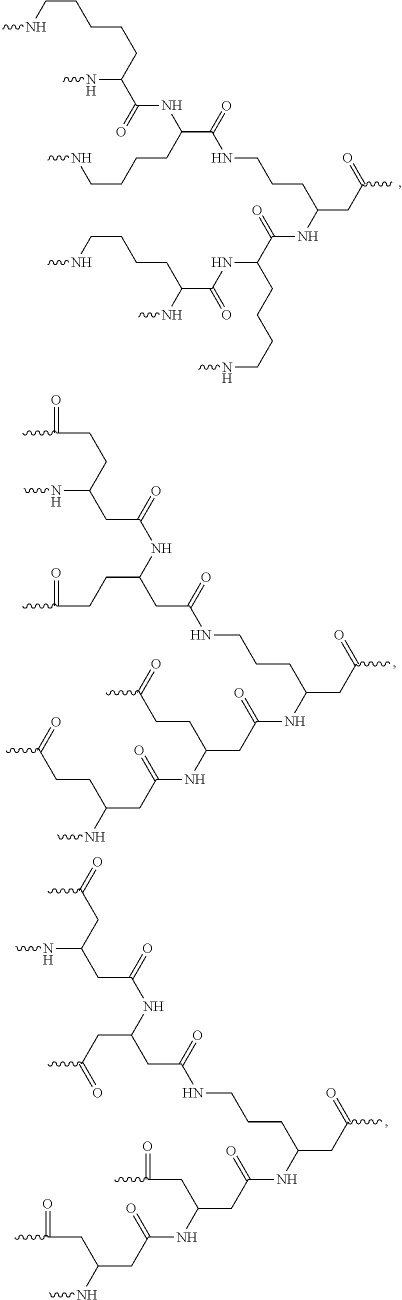

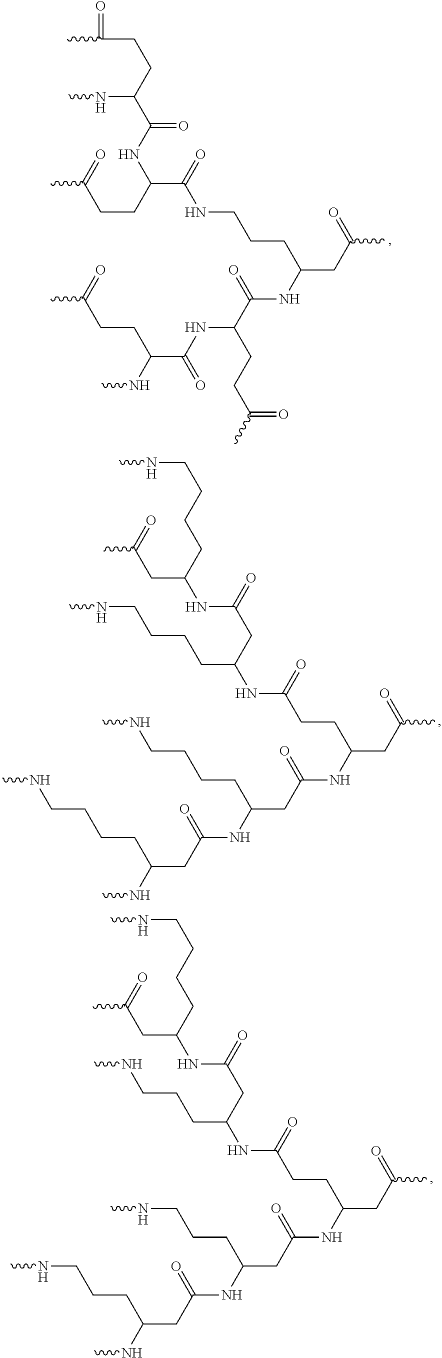

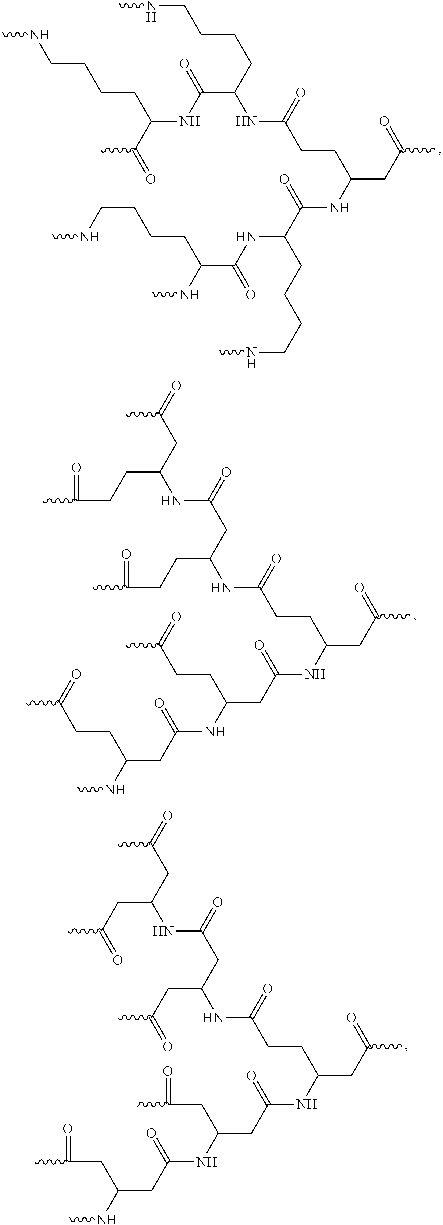

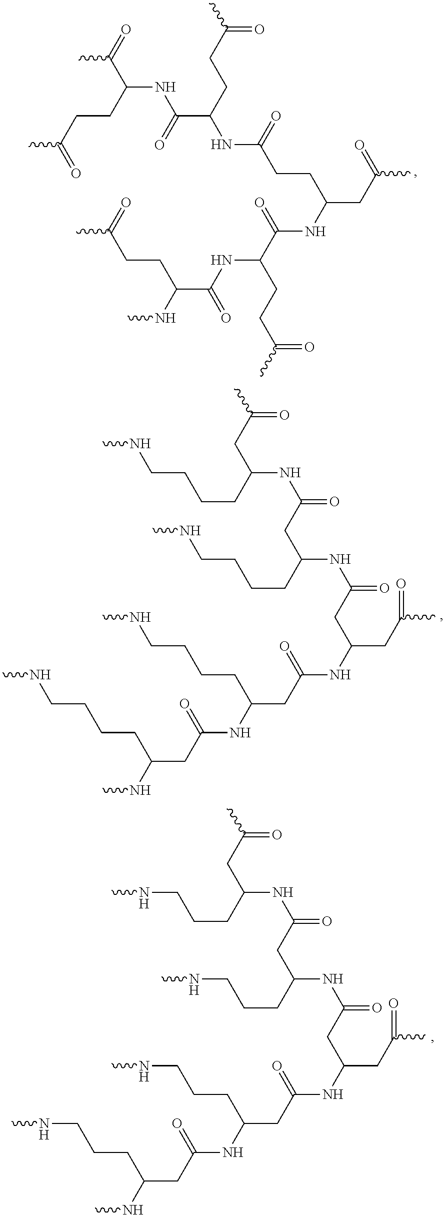

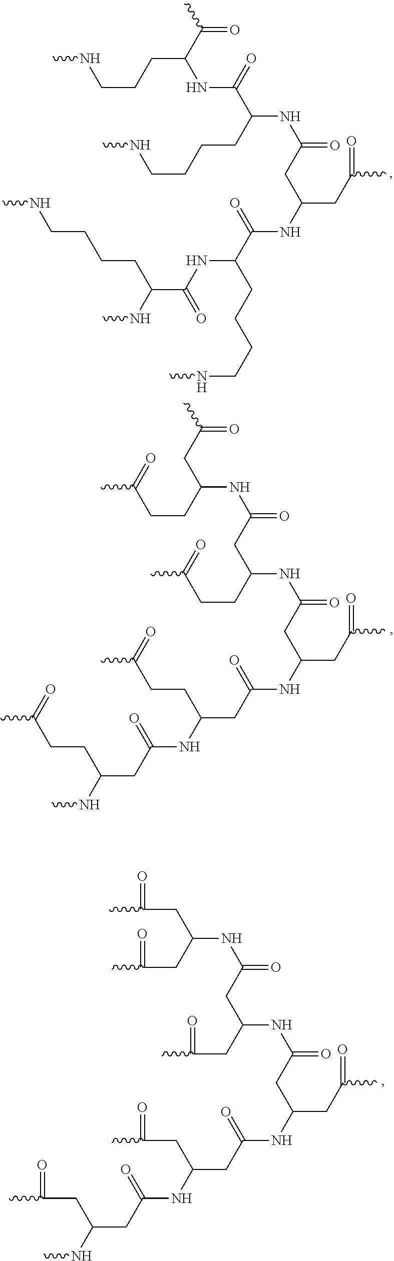

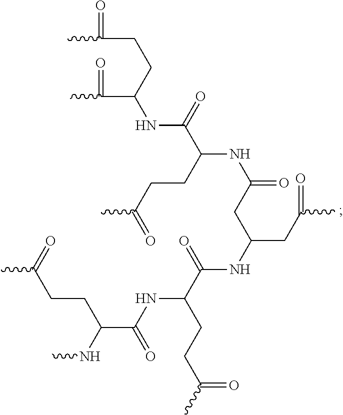

or a stereoisomer or a salt thereof.

In some embodiments, the compounds provided herein comprise one or more of following formulae: hexavalent 2+2+2

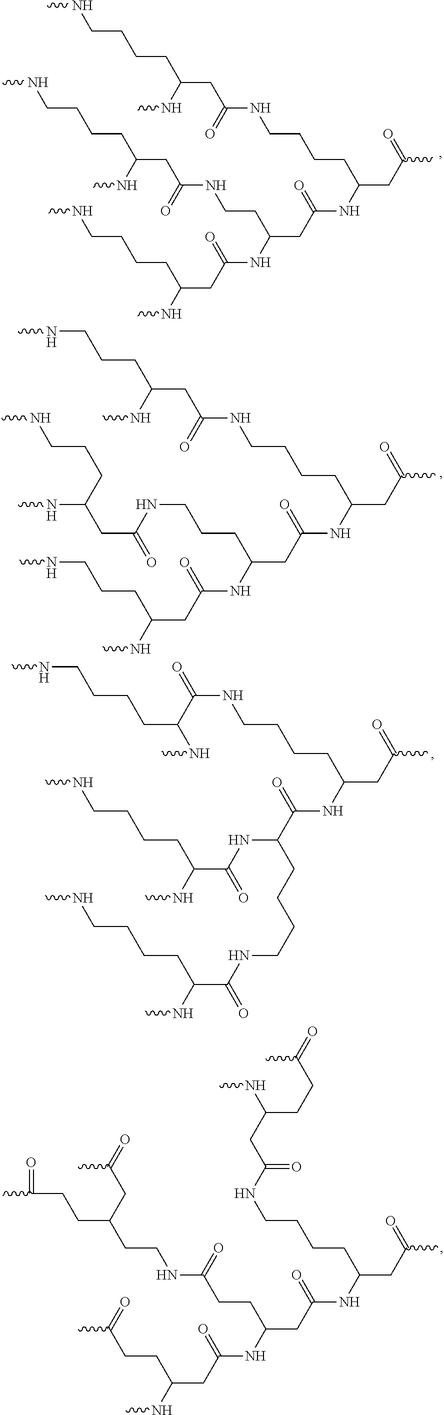

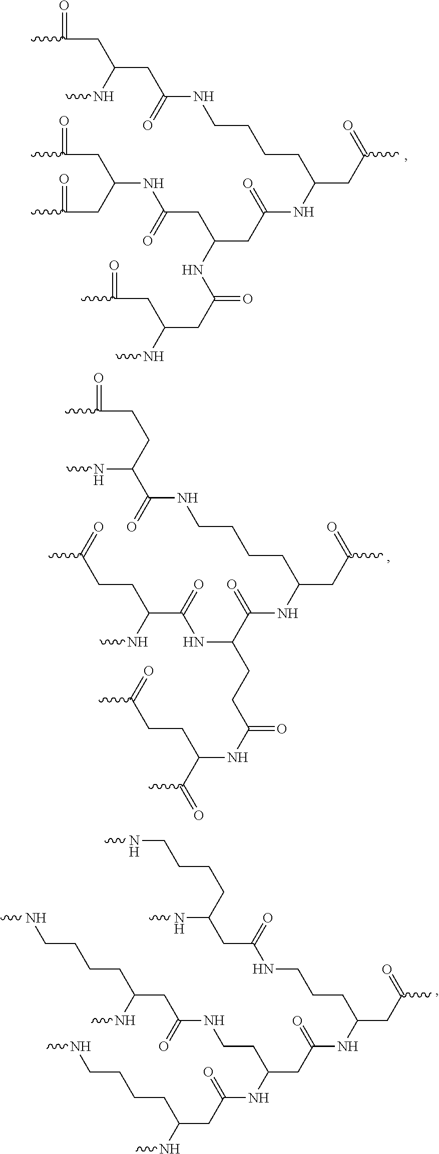

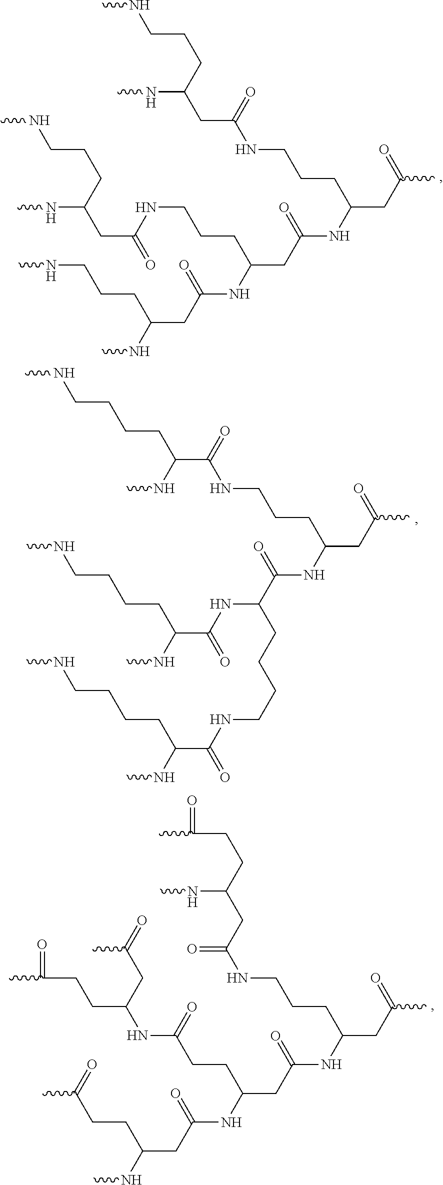

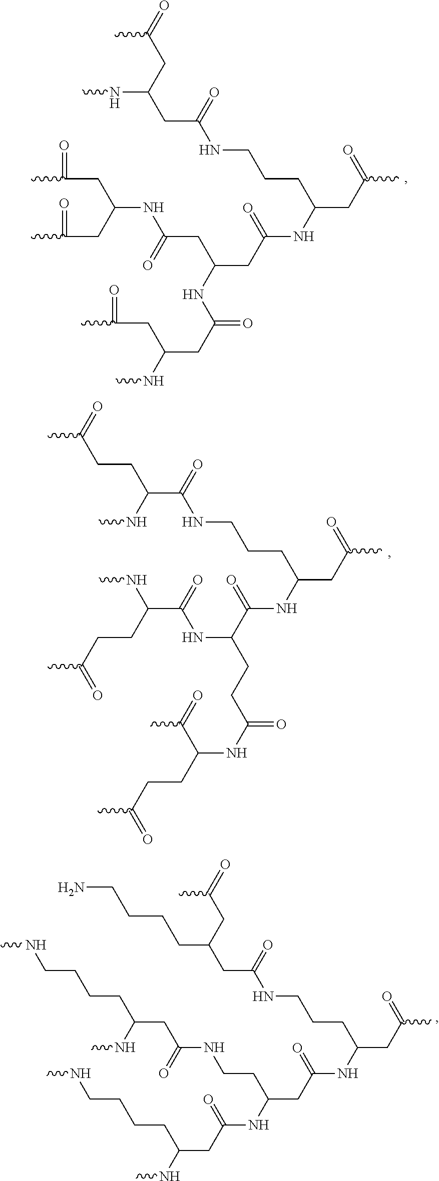

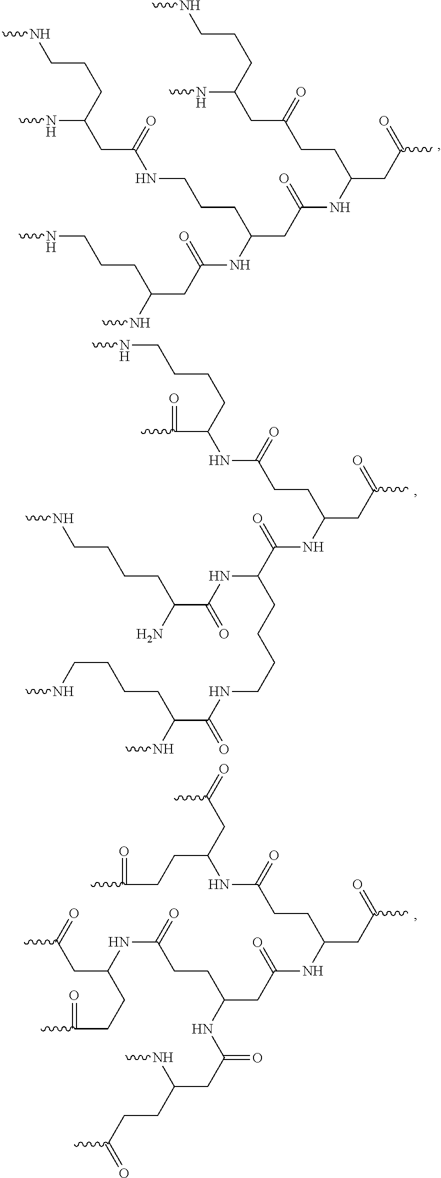

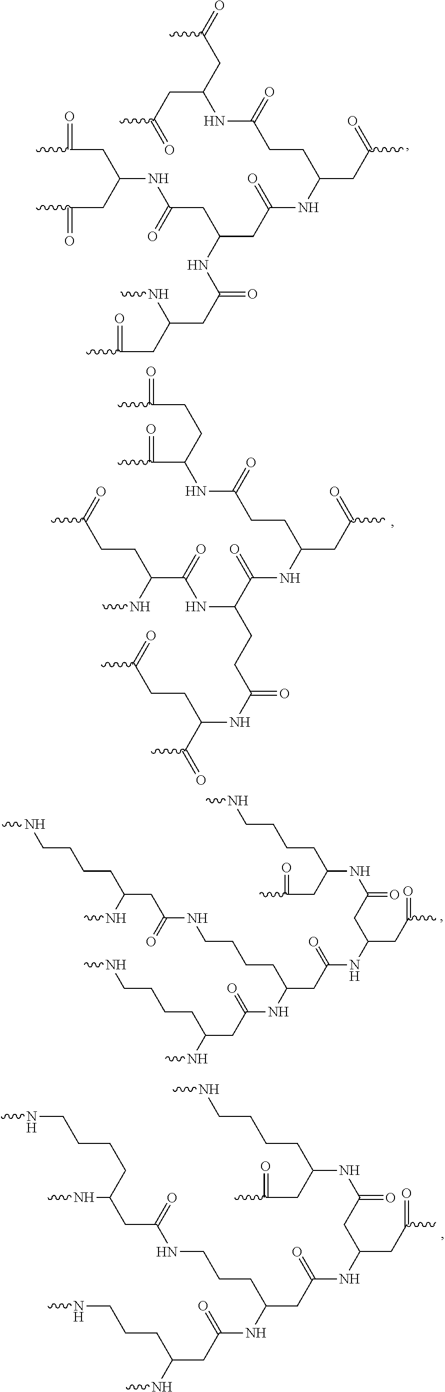

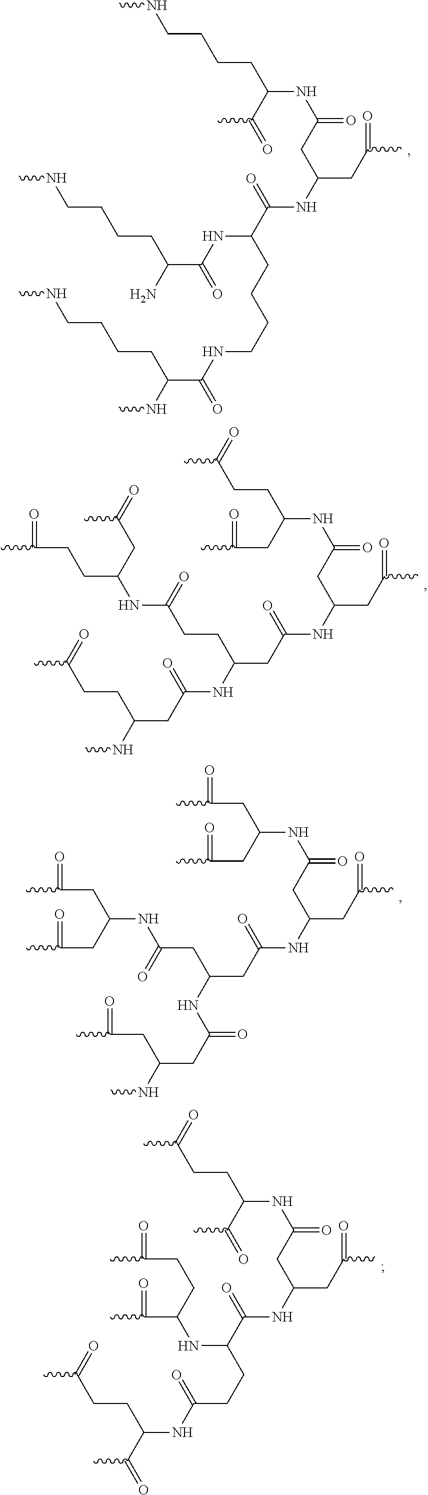

or a stereoisomer or a salt thereof.

In some embodiments, the compounds provided herein comprise one or more of following formulae:

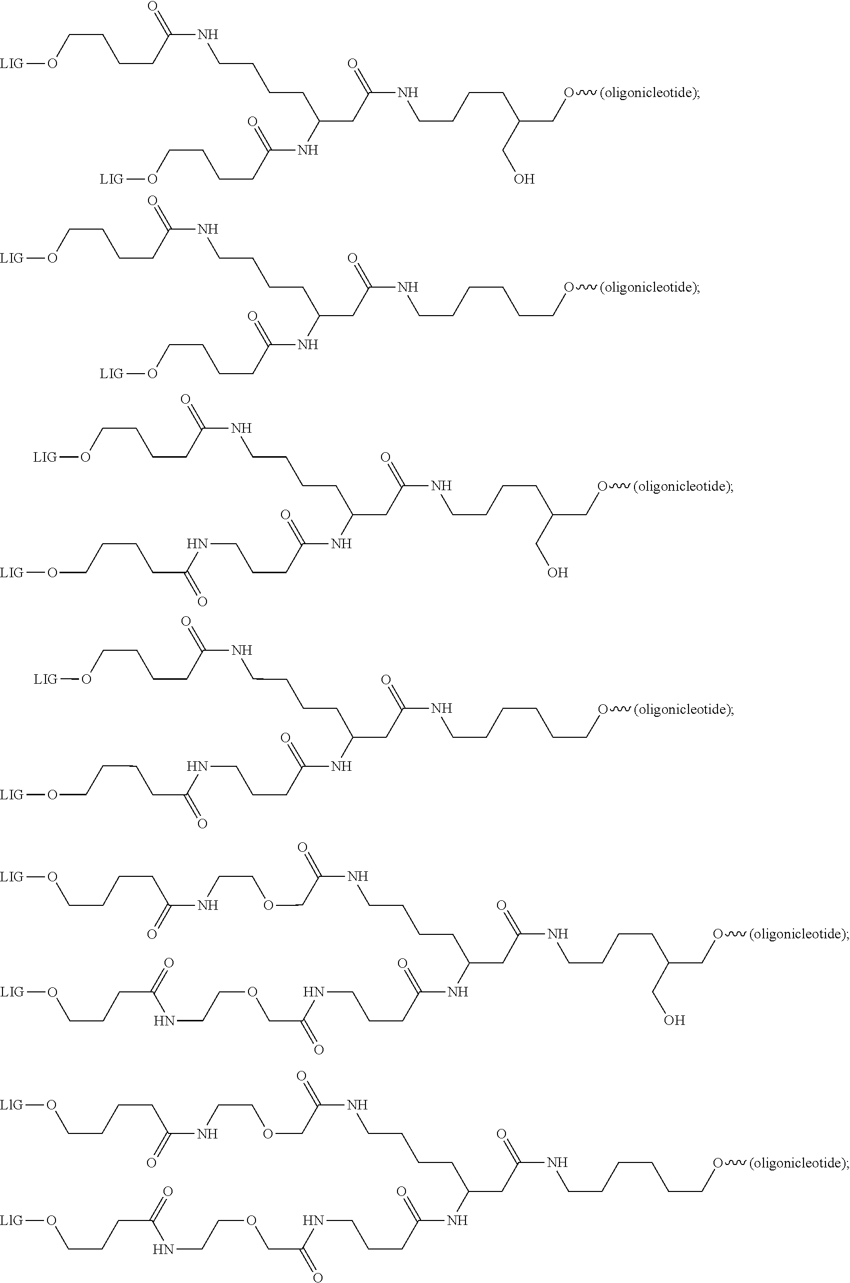

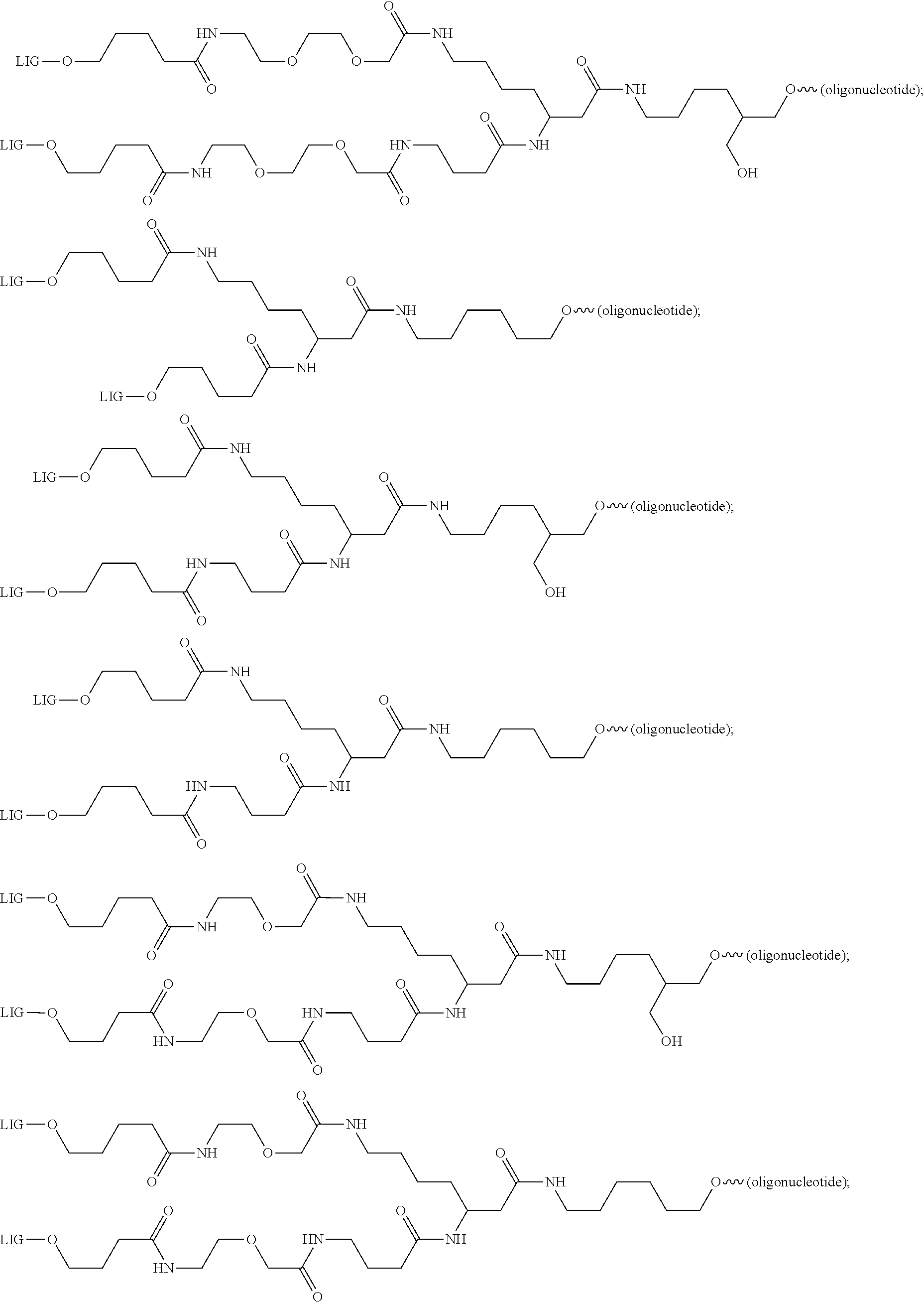

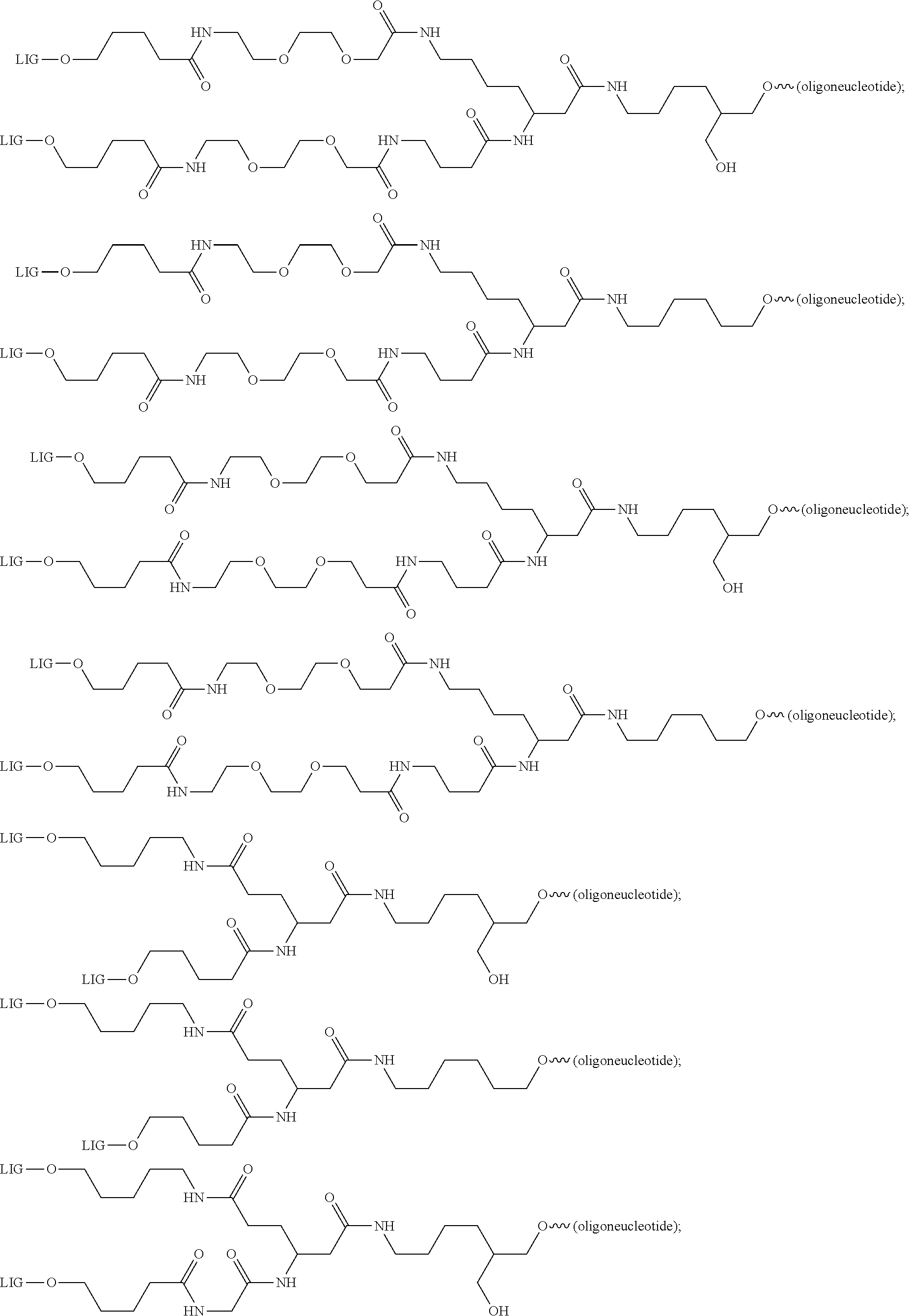

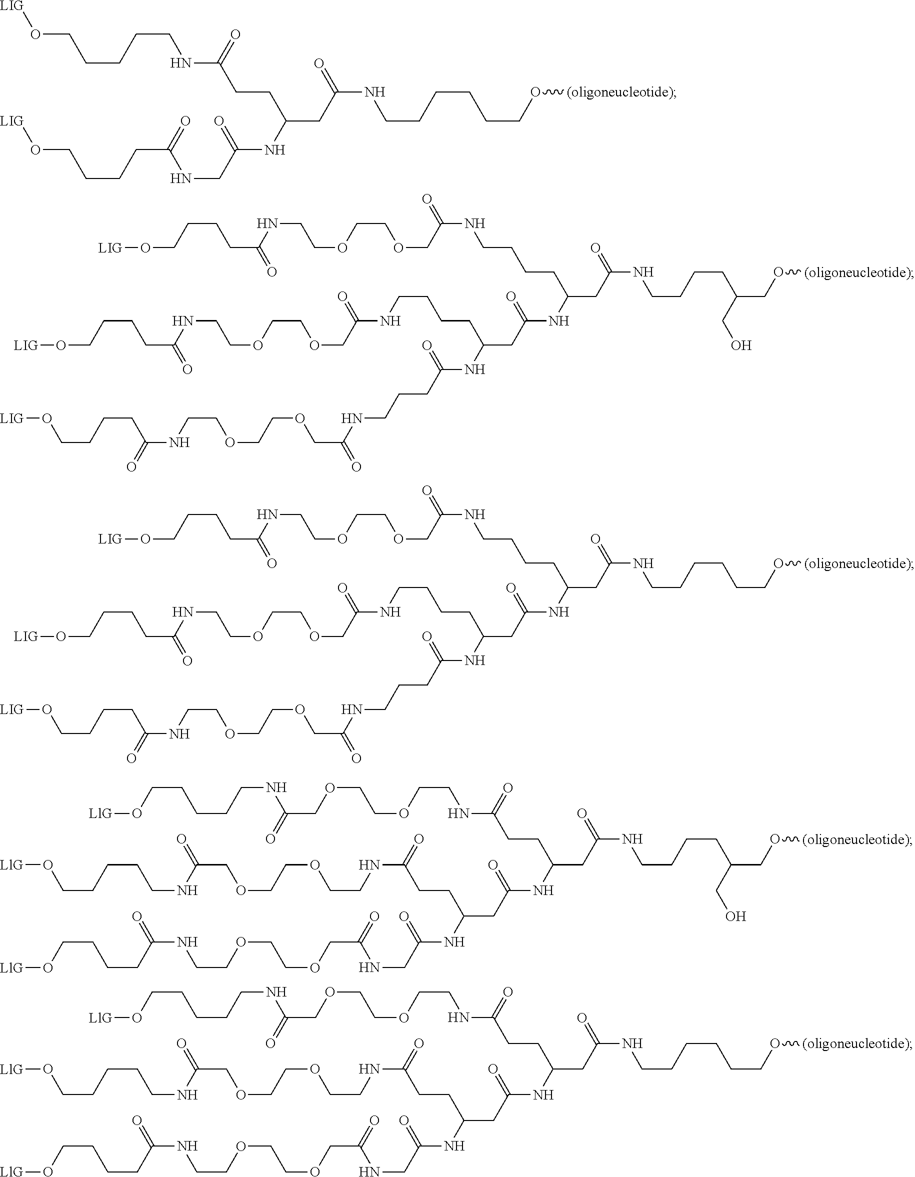

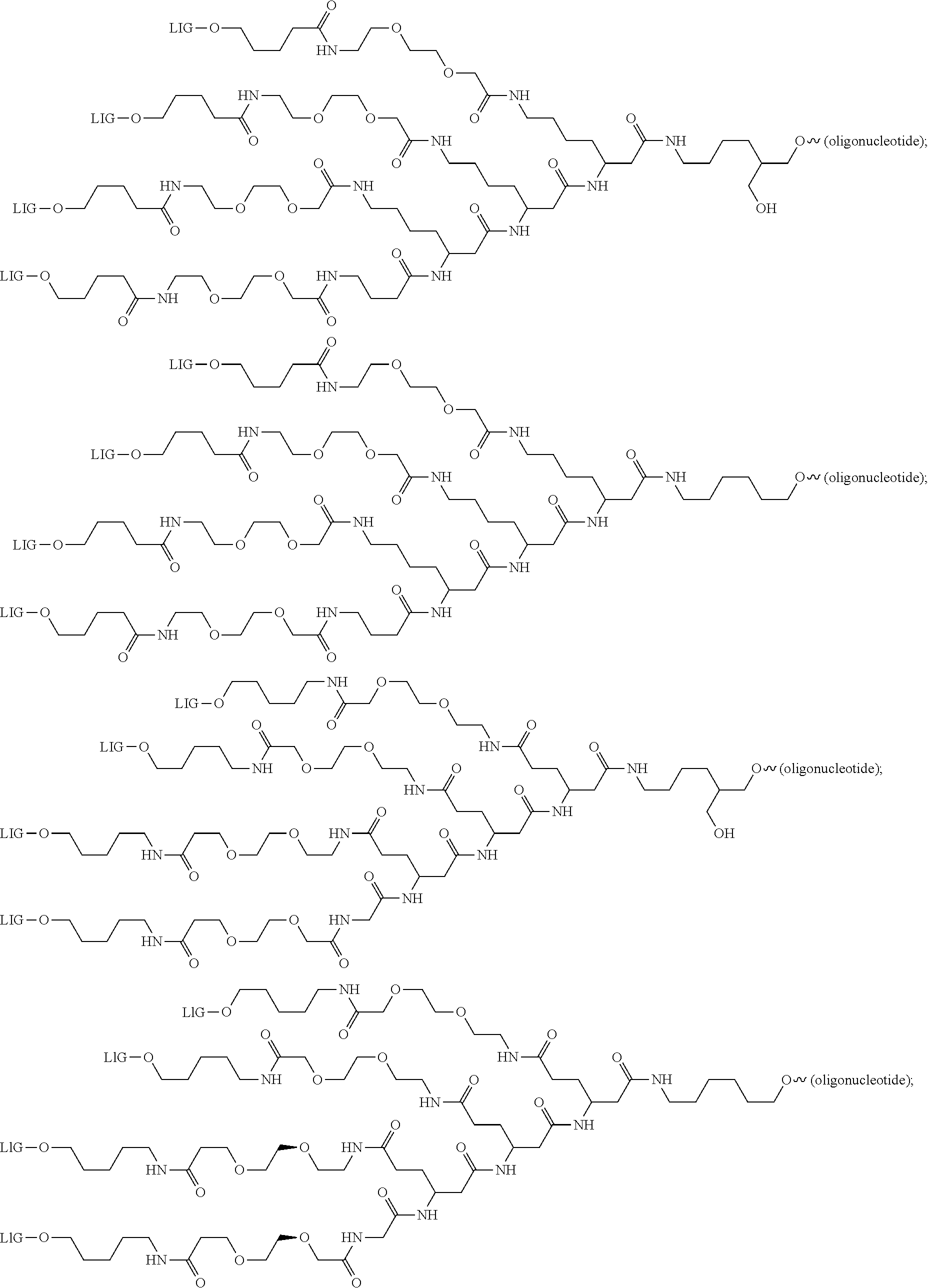

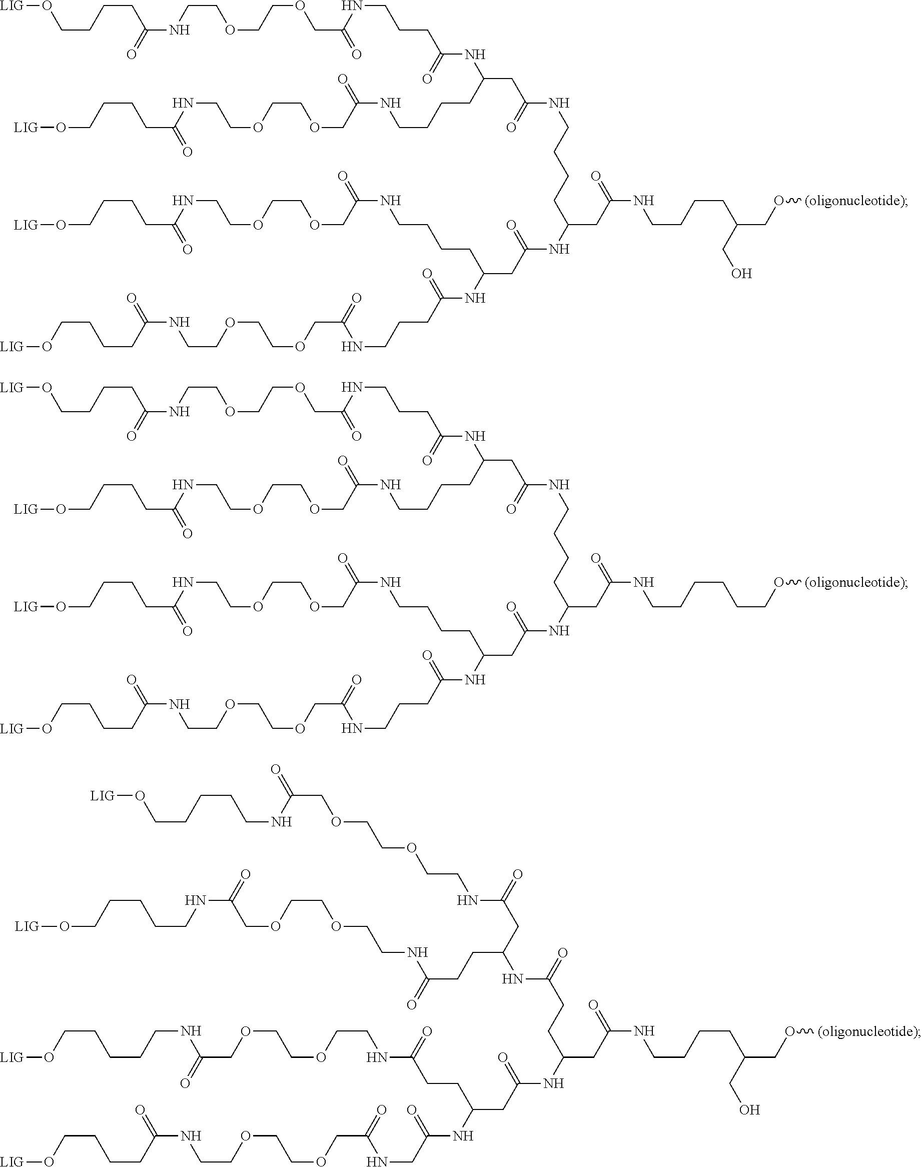

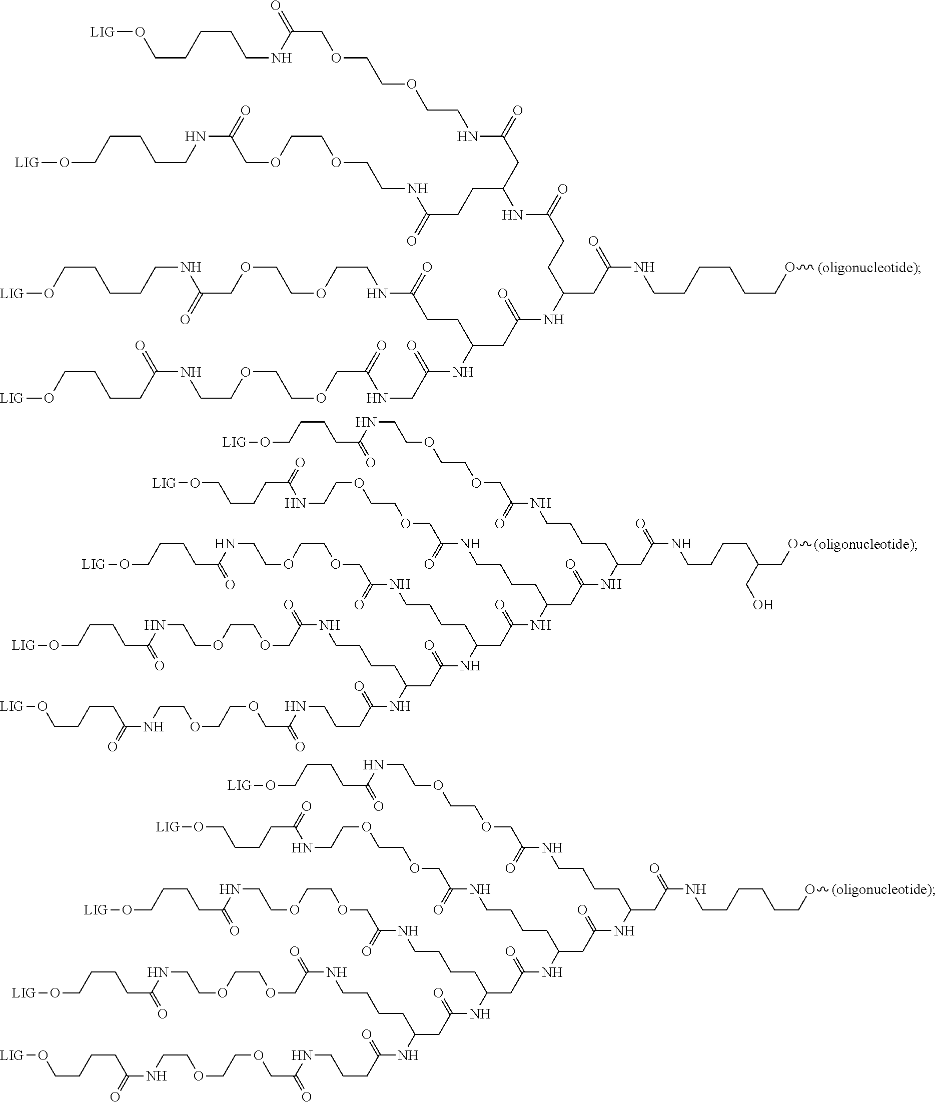

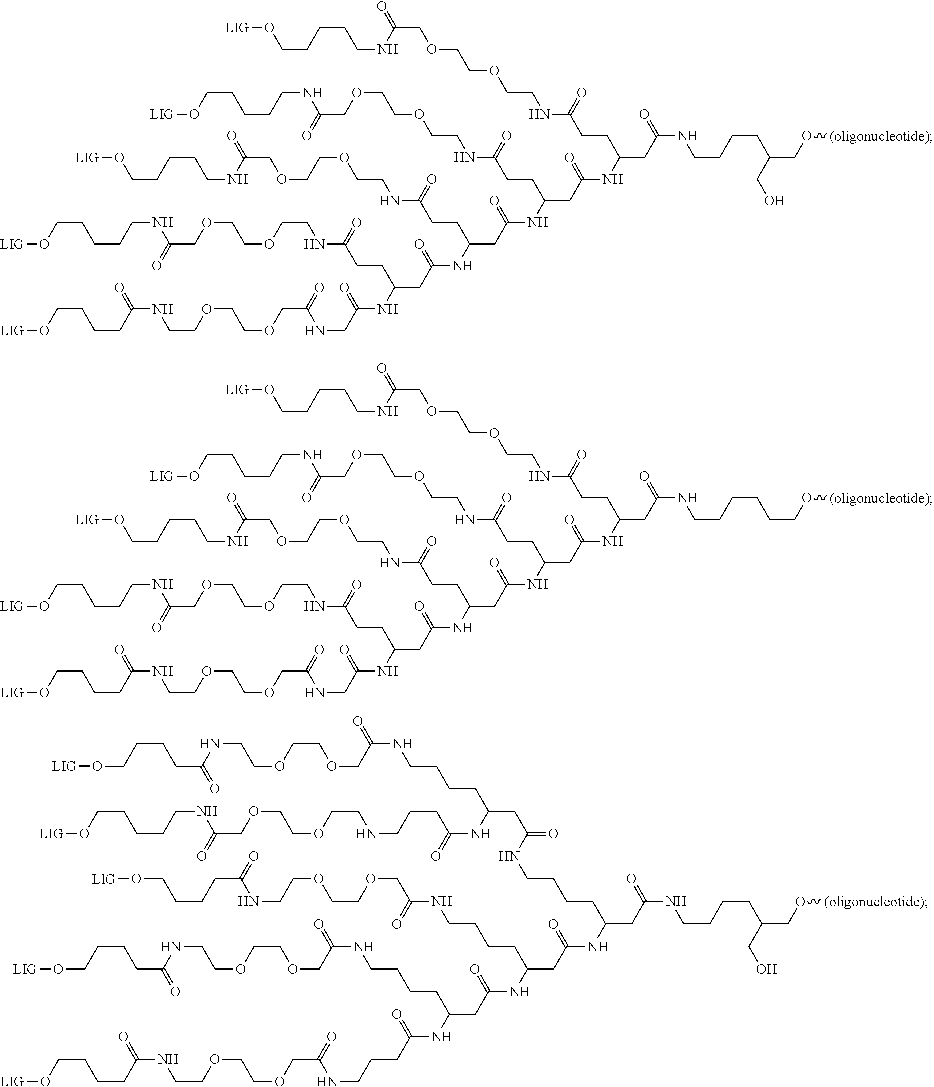

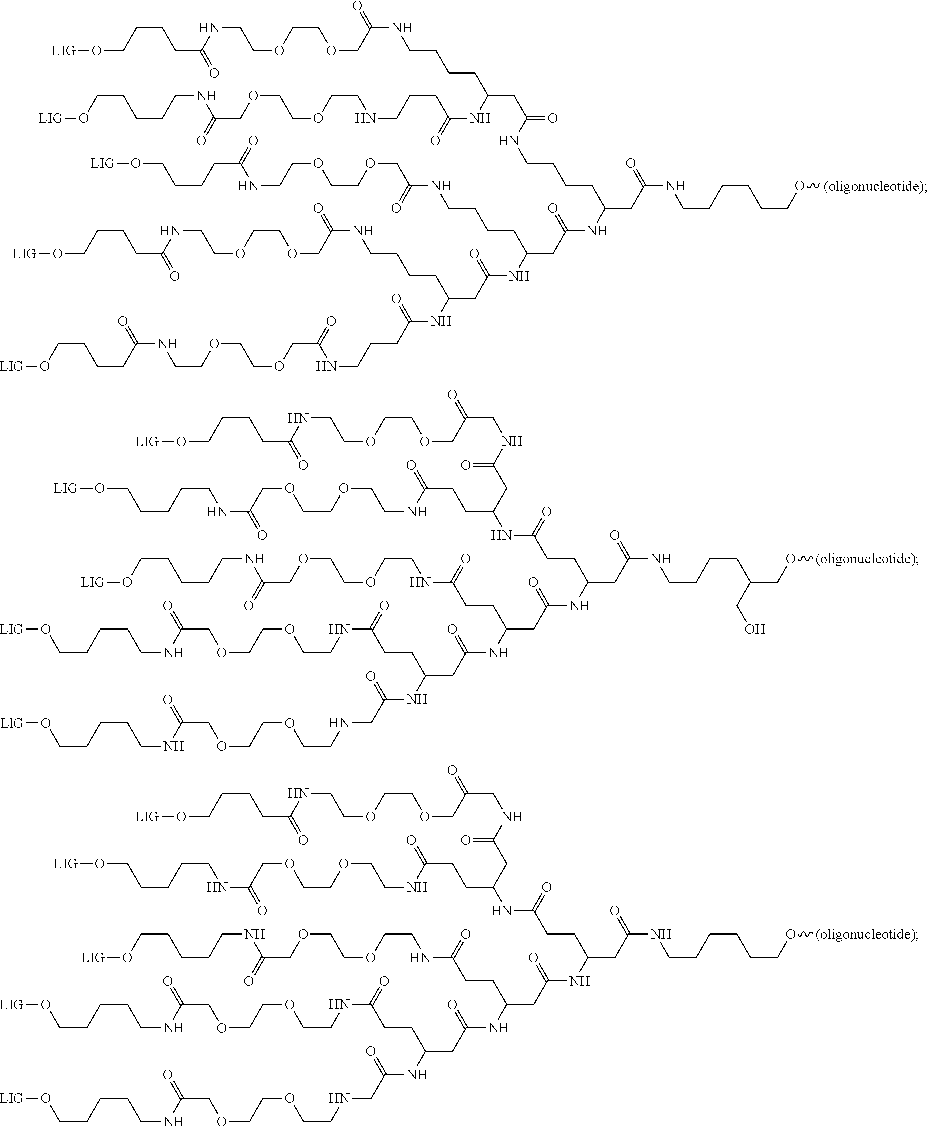

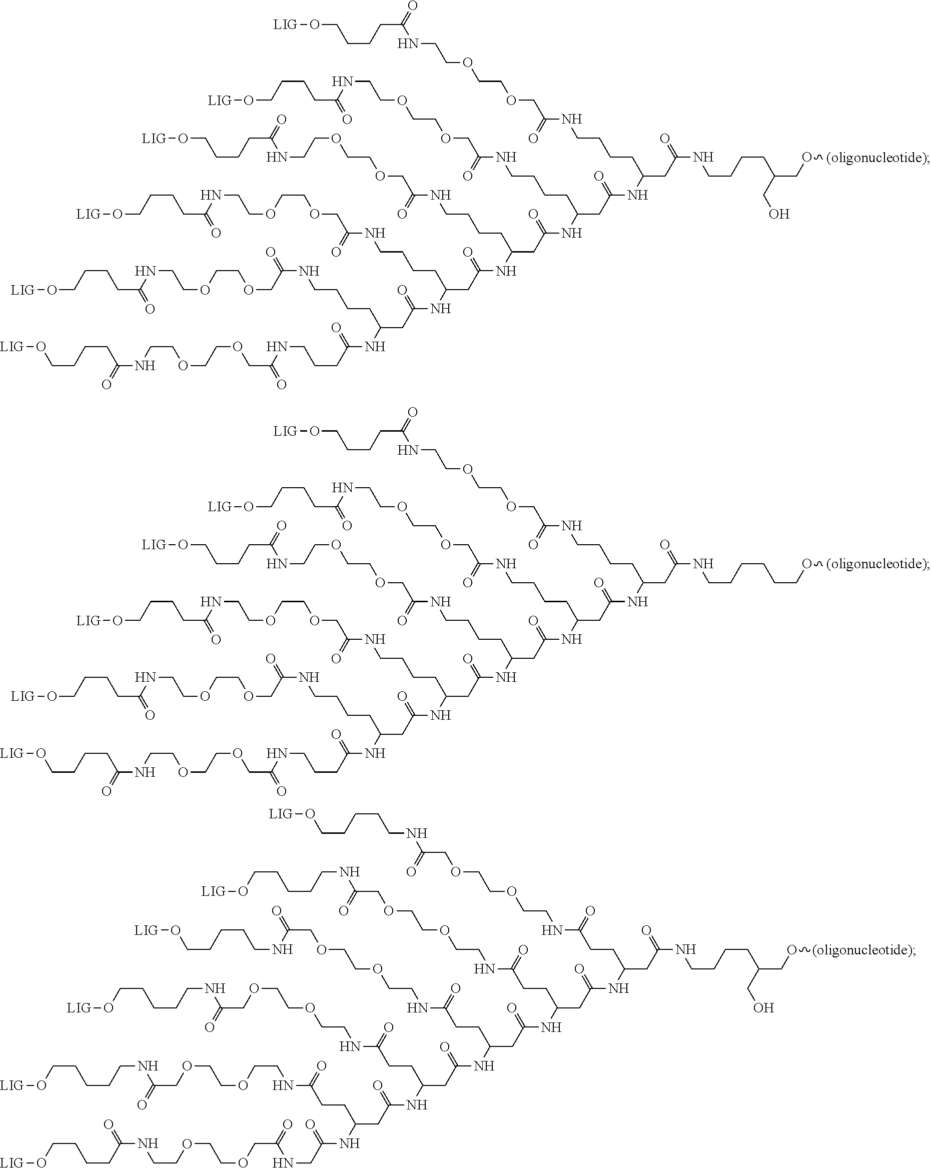

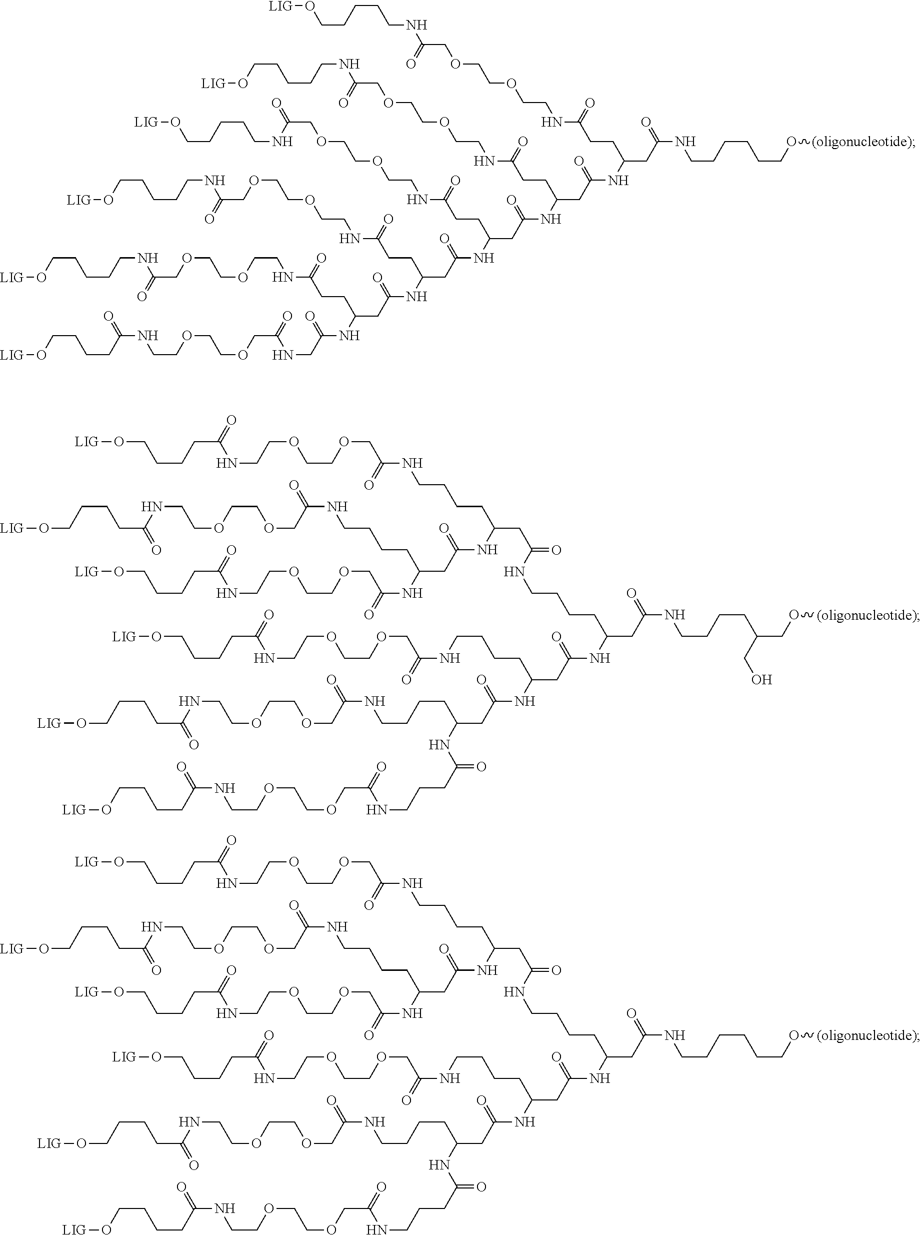

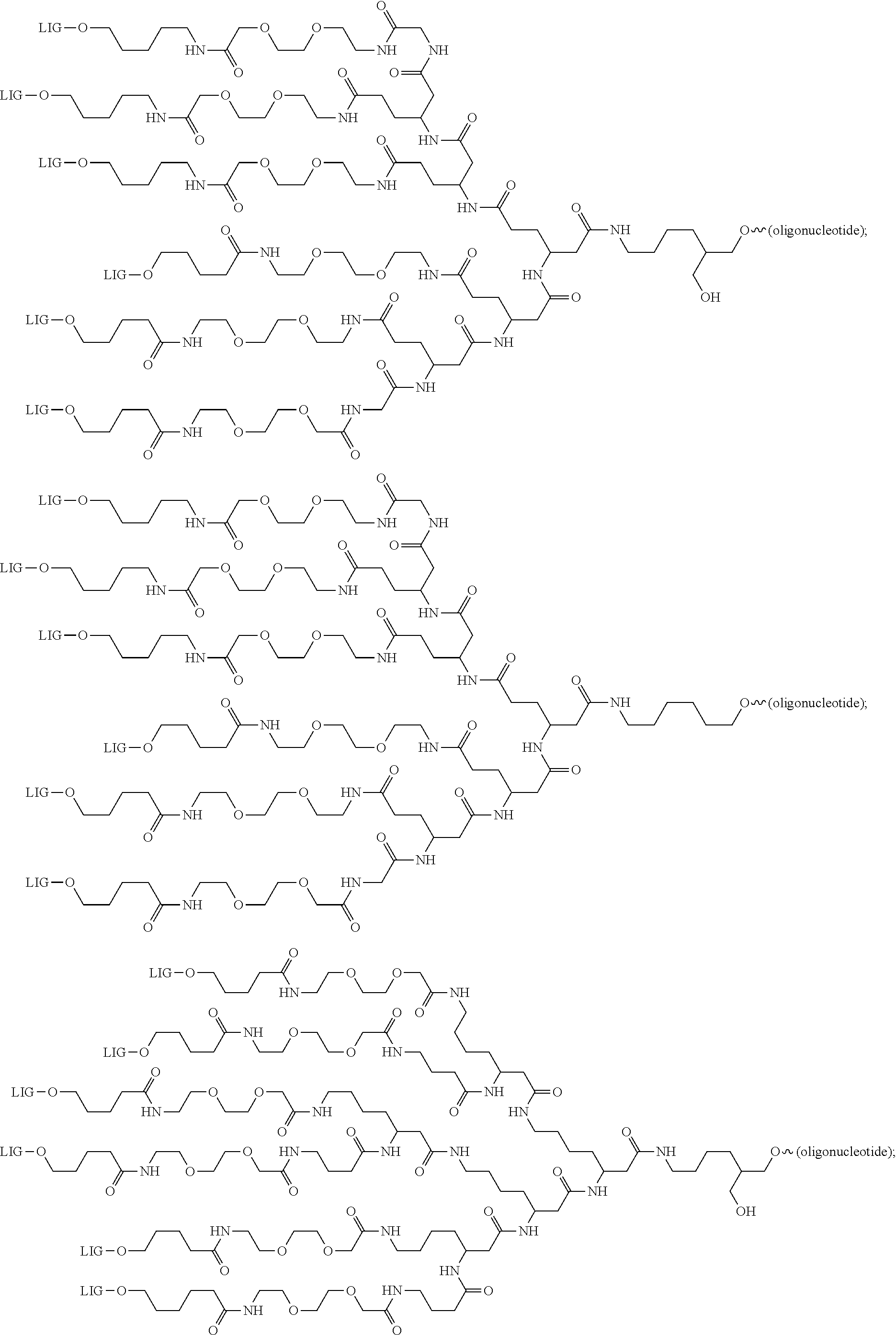

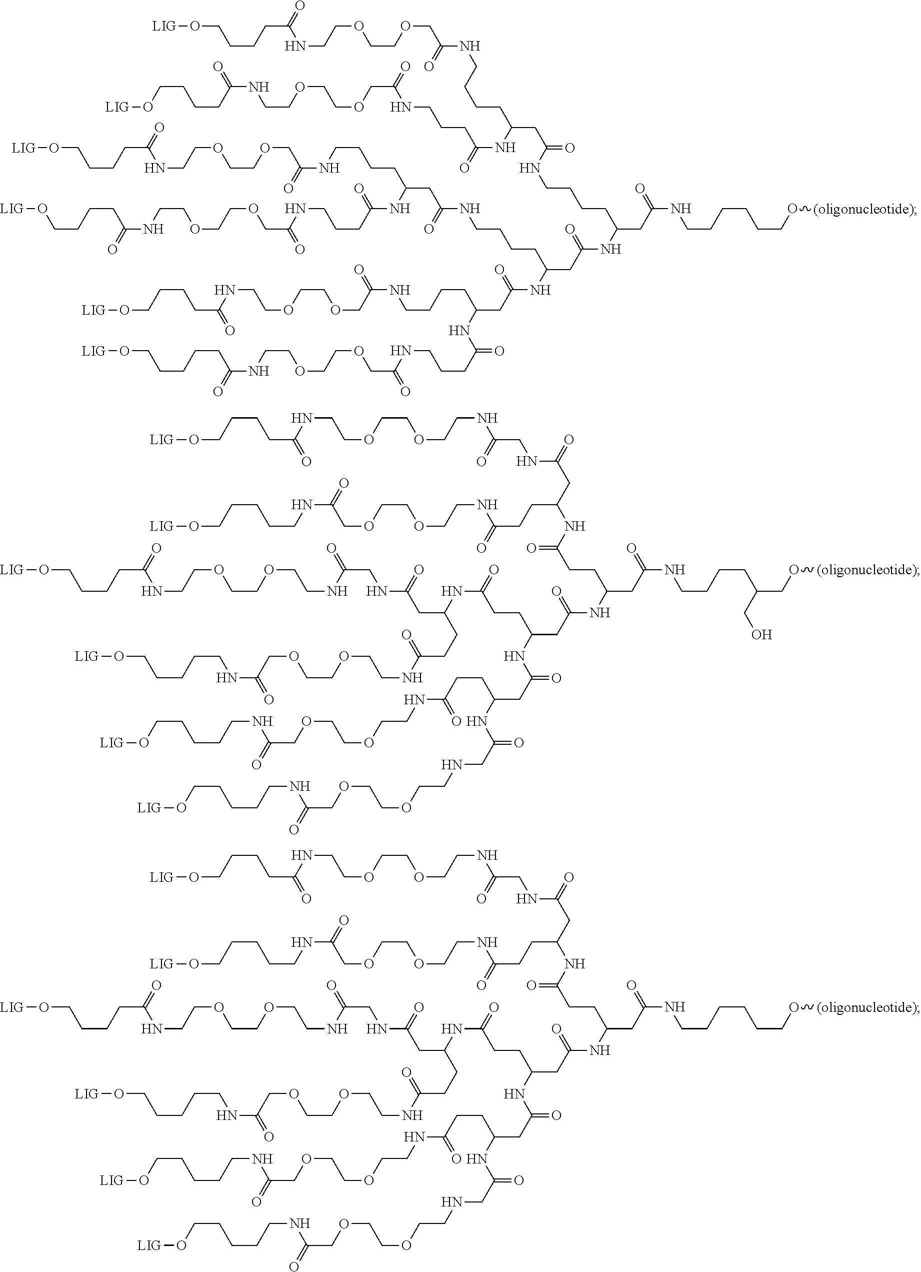

or a stereoisomer or a salt thereof,
wherein
each LIG is, independently, selected from carbohydrate receptor ligands such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies.

In some embodiments, the compounds provided herein comprise one or more of following formulae:

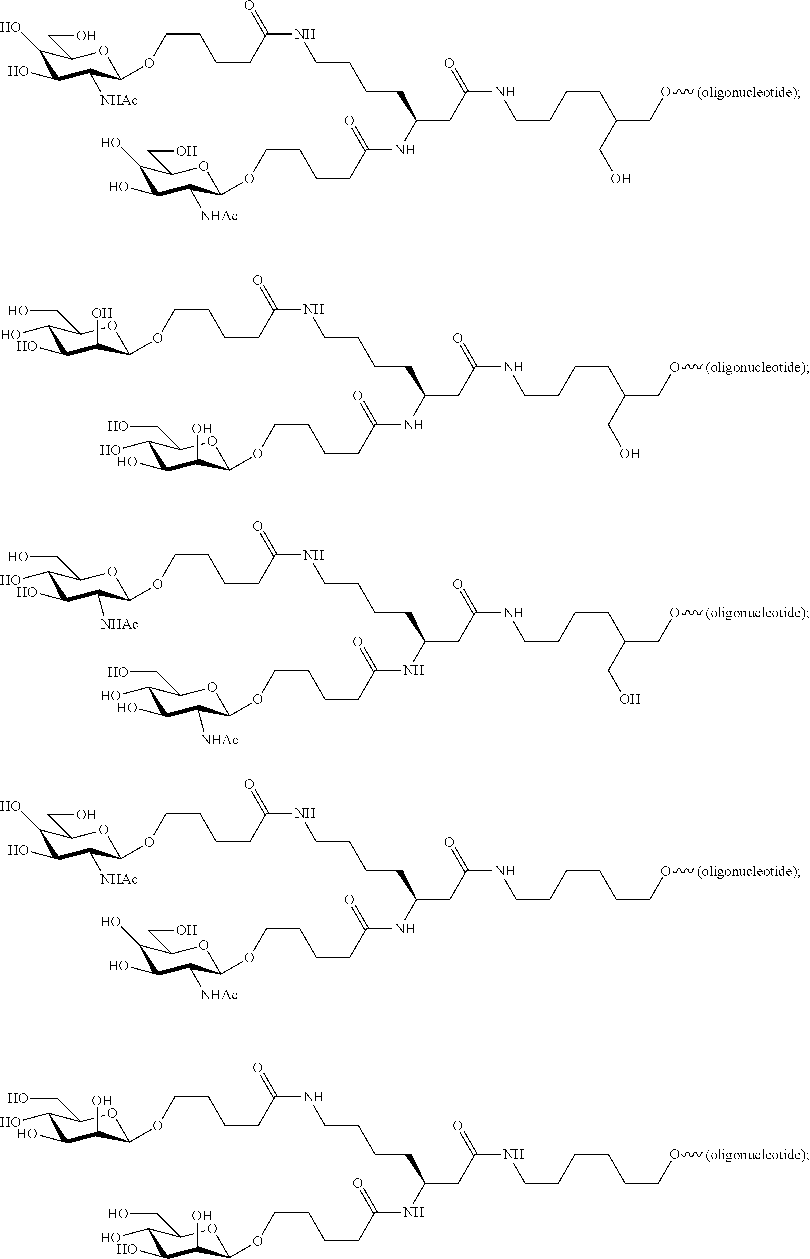

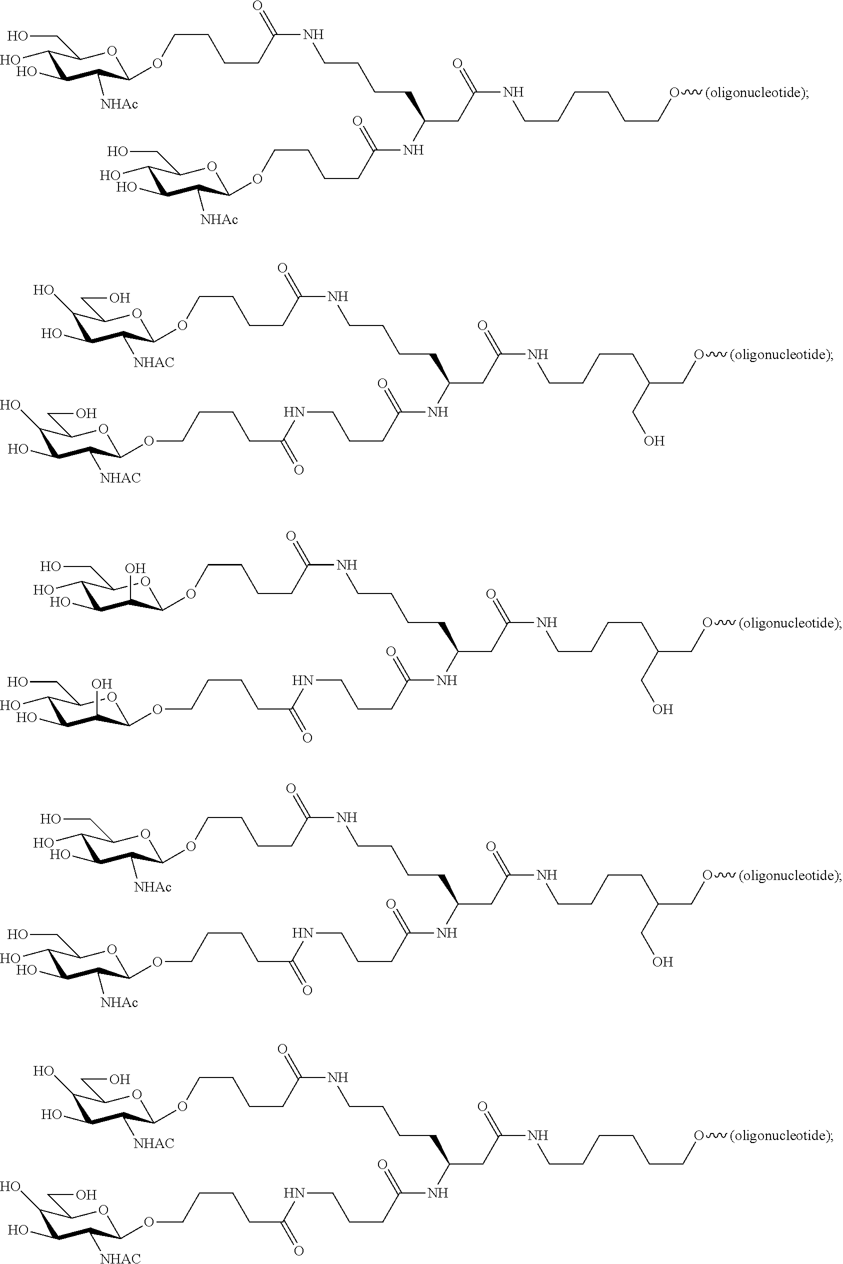

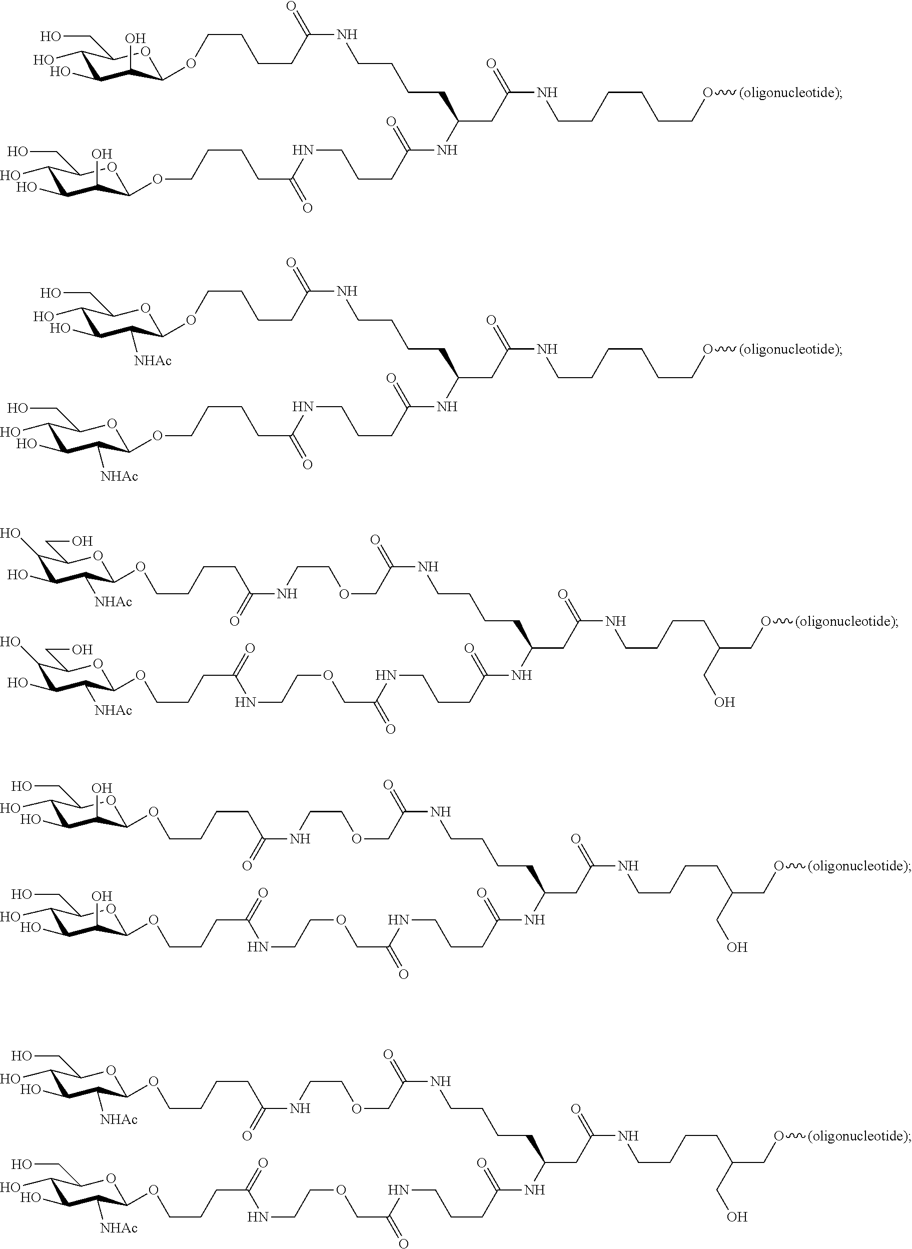

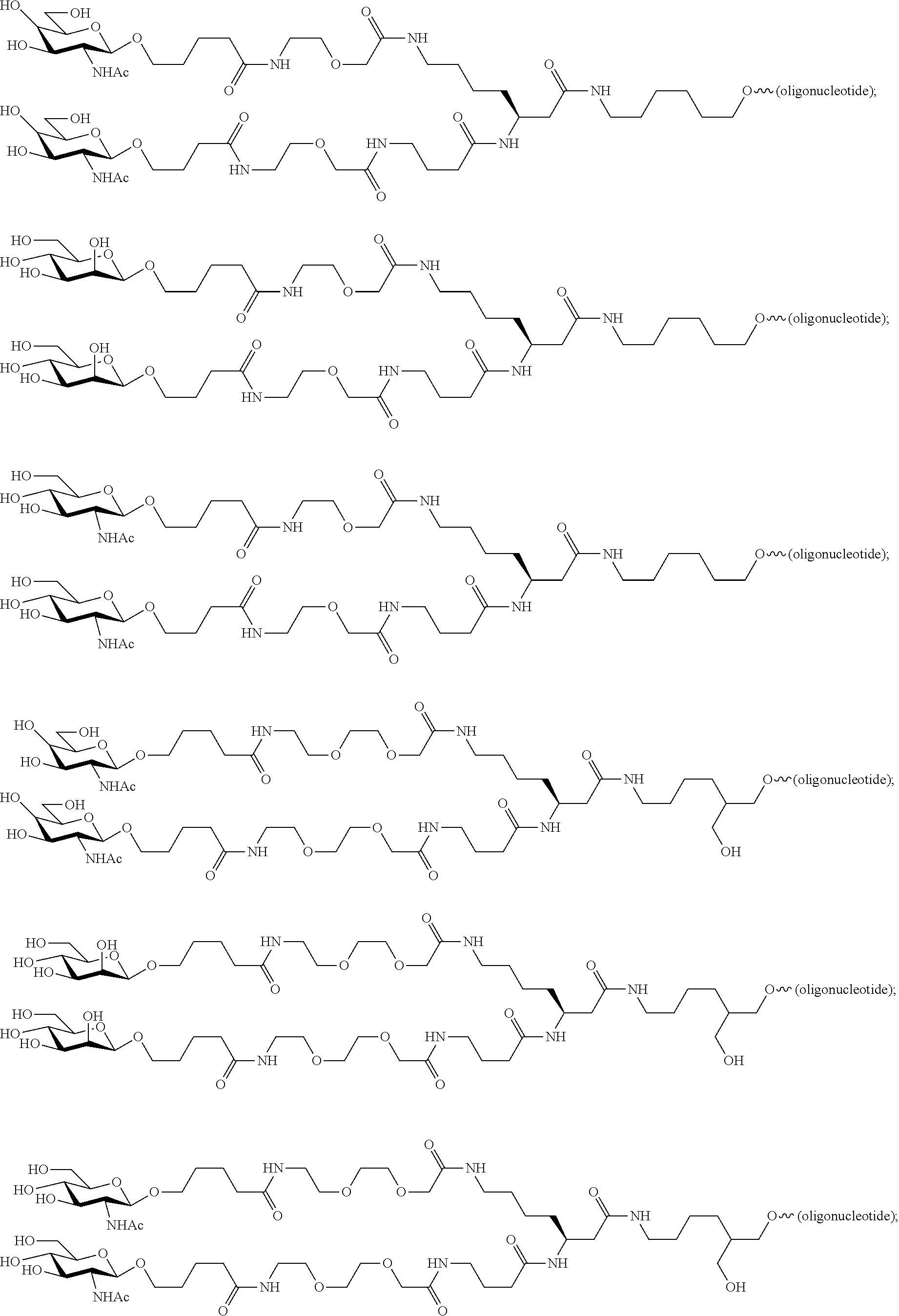

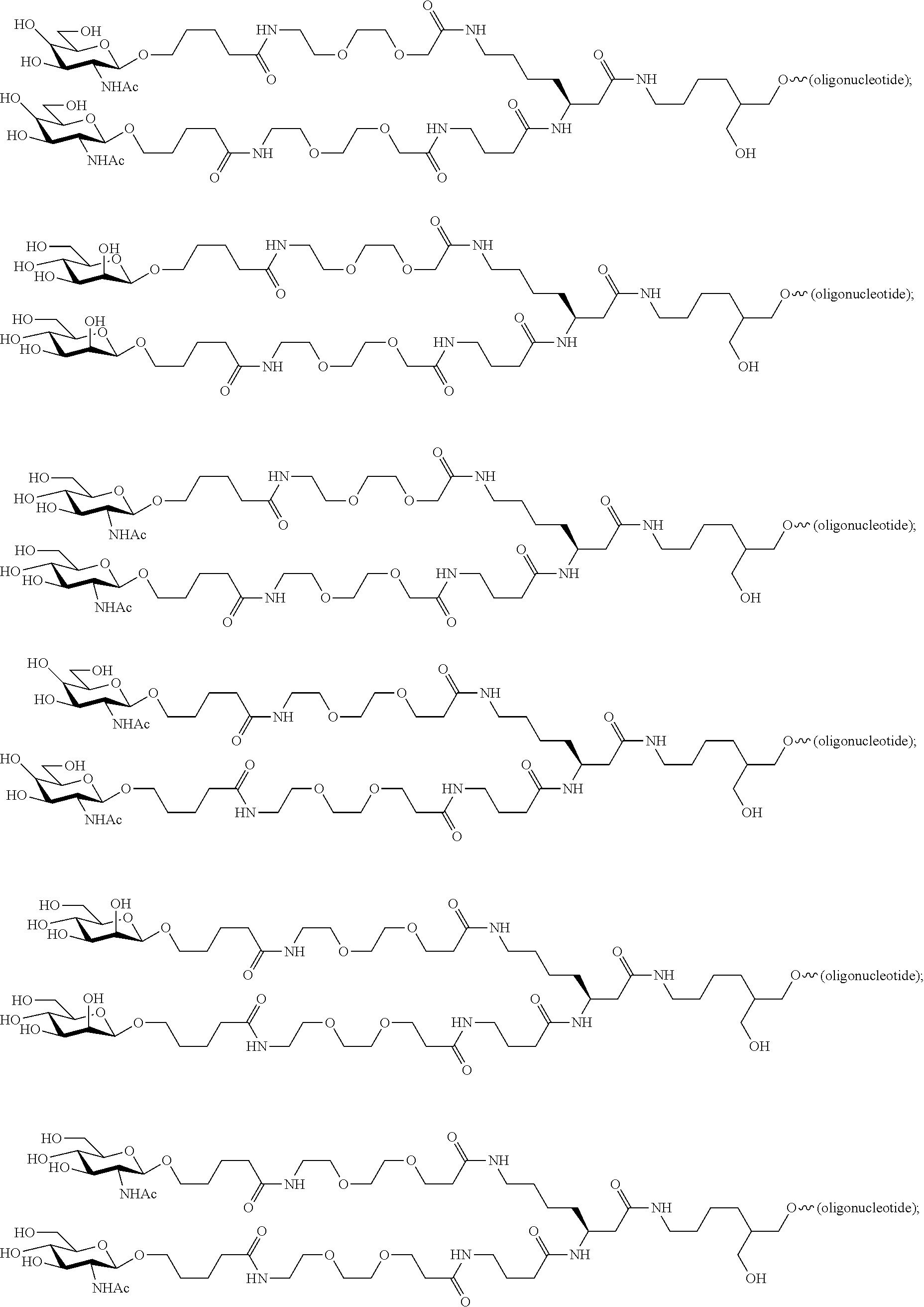

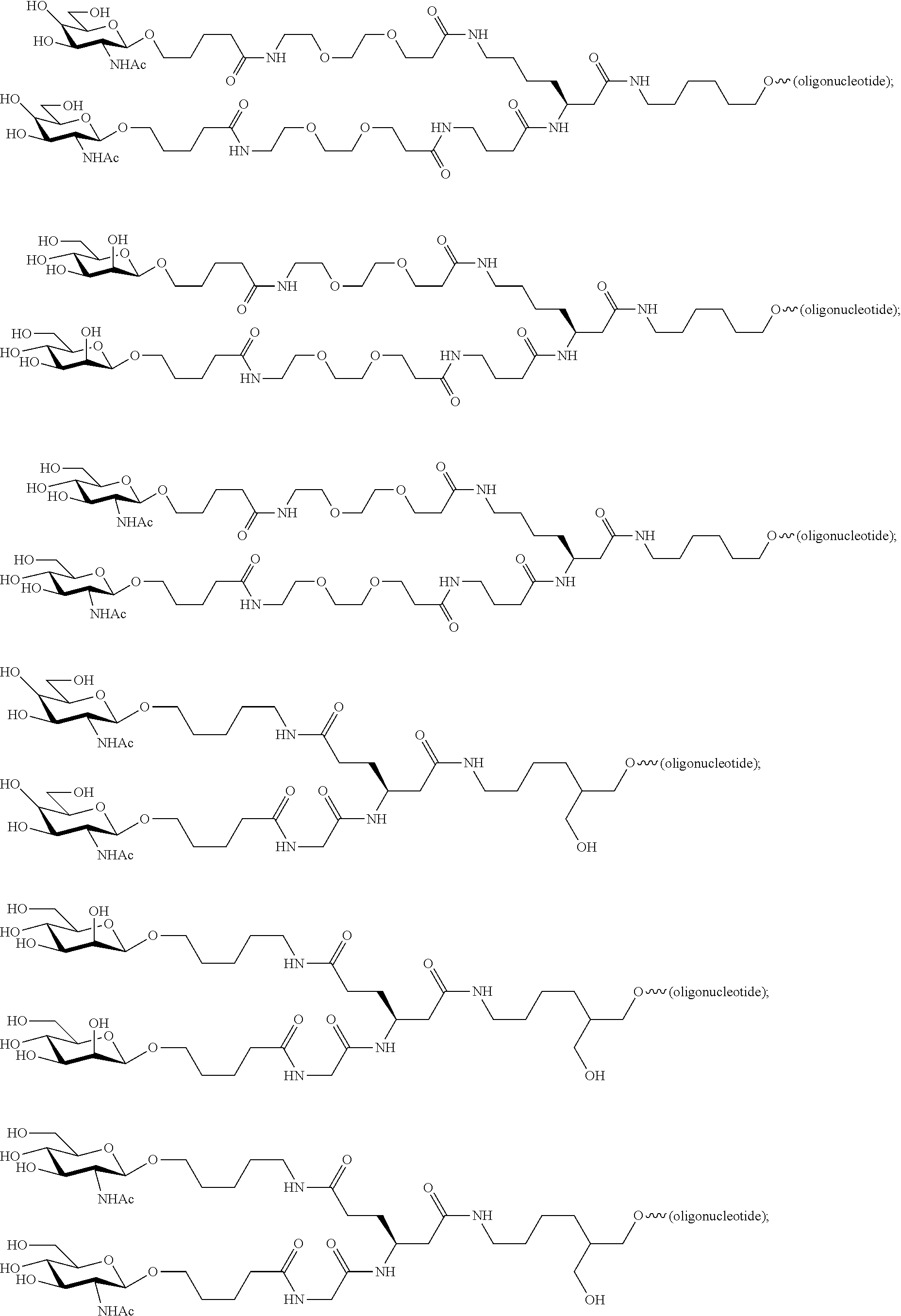

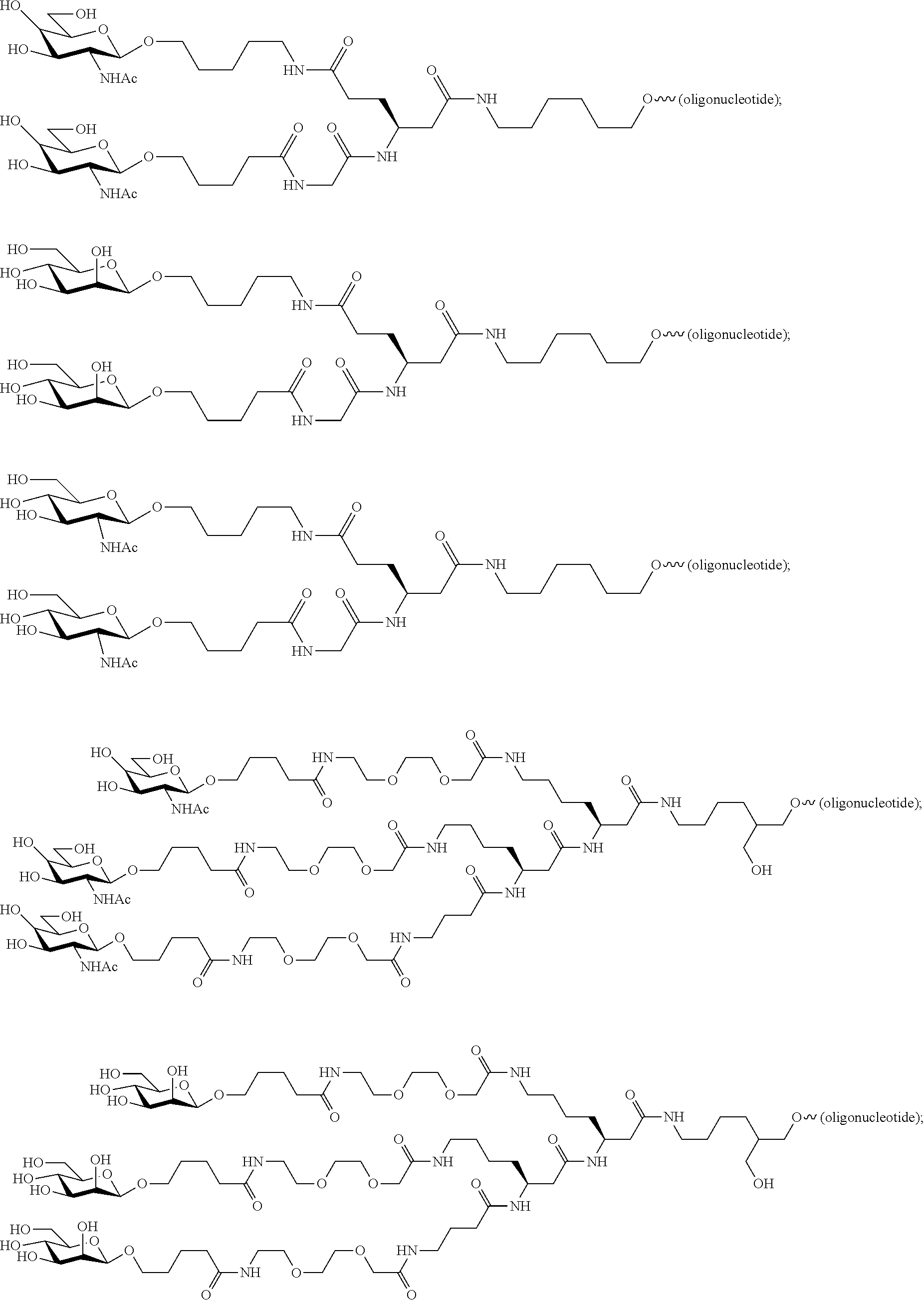

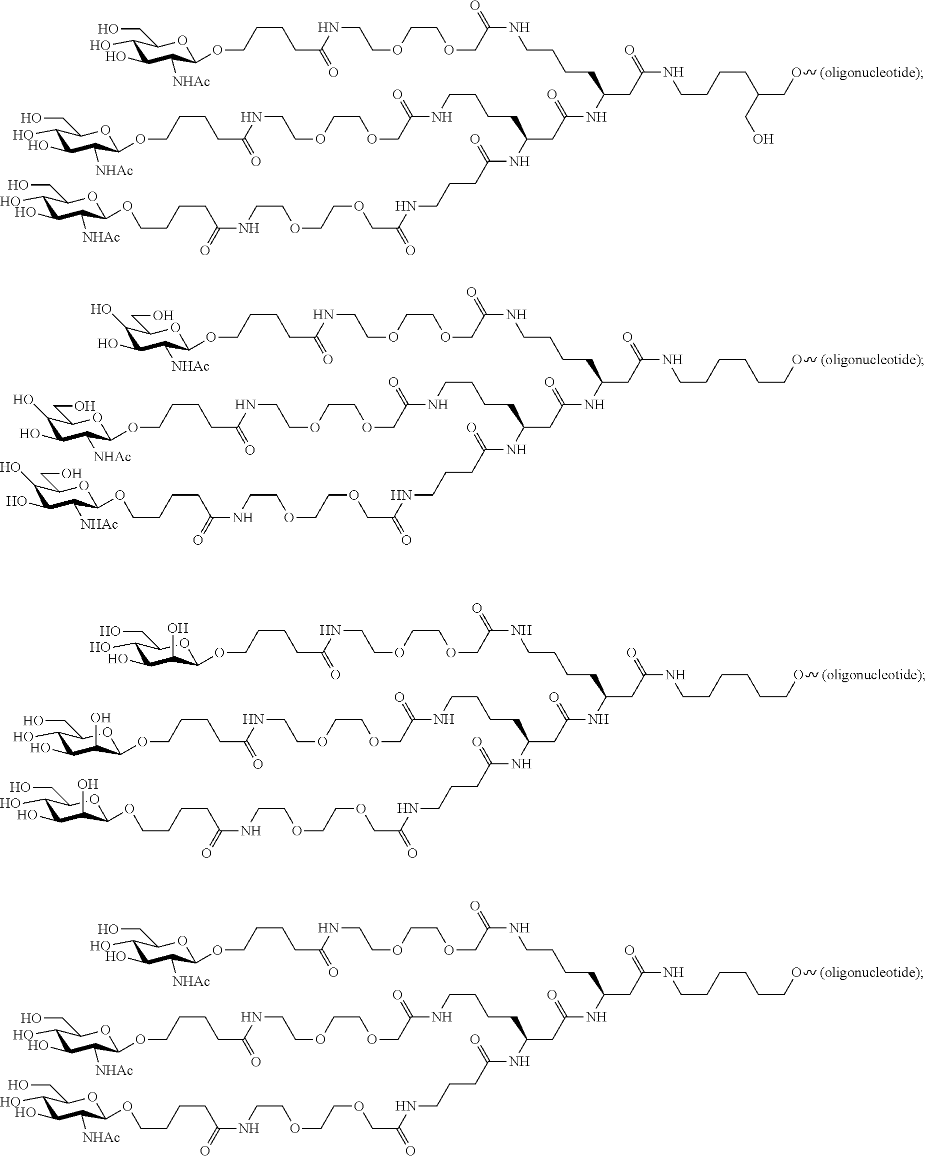

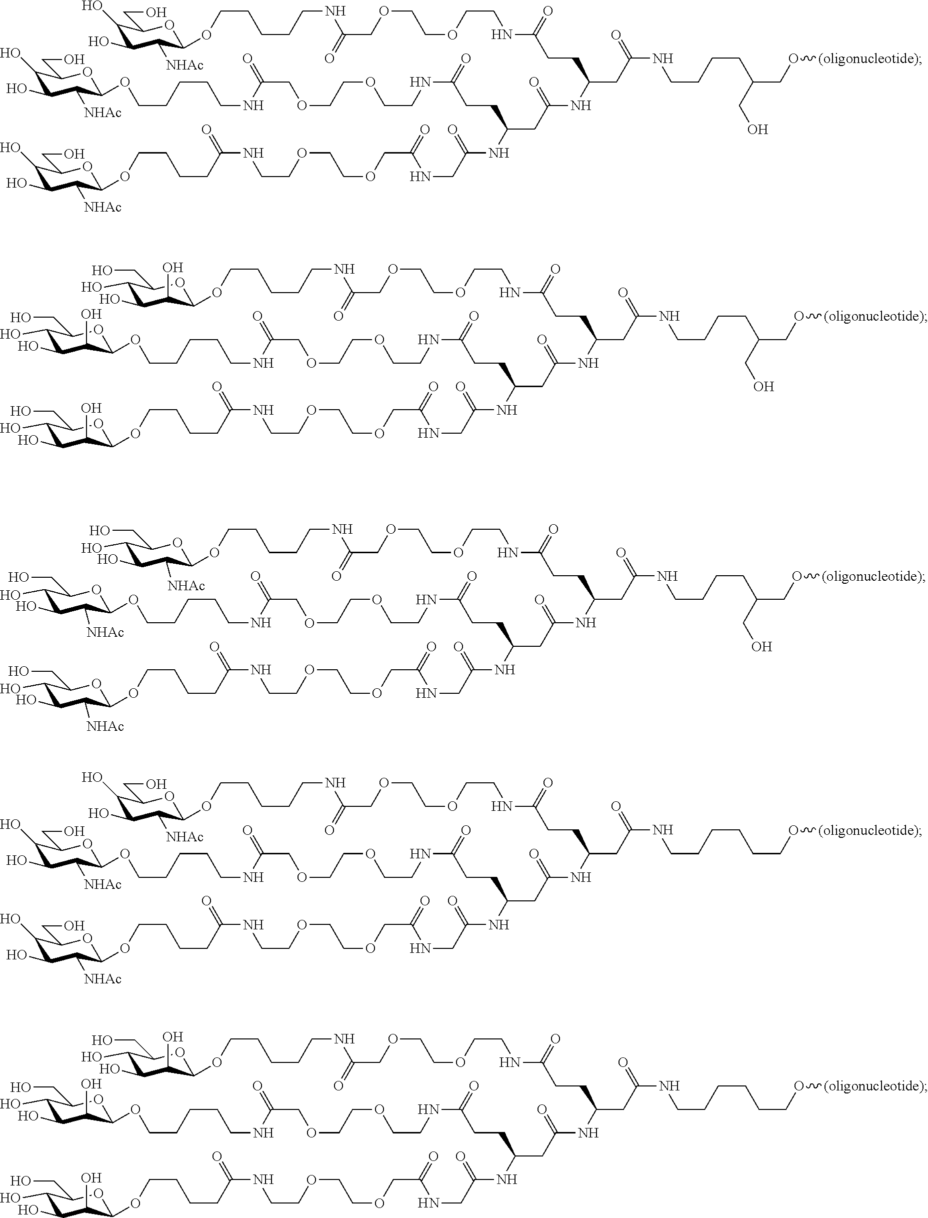

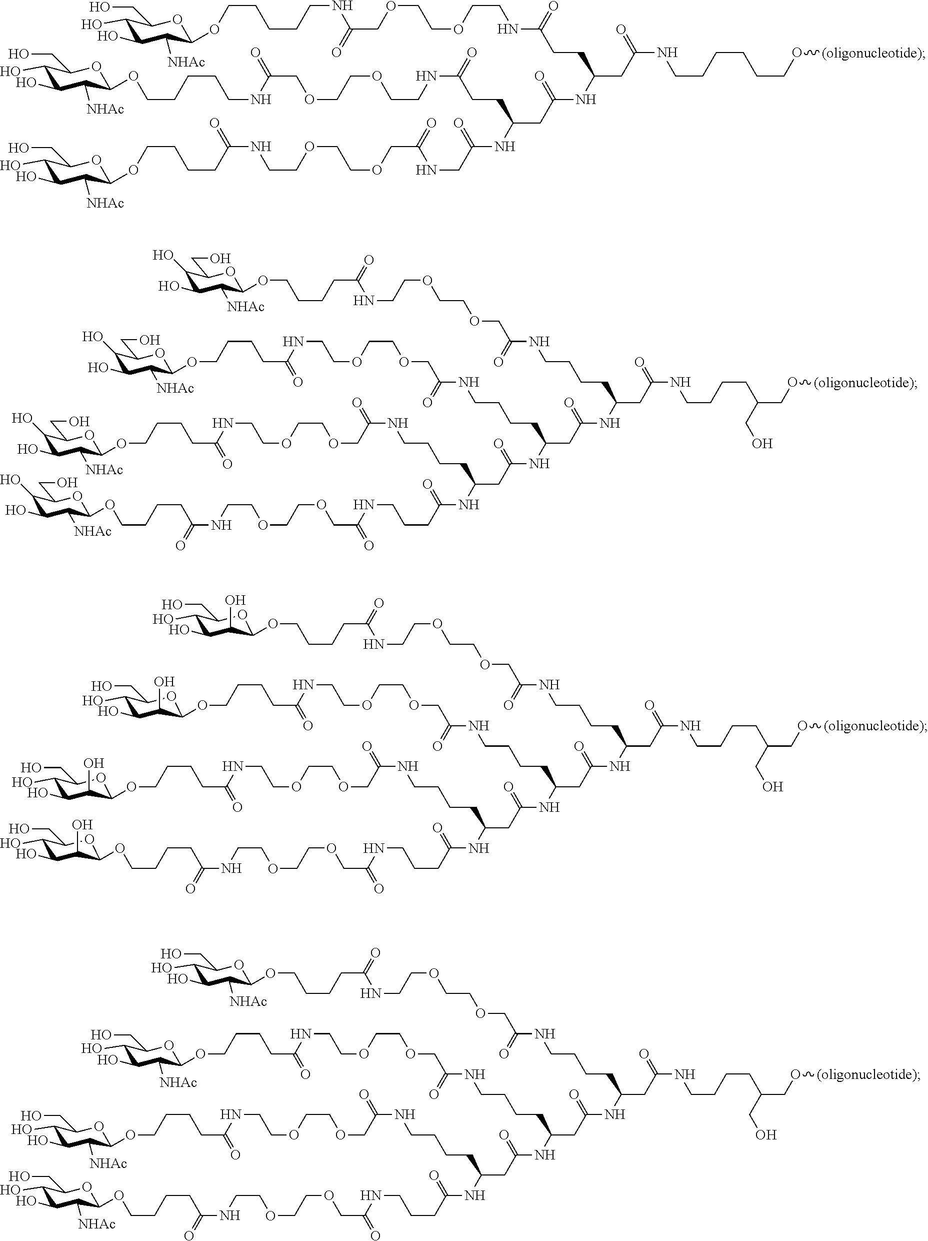

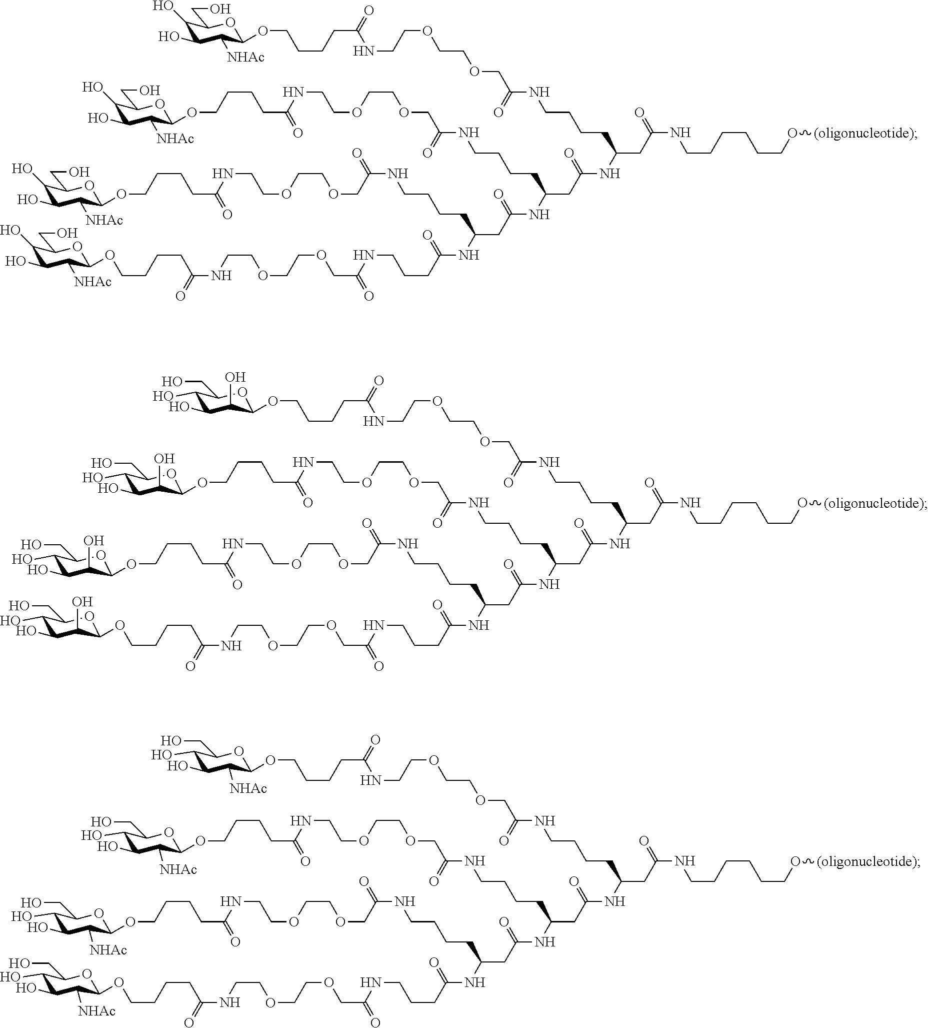

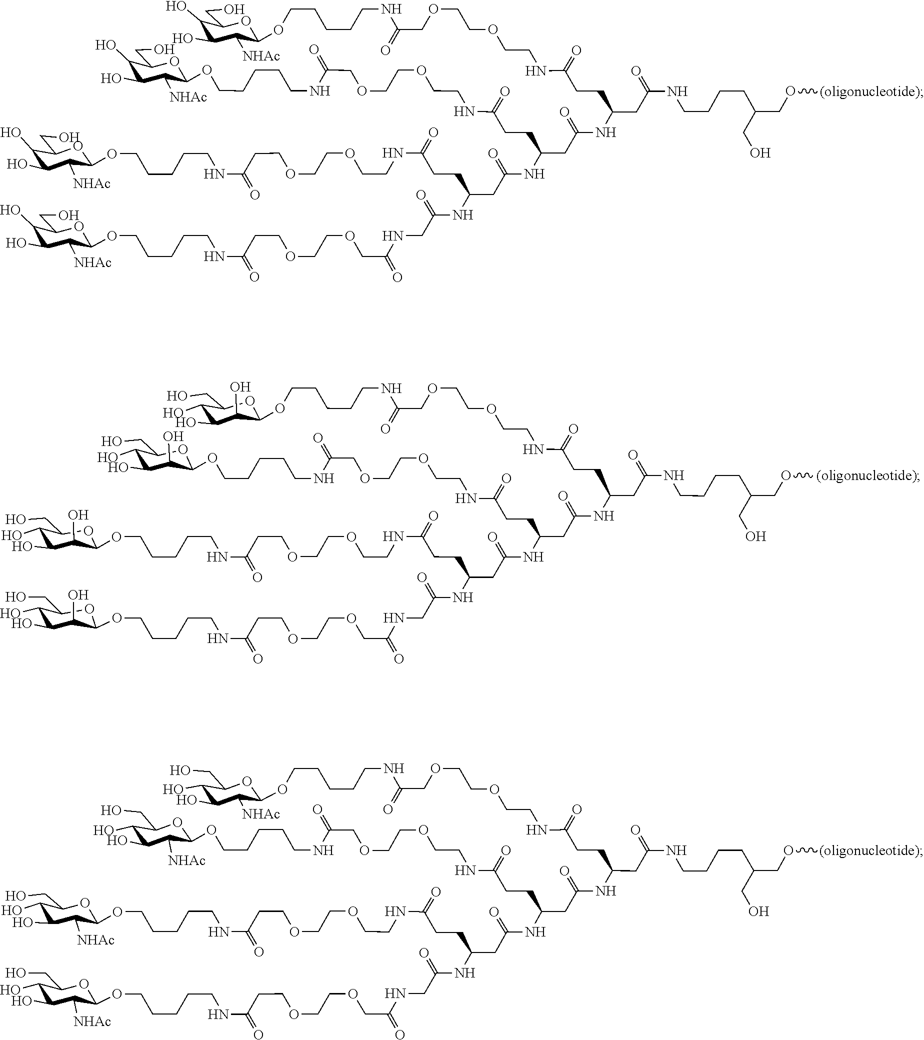

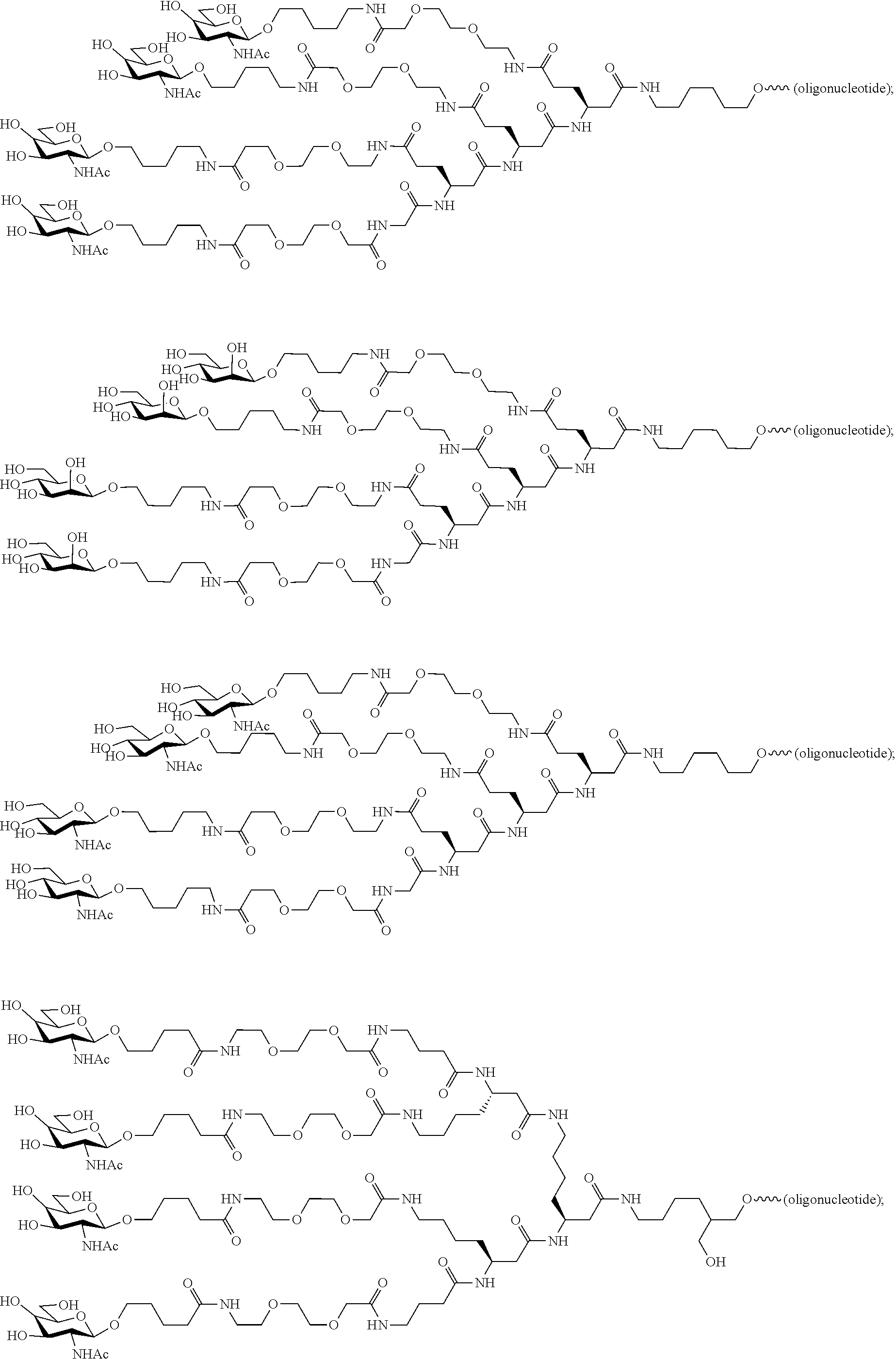

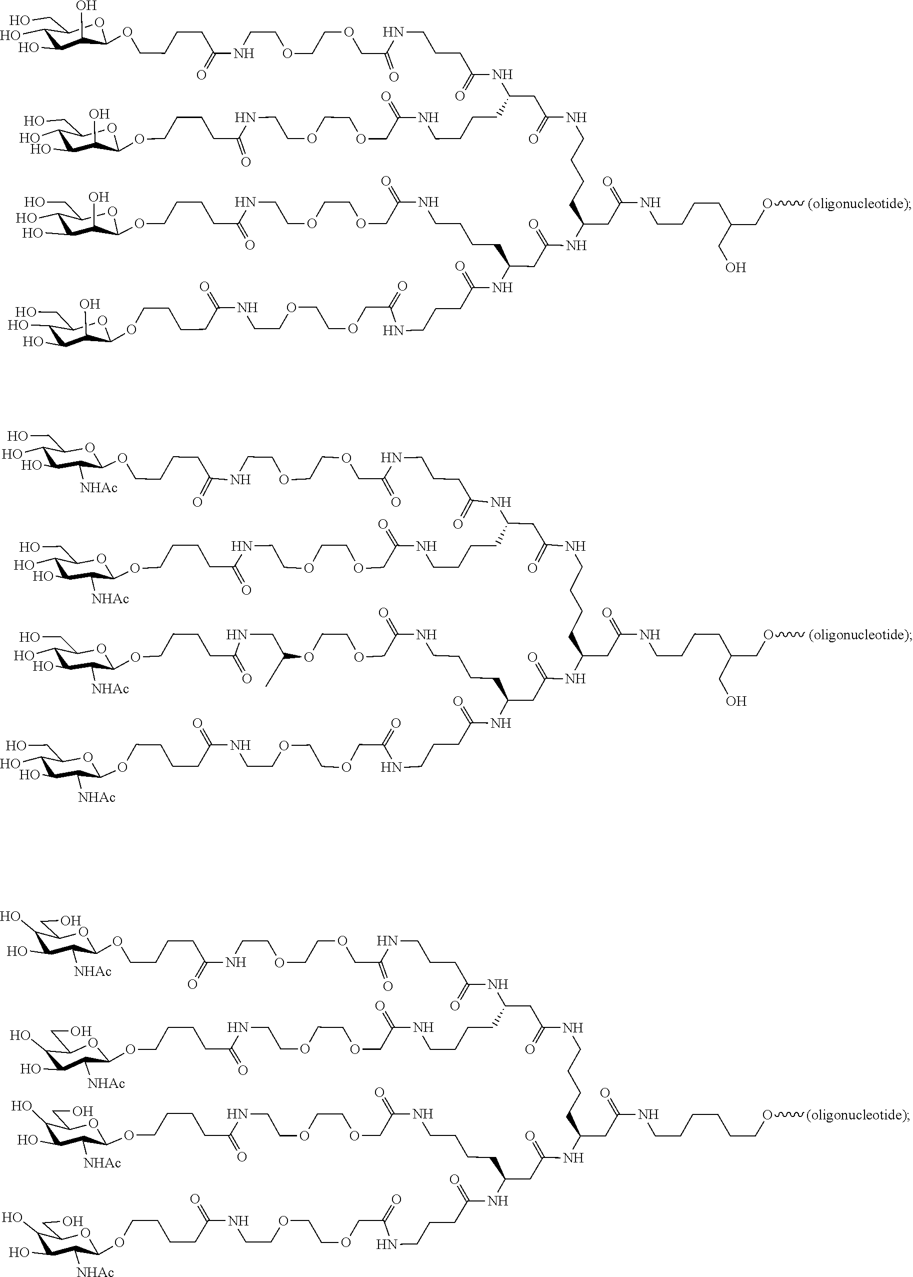

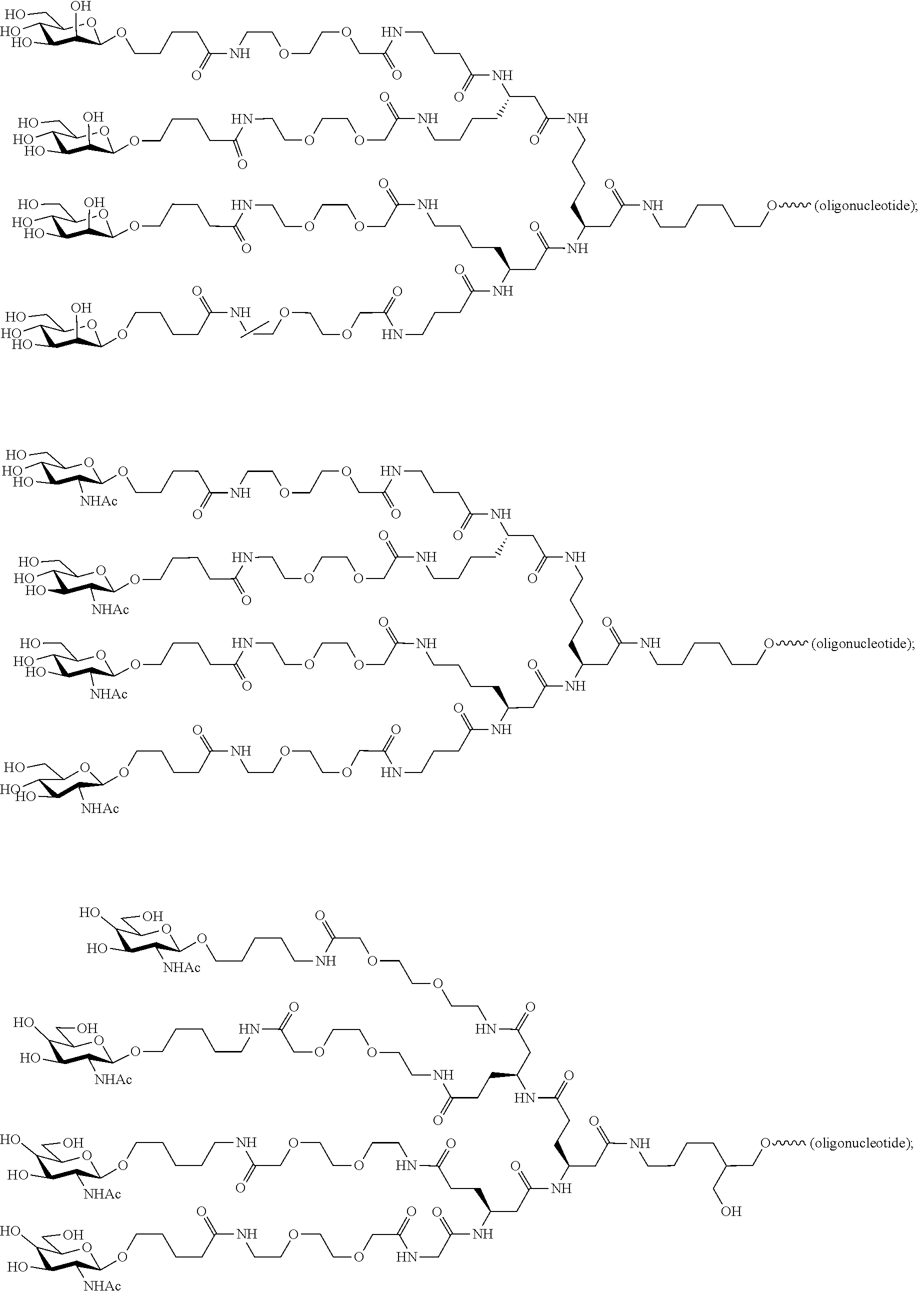

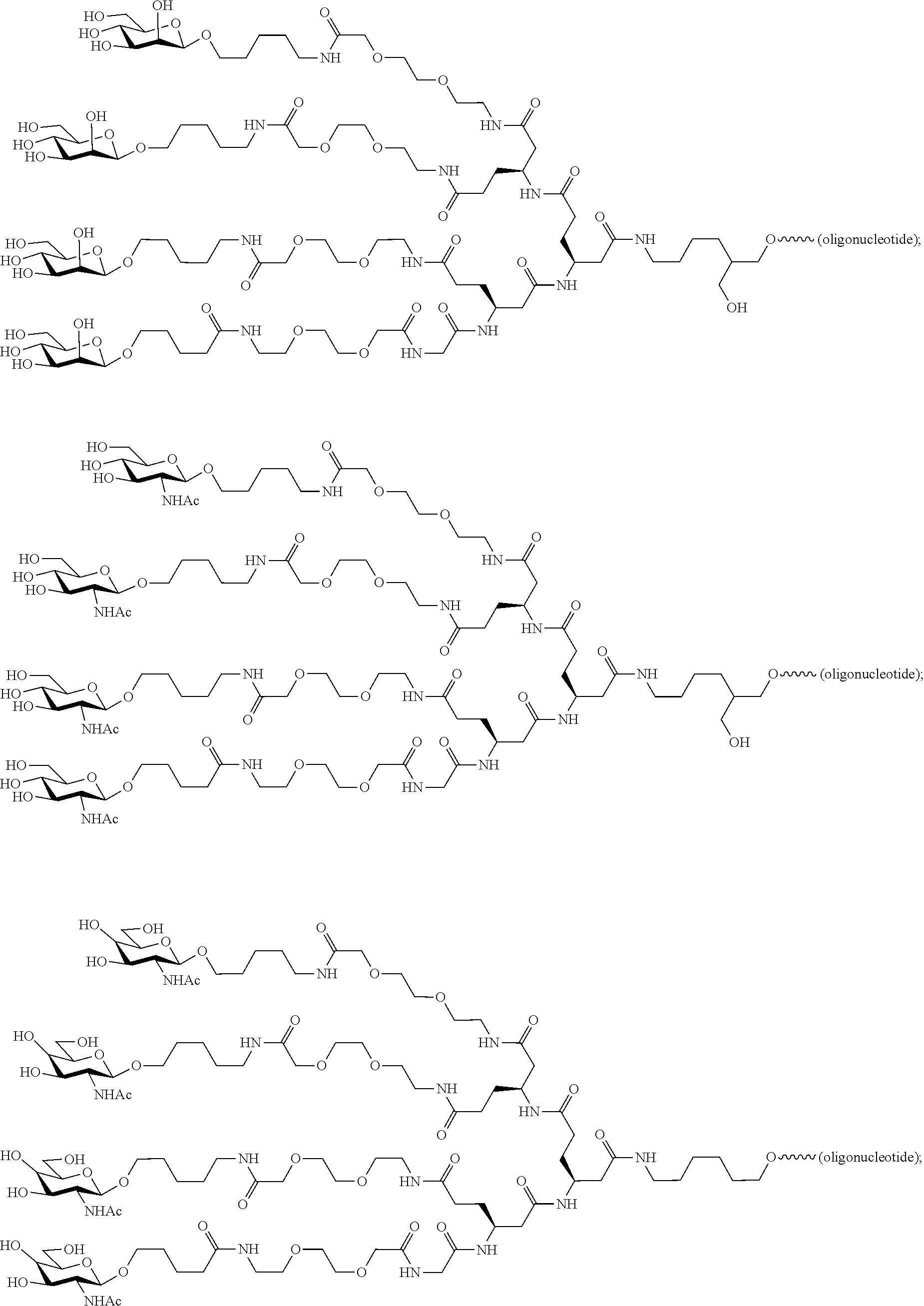

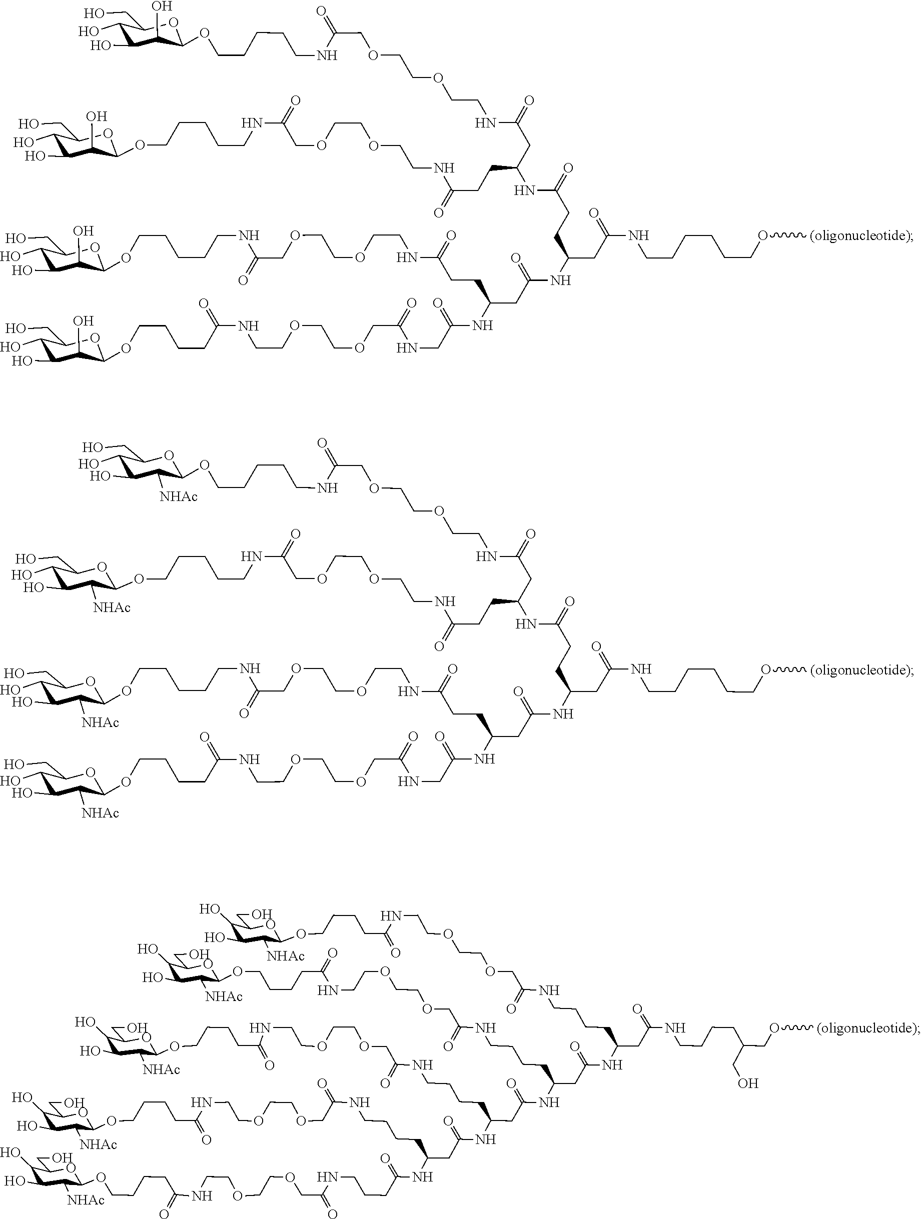

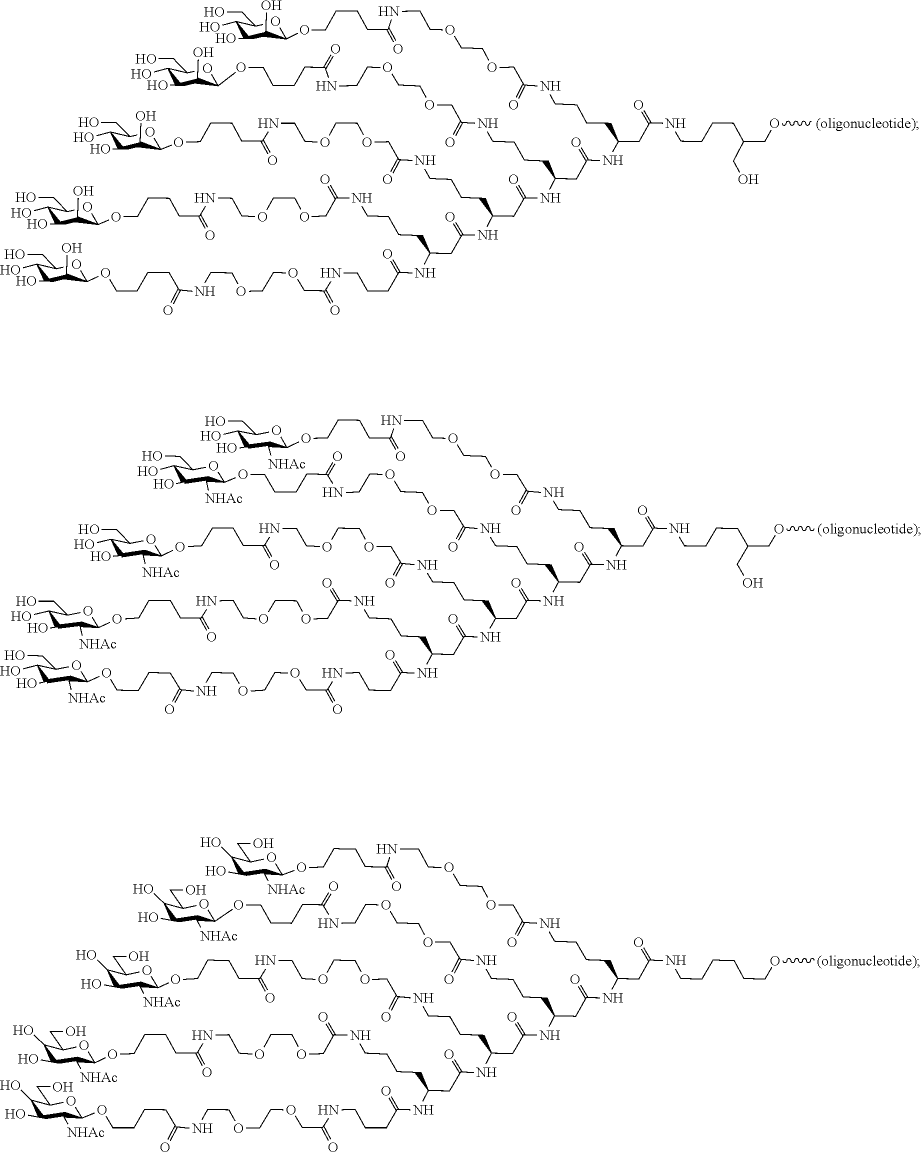

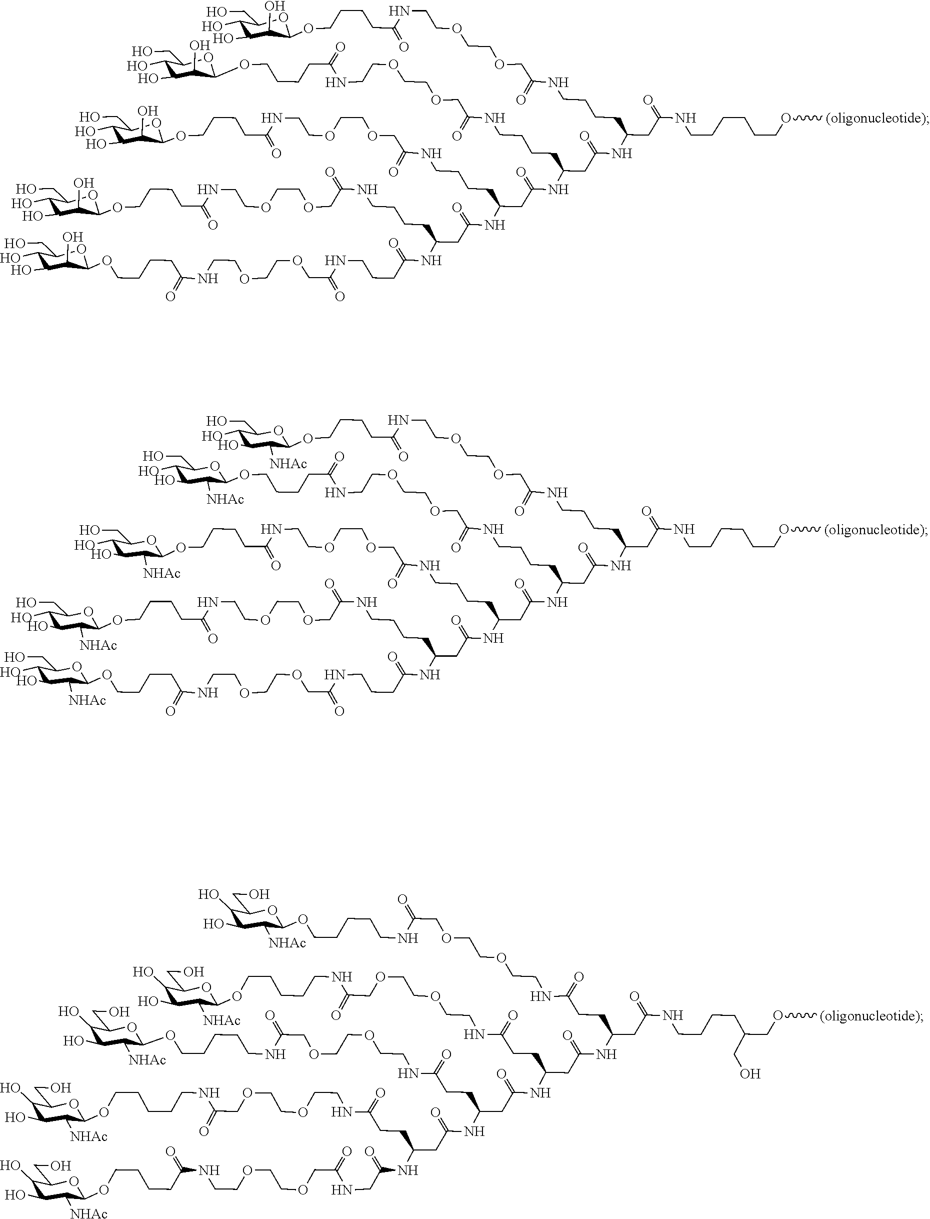

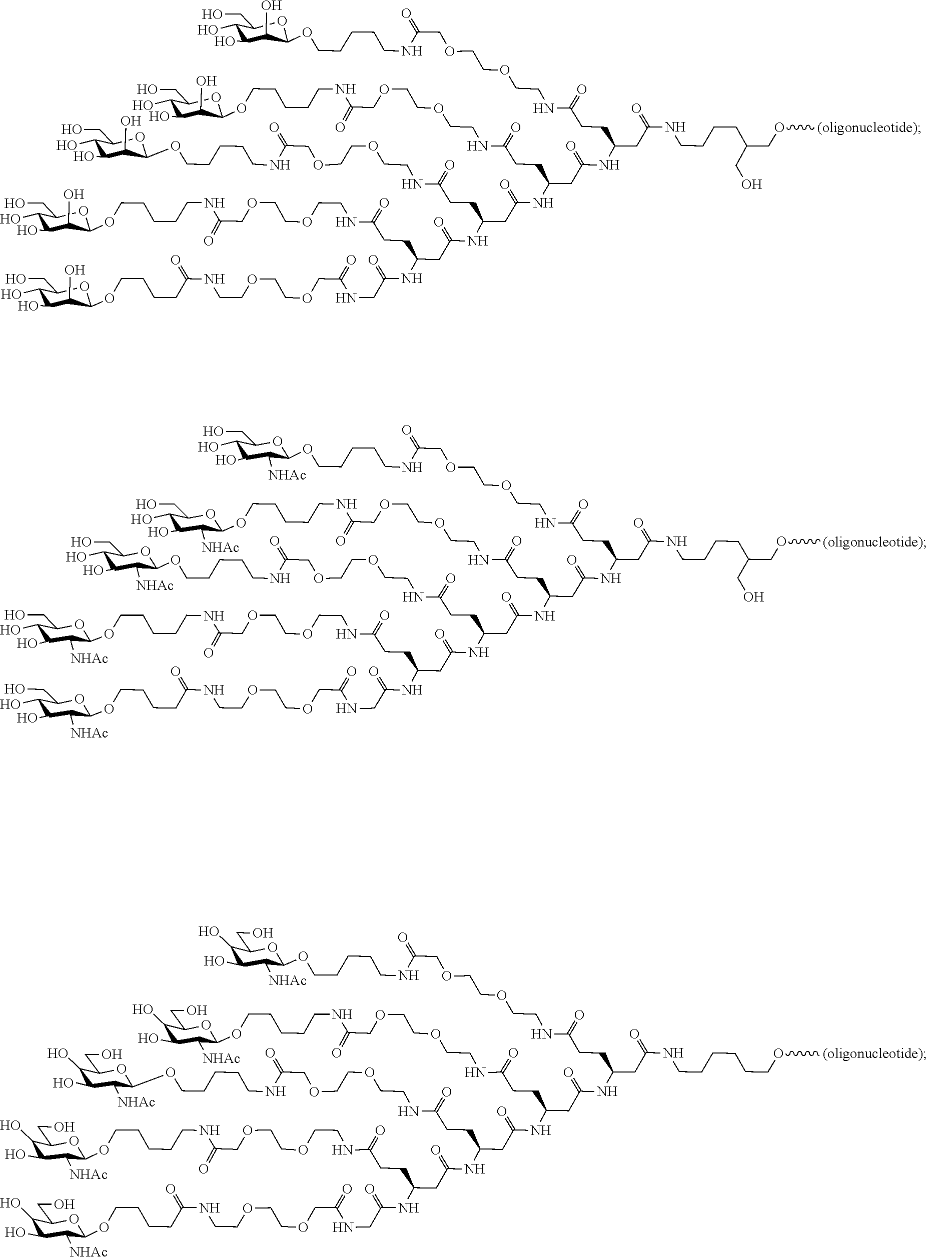

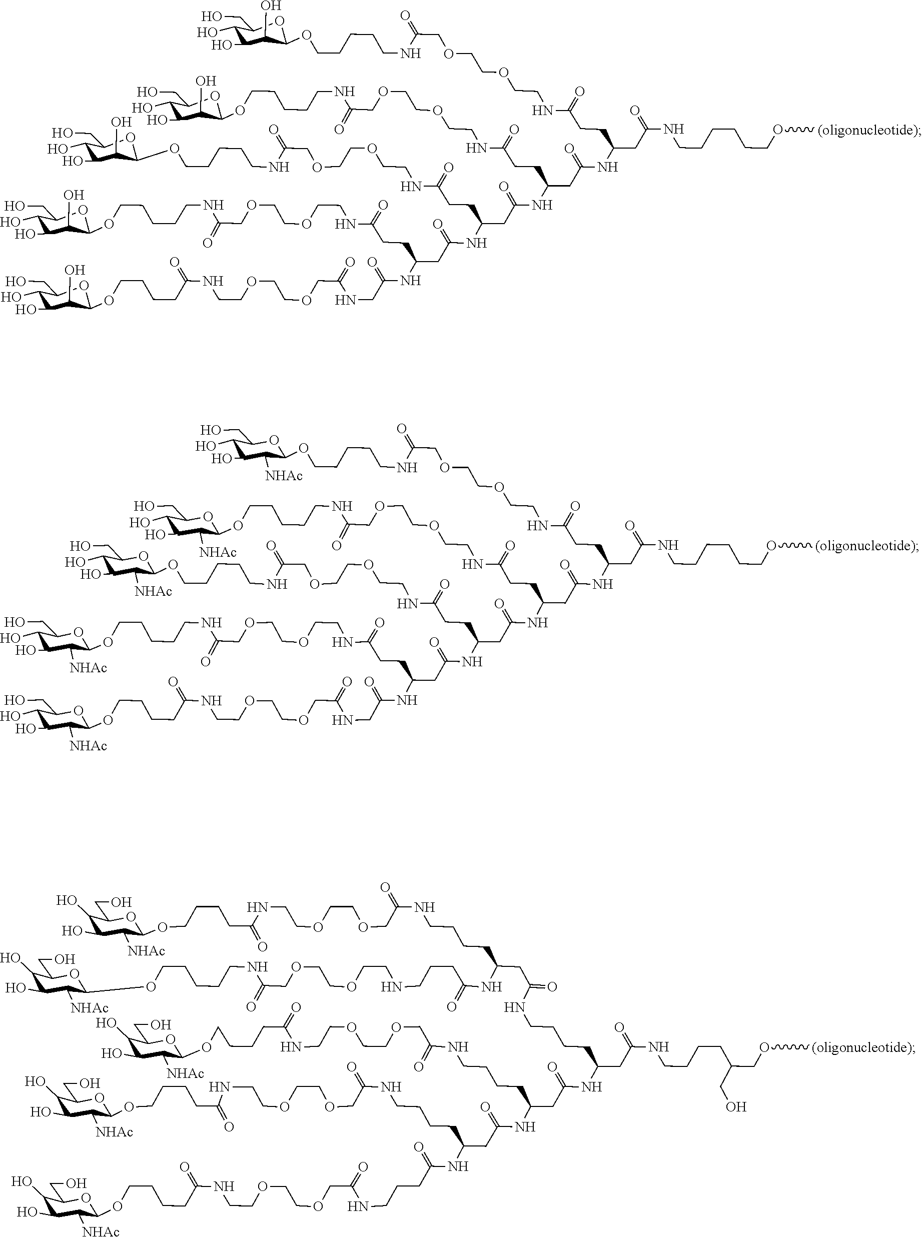

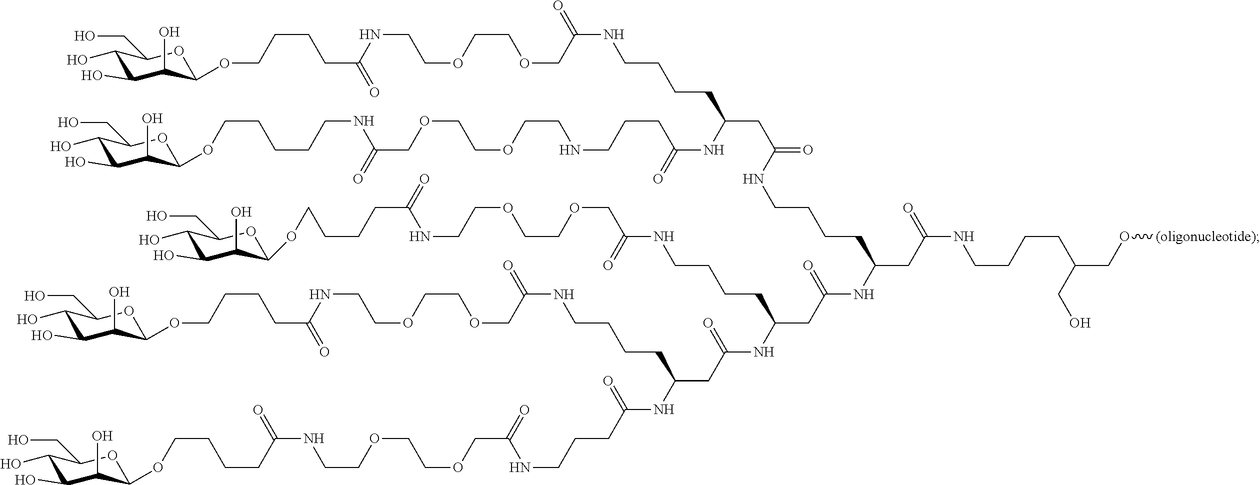

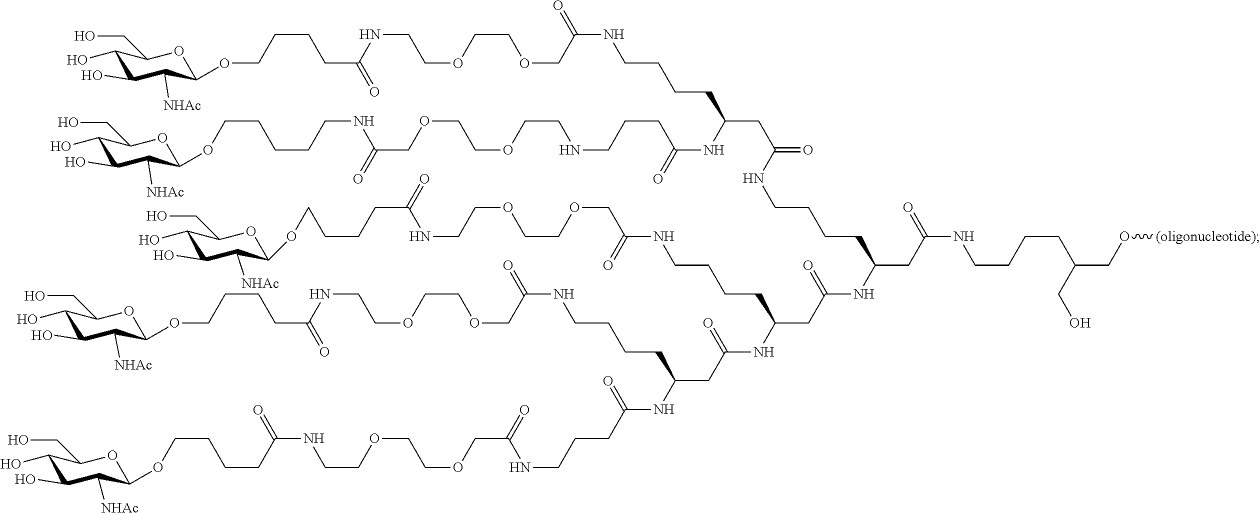

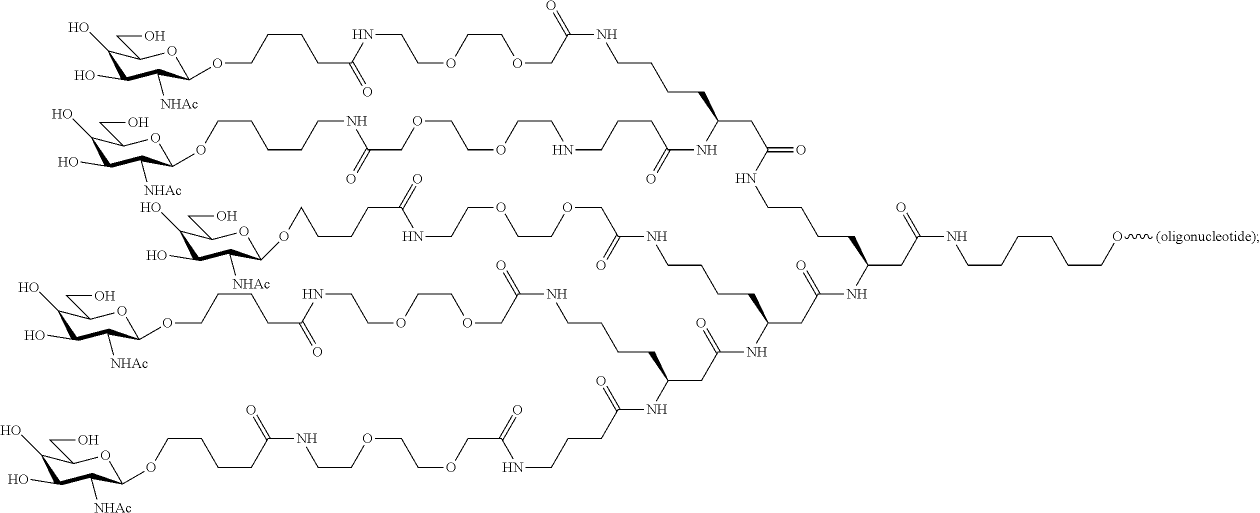

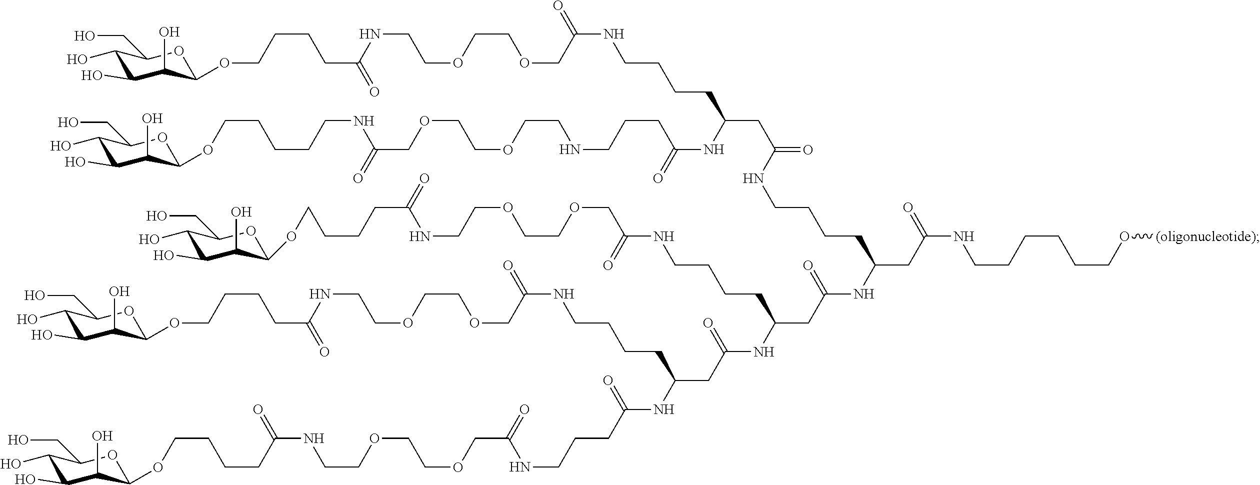

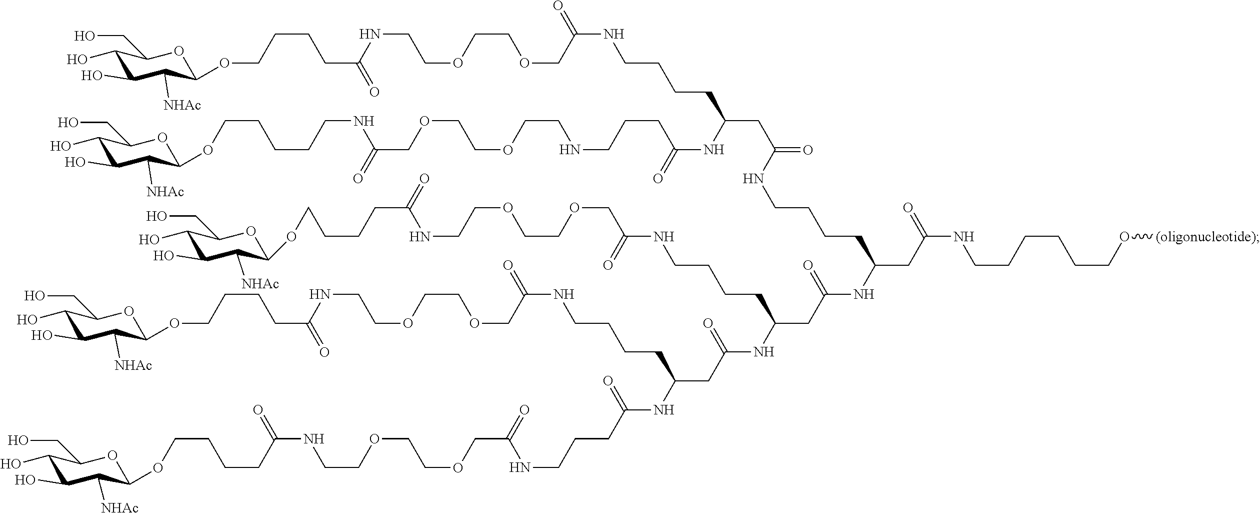

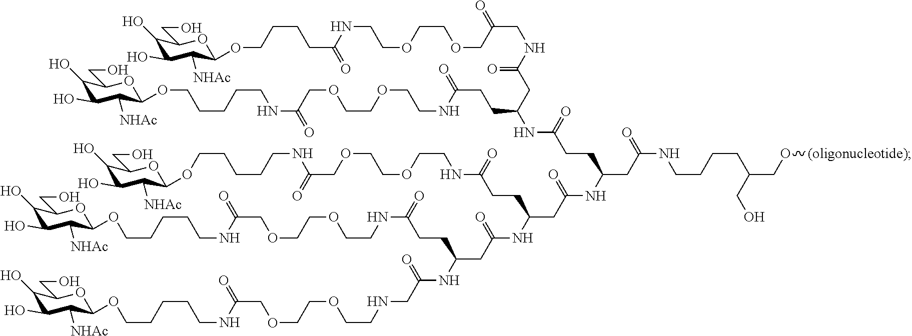

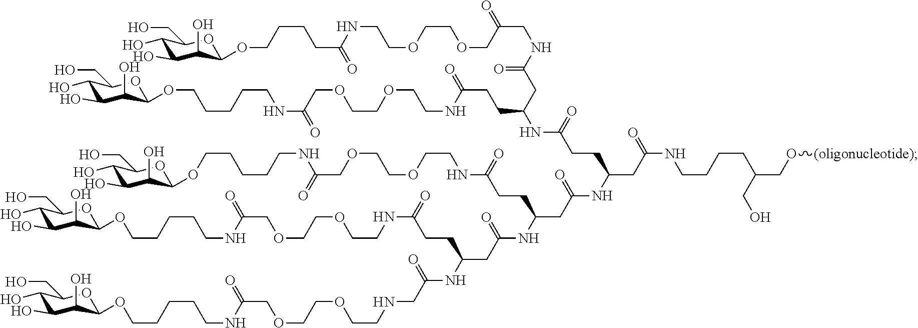

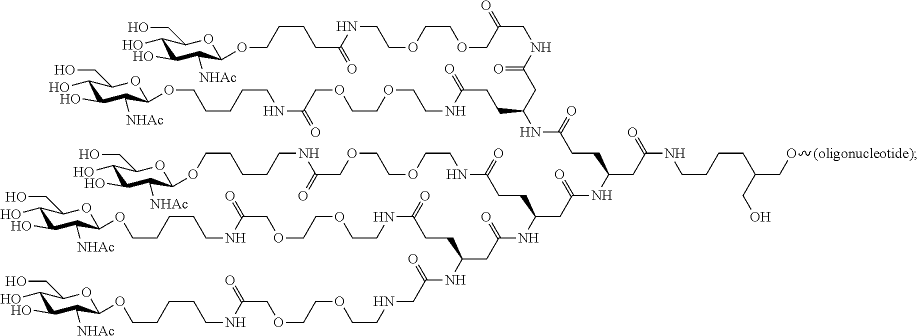

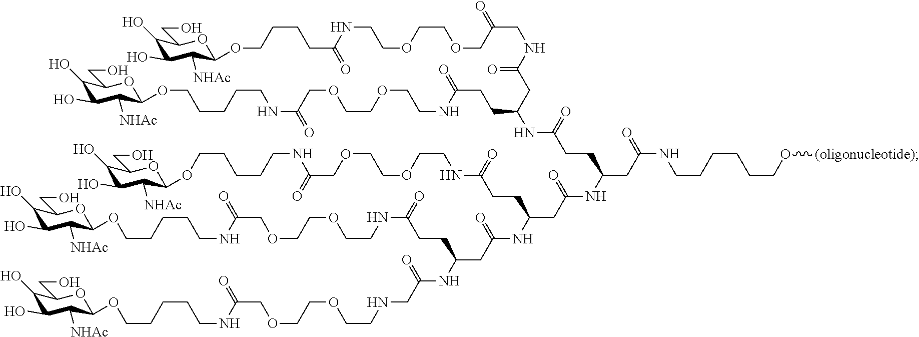

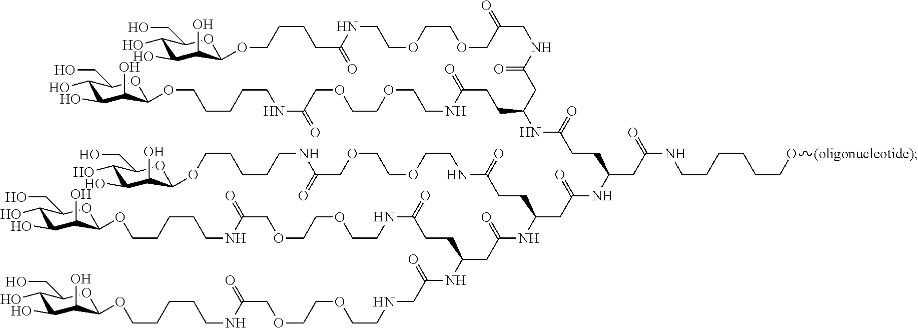

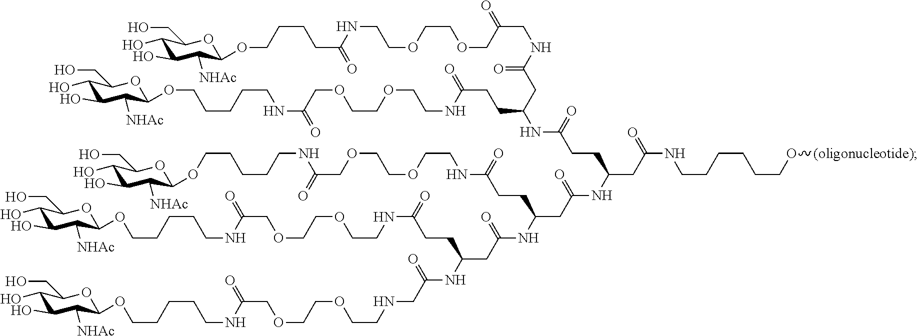

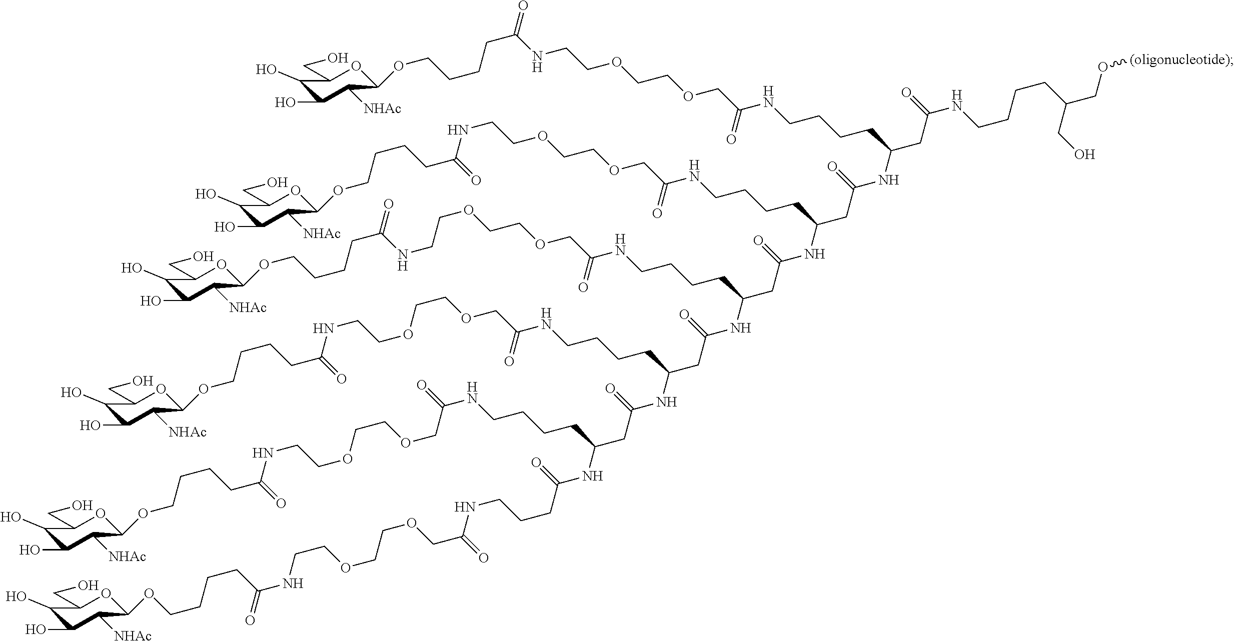

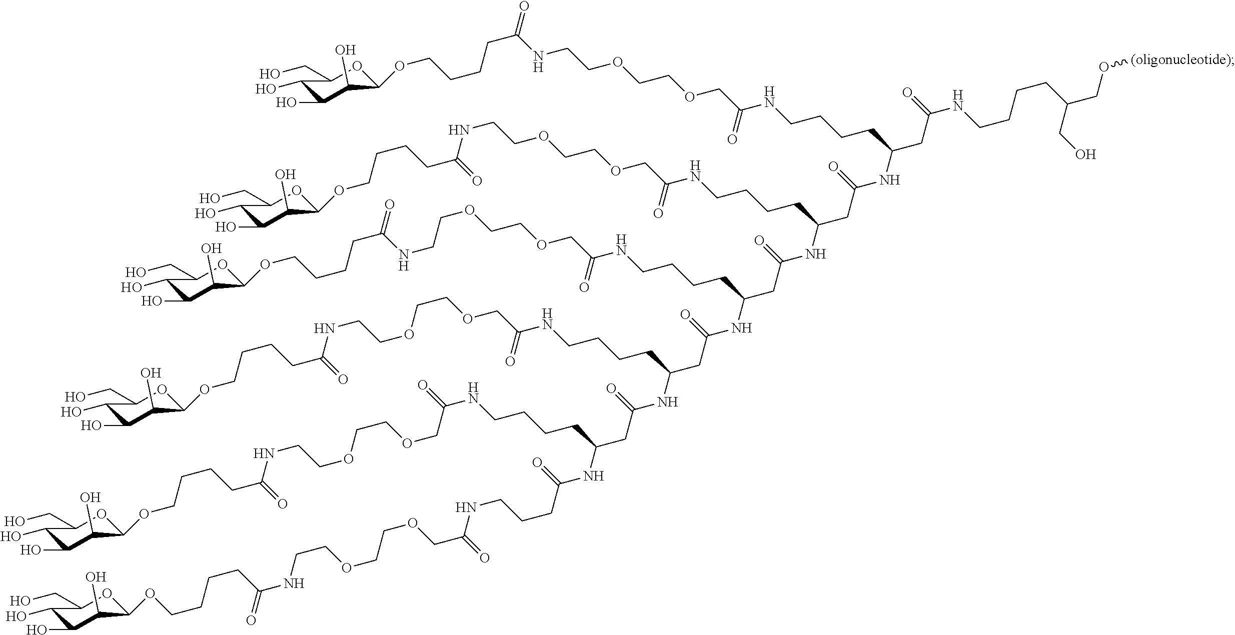

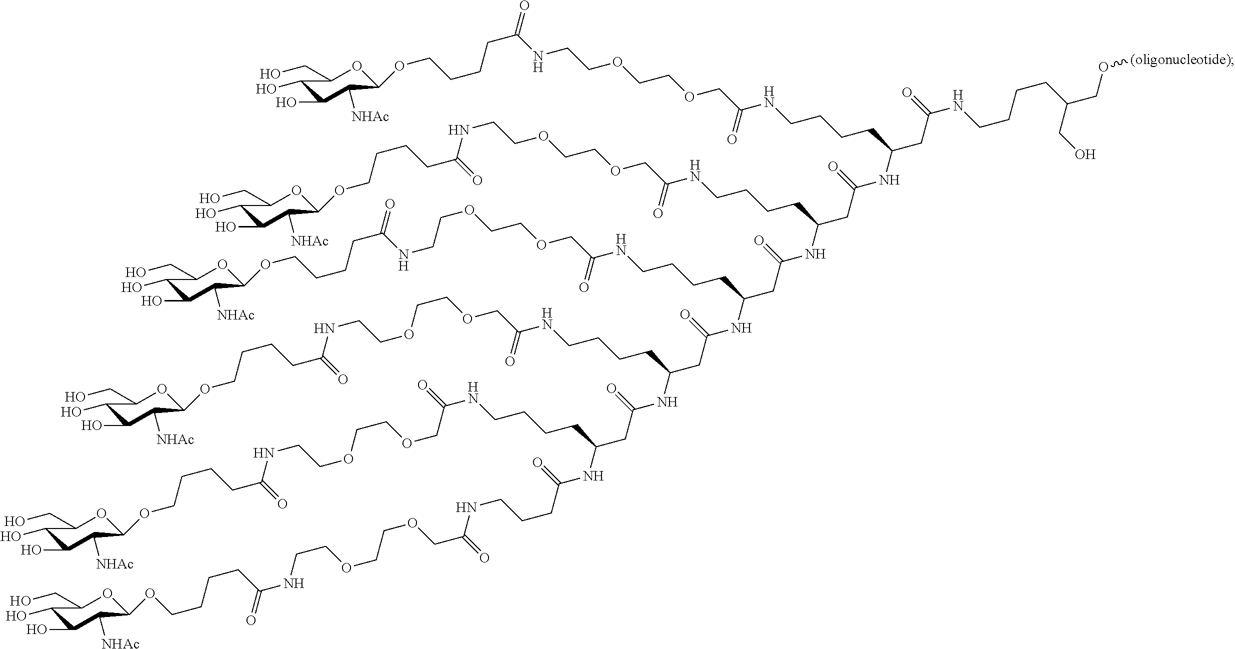

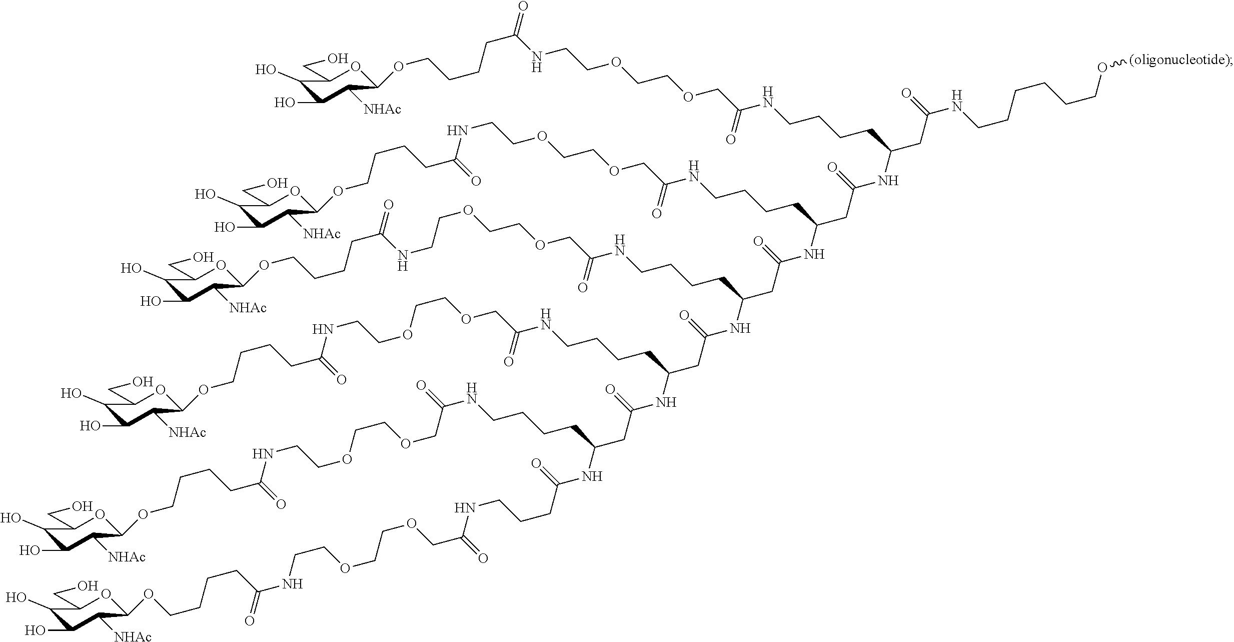

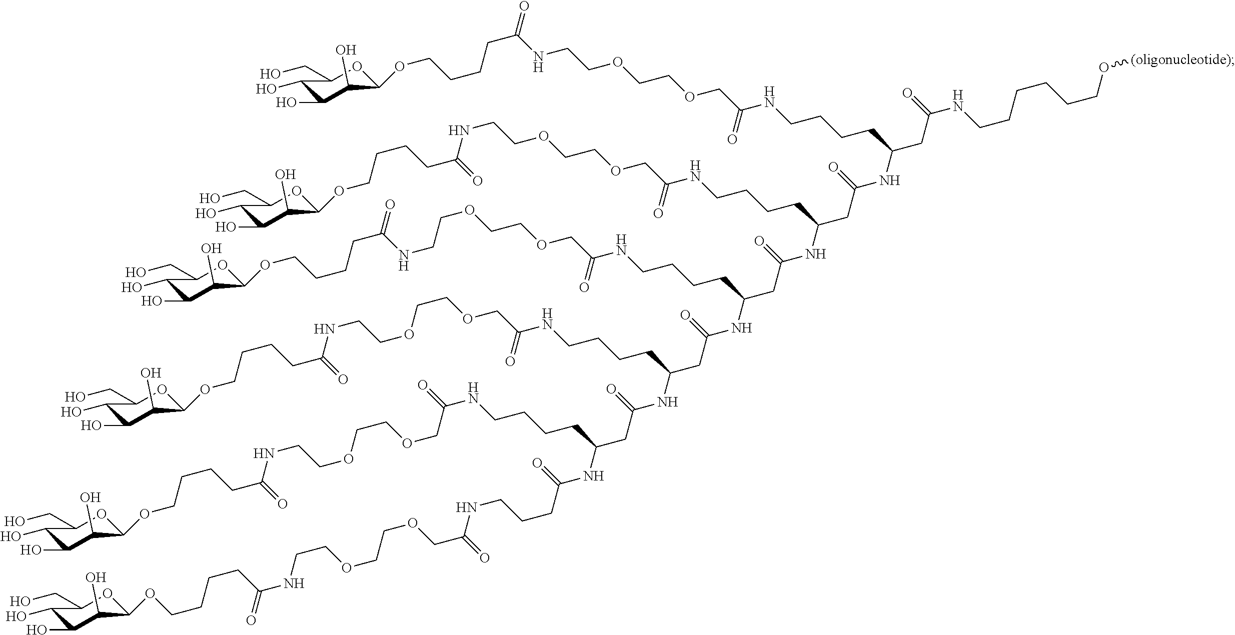

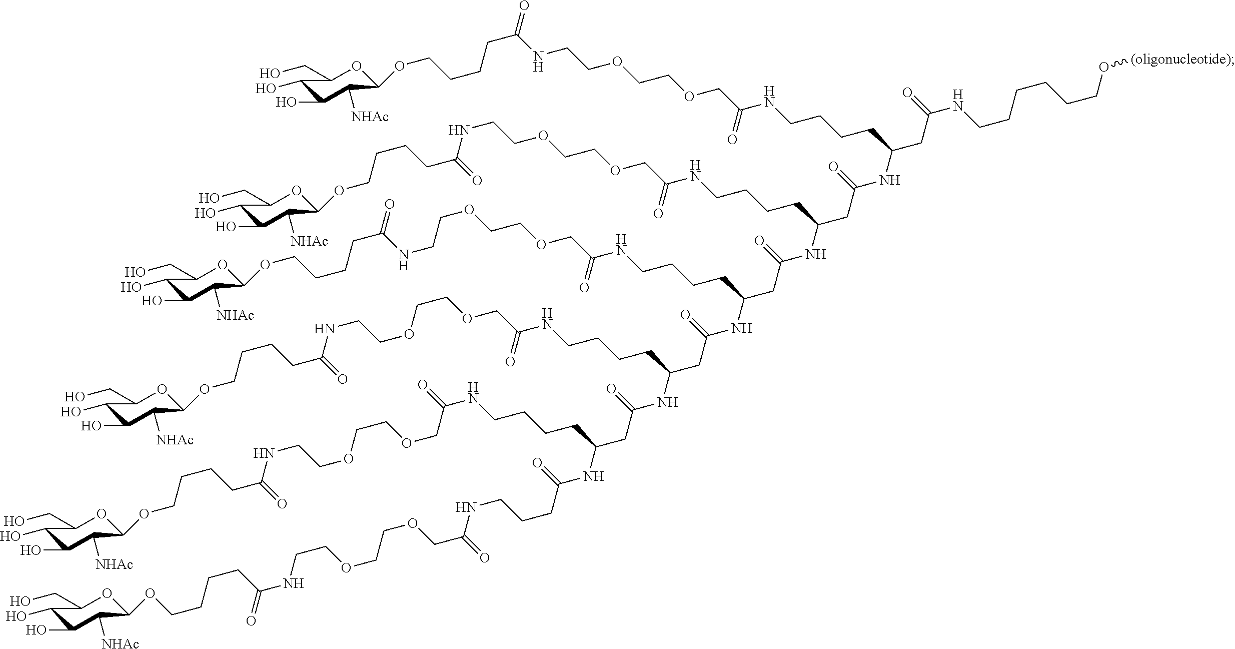

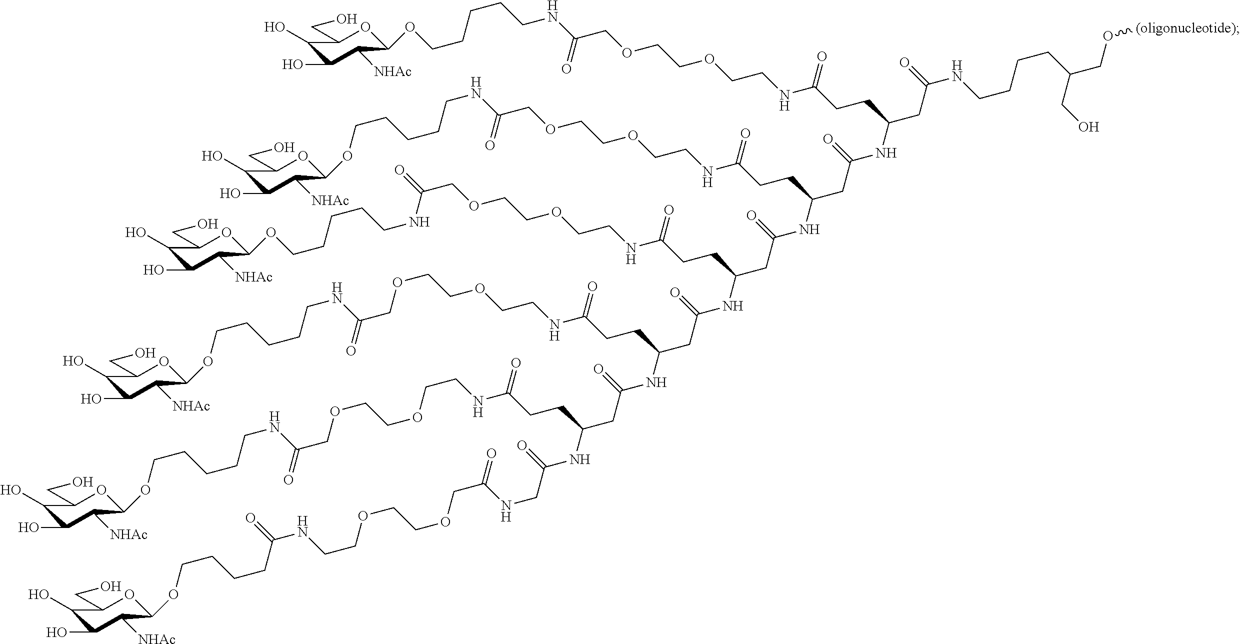

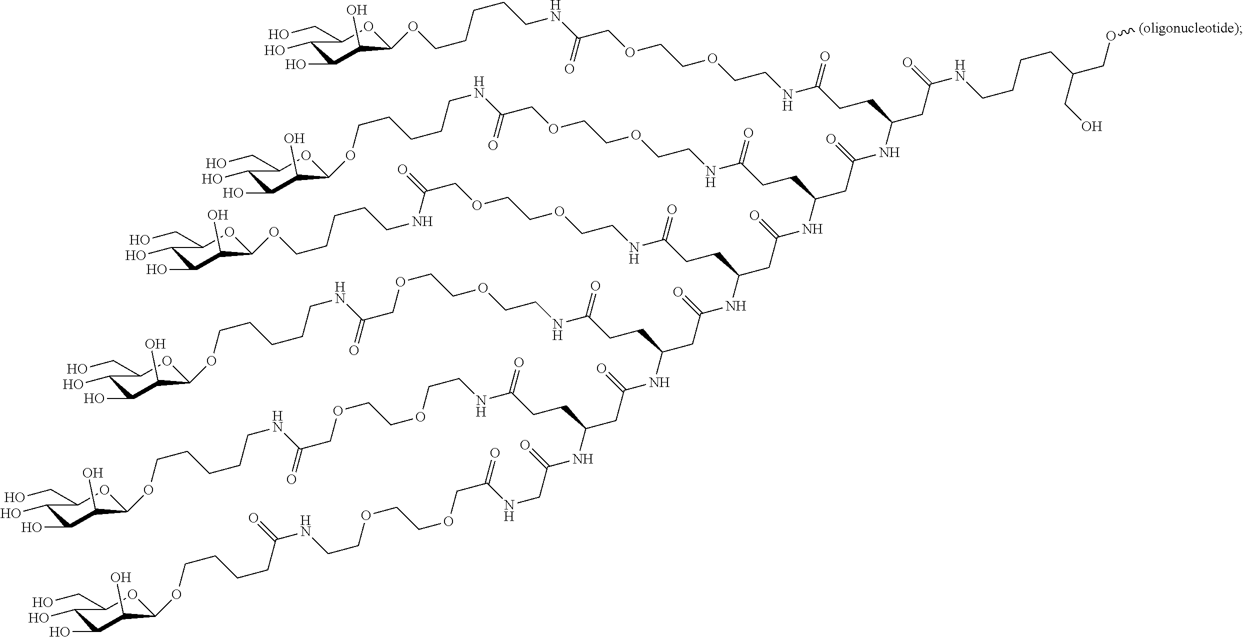

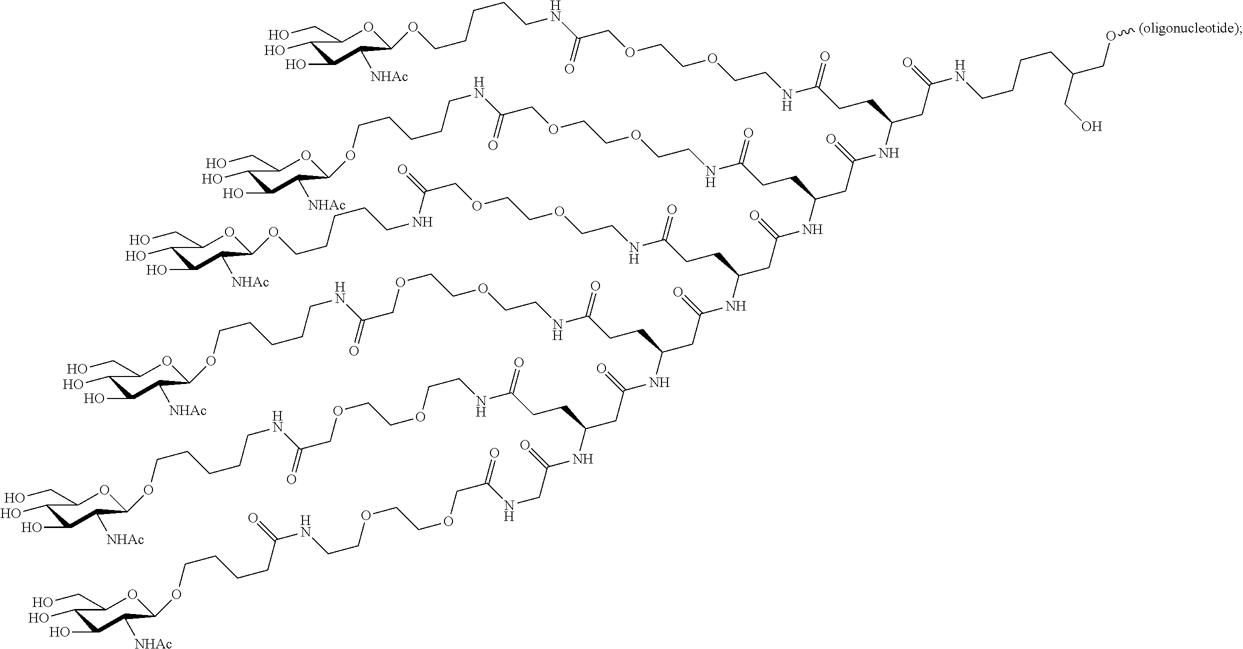

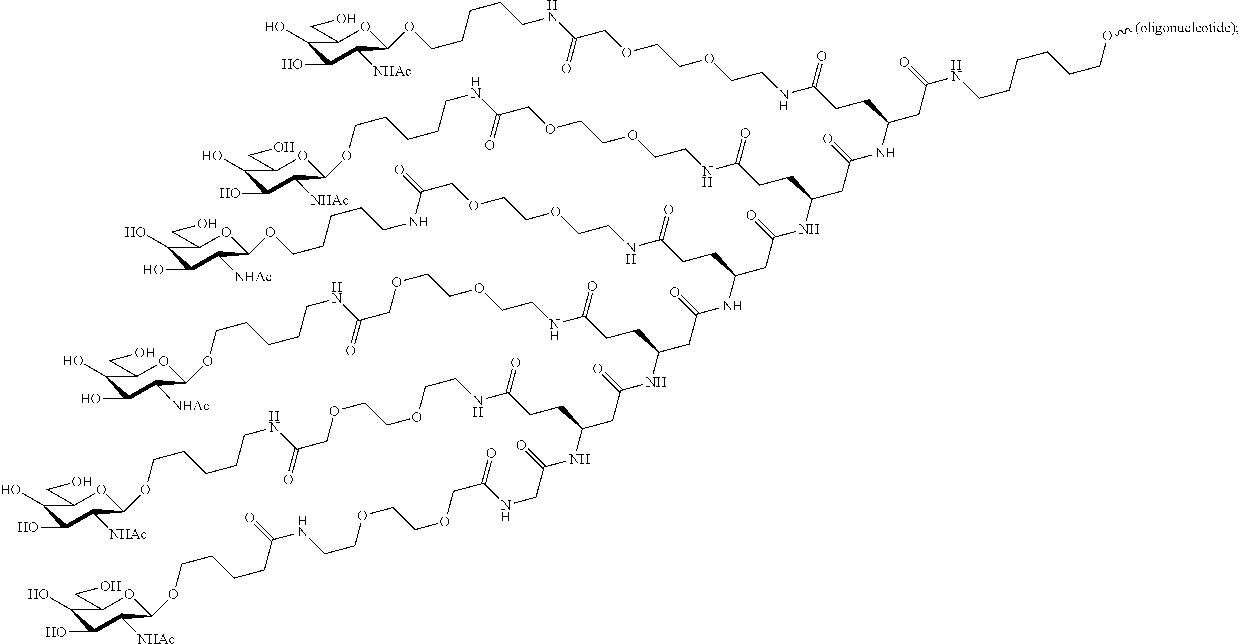

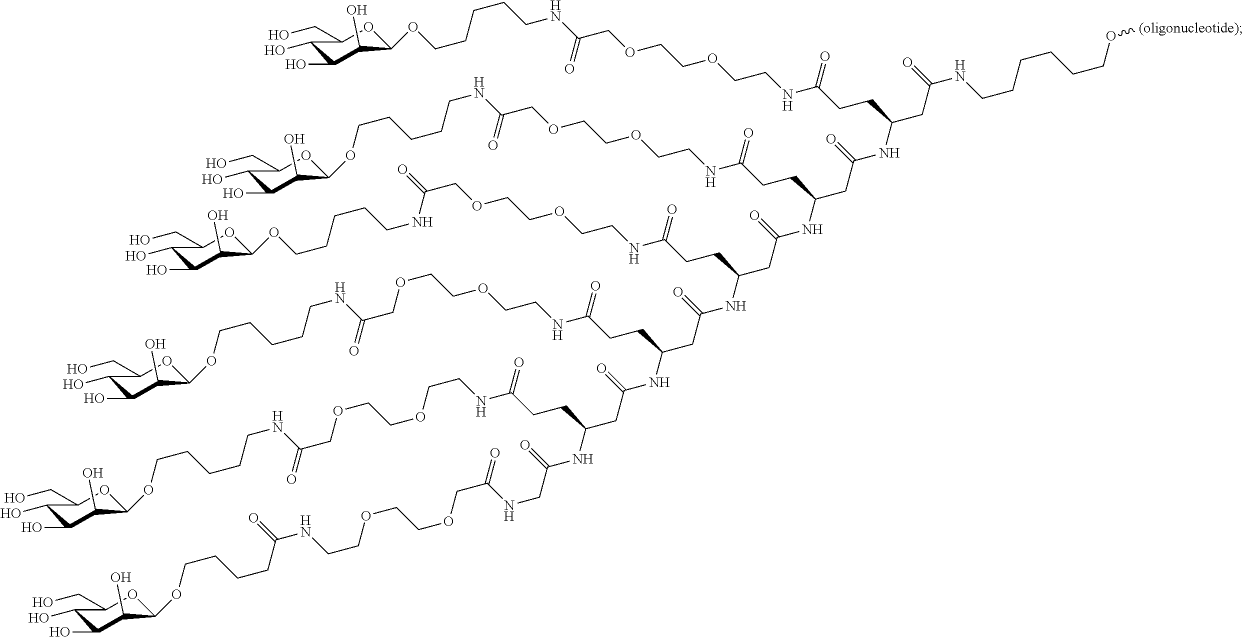

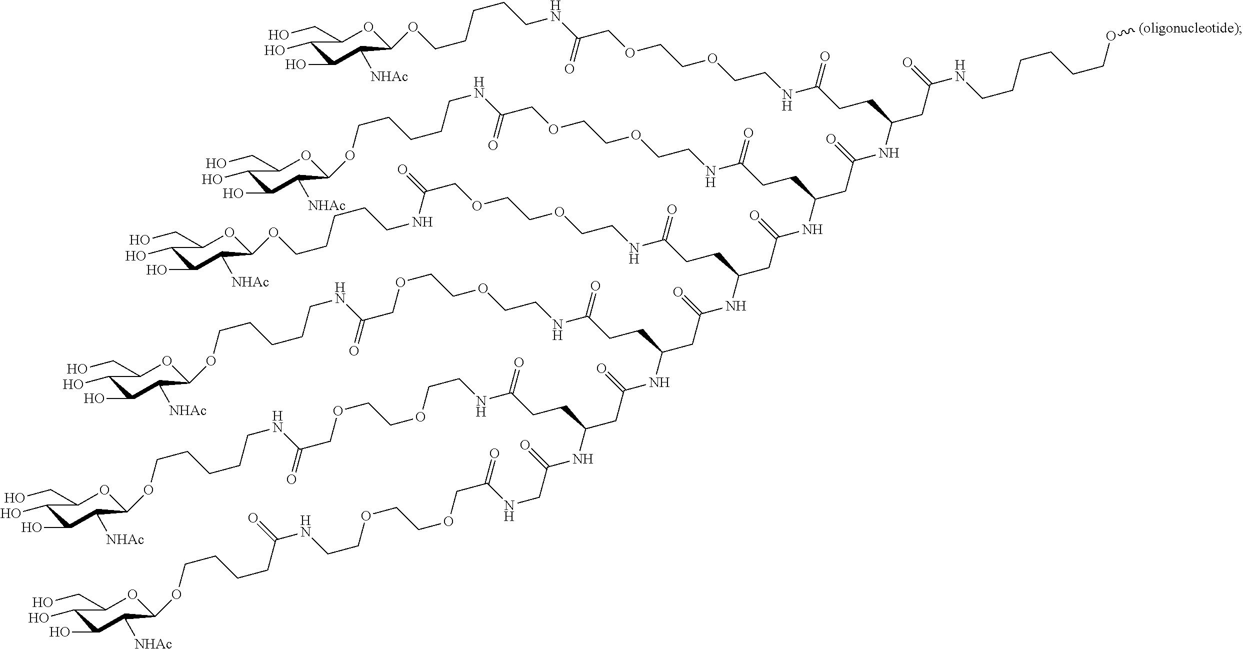

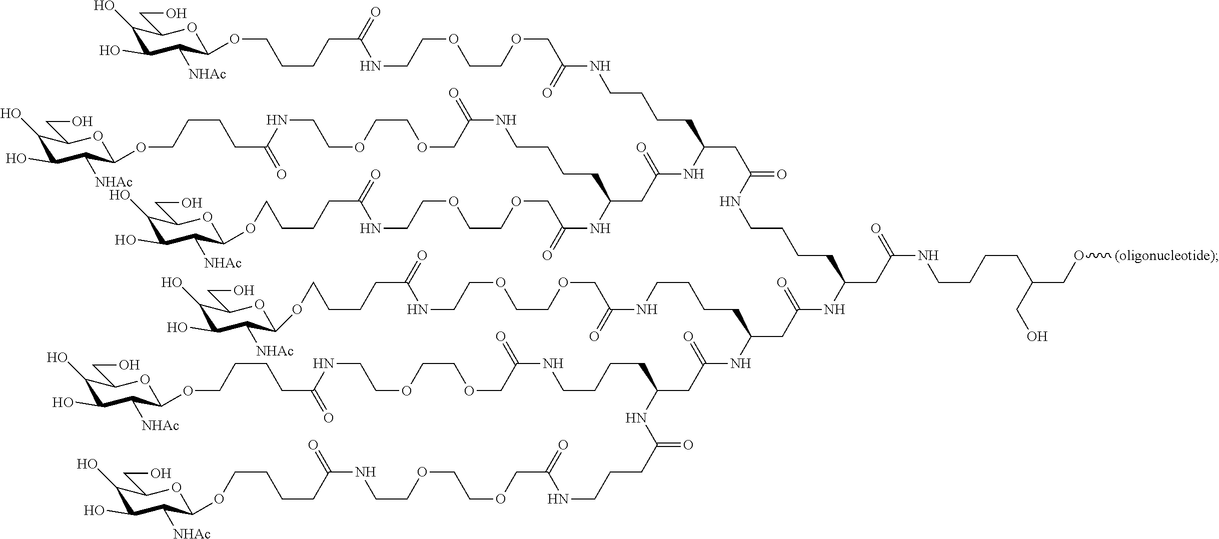

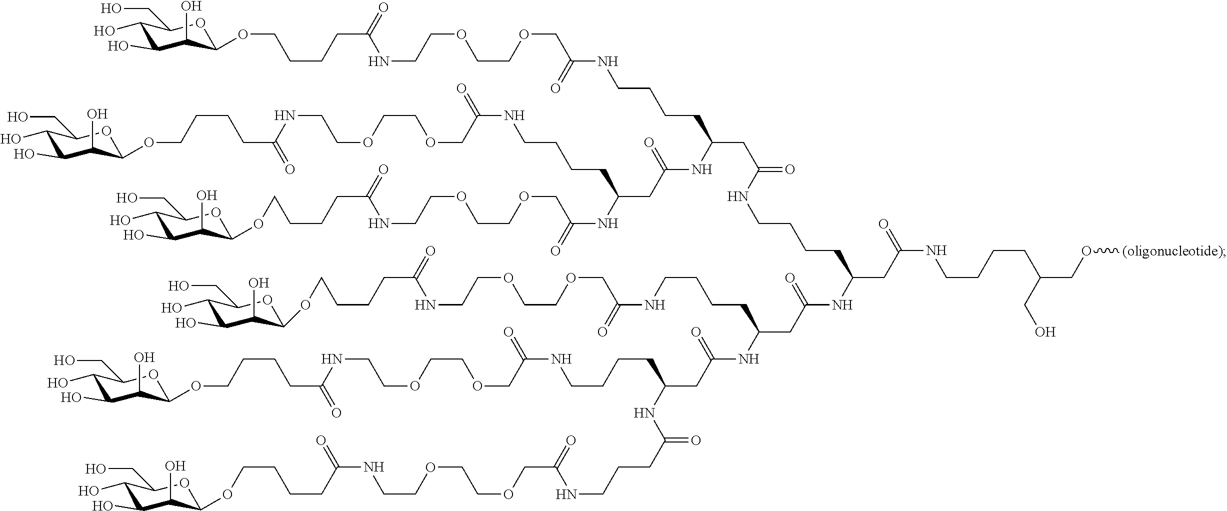

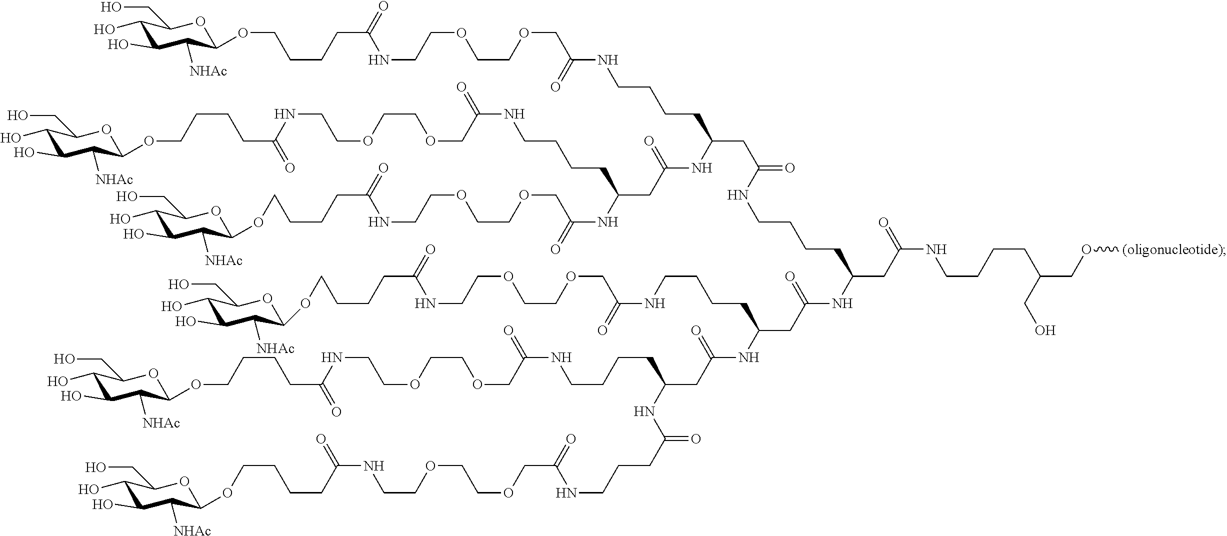

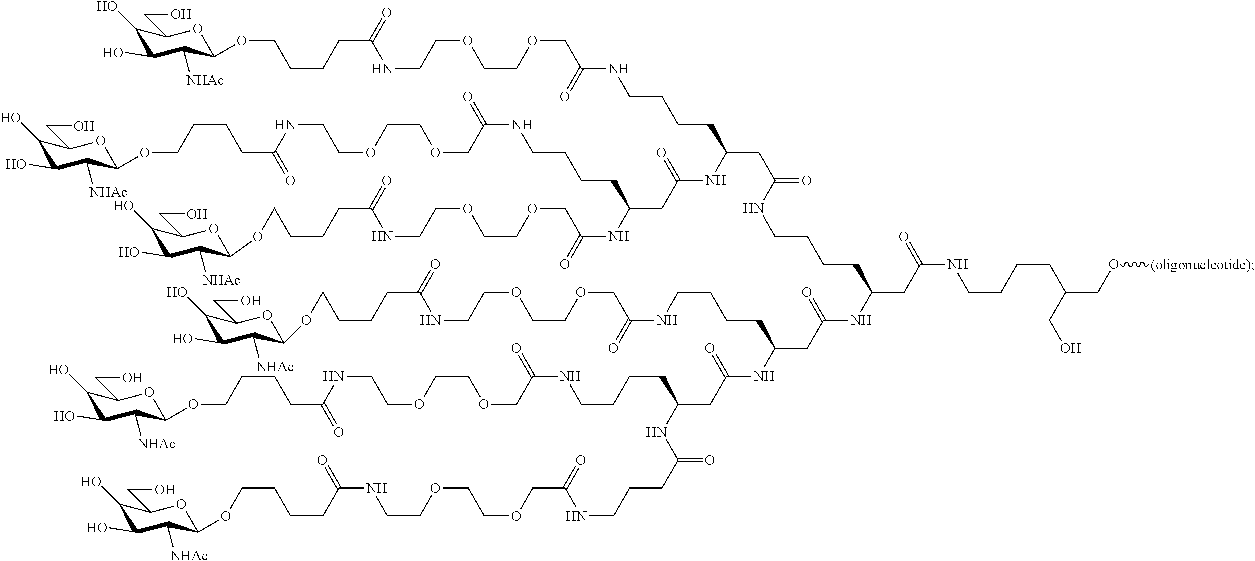

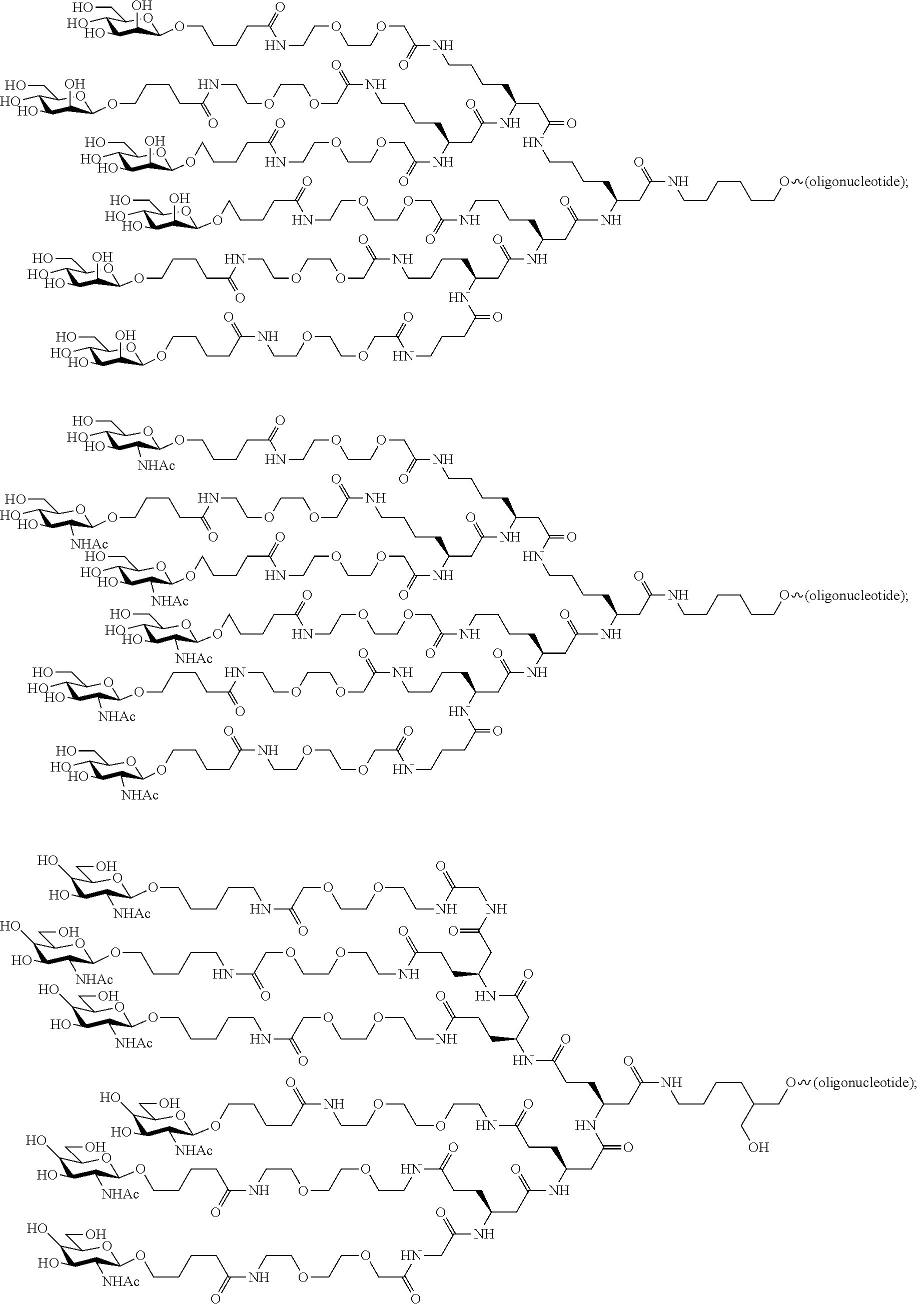

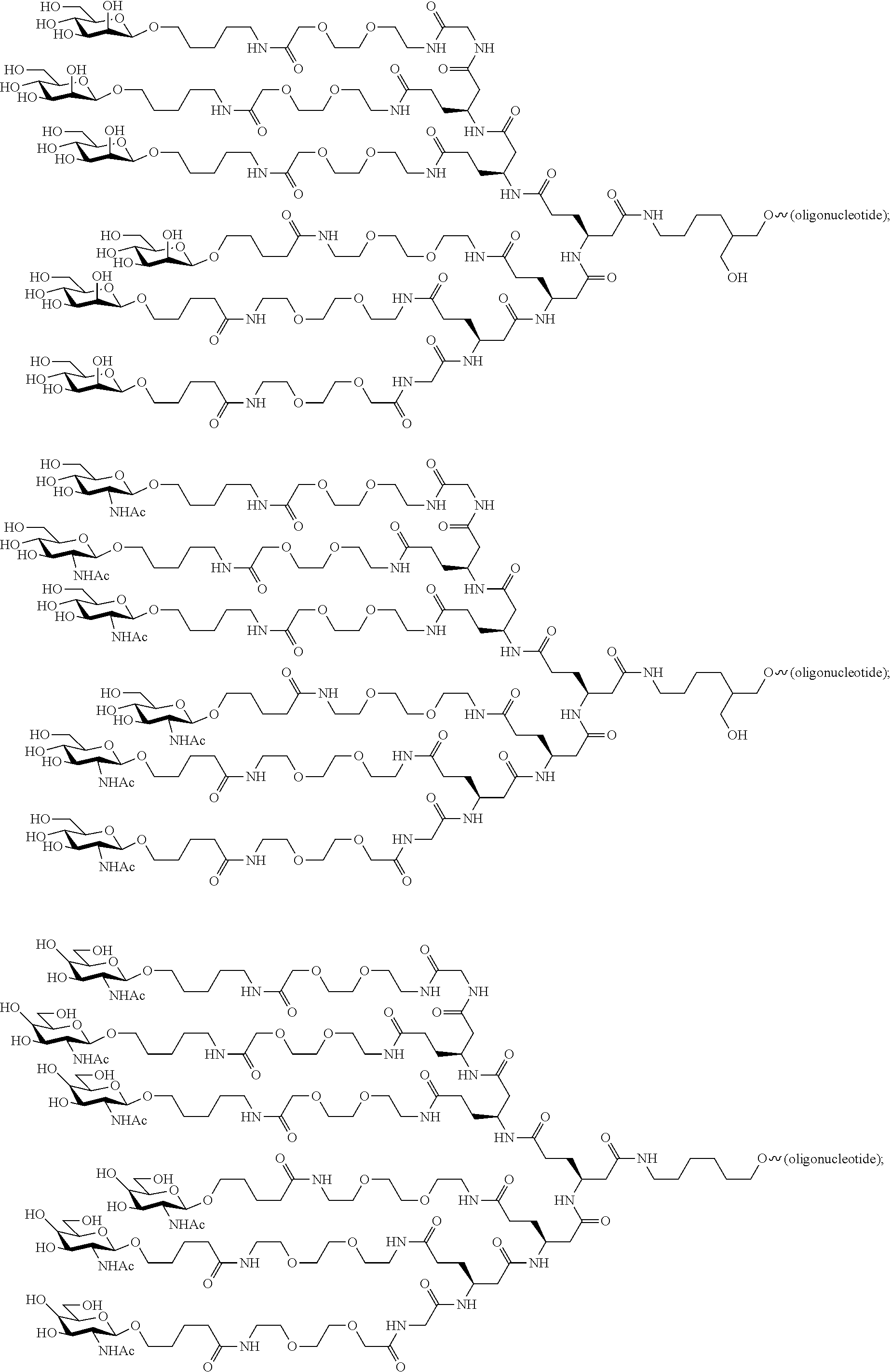

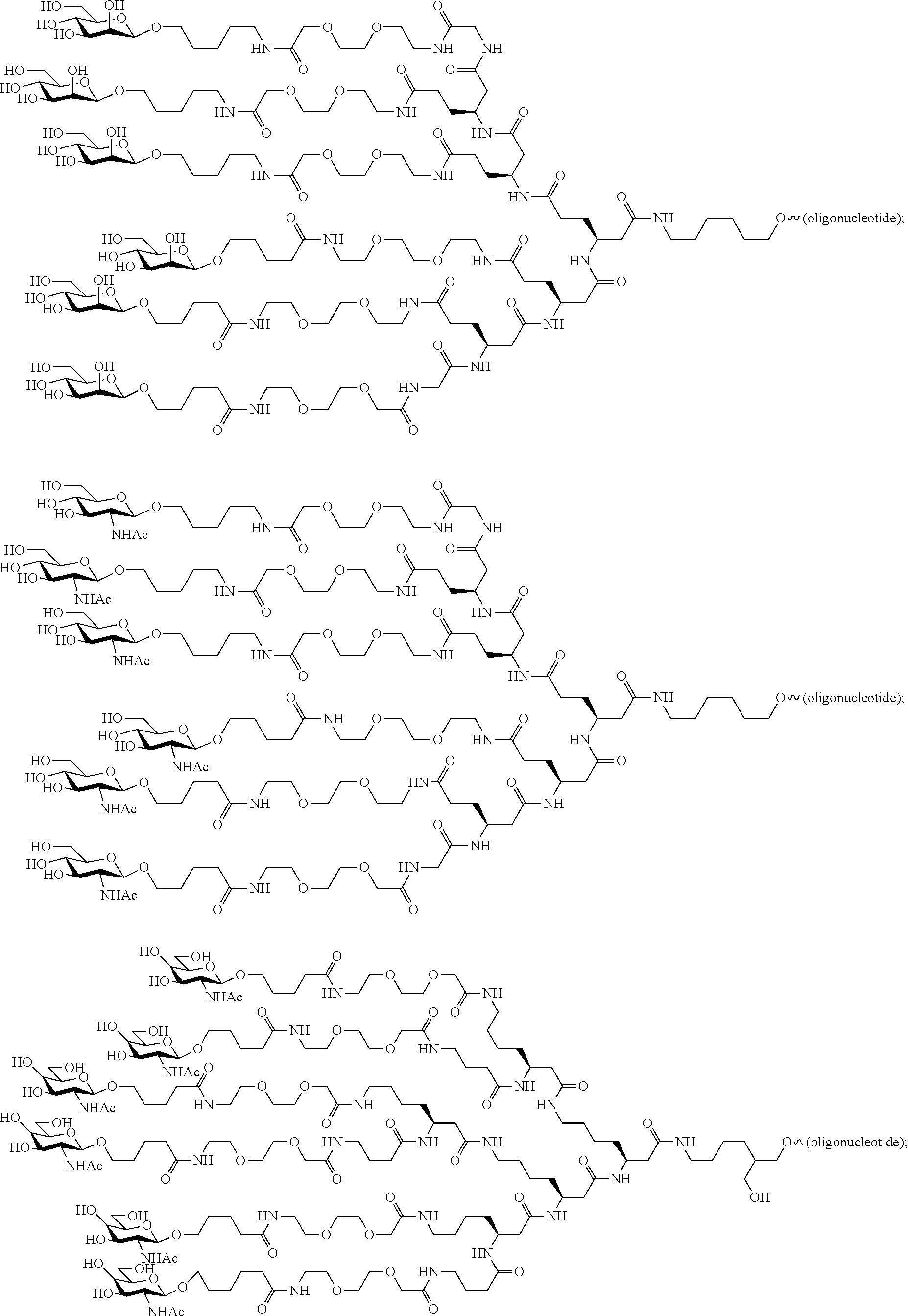

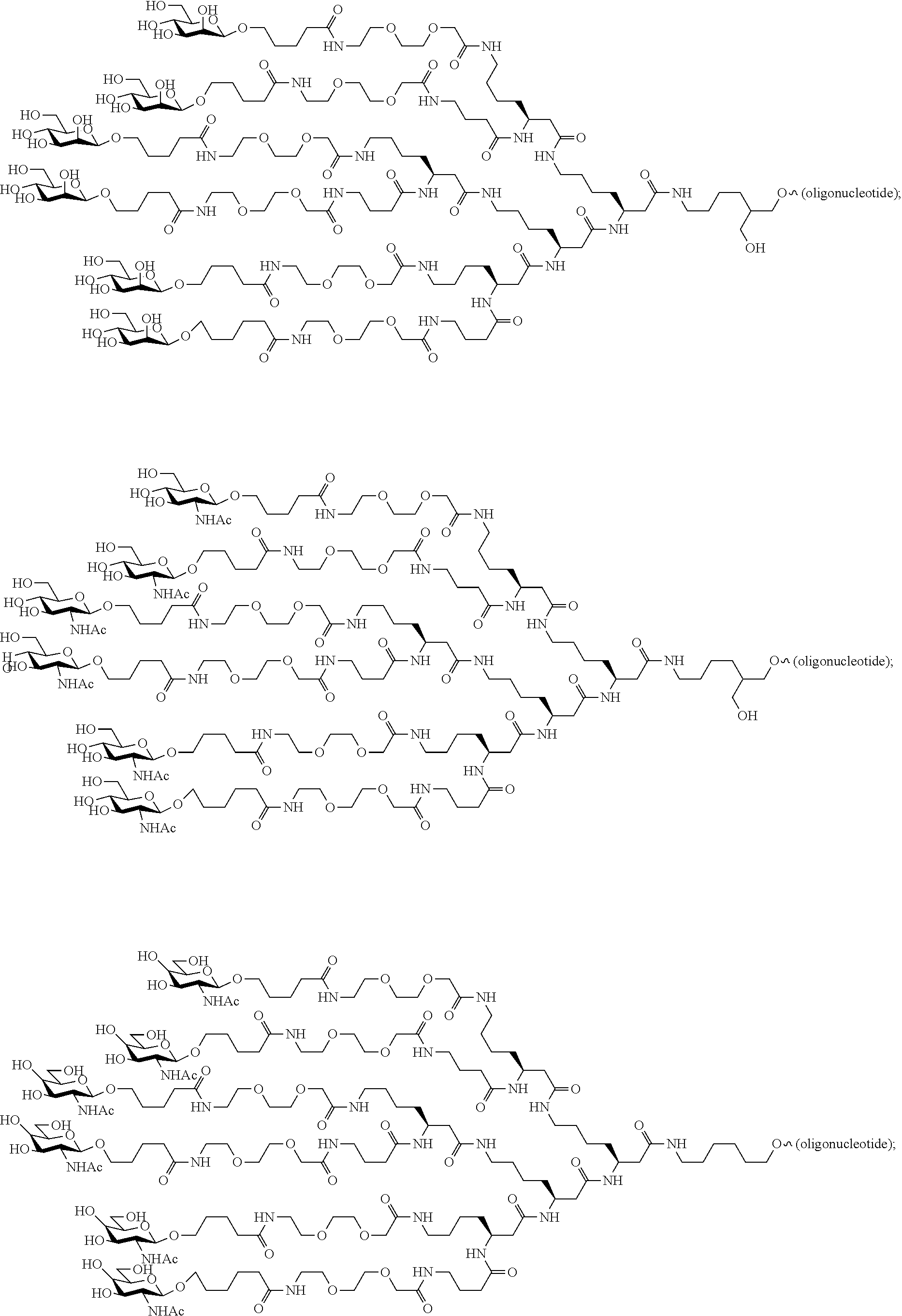

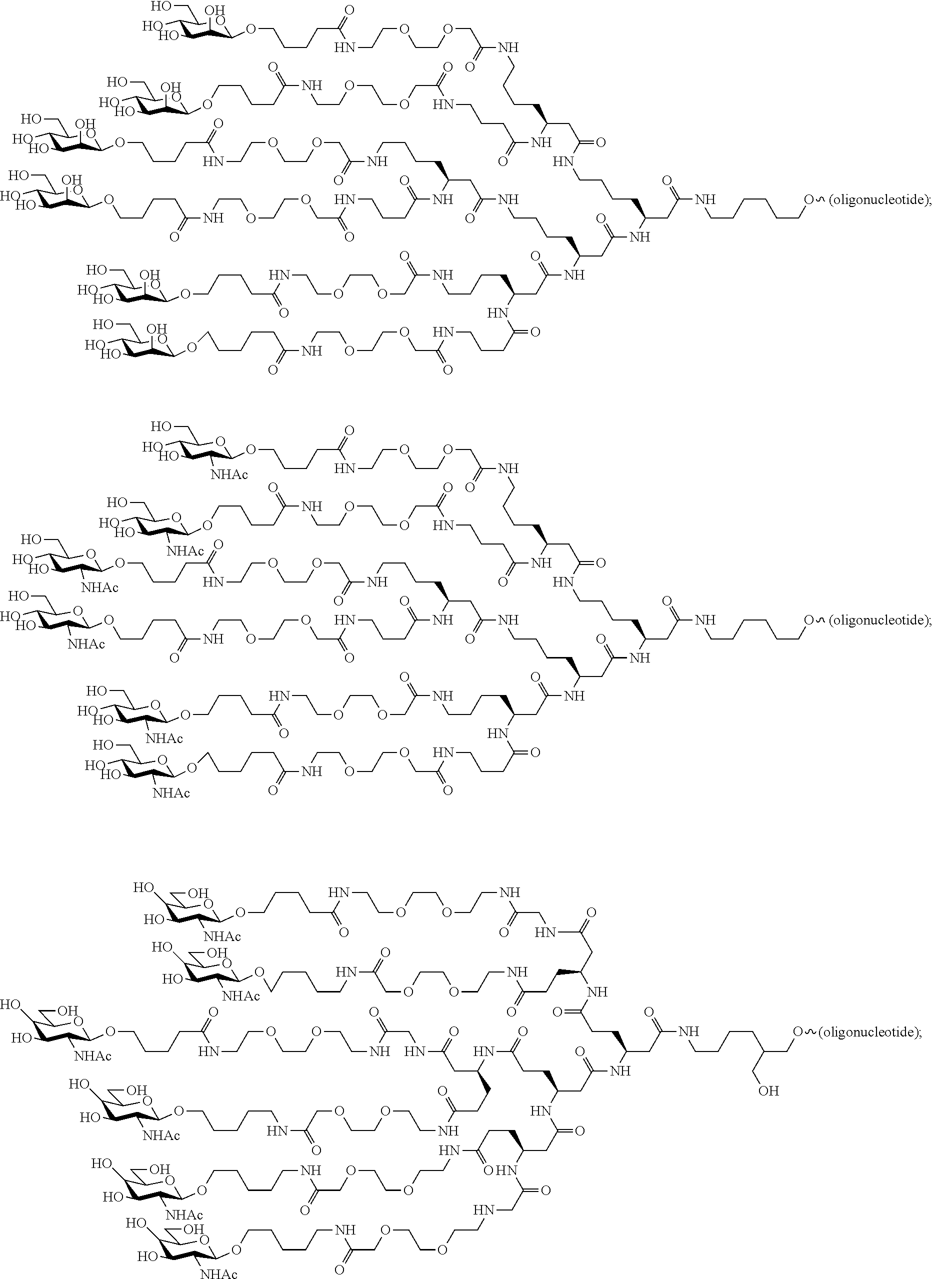

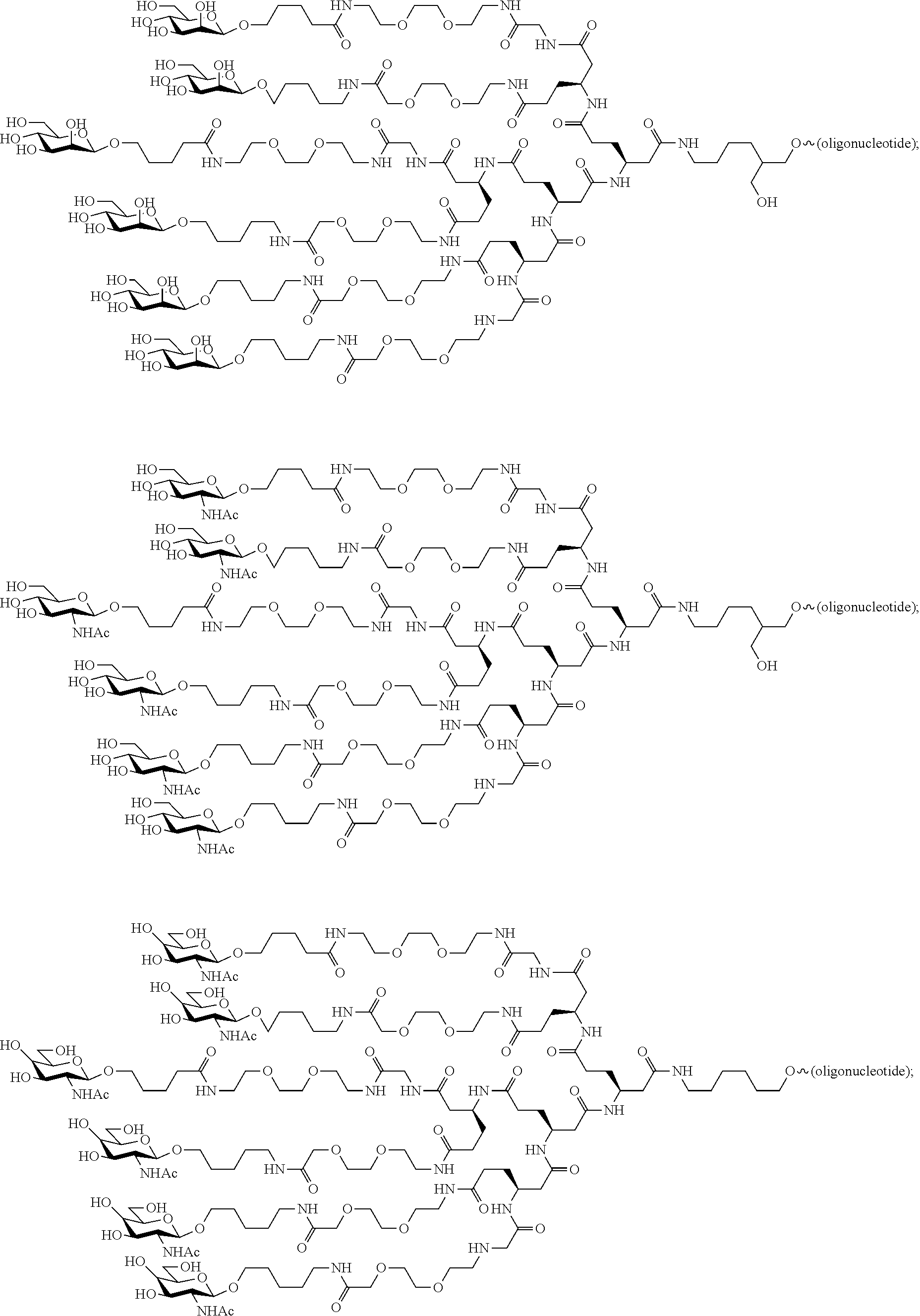

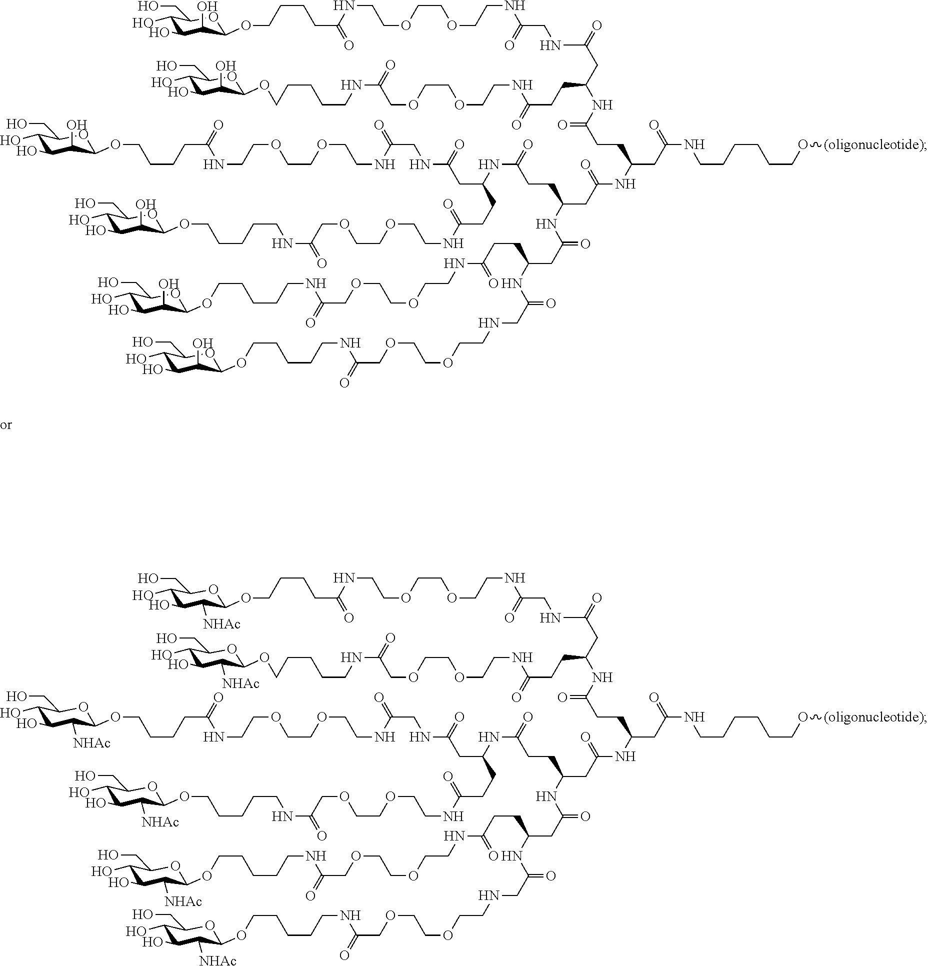

In some embodiments, the compounds provided herein comprise one or more of following formulae:

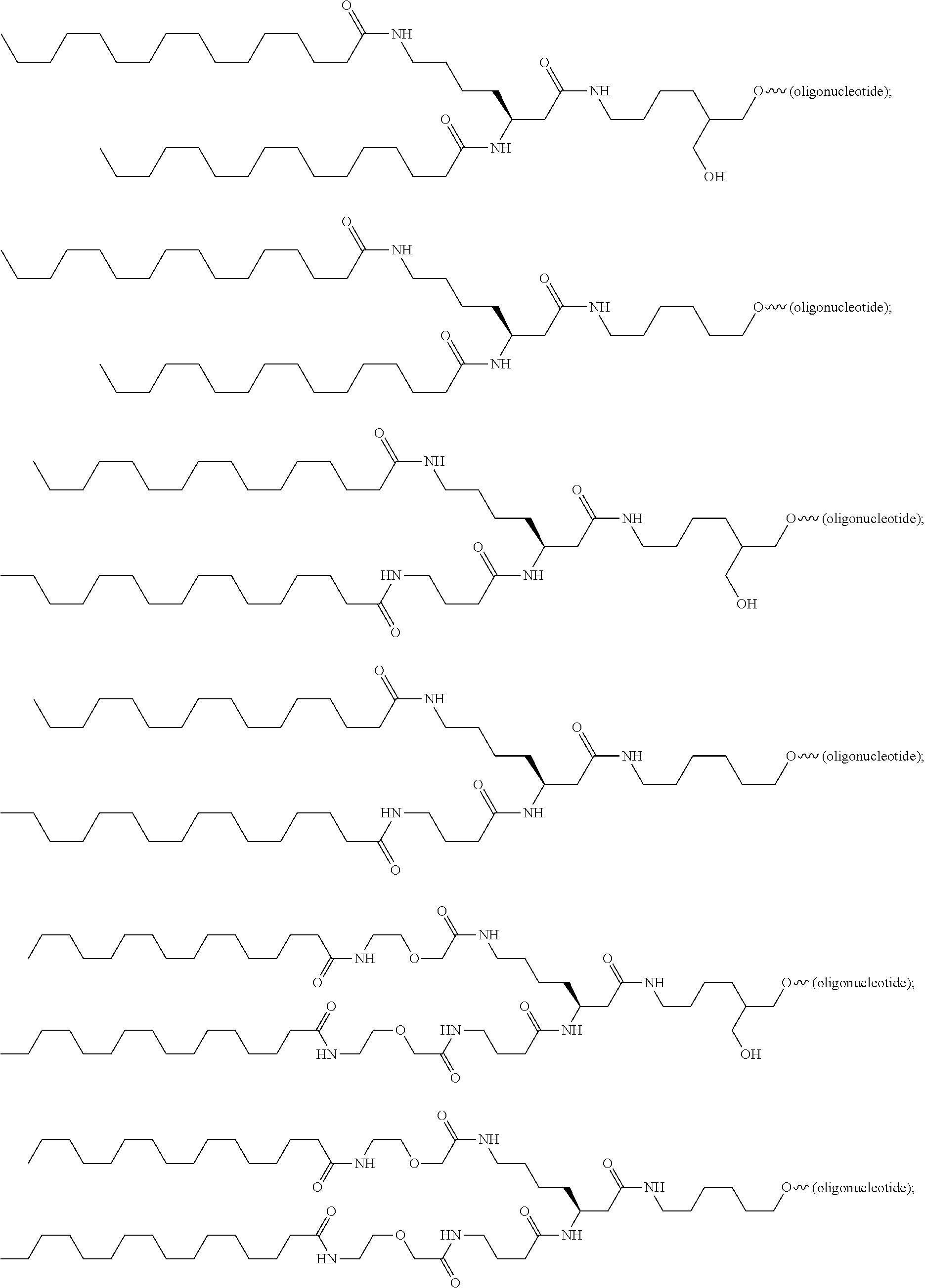

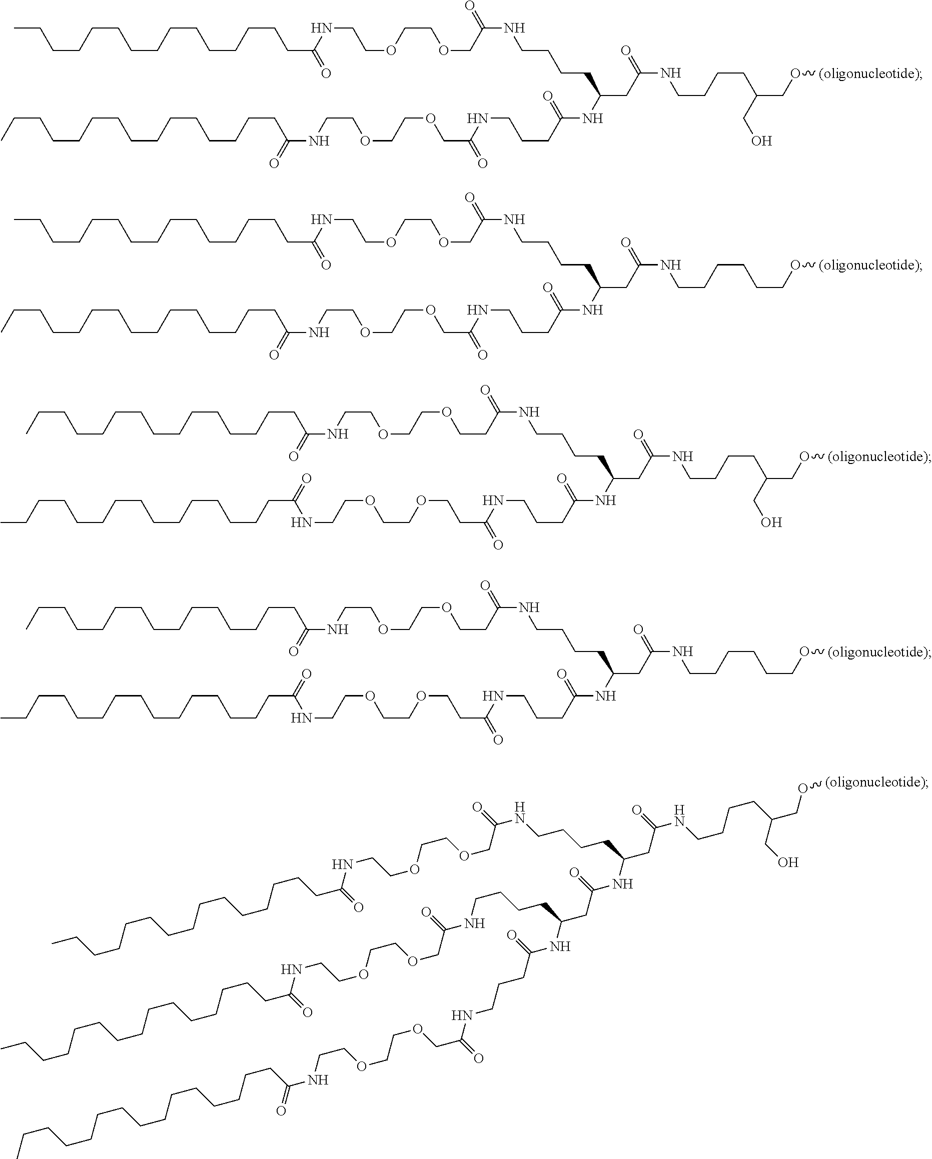

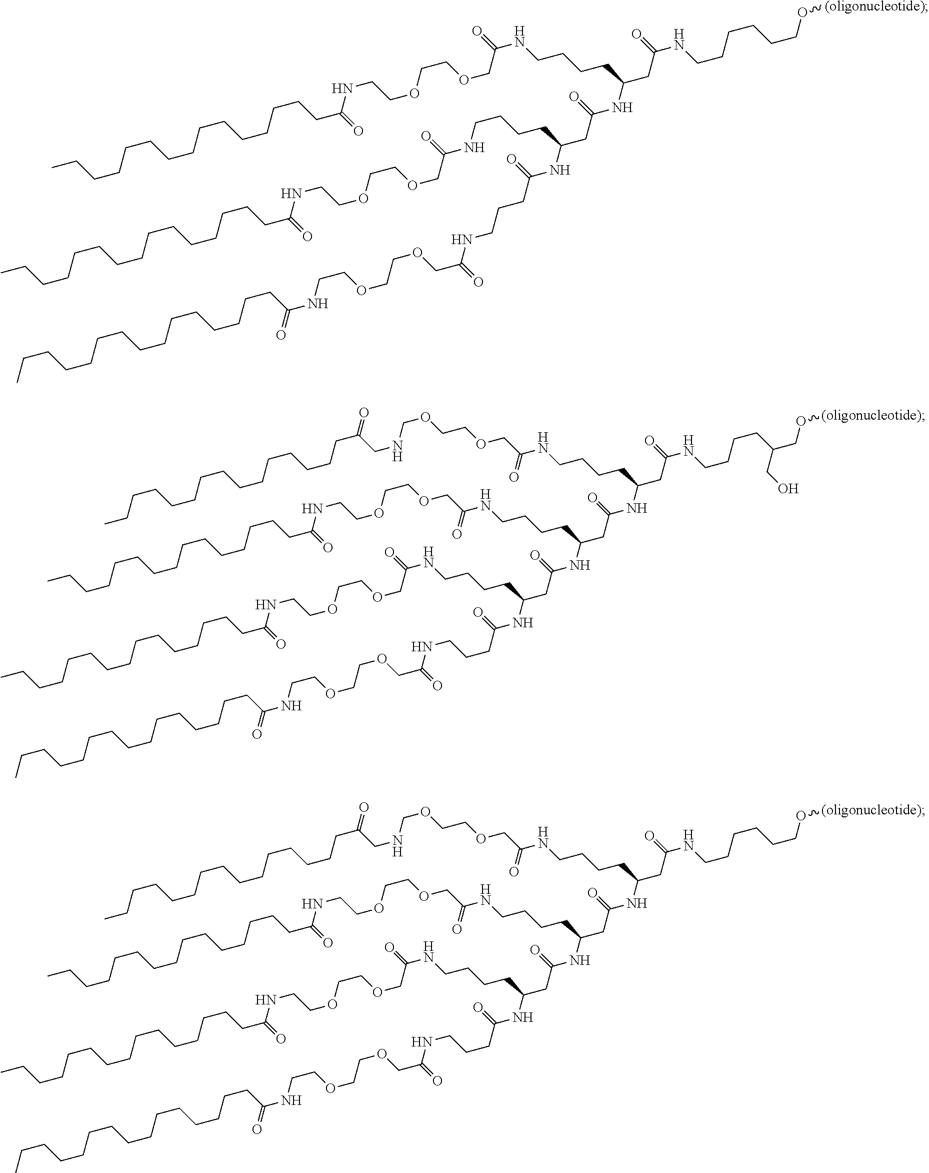

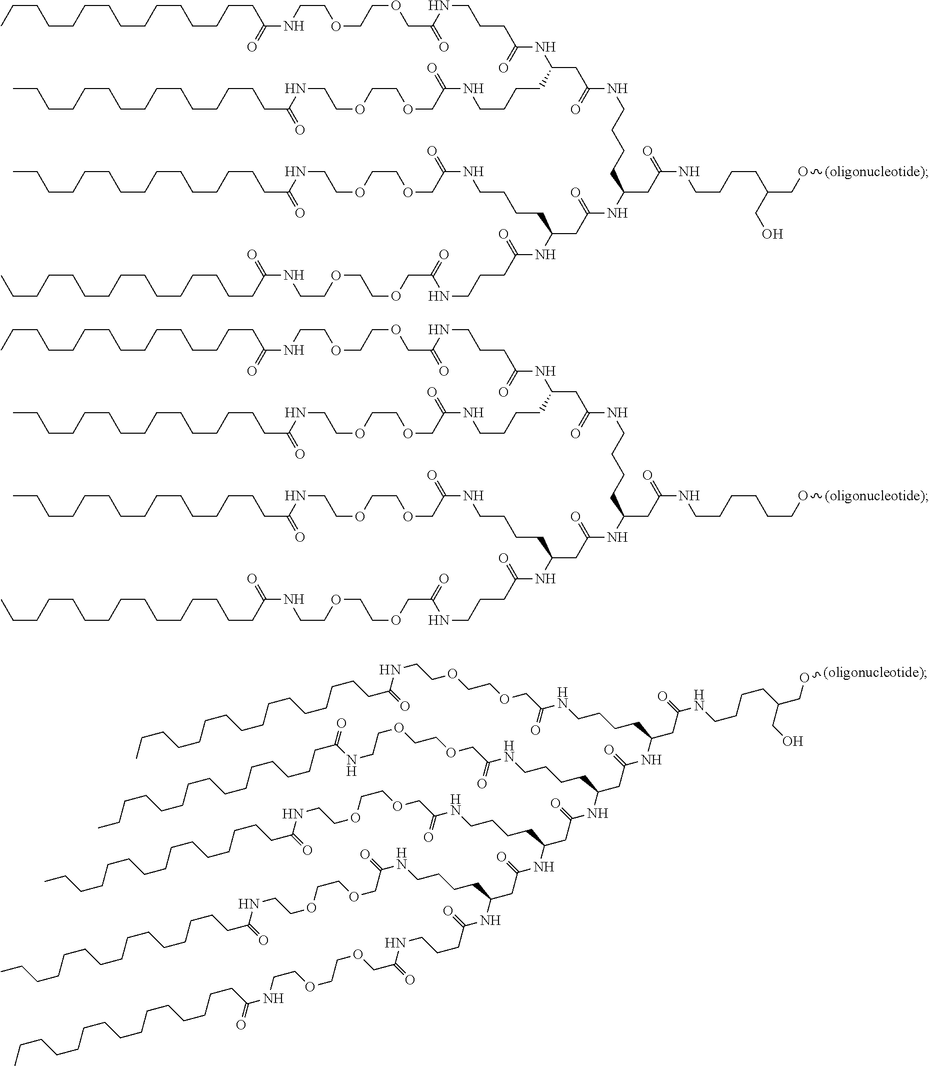

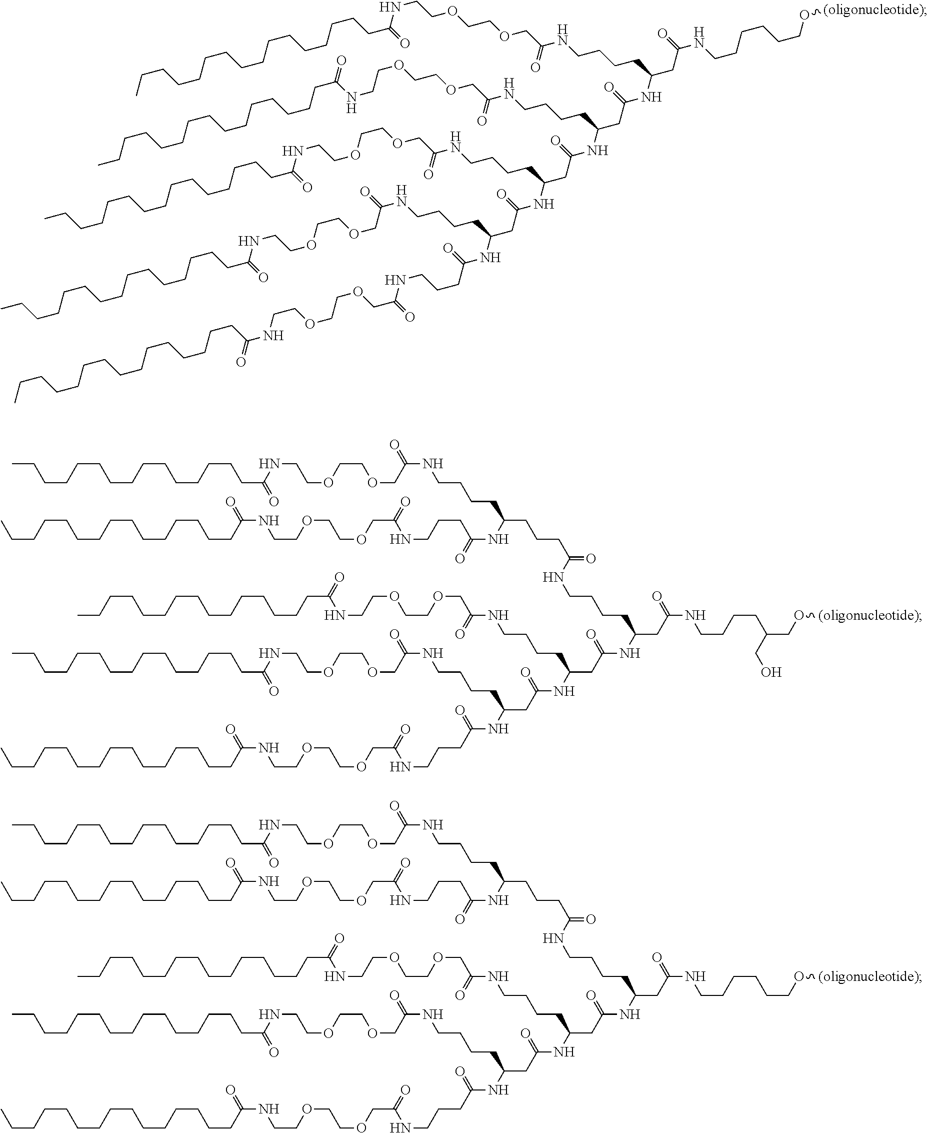

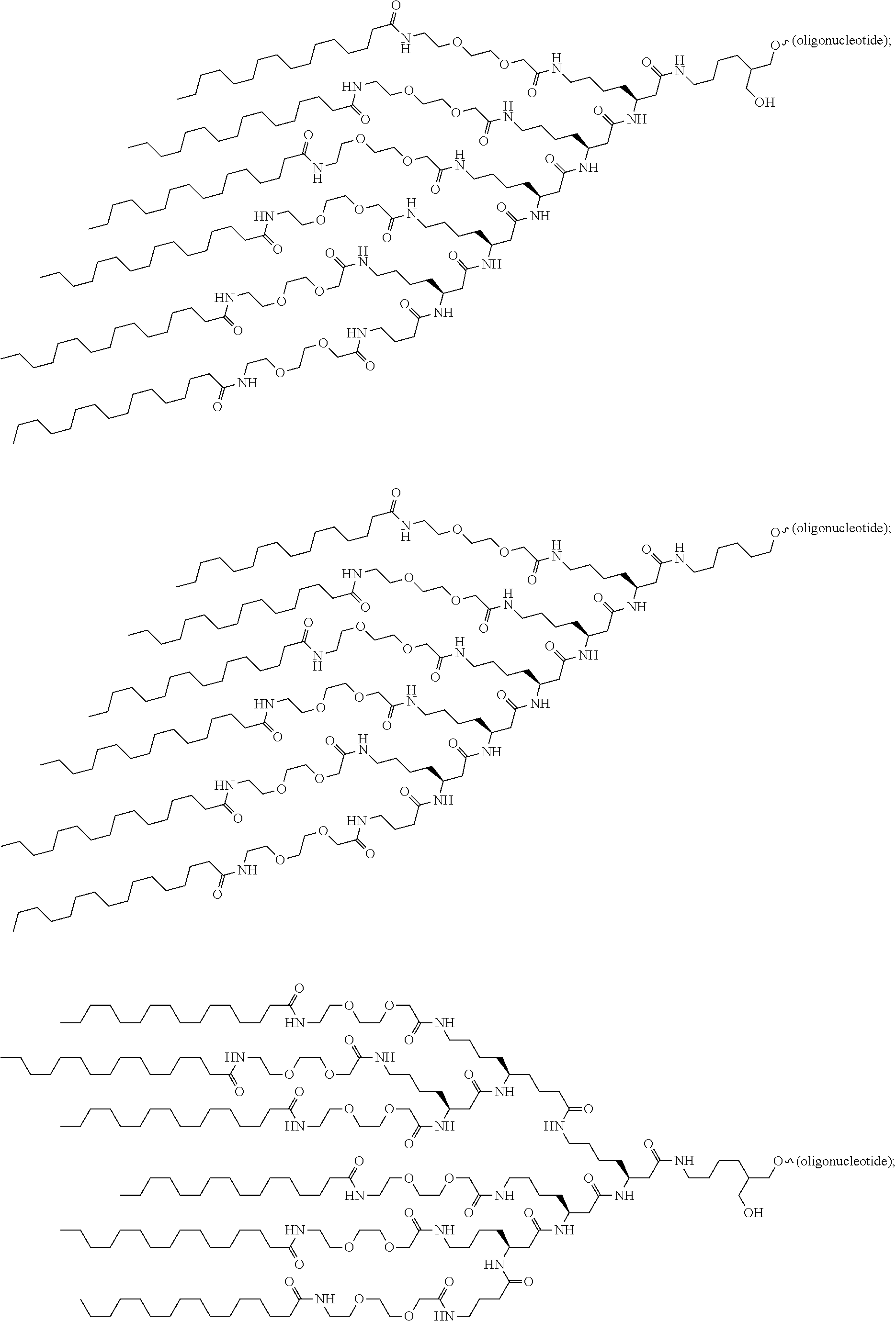

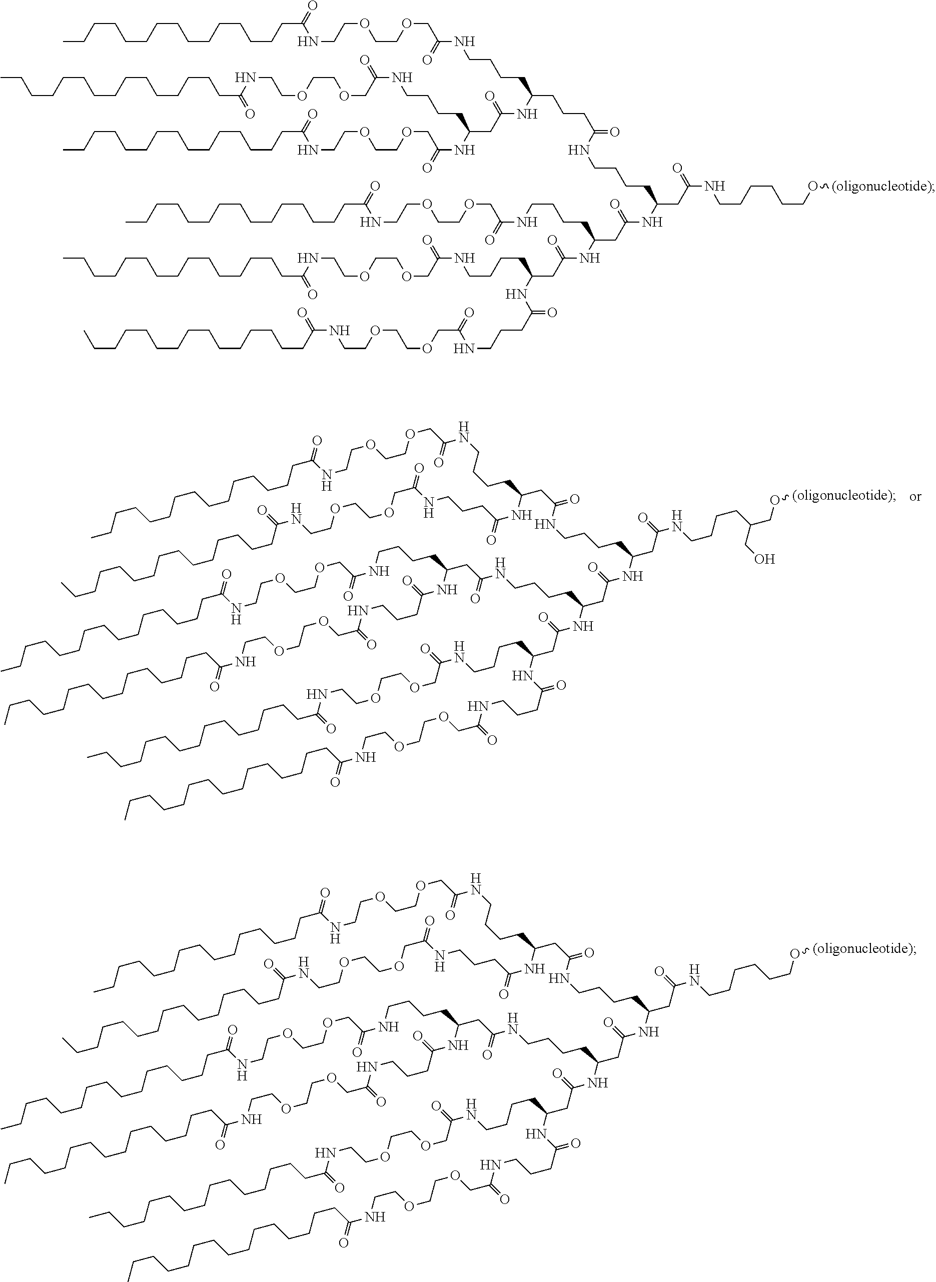

or a stereoisomer or a salt thereof.

In some embodiments, the compounds provided herein comprise one or more of following formulae:

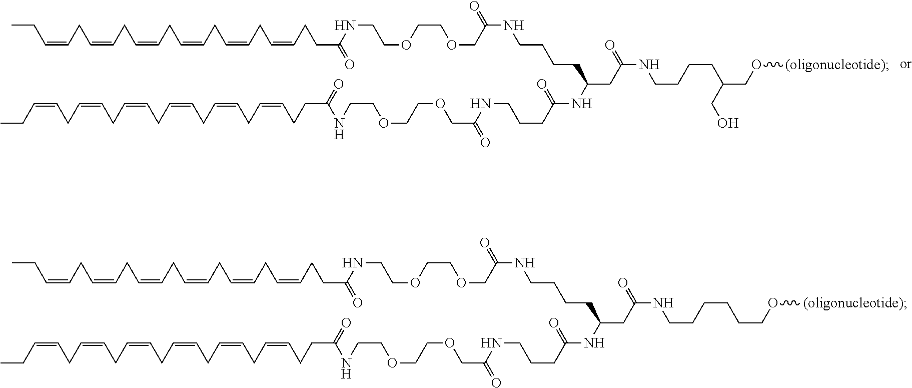

or a stereoisomer or a salt thereof.

In some embodiments, the compounds provided herein comprise one or more of following formulae:

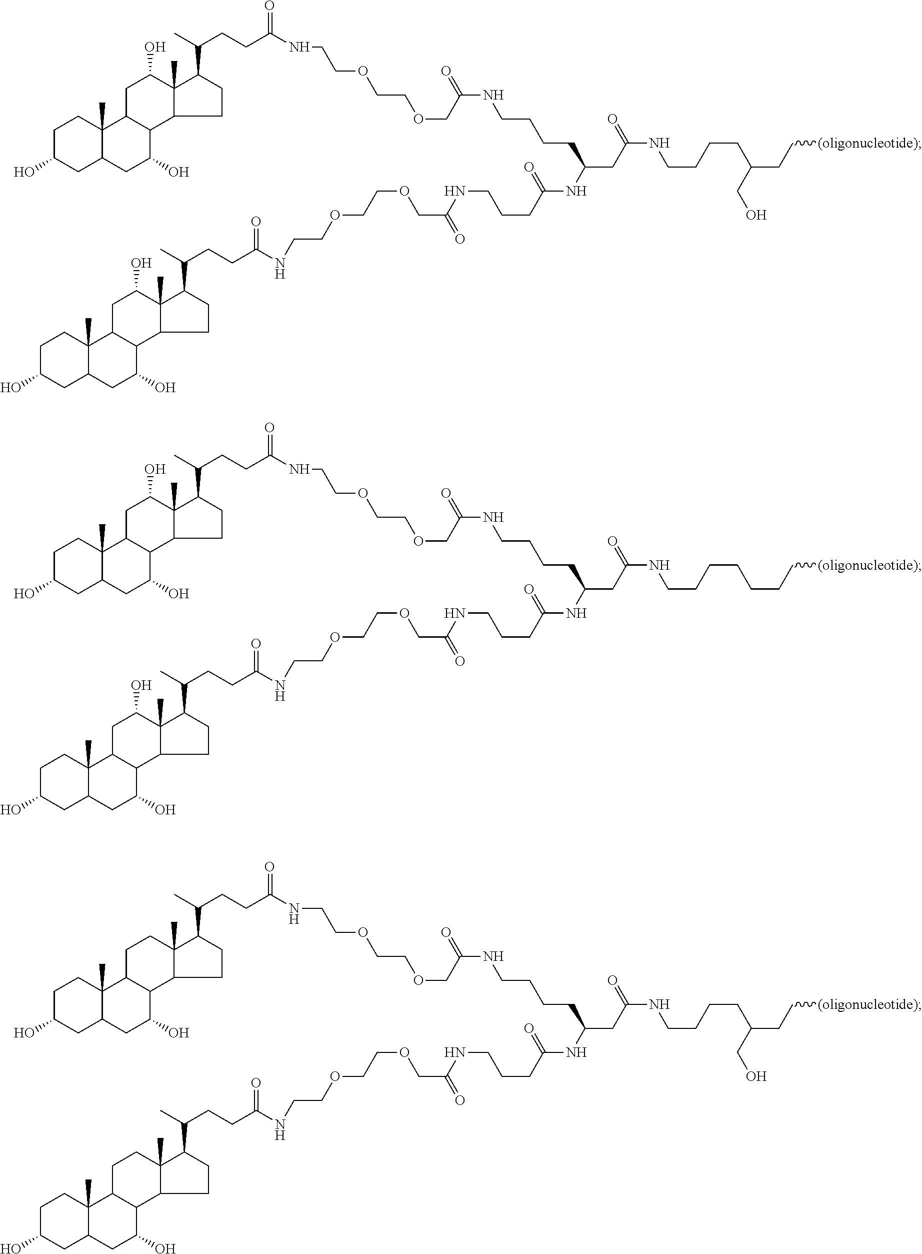

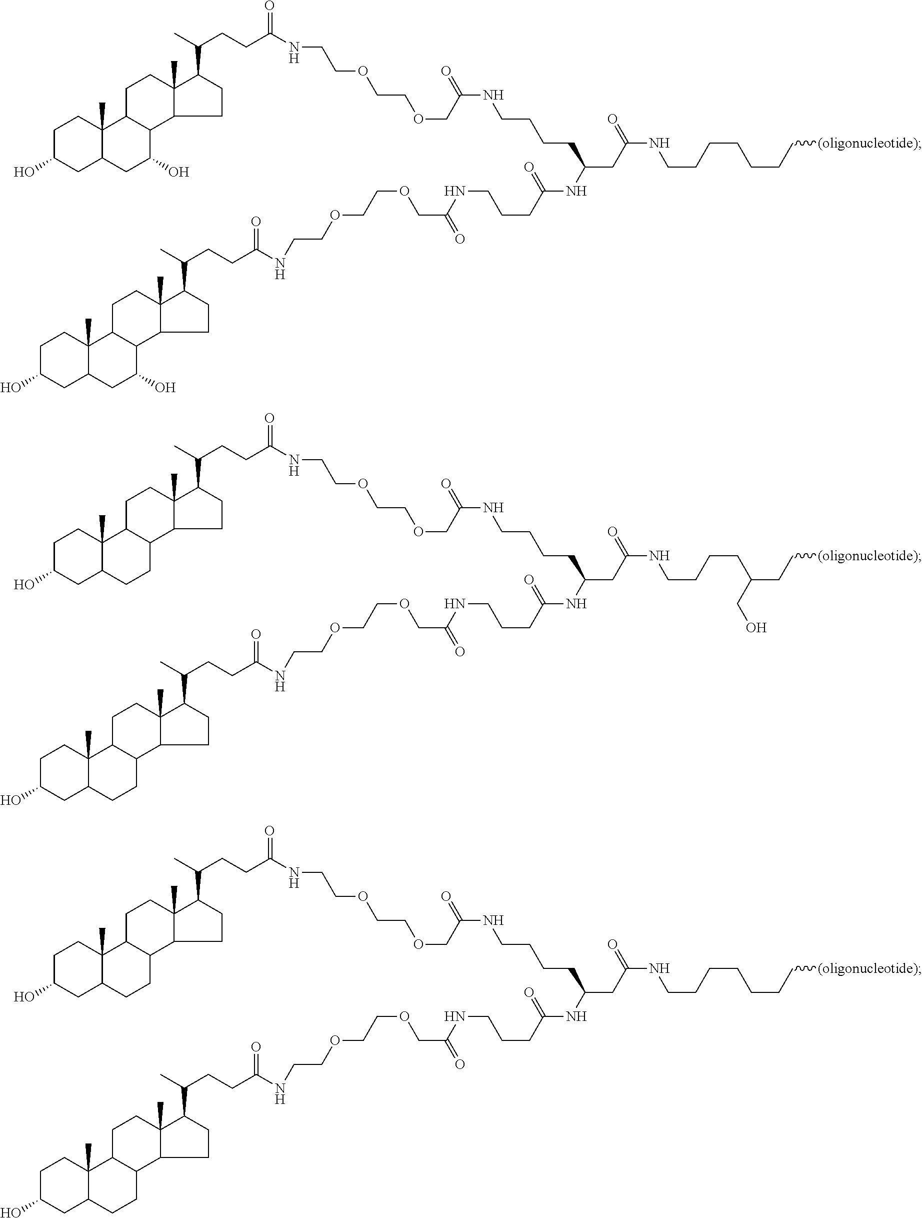

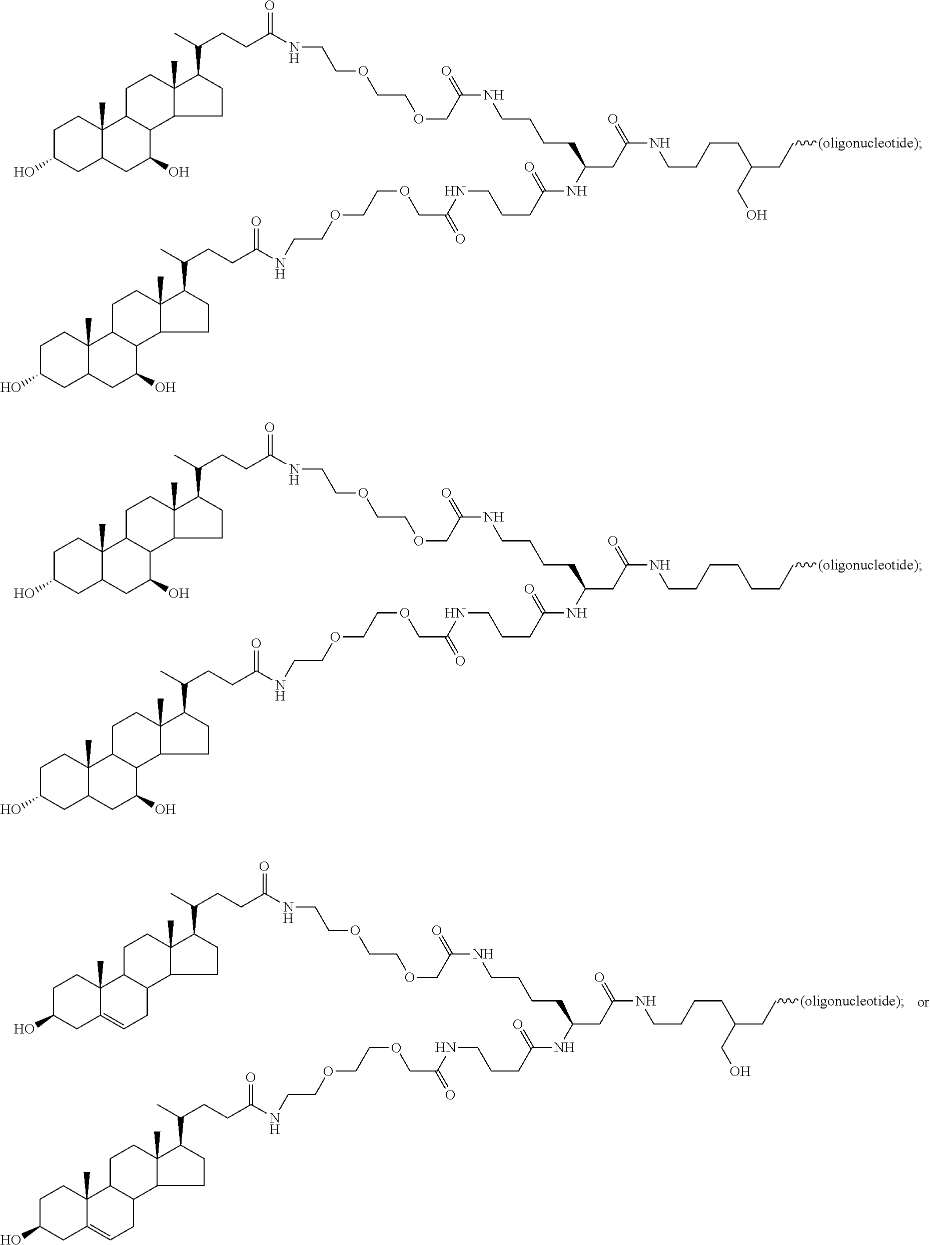

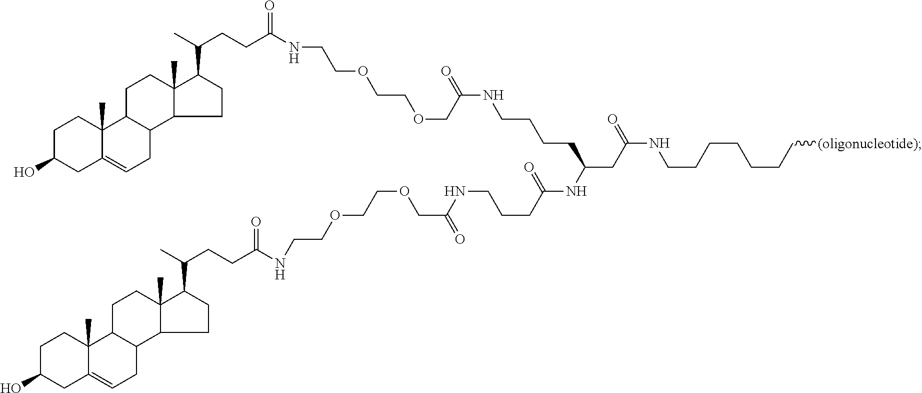

or a stereoisomer or a salt thereof.

In some embodiments, the compounds provided herein comprise one or more of following formulae:

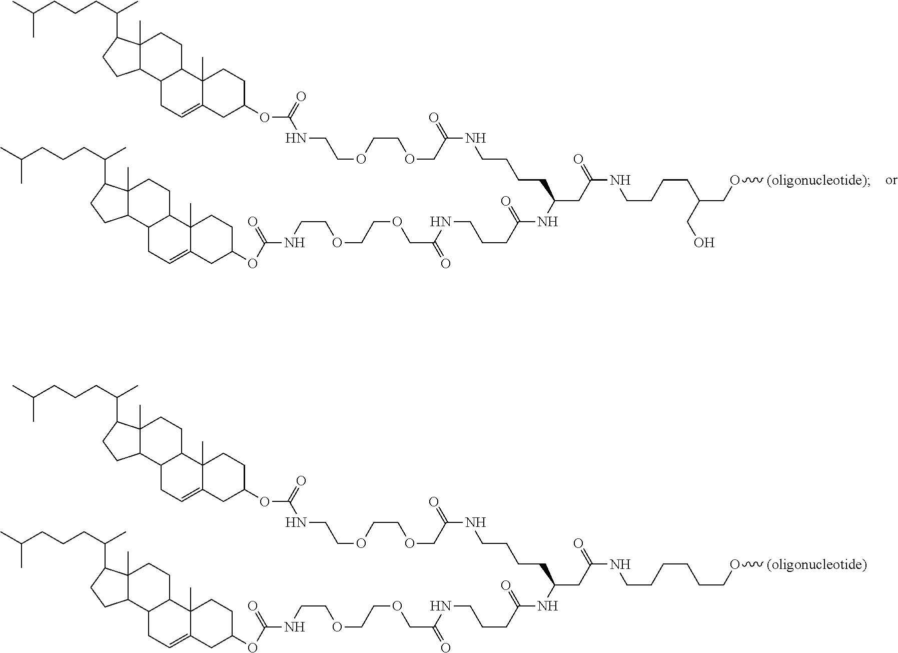

or a stereoisomer or a salt thereof.

In some embodiments of the formulae provided herein, the macromolecule, Z¹, or (oligonucleotide) comprises SEQ ID NO:1. In some embodiments of the formulae provided herein, (oligonucleotide) comprises SEQ ID NO:2. In some embodiments of the formulae provided herein, (oligonucleotide) comprises SEQ ID NO:3. In some embodiments of the formulae provided herein, (oligonucleotide) comprises SEQ ID NO:4. In some embodiments of the formulae provided herein, (oligonucleotide) comprises SEQ ID NO:5. In some embodiments of the formulae provided herein, (oligonucleotide) comprises SEQ ID NO:6. In some embodiments of the formulae provided herein, (oligonucleotide) comprises SEQ ID NO:7. In some embodiments of the formulae provided herein, (oligonucleotide) comprises SEQ ID NO:8. In some embodiments of the formulae provided herein, (oligonucleotide) comprises SEQ ID NO:9. In some embodiments of the formulae provided herein, (oligonucleotide) comprises SEQ ID NO:10. In some embodiments of the formulae provided herein, (oligonucleotide) comprises SEQ ID NO:11.

In some embodiments of the formulae provided herein, the macromolecule, Z¹, or (oligonucleotide) comprises an mRNA or siRNA, optionally wherein the mRNA or siRNA is at least 85% or at least 90% pure.

In some embodiments, the macromolecule, Z¹, or (oligonucleotide) comprises a polymer of nucleotides of any length, including ribonucleotides, deoxyribonucleotides, analogs thereof, or mixtures thereof. In some embodiments, the oligonucleotide comprises single-, double-, or triple-stranded oligonucleotide, including, without limitation, single-, double-, or triple-stranded deoxyribonucleic acid (“DNA”), single-, double-, or triple-stranded ribonucleic acid (“RNA”). In some embodiments, the oligonucleotide may include one or more modification, including, without limitation, alkylation or a capping moiety, in addition to unmodified forms of the oligonucleotide. In some embodiments, the oligonucleotide includes polydeoxyribonucleotides (containing 2-deoxy-D-ribose), polyribonucleotides (containing D-ribose), including tRNA, rRNA, hRNA, siRNA, or mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the macromolecule, Z¹, or (oligonucleotide) comprises a regulatory RNA, including, without limitation, micro RNA, long non-coding RNA, enhancer RNA, CRISPR RNA. In some embodiments, the macromolecule, Z¹, or (oligonucleotide) comprises a processing RNA, including, without limitation, a small nuclear RNA, or small nucleolar RNA. In some embodiments, the macromolecule, Z¹, or (oligonucleotide) comprises an RNA involved in protein synthesis, including, without limitation, Messenger RNA, Ribosomal RNA, Signal recognition particle RNA, Transfer RNA, or Transfer-messenger RNA. In some embodiments, the macromolecule, Z¹, or (oligonucleotide) comprises an RNA involved in post-transcriptional modification or DNA replication, including, without limitation, Small nuclear RNA, Small nucleolar RNA, SmY RNA, Small Cajal body-specific RNA, Guide RNA, Ribonuclease P RNA, Ribonuclease MRP RNA, Y RNA, Telomerase RNA Component RNA, or Spliced Leader RNA. In some embodiments, the macromolecule, Z¹, or (oligonucleotide) comprises a regulatory RNA, including, without limitation, Antisense RNA, Cis-natural antisense transcript RNA, CRISPR RNA, Long noncoding RNA, MicroRNA, Piwi-interacting RNA, Small interfering RNA, Short hairpin RNA, Trans-acting siRNA, Repeat associated siRNA, 7SK RNA, or Enhancer RNA. In some embodiments, the macromolecule, Z¹, or (oligonucleotide) comprises a parasitic RNA, including, without limitation, a retrotransposon RNA, a viral genome RNA, a viroid RNA, or a satellite RNA. In some embodiments, the macromolecule, Z¹, or (oligonucleotide) comprises a vault RNA. In some embodiments, the macromolecule, Z¹, or (oligonucleotide) comprises an RNA selected from non coding RNA, non messenger RNA, small RNA, small non messenger RNA, transfer RNA, soluble RNA, messenger RNA, protein coding RNA, ribosomal RNA, 5S ribosomal RNA, 5.8S ribosomal RNA, small subunit ribosomal RNA, large subunit ribosomal RNA, nucleolar remodeling complex associated RNA, promoter RNA, 6S RNA, antisense RNA, antisense micro RNA, cis-natural antisense transcript RNA, CRISPR RNA, trans-activating crRNA, CRISPR-Cas RNA, DNA damage response RNA, DSB-induced small RNA, double stranded RNA, endogenous small interfering RNA, extracellular RNA, guide RNA, heterochromatic small interfering RNA, heterochromatic small interfering RNA, heterogeneous nuclear RNA, RNA interference RNA, long intergenic non-coding RNA, long non coding RNA, micro RNA, natural antisense short interfering RNA, natural antisense short interfering RNA, oxidative stress response RNA, piwi-interacting RNA, QDE-2 interfering RNA, Repeat associated siRNA, mitochondrial RNA processing ribonuclease RNA, ribonuclease P RNA, small Cajal body-specific RNA, small-scan RNA, small cytoplasmic RNA, small conditional RNA, sugar transport-related sRNA, short hairpin RNA, small interfering RNA, spliced leader RNA, mRNA trans-splicing RNA, small nucleolar RNA, small nuclear RNA, small nuclear ribonucleic proteins RNA, 5′ small nucleolar RNA capped and 3′ polyadenylated long noncoding RNA, signal recognition particle RNA, single stranded RNA, small temporal RNA, trans-acting siRNA, transfer-messenger RNA, U spliceosomal RNA, vault RNA, X-inactive specific transcript RNA, Y RNA, natural antisense transcript RNA, precursor messenger RNA, circular RNA, multicopy single-stranded RNA, or cell-free RNA. In some embodiments, the oligonucleotide comprises a circular oligonucleotide, including, without limitation, a viroid, a plasmid, a covalently closed circular DNA (cccDNA), a circular bacterial chromosome, a mitochondrial DNA (mtDNA), a chloroplast DNA (cpDNA), or an extrachromosomal circular DNA (eccDNA). In some embodiments, the circular oligonucleotide is circularized by overlapping base pairing rather than covalently closed circular oligonucleotide. In some embodiments, the oligonucleotide comprises an mRNA. In some embodiments, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the oligonucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

In some embodiments, one or more phosphoramidite provided herein including an amino-acid cluster, having ligands described herein, is conjugated via standard amidite conjugation conditions, including under inert (e.g., anhydrous) conditions, to a macromolecule in solution at one or more free hydroxyl or primary amine moieties in the macromolecule. Thus, provided herein is a reaction product formed by conjugation of a macromolecule comprising one or more of hydroxyl or primary amine moieties with one, two, three, four, or more equivalents (relative to molar amount of macromolecule) of one or more phosphoramidite of an amino-acid cluster provided herein. In some embodiments, the macromolecule reaction product includes an oligonucleotide macromolecule or a peptide or protein macromolecule.

In some embodiments, provided herein are compositions, comprising one or more compounds provided herein. The compositions may include one or more carriers, including, without limitation, one or more solvents. In some embodiments, provided herein are pharmaceutical compositions comprising one or more of the compounds provided herein, and at least one pharmaceutically acceptable carrier. In some embodiments, the composition is a solid composition. In some embodiments, the composition is an implantable composition. In some embodiments, the composition is an inhalable composition. In some embodiments, the composition is an orally ingestible composition. In some embodiments, the composition is an injectable composition. In some embodiments, the composition is a flowable powder composition. In some embodiments, the composition is a liquid composition, including, without limitation, a suspension or emulsion of the compound therein. In some embodiments, the composition is a gel, cream, or ointment comprising the compound.

Methods

The amino-acid clusters herein, except the corresponding phosphoramidite compounds, may be useful as components of therapeutic applications. Thus, it is understood that such compounds are administrable in conjunction with methods of treatment in a subject in need thereof. Thus, provided herein are, at least, methods, comprising administering the compound to a subject. Routes of administration may be via any route suitable for delivery of the compounds herein to a subject, including those described herein.

Kits

In some embodiments, provided herein are packaged forms of a compound provided herein, packaged compositions, or packaged pharmaceutical compositions comprising a container holding a therapeutically effective amount of a compound described herein, and instructions for using the compound in accordance with one or more of the methods provided herein.

The present compounds and associated materials can be finished as a commercial product by the usual steps performed in the present field, for example by appropriate sterilization and packaging steps. For example, at doses of 25-35 kGy, both e-beams and gamma radiation may effectively sterilize pharmaceuticals. Alternatively, the material can be treated by UV/vis irradiation (200-500 nm), for example using photo-initiators with different absorption wavelengths (e.g., Irgacure 184, 2959), preferably water-soluble initiators (e.g., Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.). The compounds may also be packaged under inert conditions (e.g., de-oxygenated or dehydrated atmosphere, e.g., nitrogen or argon atmosphere), to preserve the compound from degradation.

According to further embodiments, the present compounds can also be provided in kit form combined with other components, including without limitation, those necessary for use of the material for synthetic methods or administration of the material to the patient. For example, disclosed kits, such as for use in treatments, can further comprise, for example, administration materials.

The compounds or compositions provided herein may be prepared and placed in a container for storage at ambient or elevated temperature. When the compound or composition is stored in a polyolefin plastic container as compared to, for example, a polyvinyl chloride plastic container, discoloration of the compound or composition may be reduced, whether suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container, including without limitation an amber colored glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include phosphoramidites described herein or dosage forms that include a form of the compounds or compositions described herein. The compounds or compositions provided herein may need storage lasting up to, or longer than, three months; in some cases up to, or longer than one year. The containers may be in any form suitable to contain the contents—for example, a bag, a bottle, or a box, or any combination thereof.

The compounds and processes described herein will be better understood by reference to the following examples, which are intended as an illustration of and not a limitation upon the scope of the present description.

### EXAMPLES

The following selected examples describe certain techniques for producing specific and general synthetic methods of pharmaceutically stability improved functional moieties and their siRNA conjugates as described herein, as well as certain analyses of stability and activities of certain compounds described herein.

### Example 1. General Method for the Synthesis of Oligonucleotide Containing Multivalent Ligand

A. General Method for the Synthesis of Multivalent Ligand Solid Supports from Fmoc or ivDde AmC7 (DMT) CPG (Controlled Pore Glass) or PS (Polystyrene) or CPSG (Controlled Porosity Silica Gel).

Fmoc or ivDde protected AmC7 (DMT) CPG is placed in solid phase reactor and rinsed with DCM and DMF. Fmoc protection group is removed by 20% 4-methylpiperidine in DMF and ivDde protection group is removed by 4% hydrazine in DMF. The first beta-amino acid is coupled under the condition with HATU, DIPEA in DMF. Then, the next amino acids are sequentially coupled on the backbone and/or side chain by repeating the N-terminal deprotection of Fmoc or ivDde protection group and coupling reaction under the condition with HATU, DIPEA in DMF until the targeted multivalent ligand is obtained. Loading capacity is measured by DMT quantification.

B. General Method for the Synthesis of Oligonucleotides with Multivalent Ligand Solid Supports

A functionalized oligonucleotide is synthesized on multivalent ligand solid supports by automated oligonucleotide solid phase synthesizer. Oligonucleotides containing multivalent ligands are synthesized by standard process using phosphoramidite technology on multivalent ligand solid supports. Depending on the scale either a MerMade 12 (Bioautomation) or a Dr. Oligo 48 (Biolytic) or OligoPilot 100 (Cytiva) is used. All phosphoramidites are purchased from, but not limited to, ChemGenes and Glen Research. All amidities are dissolved in anhydrous acetonitrile and/or DMF and/or DCM in adequate concentration. Deblock solution is selected from, but not limited to, acetic acid, chloroacetic acid, dichloroacetic acid, trichloroacetic acid, or trifluoroacetic acid in an inert solvent such as DCM or toluene. Activator solution is selected from, but not limited to, acidic azole catalysts including 1H-tetrazole, 5-ethylthio-1H-tetrazole (ETT) and 2-benzylthio-1H-tetrazole (BTT) or 4,5-dicyanoimidazole (DCI) or a number of similar compounds which is dissolved in anhydrous acetonitrile in adeauate concentration. Capping solution is selected from, but not limited to, a mixture of acetic anhydride and pyridine in THF and N-methylimidazole in acetonitrile. Oxidizing solution is selected from, but not limited to iodine in water, pyridine and THF and tert-butyl hydroperoxidie, (1S)-(+)-(10-camphorsulfonyl)-oxaziridine (CSO). Sulfurization solution is selected from, but not limited to, 3-(dimethylaminomethylidene)amino-3H-1,2,4-dithiazole-3-thione (DDTT), 3H-1,2-benzodithiol-3-one 1,1-dioxide (Beaucage reagent), or N,N,N′,N′-tetraethylthiramdisulfide (TETD).

C. General Method for the Synthesis of Multivalent Ligand Phosphoramidite

Fmoc or ivDde protected AmC7 (DMT) solid support is placed in solid phase reactor and rinsed with DCM and DMF. Fmoc protection group is removed by 20% 4-methylpiperidine in DMF and ivDde protection group is removed by 4% hydrazine in DMF. The first beta-amino acid is coupled under the condition with HATU, DIPEA in DMF. Then, the next amino acids are sequentially coupled on the backbone and/or side chain by repeating the N-terminal deprotection of Fmoc or ivDde protection group and coupling reaction under the condition with HATU, DIPEA in DMF until the targeted multivalent ligand is obtained. Loading capacity is measured by DMT quantification. Then, solid support is removed by ammonium hydroxide solution, and the resulting alcohol compound is transformed into multivalent ligand phosphoramidite by phosphitylation reaction.

D. General Method for the Synthesis of Oligonucleotides with Multivalent Ligand Phosphoramidite

UnyLinker CPG is placed in synthetic column and a functionalized oligonucleotide is synthesized on solid support by automated oligonucleotide solid phase synthesizer. Multivalent ligand phosphoramidite is dissolved in anhydrous acetonitrile and/or DCM and/or DMF in adequate concentration. Oligonucleotide synthesis follows the general method for the synthesis of oligonucleotide shown in B.

E. General Method for the Reverse Synthesis of Oligonucleotides Followed by Multivalent Ligand Post-Synthesis

A functionalized oligonucleotide is reverse-synthesized by automated oligonucleotide solid phase synthesizer, followed by post-synthesis using step-by-step conjugation with beta-amino acid, amino acid, and ligands under the condition of HATU, DIPEA and DMF. Oligonucleotides are reverse-synthesized by standard process using phosphoramidite technology on UnyLinker solid supports. Depending on the scale either a MerMade 12 (Bioautomation) or a Dr. Oligo 48 (Biolytic) or OligoPilot 100 (Cytiva) is used. All reverse-phosphoramidites are purchased from, but not limited to, ChemGenes and Glen Research. All reverse-phosphoramidites are dissolved in anhydrous acetonitrile and/or DMF and/or DCM in adequate concentration. Deblock solution is selected from, but not limited to, acetic acid, chloroacetic acid, dichloroacetic acid, trichloroacetic acid, or trifluoroacetic acid in an inert solvent such as DCM or toluene. Activator solution is selected from, but not limited to, acidic azole catalysts including 1H-tetrazole, 5-ethylthio-1H-tetrazole (ETT) and 2-benzylthio-1H-tetrazole (BTT) or 4,5-dicyanoimidazole (DCI) or a number of similar compounds which is dissolved in anhydrous acetonitrile in adeauate concentration. Capping solution is selected from, but not limited to, a mixture of acetic anhydride and pyridine in THF and N-methylimidazole in acetonitrile. Oxidizing solution is selected from, but not limited to iodine in water, pyridine and THF and tert-butyl hydroperoxidie, (1S)-(+)-(10-camphorsulfonyl)-oxaziridine (CSO). Sulfurization solution is selected from, but not limited to, 3-(dimethylaminomethylidene)amino-3H-1,2,4-dithiazole-3-thione (DDTT), 3H-1,2-benzodithiol-3-one 1,1-dioxide (Beaucage reagent), or N,N,N′,N′-tetraethylthiramdisulfide (TETD).

H. Duplexation of Single Strand RNAs

Sense and antisense strands are carefully mixed in equal molar amount and vortexed for at least 30 seconds. After quantification of sense and antisense strands by in process analysis, the sense or antisense strand is adjusted to make sure no residual single stranded material. The duplex solution is heated to 85° C. for 3 minutes and gradually cooled to room temperature, followed by lyophilization.

Sequences of oligonucleotide examples used herein are shown in Table 1, supra. Examples of certain amino-acid cluster conjugates described herein are shown in Tables 2-10, supra. Conjugation of amino-acid clusters including ligands provided herein may be accomplished under appropriate solid phase conditions suitable for peptide or carbohydrate synthesis.

### Example 2. Stability Test 1

The stability of oligonucleotides containing tri-GalNAc conjugate was tested under a protein digestion condition: oligonucleotide-amino-acid ligand cluster conjugate in a mixture shown in Table 11 was incubated at 37° C. for 1 hour, about 5 days, or about 7 days. After adding 2.5 μL of 3 M KCl, the sample of Table 11 was mixed well and vortexed, followed by incubation on ice for 10 minutes to precipitate SDS. After centrifugation for 10 minutes at 10000 g at 4° C., supernatant (40 μL) was transferred to a clean pre-chilled tube. Then, oligonucleotide sample 10 μL was mixed with 6× loading dye (Promega, G190A) 2 μL. Total 12 μL was loaded on 12% Native PAGE at 120 V constant for 30 minutes, followed by staining with GelRed (Biotuum, 41003) for 15 minutes.

TABLE 11
Protein digestion sample.

| | Condition 1 |
|---|---|
| Oligonucleotide-Cluster | 10 μL |
| Conjugate Sample (10 | |
| μM) | |
| Lysis buffer (LGC | 32.5 μL |
| Biosearch Technologies | |
| #MTC096H) | |
| Proteinase K (50 mg/mL) | 2.5 μL |
| Total (μL) | 45 μL |

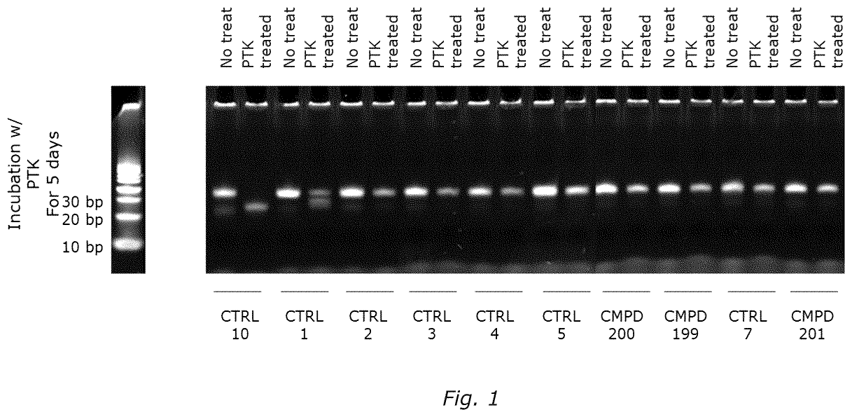

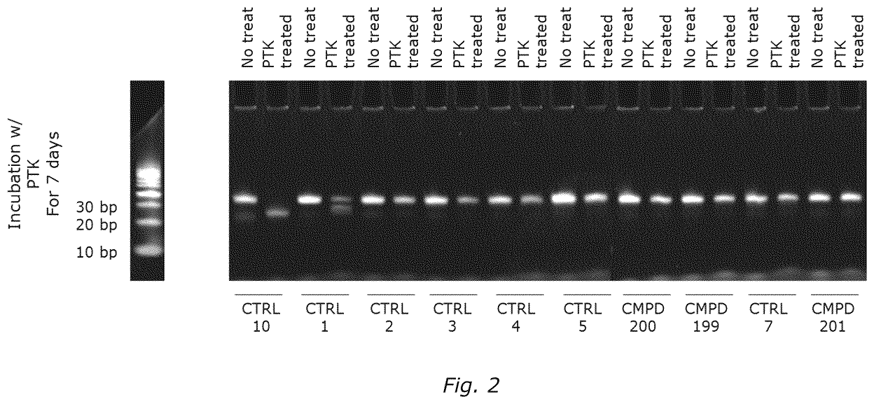

All oligonucleotide samples containing β-amino acid conjugated ligands showed better stability under the condition of protein digestion than oligonucleotide samples containing only D- or L-amino acid moieties. Results are shown in FIG. 1 and FIG. 2.

Test materials were prepared by duplexation with sense strand and antisenses, selected from Compound Nos. 197 and 199-216, where the Compound 199 contained (GalNAc-C5)₃-[(GABA)-(βH-Lys)-(βH-Lys)]-AmC7 conjugation, the Compound 200 contained (GalNAc-C5)₃-[(GABA)-(L-Lys)-(βH-Lys)]-AmC7 conjugation, and the Compound 201 contained (GalNAc-C5)₃-[(GABA)-(D-Lys)-(βH-Lys)]-AmC7 conjugation at the 3′-end of sense strand.

### Example 3. Stability Test 2

The stability of oligonucleotides containing tri-GalNAc conjugate was tested under the conditions of mouse plasma, mouse serum, and rat tritosome.
Mouse: C57BL/6, male, 10 weeks old
Plasma isolation (with EDTA): blood centrifugation at 2500 g for 15 minutes at RT
Serum isolation: blood centrifugation at 2500 g for 15 minutes at RT
Rat Tritosome was prepared under the conditions shown in Table 12.

TABLE 12

| | Condition 1 | Condition 2 |
|---|---|---|
| Rat tritosome (0.5 mg/mL) | 0 | 5 |
| GalNAc-asiRNA (10 μM, μL) | 1 | 1 |
| Catabolic buffer (10×) | 0 | 1 |
| UPW | 0 | 3 |
| 1× PBS (μL) | 9 | 0 |
| Total (μL) | 10 | 10 |

Test materials were incubated at 37° C. for 17 hours under the conditions shown in Table 13.

TABLE 13

| | Condition 1 | Condition 2 | Condition 3 |
|---|---|---|---|
| Mouse plasma (100%, μL) | 0 | 9 | 0 |
| Mouse serum (100%, μL) | 0 | 0 | 9 |
| SIRNA (10 μM, μL) | 1 | 1 | 1 |
| 1x PBS (μL) | 9 | 0 | 0 |
| Total (μL) | 10 | 10 | 10 |

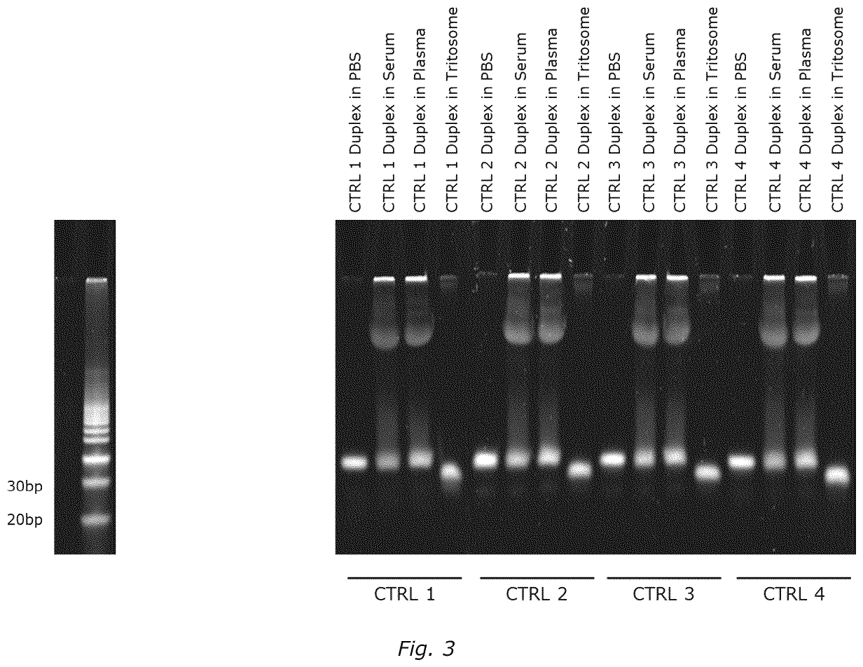

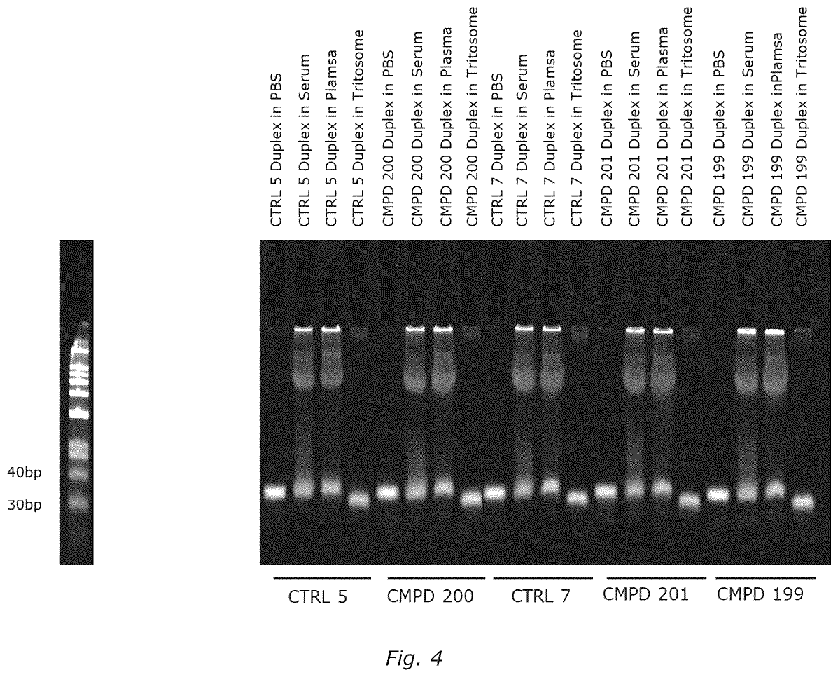

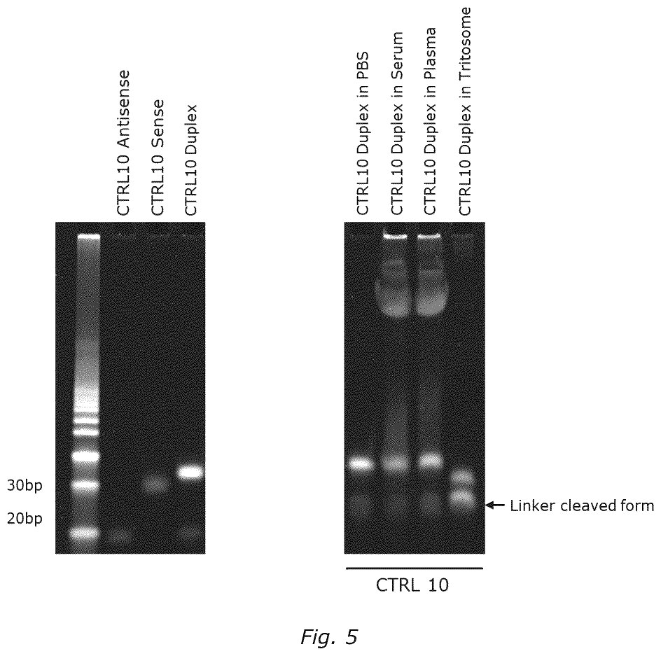

Then, oligonucleotide sample 10 μL was mixed with 6× loading dye (Promega, G190A) 2 μL Total 12 μL was loaded on 12% Native PAGE at 120 V constant for 30 minutes, followed by staining with GelRed (Biotuum, 41003) for 15 minutes. All oligonucleotide samples containing beta-amino acid conjugated ligands showed greater stability under mouse plasma and serum than oligonucleotide samples containing natural amino acid moieties. Oligonucleotides with beta-amino acid conjugated ligands showed some cleavage of conjugate under rat tritosome, but less cleavage compared to oligonucleotides with D or L-amino acid conjugated ligands. Results are shown in FIG. 3, FIG. 4, and FIG. 5.

Test materials were prepared by duplexation with sense strand and antisenses, selected from Compound 197 and 199-216 series, where the Compound 199 contained (GalNAc-C5)₃-[(GABA)-(βH-Lys)-(βH-Lys)]-AmC7 conjugation, the Compound 200 contained (GalNAc-C5)₃-[(GABA)-(L-Lys)-(βH-Lys)]-AmC7 conjugation, and the Compound 201 contained (GalNAc-C5)₃-[i(GABA)-(D-Lys)-(3H-Lys)]-AmC7 conjugation at the 3′-end of sense strand.

### Example 4. Comparative Example

Oligonucleotides with D- and/or L-amino acid conjugated ligands were prepared as shown in Table 14. These examples do not include a β-amino-acid in the ligand cluster moiety.

TABLE 14
Comparative examples.

| Control # | Structure |
|---|---|
| CTRL 1 | 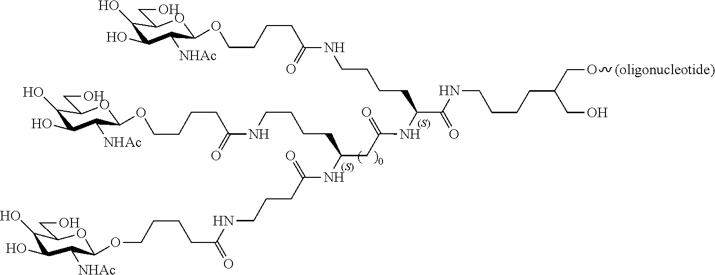 |
| CTRL 2 | 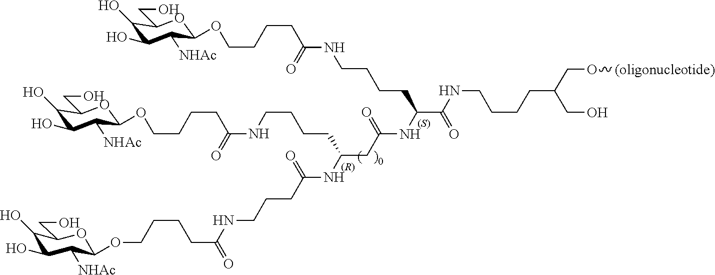 |
| CTRL 3 | 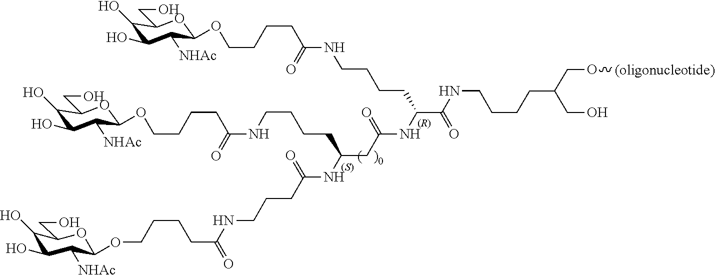 |
| CTRL 4 | 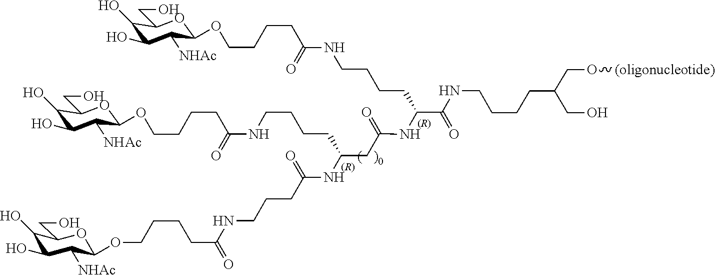 |
| CTRL 5 | 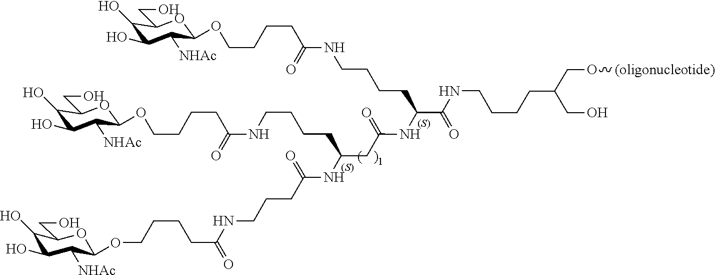 |
| CTRL 7 | 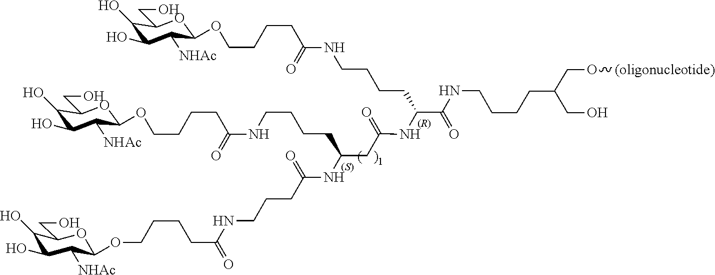 |
| CTRL 10 | 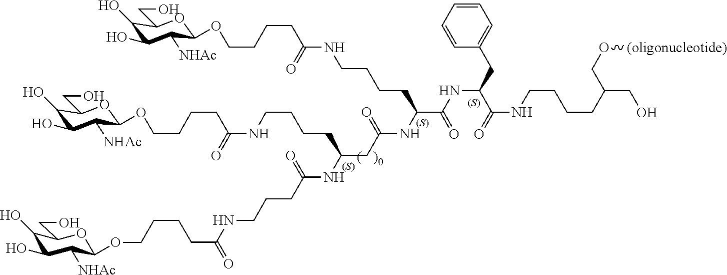 |

### Example 5. In Vitro Test 3 Under the Condition of Mouse Liver Homogenate

The stability of oligonucleotides containing tri-GalNAc conjugate were tested under the conditions of mouse liver homogenate. 6-Week C57BL/6 mouse was purchased from KOATECH (Korea, Pyeongtaek). After 3 weeks, the mouse was sacrificed and whole liver (about 2.5 g) was separated. To prepare liver homogenate, the whole liver was fully homogenized and placed in 50 mL polycarbonate centrifuge tubes including 10 mL of homogenization buffer (100 mM Tris, 1 mM magnesium acetate, pH 8.0). 1 μL of 10 μM diluted test materials were added into 9 μL of liver homogenates, and incubated at 37° C. for 24 hours, 48 hours, and 72 hours. The liver homogenate was pre-incubated at 37° C. for 72 hours before adding the test materials. Test materials were prepared with 1×PBS (Gibco, 10010-023). After incubation, the homogenate samples were mixed with 6× loading dye (Promega, G190A) and heated at 65° C. for 10 minutes. 3 μL of samples were loaded on 10% Native PAGE at 100 V constant for 30 minutes, followed by staining with GelRed (Biotuum, 41003) for 5 minutes.

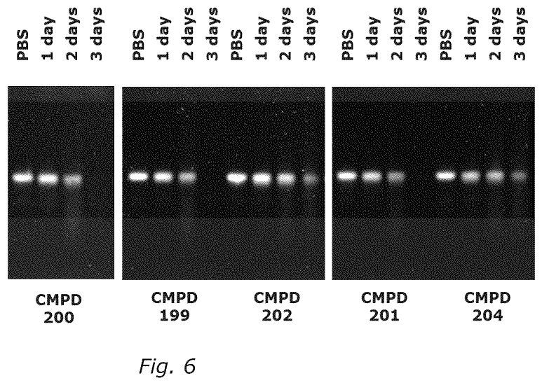

Test materials were prepared by duplexation with sense strand and antisenses, selected from Compound Nos. 197 and 199-216, where the Compound 199 contained (GalNAc-C5)₃-[(GABA)-(βH-Lys)-(βH-Lys)]-AmC7 conjugation, the Compound 200 contained (GalNAc-C5)₃-[(GABA)-(L-Lys)-(βH-Lys)]-AmC7 conjugation, and the Compound 201 contained (GalNAc-C5)₃-[(GABA)-(D-Lys)-(βH-Lys)]-AmC7 conjugation at the 3′-end of sense strand Seq. ID NO:1. Compounds 202-204 contained the same conjugation linker as Compounds 199-201 at the 3′-end of sense strand Seq. ID NO:5. Compounds 205-207 contained the same conjugation liker as Compounds 199-201 at the 3′-end of sense strand Seq. ID NO:6. Results are shown in FIG. 6.

### Example 6. In Vivo Test 1 for Tri-GalNAc Conjugated Oligonucleotide Duplexes

6-Week C57BL/6 Mouse was purchased from KOATECH (Korea, Pyeongtaek). Each test group is n=3. After a week of the acclimation period, oligonucleotide duplexes were injected by 5 mg/kg dose SC single injection on day 0. Oligonucleotide duplexes were prepared with 1×PBS (Gibco, 10010-023). Mouse plasma was collected from the facial vein with an Animal lancet (Medipoint, GR-5). After the blood is collected, the blood is mixed with 0.109 M of trisodium citrate solution (Sigma, S1804) in a 9:1 ratio immediately. Anti-coagulated blood was centrifuged at 2,500 g, for 15 min at room temperature. Mouse plasma was collected from the supernatant, then stored at −80° C. Mouse plasma was collected on day 0 (before oligonucleotide duplex injection), 7, 14, 21, 28, 34, 39 and 62 days. The FIX level of mouse plasma was analyzed with the Biophen FIX (HYPHEN BioMed, 221806-RUO) by following the manufacturer's instructions. Each Mouse's FIX level from a different day point was normalized to day 0 FIX level of same individual.

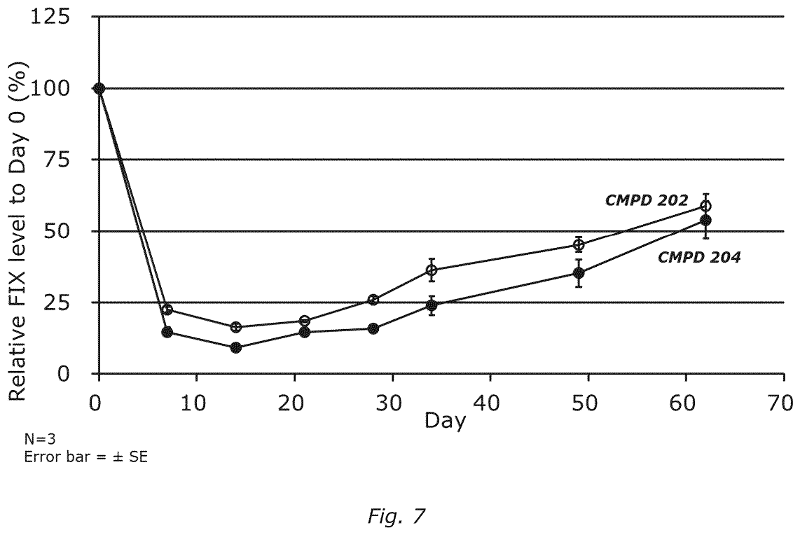

Test materials were prepared by duplexation with sense strand and antisenses, selected from Compounds 197 and 199-216, where the Compound 199 contained (GalNAc-C5)₃-[(GABA)-(βH-Lys)-(βH-Lys)]-AmC7 conjugation, the Compound 200 contained (GalNAc-C5)₃-[(GABA)-(L-Lys)-(βH-Lys)]-AmC7 conjugation, and the Compound 201 contained (GalNAc-C5)₃-[(GABA)-(D-Lys)-(βH-Lys)]-AmC7 conjugation at the 3′-end of sense strand Seq. ID NO:1. Compounds 202-204 contained the same conjugation linker as Compounds 199-201 at the 3′-end of sense strand Seq. ID NO:5. Compounds 205-207 contained the same conjugation liker as Compounds 199-201 at the 3′-end of sense strand Seq. ID NO:6. Results are shown in FIG. 7.

### Example 7. In Vivo Test 2 for Tri-GalNAc Conjugated Oligonucleotide Duplexes

6-Week C57BL/6 Mouse was purchased from KOATECH (Korea, Pyeongtaek). Each test group is n=3. After a week of the acclimation period, oligonucleotide duplexes were injected by 2 mg/kg dose SC single injection on day 0. Oligonucleotide duplexes were prepared with 1×PBS (Gibco, 10010-023). Mouse plasma was collected from the facial vein with an Animal lancet (Medipoint, GR-5). After the blood is collected, the blood is mixed with 0.109 M of trisodium citrate solution (Sigma, S1804) in a 9:1 ratio immediately. Anti-coagulated blood was centrifuged at 2,500 g, for 15 min at room temperature. Mouse plasma was collected from the supernatant, then stored at −80° C. Mouse plasma was collected on day 0 (before oligonucleotide duplex injection), 7, 14, 21, 28, and 42 days. The FVII level of mouse plasma was analyzed with the Biophen FVII (HYPHEN BioMed, 221304-RUO) by following the manufacturer's instructions. Each Mouse's FVII level from a different day point was normalized to day 0 FVII level of same individual.

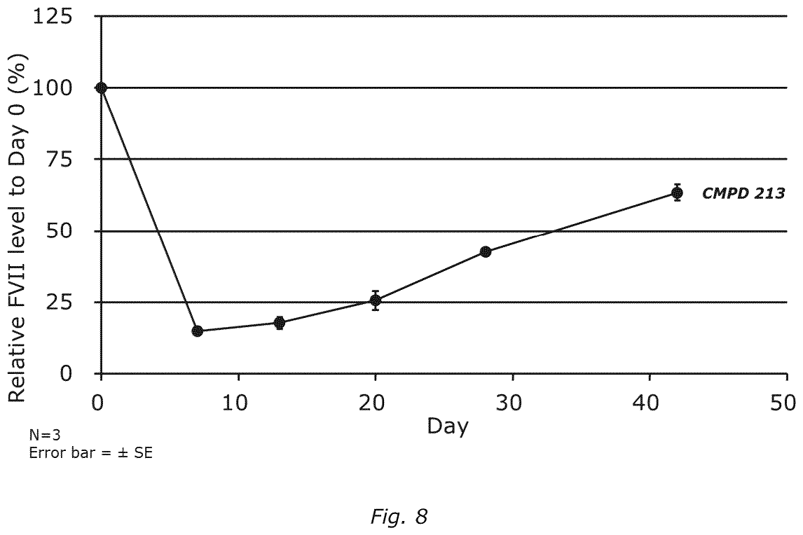

Test materials were prepared by duplexation with sense strand and antisenses, selected from Compounds 197 and 199-216, where the Compound 208 contained (GalNAc-C5)₃-[(GABA)-βH-Lys)-(βH-Lys)]-AmC7 conjugation, the Compound 209 contained (GalNAc-C5)₃-[(GABA)-(L-Lys)-(βH-Lys)]-AmC7 conjugation, and the Compound 210 contained (GalNAc-C5)₃-[(GABA)-(D-Lys)-(βH-Lys)]-AmC7 conjugation at the 3′-end of sense strand Seq. ID NO:7. Compounds 211-213 contained the same conjugation linker as Compounds 208-210 at the 3′-end of sense strand Seq. ID NO:8. Compounds 214-216 contained the same conjugation liker as Compounds 208-210 at the 3′-end of sense strand Seq. ID NO:9. Results are shown in FIG. 8.

Abbreviations used herein include those of Table 15. In context, use of abbreviations may refer to an “yl” or “di-yl” or corresponding “ate” of the reference compound. For example, GalNAc, which refers to 2-(acetylamino)-2-deoxy-D-galactose parent compound, may also refer to 2-(acetylamino)-2-deoxy-D-galactosyl moiety, and CA, which refers to decanoic acid, may also refer to dacanoyl or decanoate. Structures of certain abbreviations are also shown in Table 16 for convenience.

TABLE 15

| Abbreviation | Common Name | IUPAC Name/Structure |
|---|---|---|
| GalNAc | N-acetyl-D-galactosamine | 2-(acetylamino)-2-deoxy-D-galactose |
| GluNAc | N-acetyl-D-glucosamine | ß-D-(acetylamino)-2-deoxy-D- |
| | | glycopyranose |
| GABA | Y-Aminobutaric acid | 4-aminobutanoic acid |
| | | 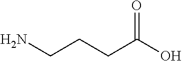 |
| AEA | Aminoethoxyacetic acid | 2-(2-aminoethoxy)acetic acid |
| | | 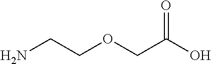 |
| AEEA | Aminoethoxyethoxyacetic | 2-(2-(2-Aminoethoxy)ethoxy)acetic acid |
| | acid | |
| | | 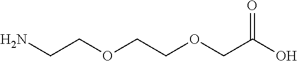 [SMILES: NCCOCCOCC(=O)O] |
| AEEP | Aminoethoxyethoxypropanoic | 3-(2-(2-Aminoethoxy)ethoxy)propanoic |
| | acid | acid |
| | | 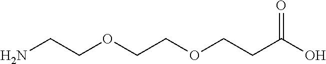 [SMILES: NCCOCCOCCC(=O)O] |
| GLY | Glycine | Aminoacetic acid |
| | | 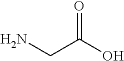 |
| βH-Lys | Beta-homolysine | (S)-3,7-Diaminoheptanoic acid |
| βH-Glu | Beta-homoglutamic acid | (S)-3-Aminohexanedioic acid |
| D-Lys | D-Lysine | (2S)-2,6-Diaminohexanoic acid |
| L-Lys | L-Lysine | (2R)-2,6-Diaminohexanoic acid |
| PGA | Pelargonic acid | Nanoanoic acid |
| CA | Capric acid | Decanoic acid |
| UDA | Hendecanoic acid | Undecanoic acid |
| DDA | Lauric acid or Dodecylic acid | Dodecanoic acid |
| DDA 12-OH | N/A | 12-Hydroxydodecanoic acid |
| TDA | Tridecylic acid | Tridecanoic acid |
| MA | Myristic acid | Tetradecanoic acid |
| PDA | Pentadecylic acid | Pentadecanoic acid |
| PA | Palmitic acid | Hexadecanoic acid |
| HDA | Heptadecylic acid | Heptadecanoic acid |
| SA | Stearic acid | Octadecanoic acid |
| SA 18-OH | N/A | 18-Hydroxystearic acid |
| SA 12-OH | N/A | 12-Hydroxystearic acid |
| SA 2-OH | N/A | 2-Hydroxystearic acid |
| ACA | Arachidic acid | Icosanoic acid |
| BA | Behenic acid | Docosanoic acid |
| DHA | Cervonic acid | (4Z,7Z,10Z,13Z,16Z,19Z)-Docosa- |
| | | 4,7,10,13,16,19-hexaenoic acid |
| ARA | Arachidonic acid | (5Z,8Z,11Z,14Z)-Eicosa-5,8,11,14- |
| | | tetraenoic acid |
| EPA | EPA | (5Z,8Z,11Z,14Z,17Z)-eicosa-5,8,11,14- |
| | | pentaenoic acid |
| ALA | α-Linolenic acid | (9Z,12Z,15Z)-octadeca-9,12,15-trienoic |
| | | acid |
| GLA | γ-Linolenic acid | (6Z,9Z,12Z)-octadeca-6,9,12-trienoic |
| | | acid |
| RA | Retinoic acid or vitamin A | (2E,4E,6E,8E)-3,7-Dimethyl-9-(2,6,6- |
| | | trimethylcyclohex-1-en-1-yl)nona- |
| | | 2,4,6,8-tetraenoic acid |
| OA | Oleic acid | (9Z)-Octadec-9-enoic acid |
| EA | Elaidic acid | (E)-Octadec-9-enoic acid |
| LA | Linoleic acid | (9Z,12Z)-octadeca-9,12-dienoic acid |
| C5 | Valeric acid or Pentyl amine | Pentanoic acid |
| | | 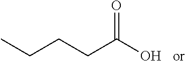 |
| | | or |
| | | 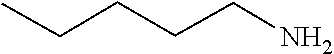 |

TABLE 16

| Abbreviation | Structure |
|---|---|
| AEA-GABA | 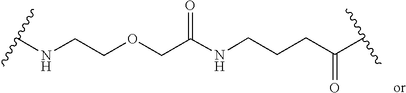 [SMILES: *NCCOCC(=O)NCCCC(*)=O ; "or"] |
| | 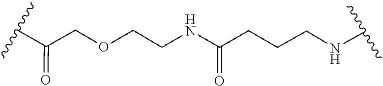 [SMILES: *NCCCC(=O)NCCOCC(*)=O] |
| AEEA-GABA | 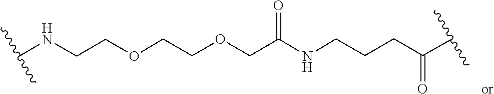 [SMILES: *NCCOCCOCC(=O)NCCCC(*)=O ; "or"] |
| | 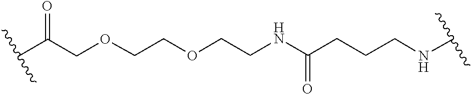 [SMILES: *NCCCC(=O)NCCOCCOCC(*)=O] |
| AEEP-GABA | 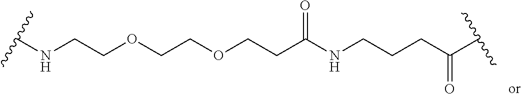 [SMILES: *NCCOCCOCCC(=O)NCCCC(*)=O ; "or"] |
| | 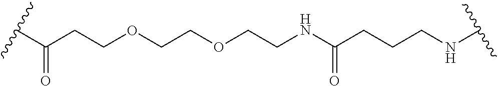 [SMILES: *NCCCC(=O)NCCOCCOCCC(*)=O] |
| C5 | 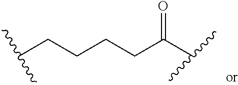 [SMILES: *CCCCC(*)=O ; "or"] |
| | 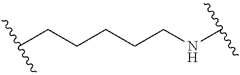 [SMILES: *CCCCCN*] |
| C5-AEA-GABA | 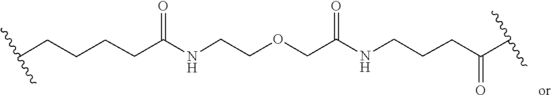 [SMILES: *CCCCC(=O)NCCOCC(=O)NCCCC(*)=O ; "or"] |
| | 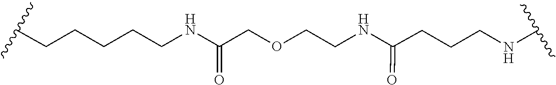 [SMILES: *CCCCCNC(=O)COCCNC(=O)CCCN*] |
| C5-AEEA-GABA | 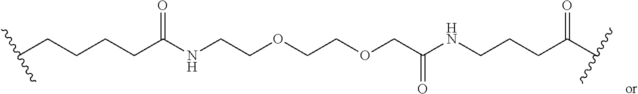 [SMILES: *CCCCC(=O)NCCOCCOCC(=O)NCCCC(*)=O ; "or"] |
| | 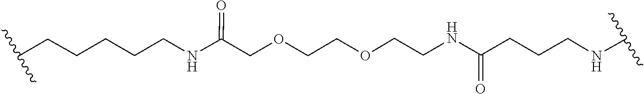 [SMILES: *CCCCCNC(=O)COCCOCCNC(=O)CCCN*] |
| C5-AEEA-GLY | 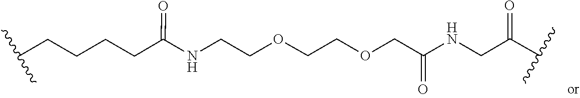 [SMILES: *CCCCC(=O)NCCOCCOCC(=O)NCC(*)=O] |
| | 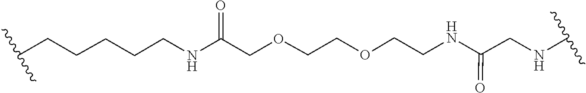 [SMILES: *CCCCCNC(=O)COCCOCCNC(=O)CN*] |
| C5-AEEP-GABA | 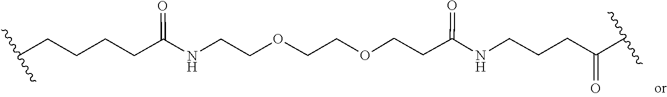 [SMILES: *CCCCC(=O)NCCOCCOCCC(=O)NCCCC(*)=O] |
| | 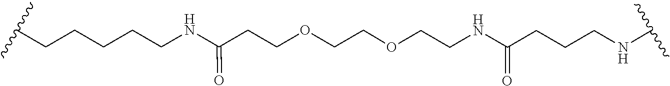 [SMILES: *CCCCCNC(=O)CCOCCOCCNC(=O)CCCN*] |
| C5-GABA | 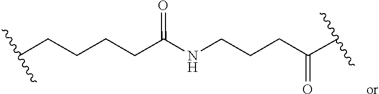 [SMILES: *CCCCC(=O)NCCCC(*)=O ; "or"] |
| | 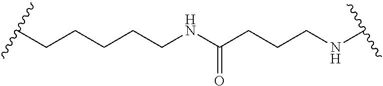 [SMILES: *CCCCCNC(=O)CCCN*] |
| C5-Gly | 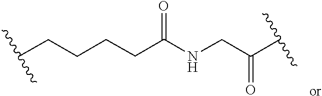 [SMILES: *CCCCC(=O)NCC(*)=O ; "or"] |
| | 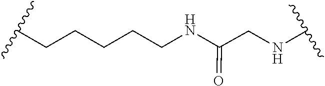 [SMILES: *CCCCCNC(=O)CN*] |
| GABA | 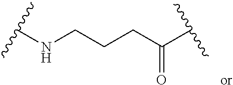 [SMILES: *NCCCC(*)=O ; "or"] |
| | 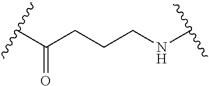 [SMILES: *NCCCC(*)=O] |

1. A compound, comprising the formula: 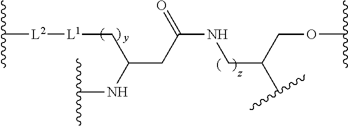
wherein:
y is 0, 1, 2, 3, 4, 5, or 6;
z is 0, 1, 2, 3, 4, 5, or 6; and
L¹ is N(H) and L² is C(O), or L¹ is C(O) and L² is N(H).

2. The compound of claim 1, wherein y is 2, 3, 4, 5, or 6, and z is 4, 5, or 6.

3. The compound of claim 1, wherein

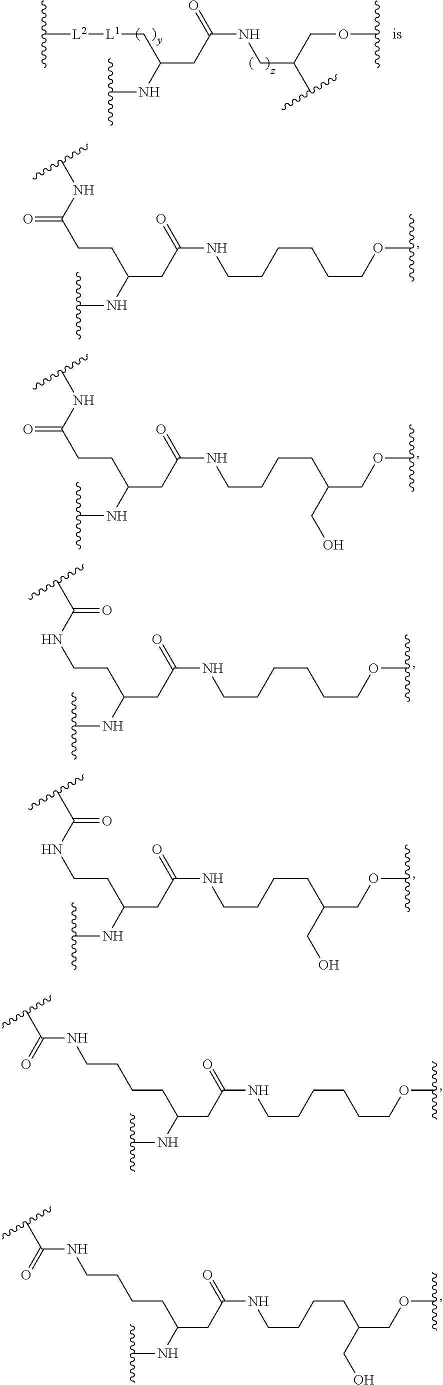

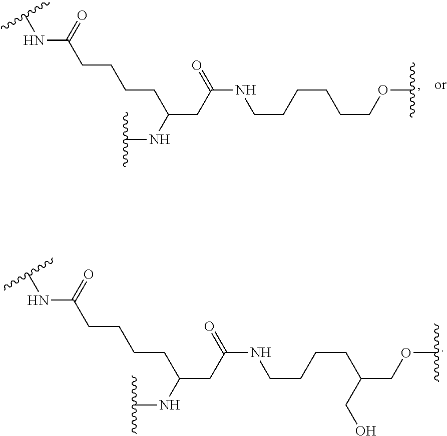 [SMILES: *NC(=O)CCCCC(CC(=O)NCCCCCCO*)N* ; *NC(=O)CCCCC(CC(=O)NCCCCC(CO)CO*)N*]

4. The compound of claim 1, wherein 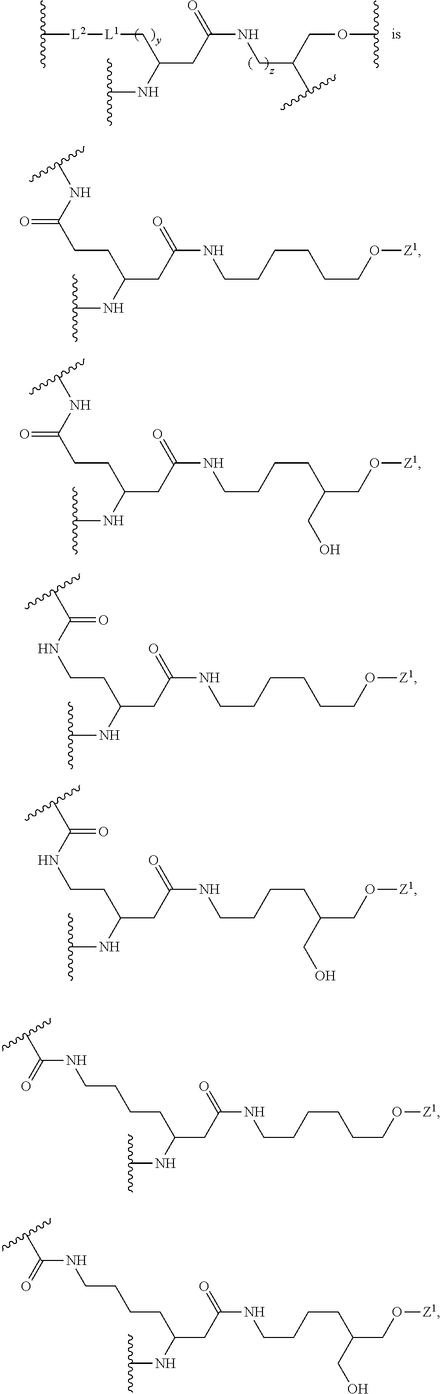 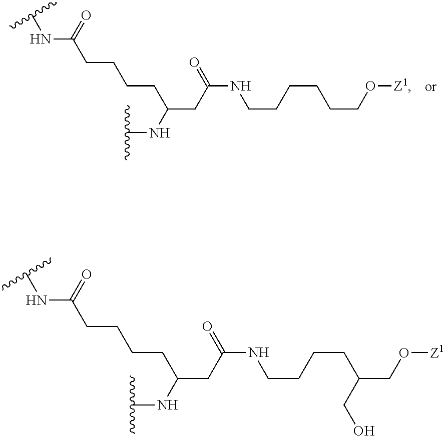
wherein Z¹ comprises a macromolecule.

5. The compound of claim 1, wherein 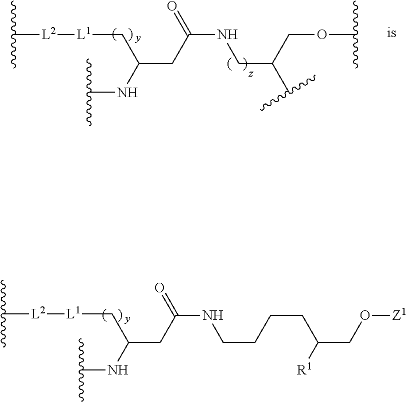
wherein:
y is 2, 3, 4, 5, or 6;
R¹ is H or CH₂OH; and
Z¹ comprises a macromolecule.

6. The compound of claim 1, comprising a formula selected from: 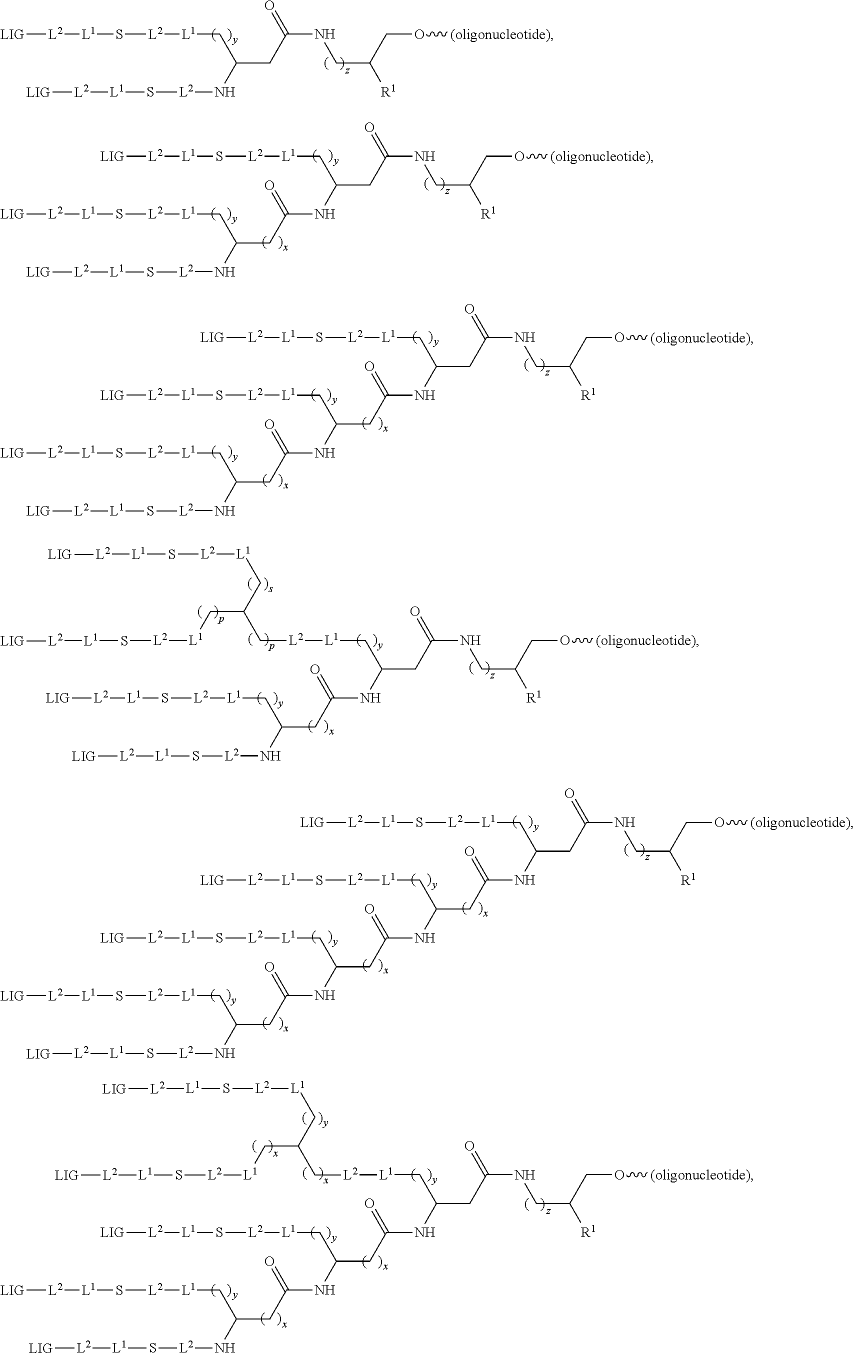 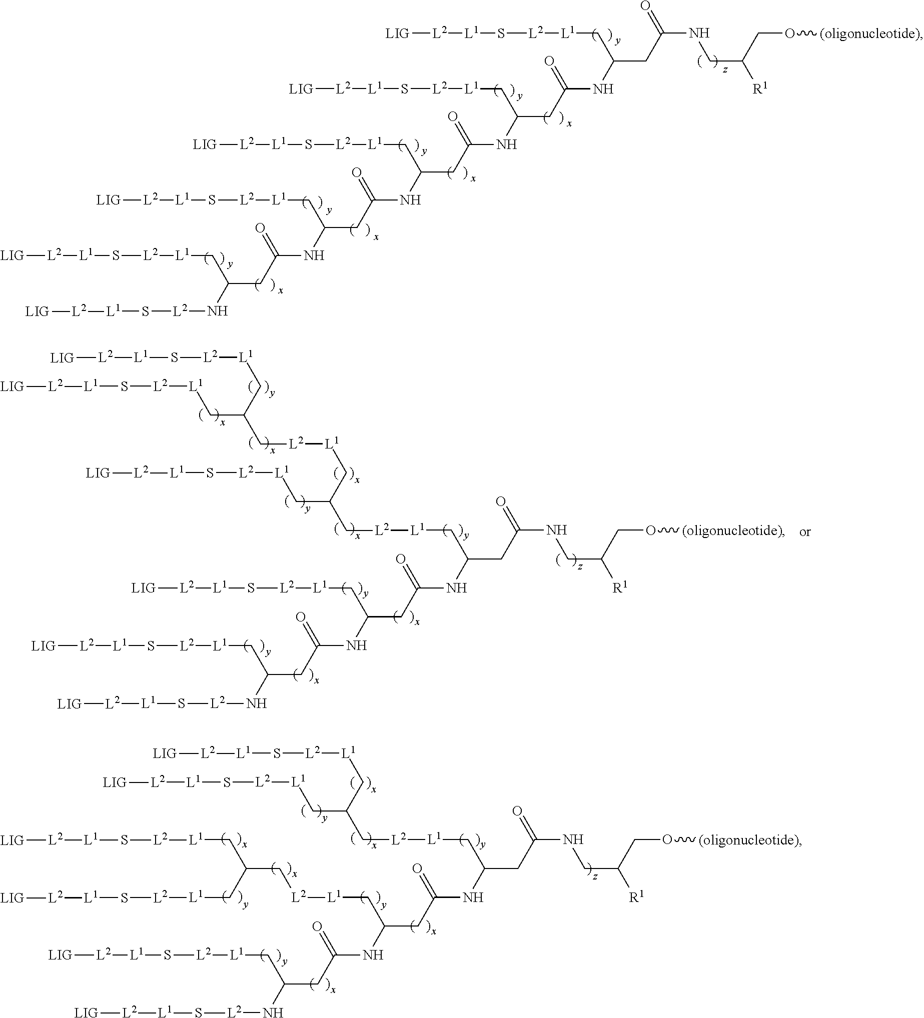
or a stereoisomer or a salt thereof,
wherein
each x is, independently, 0, 1, 2, 3, 4, 5, or 6;
each y is, independently, 0, 1, 2, 3, 4, 5, or 6;
each z is, independently, 0, 1, 2, 3, 4, 5, or 6;
each R¹ is, independently, H or CH₂H;
each L¹ is, independently, N(H) or C(O);
each L² is, independently, N(H) or C(O);
each S is, independently, a bond (null), C₁₋₂₀ alkylene, C₂₋₂₀ alkenylene, C₂₋₂₀ alkynylene, C₃₋₂₀ cycloalkylene, C₄₋₂₀ cycloalkenylene, C₅₋₂₀ cycloalkynylene, C₁₋₂₀ heterocycloalkylene, C₂₋₂₀ heterocycloalkenylene, C₂₋₂₀ heterocycloalkynylene, or a polyalkylene glycol; and
each LIG is, independently, a carbohydrate receptor ligand, a lipid, a cell penetrating peptide, a cell-targeting molecule, a polymer, an aptamer, or an antibody.

7. The compound of claim 6, wherein the polyalkylene glycol is (CH₂CH₂O)ₙ, (CH₂CH₂CH₂O)ₙ, or (CH₂CH₂CH₂CH₂O)ₙ, wherein n is 1, 2, 3, 4, 5, or 6, optionally wherein (oligonucleotide) comprises at least one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

8. The compound of claim 6, wherein the compound comprises more than one of the formula.

9. The compound of claim 1, wherein the compound comprises a molecular weight of at least 3,500 Da.

10. The compound of claim 1, wherein 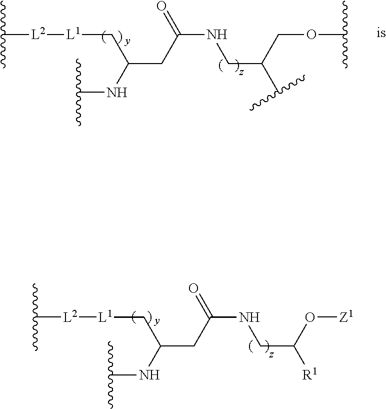
wherein Z¹ comprises a macromolecule.

11. The compound of claim 10, wherein Z¹ comprises one or more of an oligonucleotide, a polysaccharide, a solid support, or a peptide, or wherein R¹ is H, CH₂O-trityl, CH₂O-monomethoxytrityl, CH₂O-dimethoxytrityl, or CH₂O-trimethoxytrityl and Z¹ is

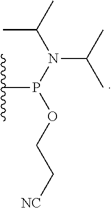 [SMILES: *P(OCCC#N)N(C(C)C)C(C)C]

12. The compound of claim 1, wherein Z¹ comprises at least one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

13. The compound of a claim 1, further comprising at least one ligand selected, independently, from a carbohydrate receptor ligand, a lipid, a cell penetrating peptide, a cell-targeting molecule, a polymer, an aptamer, or an antibody.

14. The compound of a claim 1, further comprising at least one ligand selected, independently, from an N-acylated amino monosaccharide, a monosaccharide, a bile acid ester, or a sterolyl moiety.

15. The compound of a claim 1, further comprising at least one ligand selected, independently, from:
A) N-acetylgalactosaminyl, N-acetylglucosaminyl, mannosyl, chenodeoxycholate, cholate, a cholesterolyl moiety, deoxycholate, docosohexaenoate, glycochenodeoxycholate, glycocholate, 3β-hydroxy-5-cholenate, hyocholate, lithocholate, muricholate, obeticholate, palmitate, taurochenodeoxycholate, taurocholate, or ursodeoxycholate, or
B) a C₆₋₃₀ fatty acid or hydroxy fatty acid, a partially unsaturated fatty acid, including DHA (Docosahexaenoyl), or retinoic acid (retinoyl), or
C) 2-(acetylamino)-2-deoxy-D-galactosyl, β-D-(acetylamino)-2-deoxy-D-glycopyranosyl, 4-aminobutanoyl, 2-(2-aminoethoxy)acetyl, 2-(2-(2-Aminoethoxy)ethoxy)acetyl, 3-(2-(2-Aminoethoxy)ethoxy)propanoyl, Aminoacetyl, (S)-3,7-Diaminoheptanoyl, (S)-3-Aminohexanedioyl, (2S)-2,6-Diaminohexanoyl, (2R)-2,6-Diaminohexanoyl, Nanoanoyl, Decanoyl, Undecanoyl, Dodecanoyl, 12-Hydroxydodecanoyl, Tridecanoyl, Tetradecanoyl, Pentadecanoyl, Hexadecanoyl, Heptadecanoyl, Octadecanoyl, 18-Hydroxystearyl, 12-Hydroxystearyl, 2-Hydroxystearyl, Icosanoyl, Docosanoyl, (4Z,7Z,10Z,13Z,16Z,19Z)-9 of 47 Docosa-4,7,10,13,16,19-hexaenoyl, (5Z,8Z,11Z,14Z)-Eicosa-5,8,11,14-tetraenoyl, (5Z,8Z,11Z,14Z,17Z)-eicosa-5,8,11,14-pentaenoyl, (9Z,12Z,15Z)-octadeca-9,12,15-trienoyl, (6Z,9Z,12Z)-octadeca-6,9,12-trienoyl, (2E,4E,6E,8E)-3,7-Dimethyl-9-(2,6,6-trimethylcyclohex-1-en-1-yl)nona-2,4,6,8-tetraenoyl, (9Z)-Octadec-9-enoyl, (E)-Octadec-9-enoyl, or (9Z,12Z)-octadeca-9,12-dienoyl.

16. The compound of claim 1, in a salt form.

17. The compound of claim 1, selected from:

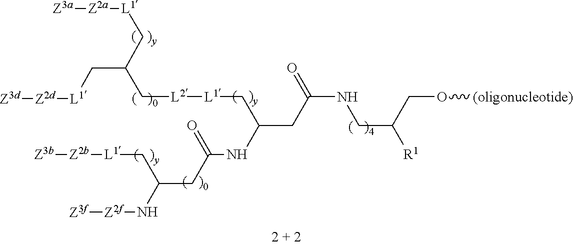

| | | | | | | | | Oligo |
|---|---|---|---|---|---|---|---|---|
| | Z^{3a}, Z^{3b}, | | | | | | | SEQ |
| # | Z^{3d}, Z^{3f} | Z²ª, Z^{2b} | Z^{2d}, Z^{2f} | L^{2′} | L^{1′} | y | R¹ | ID NO |
| 1 | GalNAc | C5-AEEA | C5-AEEA-GABA | C(O) | N(H) | 4 | CH₂OH | 1 |
| 2 | GalNAc | C5-AEEA | C5-AEEA-GABA | C(O) | N(H) | 4 | H | 1 |
| 3 | GalNAc | C5-AEEA | C5-AEEA-GLY | N(H) | C(O) | 2 | CH₂OH | 1 |
| 4 | GalNAc | C5-AEEA | C5-AEEA-GLY | N(H) | C(O) | 2 | H | 1 |
| 5 | GluNAc | C5-AEEA | C5-AEEA-GABA | C(O) | N(H) | 4 | CH₂OH | 2 |
| 6 | GluNAc | C5-AEEA | C5-AEEA-GABA | C(O) | N(H) | 4 | H | 2 |
| 7 | GluNAc | C5-AEEA | C5-AEEA-GLY | N(H) | C(O) | 2 | CH₂OH | 2 |
| 8 | GluNAc | C5-AEEA | C5-AEEA-GLY | N(H) | C(O) | 2 | H | 2 |
| 9 | Mannose | C5-AEEA | C5-AEEA-GABA | C(O) | N(H) | 4 | CH₂OH | 2 |
| 10 | Mannose | C5-AEEA | C5-AEEA-GABA | C(O) | N(H) | 4 | H | 2 |
| 11 | Mannose | C5-AEEA | C5-AEEA-GLY | N(H) | C(O) | 2 | CH₂OH | 2 |
| 12 | Mannose | C5-AEEA | C5-AEEA-GLY | N(H) | C(O) | 2 | H | 2 |
| 13 | PA | AEEA | AEEA-GABA | C(O) | N(H) | 4 | CH₂OH | 4 |
| 14 | PA | AEEA | AEEA-GABA | C(O) | N(H) | 4 | H | 4 |

or

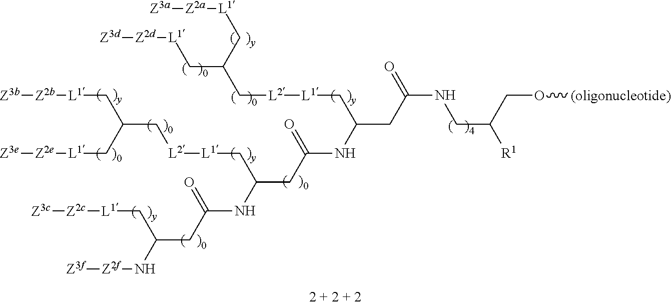

| | Z^{3a}, Z^{3b}, | | | | | | | Oligo |
|---|---|---|---|---|---|---|---|---|
| | Z^{3c}, Z³ª, | Z²ª, Z^{2b}, | | | | | | SEQ |
| # | Z^{3e}, Z^{3f} | Z^{2c} | Z^{2d}, Z^{2e}, Z^{2f} | L2′ | L^{1′} | y | R¹ | ID NO |
| 15 | GalNAc | C5-AEEA | C5-AEEA-GABA | C(O) | N(H) | 4 | CH₂OH | 1 |
| 16 | GalNAc | C5-AEEA | C5-AEEA-GABA | C(O) | N(H) | 4 | H | 1 |
| 17 | GalNAc | C5-AEEA | C5-AEEA-GLY | N(H) | C(O) | 2 | CH₂OH | 1 |
| 18 | GalNAc | C5-AEEA | C5-AEEA-GLY | N(H) | C(O) | 2 | H | 1 |
| 19 | GluNAc | C5-AEEA | C5-AEEA-GABA | C(O) | N(H) | 4 | CH₂OH | 2 |
| 20 | GluNAc | C5-AEEA | C5-AEEA-GABA | C(O) | N(H) | 4 | H | 2 |
| 21 | GluNAc | C5-AEEA | C5-AEEA-GLY | N(H) | C(O) | 2 | CH₂OH | 2 |
| 22 | GluNAc | C5-AEEA | C5-AEEA-GLY | N(H) | C(O) | 2 | H | 2 |
| 23 | Mannose | C5-AEEA | C5-AEEA-GABA | C(O) | N(H) | 4 | CH₂OH | 2 |
| 24 | Mannose | C5-AEEA | C5-AEEA-GABA | C(O) | N(H) | 4 | H | 2 |
| 25 | Mannose | C5-AEEA | C5-AEEA-GLY | N(H) | C(O) | 2 | CH₂OH | 2 |
| 26 | Mannose | C5-AEEA | C5-AEEA-GLY | N(H) | C(O) | 2 | H | 2 |
| 27 | PA | AEEA | AEEA-GABA | C(O) | N(H) | 4 | CH₂OH | 4 |
| 28 | PA | AEEA | AEEA-GABA | C(O) | N(H) | 4 | H | 4 |

or

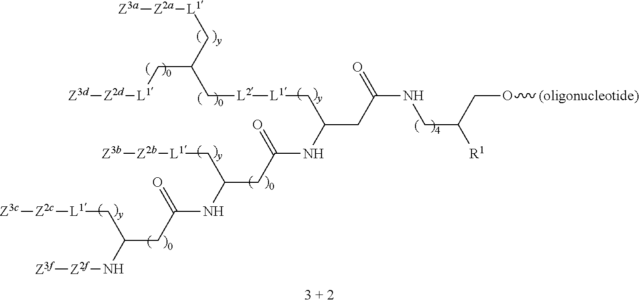

| | Z^{3a}, Z^{3b}, | | | | | | | Oligo |
|---|---|---|---|---|---|---|---|---|
| | Z^{3c}, Z^{3d}, | Z²ª, Z^{2b}, | | | | | | SEQ |
| # | Z^{3f} | Z^{2c} | Z^{2d}, Z^{2f} | L^{2′} | L^{1′} | y | R¹ | ID NO |
| 29 | GalNAc | C5-AEEA | C5-AEEA-Gly | N(H) | C(O) | 2 | CH₂OH | 1 |
| 30 | GalNAc | C5-AEEA | C5-AEEA-Gly | N(H) | C(O) | 2 | H | 1 |
| 31 | GalNAc | C5-AEEA | C5-AEEA-GABA | C(O) | N(H) | 4 | CH₂OH | 1 |
| 32 | GalNAc | C5-AEEA | C5-AEEA-GABA | C(O) | N(H) | 4 | H | 1 |
| 33 | GluNAc | C5-AEEA | C5-AEEA-Gly | N(H) | C(O) | 2 | CH₂OH | 2 |
| 34 | GluNAc | C5-AEEA | C5-AEEA-Gly | N(H) | C(O) | 2 | H | 2 |
| 35 | GluNAc | C5-AEEA | C5-AEEA-GABA | C(O) | N(H) | 4 | CH₂OH | 2 |
| 36 | GluNAc | C5-AEEA | C5-AEEA-GABA | C(O) | N(H) | 4 | H | 2 |
| 37 | Mannose | C5-AEEA | C5-AEEA-Gly | N(H) | C(O) | 2 | CH₂OH | 2 |
| 38 | Mannose | C5-AEEA | C5-AEEA-Gly | N(H) | C(O) | 2 | H | 2 |
| 39 | Mannose | C5-AEEA | C5-AEEA-GABA | C(O) | N(H) | 4 | CH₂OH | 2 |
| 40 | Mannose | C5-AEEA | C5-AEEA-GABA | C(O) | N(H) | 4 | H | 2 |
| 41 | PA | C5-AEEA | AEEA-GABA | C(O) | N(H) | 4 | CH₂OH | 4 |
| 42 | PA | C5-AEEA | AEEA-GABA | C(O) | N(H) | 4 | H | 4 |

or

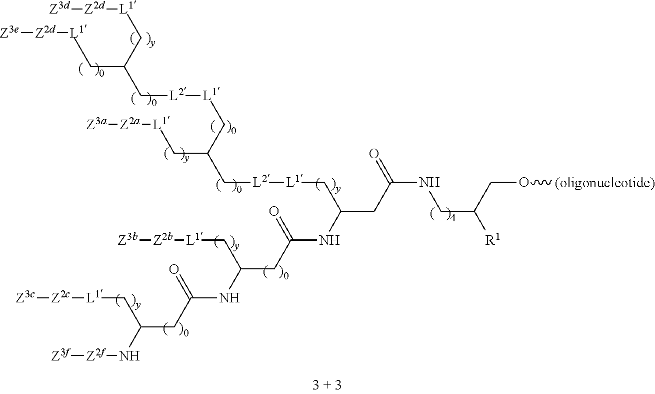

| | Z³ª, Z^{3b} | | | | | | | Oligo |
|---|---|---|---|---|---|---|---|---|
| | Z^{3c}, Z^{3d}, | Z^{3a}, Z^{3b}, | | | | | | SEQ |
| # | Z^{3e}, Z^{3f} | Z^{3c}, Z^{3d} | Z^{3e}, Z^{3f} | L^{2′} | L^{1′} | y | R¹ | ID NO |
| 43 | GalNAc | C5-AEEA | C5-AEEA-GABA | C(O) | N(H) | 4 | CH₂OH | 1 |
| 44 | GalNAc | C5-AEEA | C5-AEEA-GABA | C(O) | N(H) | 4 | H | 1 |
| 45 | GalNAc | C5-AEEA | C5-AEEA-GLY | N(H) | C(O) | 2 | CH₂OH | 1 |
| 46 | GalNAc | C5-AEEA | C5-AEEA-GLY | N(H) | C(O) | 2 | H | 1 |
| 47 | GluNAc | C5-AEEA | C5-AEEA-GABA | C(O) | N(H) | 4 | CH₂OH | 2 |
| 48 | GluNAc | C5-AEEA | C5-AEEA-GABA | C(O) | N(H) | 4 | H | 2 |
| 49 | GluNAc | C5-AEEA | C5-AEEA-GLY | N(H) | C(O) | 2 | CH₂OH | 2 |
| 50 | GluNAc | C5-AEEA | C5-AEEA-GLY | N(H) | C(O) | 2 | H | 2 |
| 51 | Mannose | C5-AEEA | C5-AEEA-GABA | C(O) | N(H) | 4 | CH₂OH | 2 |
| 52 | Mannose | C5-AEEA | C5-AEEA-GABA | C(O) | N(H) | 4 | H | 2 |
| 53 | Mannose | C5-AEEA | C5-AEEA-GLY | N(H) | C(O) | 2 | CH₂OH | 2 |
| 54 | Mannose | C5-AEEA | C5-AEEA-GLY | N(H) | C(O) | 2 | H | 2 |
| 55 | PA | C5-AEEA | AEEA-GABA | C(O) | N(H) | 4 | CH₂OH | 4 |
| 56 | PA | C5-AEEA | AEEA-GABA | C(O) | N(H) | 4 | H | 4 |

or
Linear-2

| | | | | | | | Oligo |
|---|---|---|---|---|---|---|---|
| | | | | | | | SEQ |
| # | Z^{3a}, Z^{3f} | Z^{2a} | Z^{2f} | L^{1′} | y | R¹ | ID NO |
| 57 | 3ß-hydroxy 5-cholenoic acid | AEEA | AEEA-GABA | N(H) | 4 | CH₂OH | 3 |
| 58 | 3ß-hydroxy 5-cholenoic acid | AEEA | AEEA-GABA | N(H) | 4 | H | 3 |
| 59 | ACA | — | GABA | N(H) | 4 | CH₂OH | 4 |
| 60 | ALA | AEEA | AEEA-GABA | N(H) | 4 | CH₂OH | 4 |
| 61 | ALA | AEEA | AEEA-GABA | N(H) | 4 | H | 4 |
| 62 | ARA | AEEA | AEEA-GABA | N(H) | 4 | CH₂OH | 4 |
| 63 | ARA | AEEA | AEEA-GABA | N(H) | 4 | H | 4 |
| 64 | BA | — | GABA | N(H) | 4 | CH₂OH | 4 |
| 65 | BA | — | GABA | N(H) | 4 | H | 4 |
| 66 | BA | AEA | AEA-GABA | N(H) | 4 | CH₂OH | 4 |
| 67 | BA | AEA | AEA-GABA | N(H) | 4 | H | 4 |
| 68 | BA | AEEA | AEEA-GABA | N(H) | 4 | CH₂OH | 4 |
| 69 | BA | AEEA | AEEA-GABA | N(H) | 4 | H | 4 |
| 70 | BA | AEEP | AEEP-GABA | N(H) | 4 | CH₂OH | 4 |
| 71 | BA | AEEP | AEEP-GABA | N(H) | 4 | H | 4 |
| 72 | BA | — | — | N(H) | 4 | CH₂OH | 4 |
| 73 | BA | — | — | N(H) | 4 | H | 4 |
| 74 | CA | — | GABA | N(H) | 4 | CH₂OH | 4 |
| 75 | Chenocholic acid | AEEA | AEEA-GABA | N(H) | 4 | CH₂OH | 3 |
| 76 | Chenocholic acid | AEEA | AEEA-GABA | N(H) | 4 | H | 3 |
| 77 | Cholesterol | AEEA | AEEA-GABA | N(H) | 4 | CH₂OH | 4 |
| 78 | Cholesterol | AEEA | AEEA-GABA | N(H) | 4 | H | 4 |
| 79 | Cholic acid | AEEA | AEEA-GABA | N(H) | 4 | CH₂OH | 3 |
| 80 | Cholic acid | AEEA | AEEA-GABA | N(H) | 4 | H | 3 |
| 81 | DDA | — | GABA | N(H) | 4 | CH₂OH | 4 |
| 82 | DDA 12-OH | — | GABA | N(H) | 4 | CH₂OH | 4 |
| 83 | DHA | AEEA | AEEA-GABA | N(H) | 4 | CH₂OH | 4 |
| 84 | DHA | AEEA | AEEA-GABA | N(H) | 4 | H | 4 |
| 85 | EA | AEEA | AEEA-GABA | N(H) | 4 | CH₂OH | 4 |
| 86 | EA | AEEA | AEEA-GABA | N(H) | 4 | H | 4 |
| 87 | EPA | AEEA | AEEA-GABA | N(H) | 4 | CH₂OH | 4 |
| 88 | EPA | AEEA | AEEA-GABA | N(H) | 4 | H | 4 |
| 89 | GalNAc | C5 | C5 | N(H) | 4 | CH₂OH | 1 |
| 90 | GalNAc | C5 | C5 | N(H) | 4 | H | 1 |
| 91 | GalNAc | C5-AEA | C5-AEA-GABA | N(H) | 4 | CH₂OH | 1 |
| 92 | GalNAc | C5-AEA | C5-AEA-GABA | N(H) | 4 | H | 1 |
| 93 | GalNAc | C5-AEEA | C5-AEEA-GABA | N(H) | 4 | CH₂OH | 1 |
| 94 | GalNAc | C5-AEEA | C5-AEEA-GABA | N(H) | 4 | H | 1 |
| 95 | GalNAc | C5-AEEP | C5-AEEP-GABA | N(H) | 4 | CH₂OH | 1 |
| 96 | GalNAc | C5-AEEP | C5-AEEP-GABA | N(H) | 4 | H | 1 |
| 97 | GalNAc | C5 | C5-GABA | N(H) | 4 | CH₂OH | 1 |
| 98 | GalNAc | C5 | C5-GABA | N(H) | 4 | H | 1 |
| 99 | GalNAc | C5 | C5-Gly | C(O) | 2 | CH₂OH | 1 |
| 100 | GalNAc | C5 | C5-Gly | C(O) | 2 | H | 1 |
| 101 | GLA | AEEA | AEEA-GABA | N(H) | 4 | CH₂OH | 4 |
| 102 | GLA | AEEA | AEEA-GABA | N(H) | 4 | H | 4 |
| 103 | GluNAc | C5 | C5 | N(H) | 4 | CH₂OH | 2 |
| 104 | GluNAc | C5 | C5 | N(H) | 4 | H | 2 |
| 105 | GluNAc | C5-AEA | C5-AEA-GABA | N(H) | 4 | CH₂OH | 2 |
| 106 | GluNAc | C5-AEA | C5-AEA-GABA | N(H) | 4 | H | 2 |
| 107 | GluNAc | C5-AEEA | C5-AEEA-GABA | N(H) | 4 | CH₂OH | 2 |
| 108 | GluNAc | C5-AEEA | C5-AEEA-GABA | N(H) | 4 | H | 2 |
| 109 | GluNAc | C5-AEEP | C5-AEEP-GABA | N(H) | 4 | CH₂OH | 2 |
| 110 | GluNAc | C5-AEEP | C5-AEEP-GABA | N(H) | 4 | H | 2 |
| 111 | GluNAc | C5 | C5-GABA | N(H) | 4 | CH₂OH | 2 |
| 112 | GluNAc | C5 | C5-GABA | N(H) | 4 | H | 2 |
| 113 | GluNAc | C5 | C5-Gly | C(O) | 2 | CH₂OH | 2 |
| 114 | GluNAc | C5 | C5-Gly | C(O) | 2 | H | 2 |
| 115 | HDA | — | GABA | N(H) | 4 | CH₂OH | 4 |
| 116 | LA | AEEA | AEEA-GABA | N(H) | 4 | CH₂OH | 4 |
| 117 | LA | AEEA | AEEA-GABA | N(H) | 4 | H | 4 |
| 118 | Lithocholic acid | AEEA | AEEA-GABA | N(H) | 4 | CH₂OH | 3 |
| 119 | Lithocholic acid | AEEA | AEEA-GABA | N(H) | 4 | H | 3 |
| 120 | MA | — | GABA | N(H) | 4 | CH₂OH | 4 |
| 121 | Mannose | C5 | C5 | N(H) | 4 | CH₂OH | 2 |
| 122 | Mannose | C5 | C5 | N(H) | 4 | H | 2 |
| 123 | Mannose | C5-AEA | C5-AEA-GABA | N(H) | 4 | CH₂OH | 2 |
| 124 | Mannose | C5-AEA | C5-AEA-GABA | N(H) | 4 | H | 2 |
| 125 | Mannose | C5-AEEA | C5-AEEA-GABA | N(H) | 4 | CH₂OH | 2 |
| 126 | Mannose | C5-AEEA | C5-AEEA-GABA | N(H) | 4 | H | 2 |
| 127 | Mannose | C5-AEEP | C5-AEEP-GABA | N(H) | 4 | CH₂OH | 2 |
| 128 | Mannose | C5-AEEP | C5-AEEP-GABA | N(H) | 4 | H | 2 |
| 129 | Mannose | C5 | C5-GABA | N(H) | 4 | CH₂OH | 2 |
| 130 | Mannose | C5 | C5-GABA | N(H) | 4 | H | 2 |
| 131 | Mannose | C5 | C5-Gly | C(O) | 2 | CH₂OH | 2 |
| 132 | Mannose | C5 | C5-Gly | C(O) | 2 | H | 2 |
| 133 | OA | AEEA | AEEA-GABA | N(H) | 4 | CH₂OH | 4 |
| 134 | OA | AEEA | AEEA-GABA | N(H) | 4 | H | 4 |
| 135 | PA | — | GABA | N(H) | 4 | CH₂OH | 4 |
| 136 | PA | — | GABA | N(H) | 4 | H | 4 |
| 137 | PA | C5-AEA | AEA-GABA | N(H) | 4 | CH₂OH | 4 |
| 138 | PA | C5-AEA | AEA-GABA | N(H) | 4 | H | 4 |
| 139 | PA | C5-AEEA | AEEA-GABA | N(H) | 4 | CH₂OH | 4 |
| 140 | PA | C5-AEEA | AEEA-GABA | N(H) | 4 | H | 4 |
| 141 | PA | C5-AEEP | AEEP-GABA | N(H) | 4 | CH₂OH | 4 |
| 142 | PA | C5-AEEP | AEEP-GABA | N(H) | 4 | H | 4 |
| 143 | PA | — | — | N(H) | 4 | CH₂OH | 4 |
| 144 | PA | — | — | N(H) | 4 | H | 4 |
| 145 | PA/CA | — | GABA | N(H) | 4 | CH₂OH | 4 |
| 146 | PA/CA | AEA | AEA-GABA | N(H) | 4 | CH₂OH | 4 |
| 147 | PA/CA | AEEA | AEEA-GABA | N(H) | 4 | CH₂OH | 4 |
| 148 | PA/CA | AEEP | AEEP-GABA | N(H) | 4 | CH₂OH | 4 |
| 149 | PA/CA | — | — | N(H) | 4 | CH₂OH | 4 |
| 150 | PA/DDA | — | GABA | N(H) | 4 | CH₂OH | 4 |
| 151 | PA/DDA | AEA | AEA-GABA | N(H) | 4 | CH₂OH | 4 |
| 152 | PA/DDA | AEEA | AEEA-GABA | N(H) | 4 | CH₂OH | 4 |
| 153 | PA/DDA | AEEP | AEEP-GABA | N(H) | 4 | CH₂OH | 4 |
| 154 | PA/DDA | — | — | N(H) | 4 | CH₂OH | 4 |
| 155 | PA/MA | — | GABA | N(H) | 4 | CH₂OH | 4 |
| 156 | PA/MA | AEA | AEA-GABA | N(H) | 4 | CH₂OH | 4 |
| 157 | PA/MA | AEEA | AEEA-GABA | N(H) | 4 | CH₂OH | 4 |
| 158 | PA/MA | AEEP | AEEP-GABA | N(H) | 4 | CH₂OH | 4 |
| 159 | PA/MA | — | — | N(H) | 4 | CH₂OH | 4 |
| 160 | PA/PDA | — | GABA | N(H) | 4 | CH₂OH | 4 |
| 161 | PA/PDA | AEA | AEA-GABA | N(H) | 4 | CH₂OH | 4 |
| 162 | PA/PDA | AEEA | AEEA-GABA | N(H) | 4 | CH₂OH | 4 |
| 163 | PA/PDA | AEEP | AEEP-GABA | N(H) | 4 | CH₂OH | 4 |
| 164 | PA/PDA | — | -— | N(H) | 4 | CH₂OH | 4 |
| 165 | PA/PGA | — | GABA | N(H) | 4 | CH₂OH | 4 |
| 166 | PA/PGA | AEA | AEA-GABA | N(H) | 4 | CH₂OH | 4 |
| 167 | PA/PGA | AEEA | AEEA-GABA | N(H) | 4 | CH₂OH | 4 |
| 168 | PA/PGA | AEEP | AEEP-GABA | N(H) | 4 | CH₂OH | 4 |
| 169 | PA/PGA | — | — | N(H) | 4 | CH₂OH | 4 |
| 170 | PA/TDA | — | GABA | N(H) | 4 | CH₂OH | 4 |
| 171 | PA/TDA | AEA | AEA-GABA | N(H) | 4 | CH₂OH | 4 |
| 172 | PA/TDA | AEEA | AEEA-GABA | N(H) | 4 | CH₂OH | 4 |
| 173 | PA/TDA | AEEP | AEEP-GABA | N(H) | 4 | CH₂OH | 4 |
| 174 | PA/TDA | — | — | N(H) | 4 | CH₂OH | 4 |
| 175 | PA/UDA | — | GABA | N(H) | 4 | CH₂OH | 4 |
| 176 | PA/UDA | AEA | AEA-GABA | N(H) | 4 | CH₂OH | 4 |
| 177 | PA/UDA | AEEA | AEEA-GABA | N(H) | 4 | CH₂OH | 4 |
| 178 | PA/UDA | AEEP | AEEP-GABA | N(H) | 4 | CH₂OH | 4 |
| 179 | PA/UDA | — | — | N(H) | 4 | CH₂OH | 4 |
| 180 | PDA | — | GABA | N(H) | 4 | CH₂OH | 4 |
| 181 | PGA | — | GABA | N(H) | 4 | CH₂OH | 4 |
| 182 | RA | AEEA | AEEA-GABA | N(H) | 4 | CH₂OH | 4 |
| 183 | RA | AEEA | AEEA-GABA | N(H) | 4 | H | 4 |
| 184 | SA | — | GABA | N(H) | 4 | CH₂OH | 4 |
| 185 | SA 12-OH | — | GABA | N(H) | 4 | CH₂OH | 4 |
| 186 | SA 18-OH | — | GABA | N(H) | 4 | CH₂OH | 4 |
| 187 | SA 2-OH | — | GABA | N(H) | 4 | CH₂OH | 4 |
| 188 | TDA | — | GABA | N(H) | 4 | CH₂OH | 4 |
| 189 | UDA | — | GABA | N(H) | 4 | CH₂OH | 4 |
| 190 | Ursodeoxycholic acid | AEEA | AEEA-GABA | N(H) | 4 | CH₂OH | 3 |
| 191 | Ursodeoxycholic acid | AEEA | AEEA-GABA | N(H) | 4 | H | 3 |

or

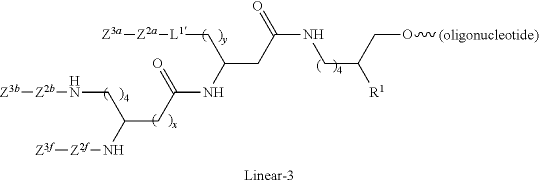

| | | | | | | | | Oligo |
|---|---|---|---|---|---|---|---|---|
| | Z^{3ª}, Z^{3b}, | | | | | | | SEQ |
| # | Z^{3f} | Z²ª, Z^{2b} | Z^{2f} | L^{1′} | y | x | R¹ | ID NO |
| 192 | BA | — | GABA | N(H) | 4 | 0 | CH₂OH | 4 |
| 193 | BA | AEA | AEA-GABA | N(H) | 4 | 0 | CH₂OH | 4 |
| 194 | BA | AEEA | AEEA-GABA | N(H) | 4 | 0 | CH₂OH | 4 |
| 195 | BA | AEEP | AEEP-GABA | N(H) | 4 | 0 | CH₂OH | 4 |
| 196 | BA | — | — | N(H) | 4 | 0 | CH₂OH | 4 |
| 197 | GalNAc | C5-AEEA | C5-AEEA-GABA | N(H) | 4 | 0 | CH₂OH | 1 |
| 198 | GalNAc | C5-AEEA | C5-AEEA-GABA | N(H) | 4 | 0 | H | 1 |
| 199 | GalNAc | C5 | C5-GABA | N(H) | 4 | 1 | CH₂OH | 1 |
| 200 | GalNAc | C5 | C5-GABA | N(H) | 4 | 0 | CH₂OH | 1 |
| 201 | GalNAc | C5 | C5-GABA | N(H) | 4 | 0 | CH₂OH | 1 |
| 202 | GalNAc | C5 | C5-GABA | N(H) | 4 | 1 | CH₂OH | 5 |
| 203 | GalNAc | C5 | C5-GABA | N(H) | 4 | 0 | CH₂OH | 5 |
| 204 | GalNAc | C5 | C5-GABA | N(H) | 4 | 0 | CH₂OH | 5 |
| 205 | GalNAc | C5 | C5-GABA | N(H) | 4 | 1 | CH₂OH | 6 |
| 206 | GalNAc | C5 | C5-GABA | N(H) | 4 | 0 | CH₂OH | 6 |
| 207 | GalNAc | C5 | C5-GABA | N(H) | 4 | 0 | CH₂OH | 6 |
| 208 | GalNAc | C5 | C5-GABA | N(H) | 4 | 1 | CH₂OH | 7 |
| 209 | GalNAc | C5 | C5-GABA | N(H) | 4 | 0 | CH₂OH | 7 |
| 210 | GalNAc | C5 | C5-GABA | N(H) | 4 | 0 | CH₂OH | 7 |
| 211 | GalNAc | C5 | C5-GABA | N(H) | 4 | 1 | CH₂OH | 8 |
| 212 | GalNAc | C5 | C5-GABA | N(H) | 4 | 0 | CH₂OH | 8 |
| 213 | GalNAc | C5 | C5-GABA | N(H) | 4 | 0 | CH₂OH | 8 |
| 214 | GalNAc | C5 | C5-GABA | N(H) | 4 | 1 | CH₂OH | 9 |
| 215 | GalNAc | C5 | C5-GABA | N(H) | 4 | 0 | CH₂OH | 9 |
| 216 | GalNAc | C5 | C5-GABA | N(H) | 4 | 0 | CH₂OH | 9 |
| 217 | GalNAc | C5 | C5-GABA | N(H) | 4 | 0 | H | 1 |
| 218 | GalNAc | C5-AEEA | C5-AEEA-GLY | C(O) | 2 | 0 | CH₂OH | 1 |
| 219 | GalNAc | C5-AEEA | C5-AEEA-GLY | C(O) | 2 | 0 | H | 1 |
| 220 | GalNAc | C5 | C5-GLY | C(O) | 2 | 0 | CH₂OH | 1 |
| 221 | GalNAc | C5 | C5-GLY | C(O) | 2 | 0 | H | 1 |
| 222 | GluNAc | C5-AEEA | C5-AEEA-GABA | N(H) | 4 | 0 | CH₂OH | 2 |
| 223 | GluNAc | C5-AEEA | C5-AEEA-GABA | N(H) | 4 | 0 | H | 2 |
| 224 | GluNAc | C5 | C5-GABA | N(H) | 4 | 0 | CH₂OH | 2 |
| 225 | GluNAc | C5 | C5-GABA | N(H) | 4 | 0 | H | 2 |
| 226 | GluNAc | C5-AEEA | C5-AEEA-GLY | C(O) | 2 | 0 | CH₂OH | 2 |
| 227 | GluNAc | C5-AEEA | C5-AEEA-GLY | C(O) | 2 | 0 | H | 2 |
| 228 | GluNAc | C5 | C5-GLY | C(O) | 2 | 0 | CH₂OH | 2 |
| 229 | GluNAc | C5 | C5-GLY | C(O) | 2 | 0 | H | 2 |
| 230 | Mannose | C5-AEEA | C5-AEEA-GABA | N(H) | 4 | 0 | CH₂OH | 2 |
| 231 | Mannose | C5-AEEA | C5-AEEA-GABA | N(H) | 4 | 0 | H | 2 |
| 232 | Mannose | C5 | C5-GABA | N(H) | 4 | 0 | CH₂OH | 2 |
| 233 | Mannose | C5 | C5-GABA | N(H) | 4 | 0 | H | 2 |
| 234 | Mannose | C5-AEEA | C5-AEEA-GLY | C(O) | 2 | 0 | CH₂OH | 2 |
| 235 | Mannose | C5-AEEA | C5-AEEA-GLY | C(O) | 2 | 0 | H | 2 |
| 236 | Mannose | C5 | C5-GLY | C(O) | 2 | 0 | CH₂OH | 2 |
| 237 | Mannose | C5 | C5-GLY | C(O) | 2 | 0 | H | 2 |
| 238 | PA | — | GABA | N(H) | 4 | 0 | CH₂OH | 4 |
| 239 | PA | — | GABA | N(H) | 4 | 0 | H | 4 |
| 240 | PA | AEA | AEA-GABA | N(H) | 4 | 0 | CH₂OH | 4 |
| 241 | PA | AEA | AEA-GABA | N(H) | 4 | 0 | H | 4 |
| 242 | PA | AEEA | AEEA-GABA | N(H) | 4 | 0 | CH₂OH | 4 |
| 243 | PA | AEEA | AEEA-GABA | N(H) | 4 | 0 | H | 4 |
| 244 | PA | AEEA | AEEP-GABA | N(H) | 4 | 0 | CH₂OH | 4 |
| 245 | PA | AEEP | AEEP-GABA | N(H) | 4 | 0 | H | 4 |
| 246 | PA | — | — | N(H) | 4 | 0 | CH₂OH | 4 |
| 247 | PA | — | — | N(H) | 4 | 0 | H | 4 |

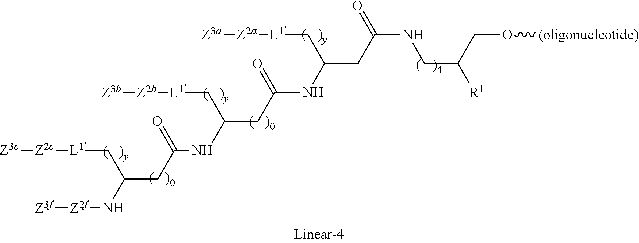
| | | | | | | | Oligo |
|---|---|---|---|---|---|---|---|
| | Z³ª, Z^{3b}, | Z²ª, Z^{2b}, | | | | | SEQ |
| # | Z^{3c}, Z^{3f} | Z^{2c} | Z^{2f} | L^{1′} | y | R¹ | ID NO |
| 248 | GalNAc | C5-AEEA | C5-AEEA-GABA | N(H) | 4 | CH₂OH | 1 |
| 249 | GalNAc | C5-AEEA | C5-AEEA-GABA | N(H) | 4 | H | 1 |
| 250 | GalNAc | C5-AEEA | C5-AEEA-GLY | C(O) | 2 | CH₂OH | 1 |
| 251 | GalNAc | C5-AEEA | C5-AEEA-GLY | C(O) | 2 | H | 1 |
| 252 | GluNAc | C5-AEEA | C5-AEEA-GABA | N(H) | 4 | CH₂OH | 2 |
| 253 | GluNAc | C5-AEEA | C5-AEEA-GABA | N(H) | 4 | H | 2 |
| 254 | GluNAc | C5-AEEA | C5-AEEA-GLY | C(O) | 2 | CH₂OH | 2 |
| 255 | GluNAc | C5-AEEA | C5-AEEA-GLY | C(O) | 2 | H | 2 |
| 256 | Mannose | C5-AEEA | C5-AEEA-GABA | N(H) | 4 | CH₂OH | 2 |
| 257 | Mannose | C5-AEEA | C5-AEEA-GABA | N(H) | 4 | H | 2 |
| 258 | Mannose | C5-AEEA | C5-AEEA-GLY | C(O) | 2 | CH₂OH | 2 |
| 259 | Mannose | C5-AEEA | C5-AEEA-GLY | C(O) | 2 | H | 2 |
| 260 | PA | AEEA | AEEA-GABA | N(H) | 4 | CH₂OH | 4 |
| 261 | PA | AEEA | AEEA-GABA | N(H) | 4 | H | 4 |

or

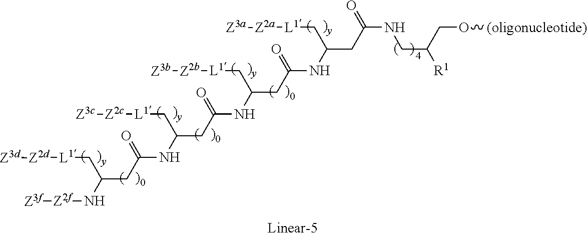

| | | | | | | | Oligo |
|---|---|---|---|---|---|---|---|
| | Z^{3a}, Z^{3b}, Z^{3c}, | Z^{2a}, Z^{2b}, | | | | | SEQ |
| # | Z^{3d}, Z^{3f} | Z^{2c}, Z^{2d} | Z^{2f} | L1′ | y | R¹ | ID NO |
| 262 | GalNAc | C5 | C5 | C(O) | 2 | CH₂OH | 1 |
| 263 | GalNAc | C5 | C5 | C(O) | 2 | H | 1 |
| 264 | GalNAc | C5-AEEA | C5-AEEA-GABA | N(H) | 4 | CH₂OH | 1 |
| 265 | GalNAc | C5-AEEA | C5-AEEA-GABA | N(H) | 4 | H | 1 |
| 266 | GluNAc | C5 | C5 | C(O) | 2 | CH₂OH | 2 |
| 267 | GluNAc | C5 | C5 | C(O) | 2 | H | 2 |
| 268 | GluNAc | C5-AEEA | C5-AEEA-GABA | N(H) | 4 | CH₂OH | 2 |
| 269 | GluNAc | C5-AEEA | C5-AEEA-GABA | N(H) | 4 | H | 2 |
| 270 | Mannose | C5 | C5 | C(O) | 2 | CH₂OH | 2 |
| 271 | Mannose | C5 | C5 | C(O) | 2 | H | 2 |
| 272 | Mannose | C5-AEEA | C5-AEEA-GABA | N(H) | 4 | CH₂OH | 2 |
| 273 | Mannose | C5-AEEA | C5-AEEA-GABA | N(H) | 4 | H | 2 |
| 274 | PA | AEEA | AEEA-GABA | N(H) | 4 | CH₂OH | 4 |
| 275 | PA | AEEA | AEEA-GABA | N(H) | 4 | H | 4 |

or

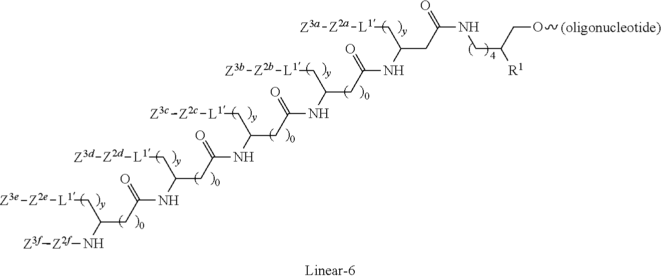

| | | | | | | | Oligo |
|---|---|---|---|---|---|---|---|
| | Z^{3a}, Z^{3b}, Z^{3c}, | Z^{2a}, Z^{2b}, Z^{2c}, | | | | | SEQ |
| # | Z^{3d}, Z^{3e}, Z^{3f} | Z^{2d}, Z^{2e} | Z^{2f} | L^{1′} | y | R¹ | ID NO |
| 276 | GalNAc | C5-AEEA | C5-AEEA-GABA | N(H) | 4 | CH₂OH | 1 |
| 277 | GalNAc | C5-AEEA | C5-AEEA-GABA | N(H) | 4 | H | 1 |
| 278 | GalNAc | C5-AEEA | C5-AEEA-GLY | C(O) | 2 | CH₂OH | 1 |
| 279 | GalNAc | C5-AEEA | C5-AEEA-GLY | C(O) | 2 | H | 1 |
| 280 | GluNAc | C5-AEEA | C5-AEEA-GABA | N(H) | 4 | CH₂OH | 2 |
| 281 | GluNAc | C5-AEEA | C5-AEEA-GABA | N(H) | 4 | H | 2 |
| 282 | GluNAc | C5-AEEA | C5-AEEA-GLY | C(O) | 2 | CH₂OH | 2 |
| 283 | GluNAc | C5-AEEA | C5-AEEA-GLY | C(O) | 2 | H | 2 |
| 284 | Mannose | C5-AEEA | C5-AEEA-GABA | N(H) | 4 | CH₂OH | 2 |
| 285 | Mannose | C5-AEEA | C5-AEEA-GABA | N(H) | 4 | H | 2 |
| 286 | Mannose | C5-AEEA | C5-AEEA-GLY | C(O) | 2 | CH₂OH | 2 |
| 287 | Mannose | C5-AEEA | C5-AEEA-GLY | C(O) | 2 | H | 2 |
| 288 | PA | AEEA | AEEA-GABA | N(H) | 4 | CH₂OH | 4 |
| 289 | PA | AEEA | AEEA-GABA | N(H) | 4 | H | 4 |

or

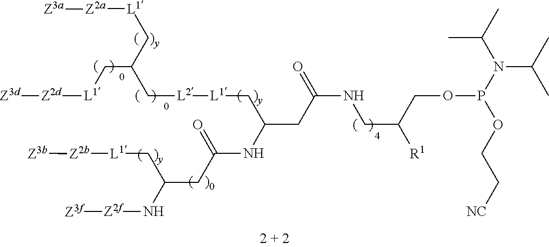

| | Z^{3a}, Z^{3b}, | | | | | | | |
|---|---|---|---|---|---|---|---|---|
| # | Z^{3d}, Z^{3f} | Z^{2a}, Z^{2b} | Z^{2d}, Z^{2f} | L^{2′} | L^{1′} | y | R¹ | Z² |
| 1a,b,c | GalNAc | C5-AEEA | C5-AEEA-GABA | C(O) | N(H) | 4 | CH₂OZ² | Tr, MMTr, |
| | | | | | | | | or DMTr |
| 2a | GalNAc | C5-AEEA | C5-AEEA-GABA | C(O) | N(H) | 4 | H | |
| 3a,b,c | GalNAc | C5-AEEA | C5-AEEA-GLY | N(H) | C(O) | 2 | CH₂OZ² | Tr, MMTr, |
| | | | | | | | | or DMTr |
| 4a | GalNAc | C5-AEEA | C5-AEEA-GLY | N(H) | C(O) | 2 | H | |
| 5a,b,c | GluNAc | C5-AEEA | C5-AEEA-GABA | C(O) | N(H) | 4 | CH₂OZ² | Tr, MMTr, |
| | | | | | | | | or DMTr |
| 6a | GluNAc | C5-AEEA | C5-AEEA-GABA | C(O) | N(H) | 4 | H | |
| 7a,b,c | GluNAc | C5-AEEA | C5-AEEA-GLY | N(H) | C(O) | 2 | CH₂OZ² | Tr, MMTr, |
| | | | | | | | | or DMTr |
| 8a | GluNAc | C5-AEEA | C5-AEEA-GLY | N(H) | C(O) | 2 | H | |
| 9a,b,c | Mannose | C5-AEEA | C5-AEEA-GABA | C(O) | N(H) | 4 | CH₂OZ² | Tr, MMTr, |
| | | | | | | | | or DMTr |
| 10a | Mannose | C5-AEEA | C5-AEEA-GABA | C(O) | N(H) | 4 | H | |
| 11a,b,c | Mannose | C5-AEEA | C5-AEEA-GLY | N(H) | C(O) | 2 | CH₂OZ² | Tr, MMTr, |
| | | | | | | | | or DMTr |
| 12a | Mannose | C5-AEEA | C5-AEEA-GLY | N(H) | C(O) | 2 | H | |
| 13a,b,c | PA | AEEA | AEEA-GABA | C(O) | N(H) | 4 | CH₂OZ² | Tr, MMTr, |
| | | | | | | | | or DMTr |
| 14a | PA | AEEA | AEEA-GABA | C(O) | N(H) | 4 | H | |

or

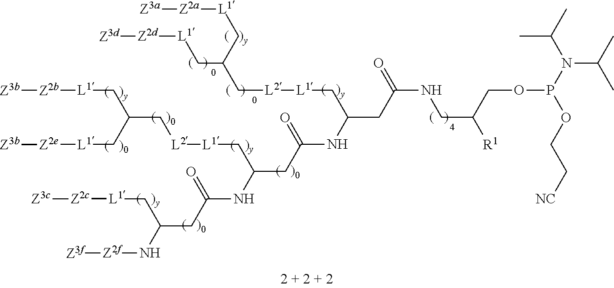

| | Z^{3a}, Z^{3b}, Z^{3c}, | | | | | | | |
|---|---|---|---|---|---|---|---|---|
| # | Z^{3d}, Z^{3e}, Z^{3f} | Z^{2a}, Z^{2b}, Z^{2c}, | Z^{2d}, Z^{2e}, Z^{2f} | L^{2′} | L^{1′} | y | R¹ | Z² |
| 15a,b,c | GalNAc | C5-AEEA | C5-AEEA-GABA | C(O) | N(H) | 4 | CH₂OZ² | Tr, MMTr, |
| | | | | | | | | or DMTr |
| 16a | GalNAc | C5-AEEA | C5-AEEA-GABA | C(O) | N(H) | 4 | H | |
| 17a,b,c | GalNAc | C5-AEEA | C5-AEEA-GLY | N(H) | C(O) | 2 | CH₂OZ² | Tr, MMTr, |
| | | | | | | | | or DMTr |
| 18a | GalNAc | C5-AEEA | C5-AEEA-GLY | N(H) | C(O) | 2 | H | |
| 19a,b,c | GluNAc | C5-AEEA | C5-AEEA-GABA | C(O) | N(H) | 4 | CH₂OZ² | Tr, MMTr, |
| | | | | | | | | or DMTr |
| 20a | GluNAc | C5-AEEA | C5-AEEA-GABA | C(O) | N(H) | 4 | H | |
| 21a,b,c | GluNAc | C5-AEEA | C5-AEEA-GLY | N(H) | C(O) | 2 | CH₂OZ² | Tr, MMTr, |
| | | | | | | | | or DMTr |
| 22a | GluNAc | C5-AEEA | C5-AEEA-GLY | N(H) | C(O) | 2 | H | |
| 23a,b,c | Mannose | C5-AEEA | C5-AEEA-GABA | C(O) | N(H) | 4 | CH₂OZ² | Tr, MMTr, |
| | | | | | | | | or DMTr |
| 24a | Mannose | C5-AEEA | C5-AEEA-GABA | C(O) | N(H) | 4 | H | |
| 25a,b,c | Mannose | C5-AEEA | C5-AEEA-GLY | N(H) | C(O) | 2 | CH₂OZ² | Tr, MMTr, |
| | | | | | | | | or DMTr |
| 26a | Mannose | C5-AEEA | C5-AEEA-GLY | N(H) | C(O) | 2 | H | |
| 27a,b,c | PA | AEEA | AEEA-GABA | C(O) | N(H) | 4 | CH₂OZ² | Tr, MMTr, |
| | | | | | | | | or DMTr |
| 28a | PA | AEEA | AEEA-GABA | C(O) | N(H) | 4 | H | |

or

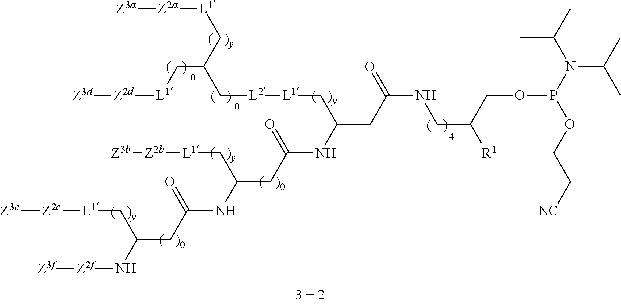

| | Z^{3a}, Z^{3b}, Z^{3c}, | Z^{2a}, Z^{2b}, | | | | | | |
|---|---|---|---|---|---|---|---|---|
| # | Z^{3d}, Z^{3f} | Z^{2c} | Z^{2d}, Z^{2f} | L^{2′} | L^{1′} | y | R¹ | Z² |
| 29a,b,c | GalNAc | C5-AEEA | C5-AEEA-Gly | N(H) | C(O) | 2 | CH₂OZ² | Tr, MMTr, |
| | | | | | | | | or DMTr |
| 30a | GalNAc | C5-AEEA | C5-AEEA-Gly | N(H) | C(O) | 2 | H | |
| 31a,b,c | GalNAc | C5-AEEA | C5-AEEA-GABA | C(O) | N(H) | 4 | CH₂OZ² | Tr, MMTr, |
| | | | | | | | | or DMTr |
| 32a | GalNAc | C5-AEEA | C5-AEEA-GABA | C(O) | N(H) | 4 | H | |
| 33a,b,c | GluNAc | C5-AEEA | C5-AEEA-Gly | N(H) | C(O) | 2 | CH₂OZ² | Tr, MMTr, |
| | | | | | | | | or DMTr |
| 34a | GluNAc | C5-AEEA | C5-AEEA-Gly | N(H) | C(O) | 2 | H | |
| 35a,b,c | GluNAc | C5-AEEA | C5-AEEA-GABA | C(O) | N(H) | 4 | CH₂OZ² | Tr, MMTr, |
| | | | | | | | | or DMTr |
| 36a | GluNAc | C5-AEEA | C5-AEEA-GABA | C(O) | N(H) | 4 | H | |
| 37a,b,c | Mannose | C5-AEEA | C5-AEEA-Gly | N(H) | C(O) | 2 | CH₂OZ² | Tr, MMTr, |
| | | | | | | | | or DMTr |
| 38a | Mannose | C5-AEEA | C5-AEEA-Gly | N(H) | C(O) | 2 | H | |
| 39a,b,c | Mannose | C5-AEEA | C5-AEEA-GABA | C(O) | N(H) | 4 | CH₂OZ² | Tr, MMTr, |
| | | | | | | | | or DMTr |
| 40a | Mannose | C5-AEEA | C5-AEEA-GABA | C(O) | N(H) | 4 | H | |
| 41a,b,c | PA | C5-AEEA | AEEA-GABA | C(O) | N(H) | 4 | CH₂OZ² | Tr, MMTr, |
| | | | | | | | | or DMTr |
| 42a | PA | C5-AEEA | AEEA-GABA | C(O) | N(H) | 4 | H | |

or

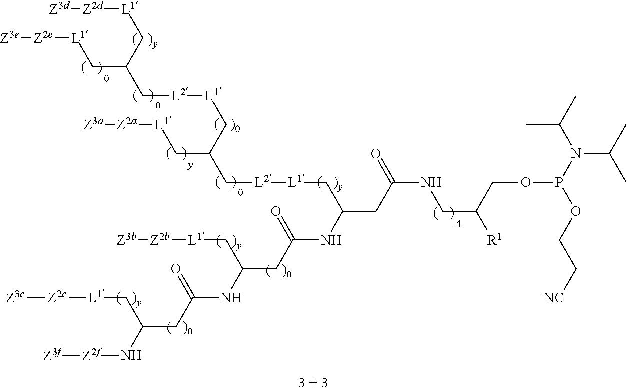

| | Z^{3a}, Z^{3b}, | | | | | | | |
|---|---|---|---|---|---|---|---|---|
| | Z^{3c}, Z^{3d} , | Z^{3a}, Z^{3b}, | | | | | | |
| # | Z^{3e}, Z^{3f} | Z^{3c}, Z^{3d} | Z^{3e}, Z^{3f} | L^{2′} | L^{1′} | y | R¹ | Z² |
| 43a,b,c | GalNAc | C5-AEEA | C5-AEEA-GABA | C(O) | N(H) | 4 | CH₂OZ² | Tr, MMTr, |
| | | | | | | | | or DMTr |
| 44a | GalNAc | C5-AEEA | C5-AEEA-GABA | C(O) | N(H) | 4 | H | |
| 45a,b,c | GalNAc | C5-AEEA | C5-AEEA-GLY | N(H) | C(O) | 2 | CH₂OZ² | Tr, MMTr, |
| | | | | | | | | or DMTr |
| 46a | GalNAc | C5-AEEA | C5-AEEA-GLY | N(H) | C(O) | 2 | H | |
| 47a,b,c | GluNAc | C5-AEEA | C5-AEEA-GABA | C(O) | N(H) | 4 | CH₂OZ² | Tr, MMTr, |
| | | | | | | | | or DMTr |
| 48a | GluNAc | C5-AEEA | C5-AEEA-GABA | C(O) | N(H) | 4 | H | |
| 49a,b,c | GluNAc | C5-AEEA | C5-AEEA-GLY | N(H) | C(O) | 2 | CH₂OZ² | Tr, MMTr, |
| | | | | | | | | or DMTr |
| 50a | GluNAc | C5-AEEA | C5-AEEA-GLY | N(H) | C(O) | 2 | H | |
| 51a,b,c | Mannose | C5-AEEA | C5-AEEA-GABA | C(O) | N(H) | 4 | CH₂OZ² | Tr, MMTr, |
| | | | | | | | | or DMTr |
| 52a | Mannose | C5-AEEA | C5-AEEA-GABA | C(O) | N(H) | 4 | H | |
| 53a,b,c | Mannose | C5-AEEA | C5-AEEA-GLY | N(H) | C(O) | 2 | CH₂OZ² | Tr, MMTr, |
| | | | | | | | | or DMTr |
| 54a | Mannose | C5-AEEA | C5-AEEA-GLY | N(H) | C(O) | 2 | H | |
| 55a,b,c | PA | C5-AEEA | AEEA-GABA | C(O) | N(H) | 4 | CH₂OZ² | Tr, MMTr, |
| | | | | | | | | or DMTr |
| 56a | PA | C5-AEEA | AEEA-GABA | C(O) | N(H) | 4 | H | |

or

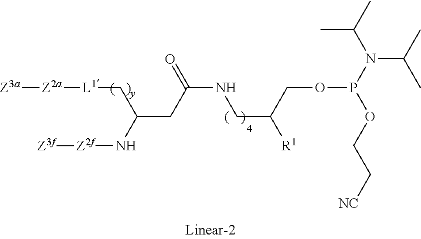

| # | Z^{3a}, Z^{3f} | Z^{2a} | Z^{2f} | L^{1′} | y | R¹ | Z² |
|---|---|---|---|---|---|---|---|
| 57a,b,c | 3β-hydroxy 5-cholenoic | AEEA | AEEA-GABA | N(H) | 4 | CH₂OZ² | Tr, MMTr, |
| | acid | | | | | | or DMTr |
| 58a | 3β-hydroxy 5-cholenoic | AEEA | AEEA-GABA | N(H) | 4 | H | |
| | acid | | | | | | |
| 59a,b,c | ACA | — | GABA | N(H) | 4 | CH₂OZ² | Tr, MMTr, |
| | | | | | | | or DMTr |
| 60a,b,c | ALA | AEEA | AEEA-GABA | N(H) | 4 | CH₂OZ² | Tr, MMTr, |
| | | | | | | | or DMTr |
| 61a | ALA | AEEA | AEEA-GABA | N(H) | 4 | H | |
| 62a,b,c | ARA | AEEA | AEEA-GABA | N(H) | 4 | CH₂OZ² | Tr, MMTr, |
| | | | | | | | or DMTr |
| 63a | ARA | AEEA | AEEA-GABA | N(H) | 4 | H | |
| 64a,b,c | BA | — | GABA | N(H) | 4 | CH₂OZ² | Tr, MMTr, |
| | | | | | | | or DMTr |
| 65a | BA | — | GABA | N(H) | 4 | H | |
| 66a,b,c | BA | AEA | AEA-GABA | N(H) | 4 | CH₂OZ² | Tr, MMTr, |
| | | | | | | | or DMTr |
| 67a | BA | AEA | AEA-GABA | N(H) | 4 | H | |
| 68a,b,c | BA | AEEA | AEEA-GABA | N(H) | 4 | CH₂OZ² | Tr, MMTr, |
| | | | | | | | or DMTr |
| 69a | BA | AEEA | AEEA-GABA | N(H) | 4 | H | |
| 70a,b,c | BA | AEEP | AEEP-GABA | N(H) | 4 | CH₂OZ² | Tr, MMTr, |
| | | | | | | | or DMTr |
| 71a | BA | AEEP | AEEP-GABA | N(H) | 4 | H | |
| 72a,b,c | BA | — | — | N(H) | 4 | CH₂OZ² | Tr, MMTr, |
| | | | | | | | or DMTr |
| 73a | BA | — | — | N(H) | 4 | H | |
| 74a,b,c | CA | — | GABA | N(H) | 4 | CH₂OZ² | Tr, MMTr, |
| | | | | | | | or DMTr |
| 75a,b,c | Chenocholic acid | AEEA | AEEA-GABA | N(H) | 4 | CH₂OZ² | Tr, MMTr, |
| | | | | | | | or DMTr |
| 76a | Chenocholic acid | AEEA | AEEA-GABA | N(H) | 4 | H | |
| 77a,b,c | Cholesterol | AEEA | AEEA-GABA | N(H) | 4 | CH₂OZ² | Tr, MMTr, |
| | | | | | | | or DMTr |
| 78a | Cholesterol | AEEA | AEEA-GABA | N(H) | 4 | H | |
| 79a,b,c | Cholic acid | AEEA | AEEA-GABA | N(H) | 4 | CH₂OZ² | Tr, MMTr, |
| | | | | | | | or DMTr |
| 80a | Cholic acid | AEEA | AEEA-GABA | N(H) | 4 | H | |
| 81a,b,c | DDA | — | GABA | N(H) | 4 | CH₂OZ² | Tr, MMTr, |
| | | | | | | | or DMTr |
| 82a,b,c | DDA 12-OH | — | GABA | N(H) | 4 | CH₂OZ² | Tr, MMTr, |
| | | | | | | | or DMTr |
| 83a,b,c | DHA | AEEA | AEEA-GABA | N(H) | 4 | CH₂OZ² | Tr, MMTr, |
| | | | | | | | or DMTr |
| 84a | DHA | AEEA | AEEA-GABA | N(H) | 4 | H | |
| 85a,b,c | EA | AEEA | AEEA-GABA | N(H) | 4 | CH₂OZ² | Tr, MMTr, |
| | | | | | | | or DMTr |
| 86a | EA | AEEA | AEEA-GABA | N(H) | 4 | H | |
| 87a,b,c | EPA | AEEA | AEEA-GABA | N(H) | 4 | CH₂OZ² | Tr, MMTr, |
| | | | | | | | or DMTr |
| 88a | EPA | AEEA | AEEA-GABA | N(H) | 4 | H | |
| 89a,b,c | GalNAc | C5 | C5 | N(H) | 4 | CH₂OZ² | Tr, MMTr, |
| | | | | | | | or DMTr |
| 90a | GalNAc | C5 | C5 | N(H) | 4 | H | |
| 91a,b,c | GalNAc | C5-AEA | C5-AEA-GABA | N(H) | 4 | CH₂OZ² | Tr, MMTr, |
| | | | | | | | or DMTr |
| 92a | GalNAc | C5-AEA | C5-AEA-GABA | N(H) | 4 | H | |
| 93a,b,c | GalNAc | C5-AEEA | C5-AEEA- | N(H) | 4 | CH₂OZ² | Tr, MMTr, |
| | | | GABA | | | | or DMTr |
| 94a | GalNAc | C5-AEEA | C5-AEEA- | N(H) | 4 | \|H | |
| | | | GABA | | | | |
| 95a,b,c | GalNAc | C5-AEEP | C5-AEEP-GABA | N(H) | 4 | CH₂OZ² | Tr, MMTr, |
| | | | | | | | or DMTr |
| 96a | GalNAc | C5-AEEP | C5-AEEP-GABA | N(H) | 4 | H | |
| 97a,b,c | GalNAc | C5 | C5-GABA | N(H) | 4 | CH₂OZ² | Tr, MMTr, |
| | | | | | | | or DMTr |
| 98a | GalNAc | C5 | C5-GABA | N(H) | 4 | H | |
| 99a,b,c | GalNAc | C5 | C5-Gly | C(O) | 2 | CH₂OZ² | Tr, MMTr, |
| | | | | | | | or DMTr |
| 100a | GalNAc | C5 | C5-Gly | C(O) | 2 | H | |
| 101a,b,c | GLA | AEEA | AEEA-GABA | N(H) | 4 | CH₂OZ² | Tr, MMTr, |
| | | | | | | | or DMTr |
| 102a | GLA | AEEA | AEEA-GABA | N(H) | 4 | H | |
| 103a,b,c | GluNAc | C5 | C5 | N(H) | 4 | CH₂OZ² | Tr, MMTr, |
| | | | | | | | or DMTr |
| 104a | GluNAc | C5 | C5 | N(H) | 4 | H | |
| 105a,b,c | GluNAc | C5-AEA | C5-AEA-GABA | N(H) | 4 | CH₂OZ² | Tr, MMTr, |
| | | | | | | | or DMTr |
| 106a | GluNAc | C5-AEA | C5-AEA-GABA | N(H) | 4 | H | |
| 107a,b,c | GluNAc | C5-AEEA | C5-AEEA- | N(H) | 4 | CH₂OZ² | Tr, MMTr, |
| | | | GABA | | | | or DMTr |
| 108a | GluNAc | C5-AEEA | C5-AEEA- | N(H) | 4 | H | |
| | | | GABA | | | | |
| 109a,b,c | GluNAc | C5-AEEP | C5-AEEP-GABA | N(H) | 4 | CH₂OZ² | Tr, MMTr, |
| | | | | | | | or DMTr |
| 110a | GluNAc | C5-AEEP | C5-AEEP-GABA | N(H) | 4 | H | |
| 111a,b,c | GluNAc | C5 | C5-GABA | N(H) | 4 | CH₂OZ² | Tr, MMTr, |
| | | | | | | | or DMTr |
| 112a | GluNAc | C5 | C5-GABA | N(H) | 4 | H | |
| 113a,b,c | GluNAc | C5 | C5-Gly | C(O) | 2 | CH₂OZ² | Tr, MMTr, |
| | | | | | | | or DMTr |
| 114a | GluNAc | C5 | C5-Gly | C(O) | 2 | H | |
| 115a,b,c | HDA | — | GABA | N(H) | 4 | CH₂OZ² | Tr, MMTr, |
| | | | | | | | or DMTr |
| 116a,b,c | LA | AEEA | AEEA-GABA | N(H) | 4 | CH₂OZ² | Tr, MMTr, |
| | | | | | | | or DMTr |
| 117a | LA | AEEA | AEEA-GABA | N(H) | 4 | H | |
| 118a,b,c | Lithocholic acid | AEEA | AEEA-GABA | N(H) | 4 | CH₂OZ² | Tr, MMTr, |
| | | | | | | | or DMTr |
| 119a | Lithocholic acid | AEEA | AEEA-GABA | N(H) | 4 | H | |
| 120a,b,c | MA | — | GABA | N(H) | 4 | CH₂OZ² | Tr, MMTr, |
| | | | | | | | or DMTr |
| 121a,b,c | Mannose | C5 | C5 | N(H) | 4 | CH₂OZ² | Tr, MMTr, |
| | | | | | | | or DMTr |
| 122a | Mannose | C5 | C5 | N(H) | 4 | H | |
| 123a,b,c | Mannose | C5-AEA | C5-AEA-GABA | N(H) | 4 | CH₂OZ² | Tr, MMTr, |
| | | | | | | | or DMTr |
| 124a | Mannose | C5-AEA | C5-AEA-GABA | N(H) | 4 | H | |
| 125a,b,c | Mannose | C5-AEEA | C5-AEEA- | N(H) | 4 | CH₂OZ² | Tr, MMTr, |
| | | | GABA | | | | or DMTr |
| 126a | Mannose | C5-AEEA | C5-AEEA- | N(H) | 4 | H | |
| | | | GABA | | | | |
| 127a,b,c | Mannose | C5-AEEP | C5-AEEP-GABA | N(H) | 4 | CH₂OZ² | Tr, MMTr, |
| | | | | | | | or DMTr |
| 128a | Mannose | C5-AEEP | C5-AEEP-GABA | N(H) | 4 | H | |
| 129a,b,c | Mannose | C5 | C5-GABA | N(H) | 4 | CH₂OZ² | Tr, MMTr, |
| | | | | | | | or DMTr |
| 130a | Mannose | C5 | C5-GABA | N(H) | 4 | H | |
| 131a,b,c | Mannose | C5 | C5-Gly | C(O) | 2 | CH₂OZ² | Tr, MMTr, |
| | | | | | | | or DMTr |
| 132a | Mannose | C5 | C5-Gly | C(O) | 2 | H | |
| 133a,b,c | OA | AEEA | AEEA-GABA | N(H) | 4 | CH₂OZ² | Tr, MMTr, |
| | | | | | | | or DMTr |
| 134a | OA | AEEA | AEEA-GABA | N(H) | 4 | H | |
| 135a,b,c | PA | — | GABA | N(H) | 4 | CH₂OZ² | Tr, MMTr, |
| | | | | | | | or DMTr |
| 136a | PA | — | GABA | N(H) | 4 | H | |
| 137a,b,c | PA | C5-AEA | AEA-GABA | N(H) | 4 | CH₂OZ² | Tr, MMTr, |
| | | | | | | | or DMTr |
| 138a | PA | C5-AEA | AEA-GABA | N(H) | 4 | H | |
| 139a,b,c | PA | C5-AEEA | AEEA-GABA | N(H) | 4 | CH₂OZ² | Tr, MMTr, |
| | | | | | | | or DMTr |
| 140a | PA | C5-AEEA | AEEA-GABA | N(H) | 4 | H | |
| 141a,b,c | PA | C5-AEEP | AEEP-GABA | N(H) | 4 | CH₂OZ² | Tr, MMTr, |
| | | | | | | | or DMTr |
| 142a | PA | C5-AEEP | AEEP-GABA | N(H) | 4 | H | |
| 143a,b,c | PA | — | — | N(H) | 4 | CH₂OZ² | Tr, MMTr, |
| | | | | | | | or DMTr |
| 144a | PA | — | — | N(H) | 4 | H | |
| 145a,b,c | PA/CA | — | GABA | N(H) | 4 | CH₂OZ² | Tr, MMTr, |
| | | | | | | | or DMTr |
| 146a,b,c | PA/CA | AEA | AEA-GABA | N(H) | 4 | CH₂OZ² | Tr, MMTr, |
| | | | | | | | or DMTr |
| 147a,b,c | PA/CA | AEEA | AEEA-GABA | N(H) | 4 | CH₂OZ² | Tr, MMTr, |
| | | | | | | | or DMTr |
| 148a,b,c | PA/CA | AEEP | AEEP-GABA | N(H) | 4 | CH₂OZ² | Tr, MMTr, |
| | | | | | | | or DMTr |
| 149a,b,c | PA/CA | — | — | N(H) | 4 | CH₂OZ² | Tr, MMTr, |
| | | | | | | | or DMTr |
| 150a,b,c | PA/DDA | — | GABA | N(H) | 4 | CH₂OZ² | Tr, MMTr, |
| | | | | | | | or DMTr |
| 151a,b,c | PA/DDA | AEA | AEA-GABA | N(H) | 4 | CH₂OZ² | Tr, MMTr, |
| | | | | | | | or DMTr |
| 152a,b,c | PA/DDA | AEEA | AEEA-GABA | N(H) | 4 | CH₂OZ² | Tr, MMTr, |
| | | | | | | | or DMTr |
| 153a,b,c | PA/DDA | AEEP | AEEP-GABA | N(H) | 4 | CH₂OZ² | Tr, MMTr, |
| | | | | | | | or DMTr |
| 154a,b,c | PA/DDA | — | — | N(H) | | CH₂OZ² | Tr, MMTr, |
| | | | | | | | or DMTr |
| 155a,b,c | PA/MA | — | GABA | N(H) | 4 | CH₂OZ² | Tr, MMTr, |
| | | | | | | | or DMTr |
| 156a,b,c | PA/MA | AEA | AEA-GABA | N(H) | 4 | CH₂OZ² | Tr, MMTr, |
| | | | | | | | or DMTr |
| 157a,b,c | PA/MA | AEEA | AEEA-GABA | N(H) | 4 | CH₂OZ² | Tr, MMTr, |
| | | | | | | | or DMTr |
| 158a,b,c | PA/MA | AEEP | AEEP-GABA | N(H) | 4 | CH₂OZ² | Tr, MMTr, |
| | | | | | | | or DMTr |
| 159a,b,c | PA/MA | — | — | N(H) | 4 | CH₂OZ² | Tr, MMTr, |
| | | | | | | | or DMTr |
| 160a,b,c | PA/PDA | — | GABA | N(H) | 4 | CH₂OZ² | Tr, MMTr, |
| | | | | | | | or DMTr |
| 161a,b,c | PA/PDA | AEA | AEA-GABA | N(H) | 4 | CH₂OZ² | Tr, MMTr, |
| | | | | | | | or DMTr |
| 162a,b,c | PA/PDA | AEEA | AEEA-GABA | N(H) | 4 | CH₂OZ² | Tr, MMTr, |
| | | | | | | | or DMTr |
| 163a,b,c | PA/PDA | AEEP | AEEP-GABA | N(H) | 4 | CH₂OZ² | Tr, MMTr, |
| | | | | | | | or DMTr |
| 164a,b,c | PA/PDA | — | — | N(H) | 4 | CH₂OZ² | Tr, MMTr, |
| | | | | | | | or DMTr |
| 165a,b,c | PA/PGA | — | GABA | N(H) | 4 | CH₂OZ² | Tr, MMTr, |
| | | | | | | | or DMTr |
| 166a,b,c | PA/PGA | AEA | AEA-GABA | N(H) | 4 | CH₂OZ² | Tr, MMTr, |
| | | | | | | | or DMTr |
| 167a,b,c | PA/PGA | AEEA | AEEA-GABA | N(H) | 4 | CH₂OZ² | Tr, MMTr, |
| | | | | | | | or DMTr |
| 168a,b,c | PA/PGA | AEEP | AEEP-GABA | N(H) | 4 | CH₂OZ² | Tr, MMTr, |
| | | | | | | | or DMTr |
| 169a,b,c | PA/PGA | — | — | N(H) | 4 | CH₂OZ² | Tr, MMTr, |
| | | | | | | | or DMTr |
| 170a,b,c | PA/TDA | — | GABA | N(H) | 4 | CH₂OZ² | Tr, MMTr, |
| | | | | | | | or DMTr |
| 171a,b,c | PA/TDA | AEA | AEA-GABA | N(H) | 4 | CH₂OZ² | Tr, MMTr, |
| | | | | | | | or DMTr |
| 172a,b,c | PA/TDA | AEEA | AEEA-GABA | N(H) | 4 | CH₂OZ² | Tr, MMTr, |
| | | | | | | | or DMTr |
| 173a,b,c | PA/TDA | AEEP | AEEP-GABA | N(H) | 4 | CH₂OZ² | Tr, MMTr, |
| | | | | | | | or DMTr |
| 174a,b,c | PA/TDA | — | — | N(H) | 4 | CH₂OZ² | Tr, MMTr, |
| | | | | | | | or DMTr |
| 175a,b,c | PA/UDA | — | GABA | N(H) | 4 | CH₂OZ² | Tr, MMTr, |
| | | | | | | | or DMTr |
| 176a,b,c | PA/UDA | AEA | AEA-GABA | N(H) | 4 | CH₂OZ² | Tr, MMTr, |
| | | | | | | | or DMTr |
| 177a,b,c | PA/UDA | AEEA | AEEA-GABA | N(H) | 4 | CH₂OZ² | Tr, MMTr, |
| | | | | | | | or DMTr |
| 178a,b,c | PA/UDA | AEEP | AEEP-GABA | N(H) | 4 | CH₂OZ² | Tr, MMTr, |
| | | | | | | | or DMTr |
| 179a,b,c | PA/UDA | — | — | N(H) | 4 | CH₂OZ² | Tr, MMTr, |
| | | | | | | | or DMTr |
| 180a,b,c | PDA | — | GABA | N(H) | 4 | CH₂OZ² | Tr, MMTr, |
| | | | | | | | or DMTr |
| 181a,b,c | PGA | — | GABA | N(H) | 4 | CH₂OZ² | Tr, MMTr, |
| | | | | | | | or DMTr |
| 182a,b,c | RA | AEEA | AEEA-GABA | N(H) | 4 | CH₂OZ² | Tr, MMTr, |
| | | | | | | | or DMTr |
| 183a | RA | AEEA | AEEA-GABA | N(H) | 4 | H | |
| 184a,b,c | SA | — | GABA | N(H) | 4 | CH₂OZ² | Tr, MMTr, |
| | | | | | | | or DMTr |
| 185a,b,c | SA 12-OH | — | GABA | N(H) | 4 | CH₂OZ² | Tr, MMTr, |
| | | | | | | | or DMTr |
| 186a,b,c | SA 18-OH | — | GABA | N(H) | 4 | CH₂OZ² | Tr, MMTr, |
| | | | | | | | or DMTr |
| 187a,b,c | SA 2-OH | — | GABA | N(H) | 4 | CH₂OZ² | Tr, MMTr, |
| | | | | | | | or DMTr |
| 188a,b,c | TDA | — | GABA | N(H) | 4 | CH₂OZ² | Tr, MMTr, |
| | | | | | | | or DMTr |
| 189a,b,c | UDA | — | GABA | N(H) | 4 | CH₂OZ² | Tr, MMTr, |
| | | | | | | | or DMTr |
| 190a,b,c | Ursodeoxycholic acid | AEEA | AEEA-GABA | N(H) | 4 | CH₂OZ² | Tr, MMTr, |
| | | | | | | | or DMTr |
| 191a | Ursodeoxycholic acid | AEEA | AEEA-GABA | N(H) | 4 | H | |

or

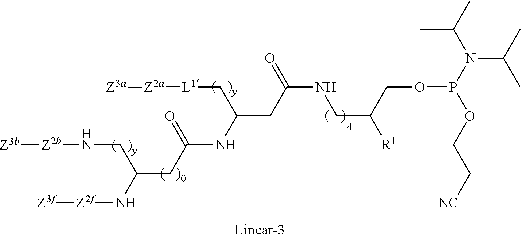

| | Z^{3a}, Z^{3b}, | | | | | | | |
|---|---|---|---|---|---|---|---|---|
| # | Z^{3f} | Z^{2a}, Z^{2b} | Z^{2f} | L^{1′} | y | x | R¹ | Z² |
| 192a,b,c | BA | — | GABA | N(H) | 4 | 0 | CH₂OZ² | Tr, MMTr, |
| | | | | | | | | or DMTr |
| 193a,b,c | BA | AEA | AEA-GABA | N(H) | 4 | 0 | CH₂OZ² | Tr, MMTr, |
| | | | | | | | | or DMTr |
| 194a,b,c | BA | AEEA | AEEA-GABA | N(H) | 4 | 0 | CH₂OZ² | Tr, MMTr, |
| | | | | | | | | or DMTr |
| 195a,b,c | BA | AEEP | AEEP-GABA | N(H) | 4 | 0 | CH₂OZ² | Tr, MMTr, |
| | | | | | | | | or DMTr |
| 196a,b,c | BA | — | — | N(H) | 4 | 0 | CH₂OZ² | Tr, MMTr, |
| | | | | | | | | or DMTr |
| 197a,b,c | GalNAc | C5-AEEA | C5-AEEA-GABA | N(H) | 4 | 0 | CH₂OZ² | Tr, MMTr, |
| | | | | | | | | or DMTr |
| 198a | GalNAc | C5-AEEA | C5-AEEA-GABA | N(H) | 4 | 0 | H | |
| 199a,b,c | GalNAc | C5 | C5-GABA | N(H) | 4 | 1 | CH₂OZ² | Tr, MMTr, |
| | | | | | | | | or DMTr |
| 200a,b,c | GalNAc | C5 | C5-GABA | N(H) | 4 | 0 | CH₂OZ² | Tr, MMTr, |
| | | | | | | | | or DMTr |
| 201a,b,c | GalNAc | C5 | C5-GABA | N(H) | 4 | 0 | CH₂OZ² | Tr, MMTr, |
| | | | | | | | | or DMTr |
| 202a,b,c | GalNAc | C5 | C5-GABA | N(H) | 4 | 1 | CH₂OZ² | Tr, MMTr, |
| | | | | | | | | or DMTr |
| 203a,b,c | GalNAc | C5 | C5-GABA | N(H) | 4 | 0 | CH₂OZ² | Tr, MMTr, |
| | | | | | | | | or DMTr |
| 204a,b,c | GalNAc | C5 | C5-GABA | N(H) | 4 | 0 | CH₂OZ² | Tr, MMTr, |
| | | | | | | | | or DMTr |
| 205a,b,c | GalNAc | C5 | C5-GABA | N(H) | 4 | 1 | CH₂OZ² | Tr, MMTr, |
| | | | | | | | | or DMTr |
| 206a,b,c | GalNAc | C5 | C5-GABA | N(H) | 4 | 0 | CH₂OZ² | Tr, MMTr, |
| | | | | | | | | or DMTr |
| 207a,b,c | GalNAc | C5 | C5-GABA | N(H) | 4 | 0 | CH₂OZ² | Tr, MMTr, |
| | | | | | | | | or DMTr |
| 208a,b,c | GalNAc | C5 | C5-GABA | N(H) | 4 | 1 | CH₂OZ² | Tr, MMTr, |
| | | | | | | | | or DMTr |
| 209a,b,c | GalNAc | C5 | C5-GABA | N(H) | 4 | 0 | CH₂OZ² | Tr, MMTr, |
| | | | | | | | | or DMTr |
| 210a,b,c | GalNAc | C5 | C5-GABA | N(H) | 4 | 0 | CH₂OZ² | Tr, MMTr, |
| | | | | | | | | or DMTr |
| 211a,b,c | GalNAc | C5 | C5-GABA | N(H) | 4 | 1 | CH₂OZ² | Tr, MMTr, |
| | | | | | | | | or DMTr |
| 212a,b,c | GalNAc | C5 | C5-GABA | N(H) | 4 | 0 | CH₂OZ² | Tr, MMTr, |
| | | | | | | | | or DMTr |
| 213a,b,c | GalNAc | C5 | C5-GABA | N(H) | 4 | 0 | CH₂OZ² | Tr, MMTr, |
| | | | | | | | | or DMTr |
| 214a,b,c | GalNAc | C5 | C5-GABA | N(H) | 4 | 1 | CH₂OZ² | Tr, MMTr, |
| | | | | | | | | or DMTr |
| 215a,b,c | GalNAc | C5 | C5-GABA | N(H) | 4 | 0 | CH₂OZ² | Tr, MMTr, |
| | | | | | | | | or DMTr |
| 216a,b,c | GalNAc | C5 | C5-GABA | N(H) | 4 | 0 | CH₂OZ² | Tr, MMTr, |
| | | | | | | | | or DMTr |
| 217a | GalNAc | C5 | C5-GABA | N(H) | 4 | 0 | H | |
| 218a,b,c | GalNAc | C5-AEEA | C5-AEEA-GLY | C(O) | 2 | 0 | CH₂OZ² | Tr, MMTr, |
| | | | | | | | | or DMTr |
| 219a | GalNAc | C5-AEEA | C5-AEEA-GLY | C(O) | 2 | 0 | H | |
| 220a,b,c | GalNAc | C5 | C5-GLY | C(O) | 2 | 0 | CH₂OZ² | Tr, MMTr, |
| | | | | | | | | or DMTr |
| 221a | GalNAc | C5 | C5-GLY | C(O) | 2 | 0 | H | |
| 222a,b,c | GluNAc | C5-AEEA | C5-AEEA-GABA | N(H) | 4 | 0 | CH₂OZ² | Tr, MMTr, |
| | | | | | | | | or DMTr |
| 223a | GluNAc | C5-AEEA | C5-AEEA-GABA | N(H) | 4 | 0 | H | |
| 224a,b,c | GluNAc | C5 | C5-GABA | N(H) | 4 | 0 | CH₂OZ² | Tr, MMTr, |
| | | | | | | | | or DMTr |
| 225a | GluNAc | C5 | C5-GABA | N(H) | 4 | 0 | H | |
| 226a,b,c | GluNAc | C5-AEEA | C5-AEEA-GLY | C(O) | 2 | 0 | CH₂OZ² | Tr, MMTr, |
| | | | | | | | | or DMTr |
| 227a | GluNAc | C5-AEEA | C5-AEEA-GLY | C(O) | 2 | 0 | H | |
| 228a,b,c | GluNAc | C5 | C5-GLY | C(O) | 2 | 0 | CH₂OZ² | Tr, MMTr, |
| | | | | | | | | or DMTr |
| 229a | GluNAc | C5 | C5-GLY | C(O) | 2 | 0 | H | |
| 230a,b,c | Mannose | C5-AEEA | C5-AEEA-GABA | N(H) | 4 | 0 | CH₂OZ² | Tr, MMTr, |
| | | | | | | | | or DMTr |
| 231a | Mannose | C5-AEEA | C5-AEEA-GABA | N(H) | 4 | 0 | H | |
| 232a,b,c | Mannose | C5 | C5-GABA | N(H) | 4 | 0 | CH₂OZ² | Tr, MMTr, |
| | | | | | | | | or DMTr |
| 233a | Mannose | C5 | C5-GABA | N(H) | 4 | 0 | H | |
| 234a,b,c | Mannose | C5-AEEA | C5-AEEA-GLY | C(O) | 2 | 0 | CH₂OZ² | Tr, MMTr, |
| | | | | | | | | or DMTr |
| 235a | Mannose | C5-AEEA | C5-AEEA-GLY | C(O) | 2 | 0 | H | |
| 236a,b,c | Mannose | C5 | C5-GLY | C(O) | 2 | 0 | CH₂OZ² | Tr, MMTr, |
| | | | | | | | | or DMTr |
| 237a | Mannose | C5 | C5-GLY | C(O) | 2 | 0 | H | |
| 238a,b,c | PA | — | GABA | N(H) | 4 | 0 | CH₂OZ² | Tr, MMTr, |
| | | | | | | | | or DMTr |
| 239a | PA | — | GABA | N(H) | 4 | 0 | H | |
| 240a,b,c | PA | AEA | AEA-GABA | N(H) | 4 | 0 | CH₂OZ² | Tr, MMTr, |
| | | | | | | | | or DMTr |
| 241a | PA | AEA | AEA-GABA | N(H) | 4 | 0 | H | |
| 242a,b,c | PA | AEEA | AEEA-GABA | N(H) | 4 | 0 | CH₂OZ² | Tr, MMTr, |
| | | | | | | | | or DMTr |
| 243a | PA | AEEA | AEEA-GABA | N(H) | 4 | 0 | H | |
| 244a,b,c | PA | AEEA | AEEP-GABA | N(H) | 4 | 0 | CH₂OZ² | Tr, MMTr, |
| | | | | | | | | or DMTr |
| 245a | PA | AEEP | AEEP-GABA | N(H) | 4 | 0 | H | |
| 246a,b,c | PA | — | — | N(H) | 4 | 0 | CH₂OZ² | Tr, MMTr, |
| | | | | | | | | or DMTr |
| 247a | PA | — | — | N(H) | 4 | 0 | H | |

or

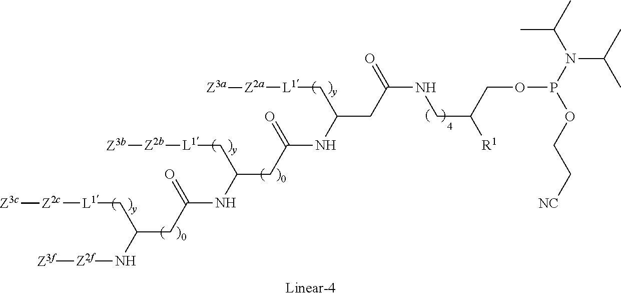

| | Z^{3a}, Z^{3b}, | Z^{2a}, Z^{2b}, | | | | | |
|---|---|---|---|---|---|---|---|
| # | Z^{3c}, Z^{3f} | Z^{2c} | Z^{2f} | L^{1′} | y | R¹ | Z² |
| 248a,b,c | GalNAc | C5-AEEA | C5-AEEA-GABA | N(H) | 4 | CH₂OZ² | Tr, MMTr, |
| | | | | | | | or DMTr |
| 249a | GalNAc | C5-AEEA | C5-AEEA-GABA | N(H) | 4 | H | |
| 250a,b,c | GalNAc | C5-AEEA | C5-AEEA-GLY | C(O) | 2 | CH₂OZ² | Tr, MMTr, |
| | | | | | | | or DMTr |
| 251a | GalNAc | C5-AEEA | C5-AEEA-GLY | C(O) | 2 | H | |
| 252a,b,c | GluNAc | C5-AEEA | C5-AEEA-GABA | N(H) | 4 | CH₂OZ² | Tr, MMTr, |
| | | | | | | | or DMTr |
| 253a | GluNAc | C5-AEEA | C5-AEEA-GABA | N(H) | 4 | H | |
| 254a,b,c | GluNAc | C5-AEEA | C5-AEEA-GLY | C(O) | 2 | CH₂OZ² | Tr, MMTr, |
| | | | | | | | or DMTr |
| 255a | GluNAc | C5-AEEA | C5-AEEA-GLY | C(O) | 2 | H | |
| 256a,b,c | Mannose | C5-AEEA | C5-AEEA-GABA | N(H) | 4 | CH₂OZ² | Tr, MMTr, |
| | | | | | | | or DMTr |
| 257a | Mannose | C5-AEEA | C5-AEEA-GABA | N(H) | 4 | H | |
| 258a,b,c | Mannose | C5-AEEA | C5-AEEA-GLY | C(O) | 2 | CH₂OZ² | Tr, MMTr, |
| | | | | | | | or DMTr |
| 259a | Mannose | C5-AEEA | C5-AEEA-GLY | C(O) | 2 | H | |
| 260a,b,c | PA | AEEA | AEEA-GABA | N(H) | 4 | CH₂OZ² | Tr, MMTr, |
| | | | | | | | or DMTr |
| 261a | PA | AEEA | AEEA-GABA | N(H) | 4 | H | |

or

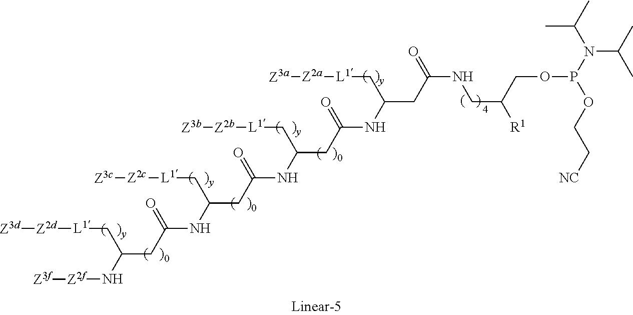

| | Z^{3a}, Z^{3b}, Z^{3c}, | Z^{2a}, Z^{2b}, | | | | | |
|---|---|---|---|---|---|---|---|
| # | Z^{3d}, Z^{3f} | Z^{2c}, Z^{2d} | Z^{2f} | L^{1′} | y | R¹ | Z² |
| 262a,b,c | GalNAc | C5 | C5 | C(O) | 2 | CH₂OZ² | Tr, MMTr, |
| | | | | | | | or DMTr |
| 263a | GalNAc | C5 | C5 | C(O) | 2 | H | |
| 264a,b,c | GalNAc | C5-AEEA | C5-AEEA-GABA | N(H) | 4 | CH₂OZ² | Tr, MMTr, |
| | | | | | | | or DMTr |
| 265a | GalNAc | C5-AEEA | C5-AEEA-GABA | N(H) | 4 | H | |
| 266a,b,c | GluNAc | C5 | C5 | C(O) | 2 | CH₂OZ² | Tr, MMTr, |
| | | | | | | | or DMTr |
| 267a | GluNAc | C5 | C5 | C(O) | 2 | H | |
| 268a,b,c | GluNAc | C5-AEEA | C5-AEEA-GABA | N(H) | 4 | CH₂OZ² | Tr, MMTr, |
| | | | | | | | or DMTr |
| 269a | GluNAc | C5-AEEA | C5-AEEA-GABA | N(H) | 4 | H | |
| 270a,b,c | Mannose | C5 | C5 | C(O) | 2 | CH₂OZ² | Tr, MMTr, |
| | | | | | | | or DMTr |
| 271a | Mannose | C5 | C5 | C(O) | 2 | H | |
| 272a,b,c | Mannose | C5-AEEA | C5-AEEA-GABA | N(H) | 4 | CH₂OZ² | Tr, MMTr, |
| | | | | | | | or DMTr |
| 273a | Mannose | C5-AEEA | C5-AEEA-GABA | N(H) | 4 | H | |
| 274a,b,c | PA | AEEA | AEEA-GABA | N(H) | 4 | CH₂OZ² | Tr, MMTr, |
| | | | | | | | or DMTr |
| 275a | PA | AEEA | AEEA-GABA | N(H) | 4 | H | |

or

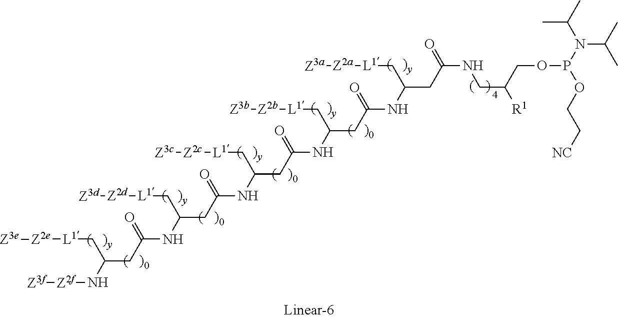

| | | Z^{2a}, Z^{2b}, | | | | | |
|---|---|---|---|---|---|---|---|
| | Z^{3a}, Z^{3b}, Z^{3c}, | Z^{2c}, Z^{2d}, | | | | | |
| # | Z^{3d}, Z^{3e}, Z^{3f} | Z^{2e} | Z^{2f} | L^{1′} | y | R¹ | Z² |
| 276a,b,c | GalNAc | C5-AEEA | C5-AEEA-GABA | N(H) | 4 | CH₂OZ² | Tr, MMTr, |
| | | | | | | | or DMTr |
| 277a | GalNAc | C5-AEEA | C5-AEEA-GABA | N(H) | 4 | H | |
| 278a,b,c | GalNAc | C5-AEEA | C5-AEEA-GLY | C(O) | 2 | CH₂OZ² | Tr, MMTr, |
| | | | | | | | or DMTr |
| 279a | GalNAc | C5-AEEA | C5-AEEA-GLY | C(O) | 2 | H | |
| 280a,b,c | GluNAc | C5-AEEA | C5-AEEA-GABA | N(H) | 4 | CH₂OZ² | Tr, MMTr, |
| | | | | | | | or DMTr |
| 281a | GluNAc | C5-AEEA | C5-AEEA-GABA | N(H) | 4 | H | |
| 282a,b,c | GluNAc | C5-AEEA | C5-AEEA-GLY | C(O) | 2 | CH₂OZ² | Tr, MMTr, |
| | | | | | | | or DMTr |
| 283a | GluNAc | C5-AEEA | C5-AEEA-GLY | C(O) | 2 | H | |
| 284a,b,c | Mannose | C5-AEEA | C5-AEEA-GABA | N(H) | 4 | CH₂OZ² | Tr, MMTr, |
| | | | | | | | or DMTr |
| 285a | Mannose | C5-AEEA | C5-AEEA-GABA | N(H) | 4 | H | |
| 286a,b,c | Mannose | C5-AEEA | C5-AEEA-GLY | C(O) | 2 | CH₂OZ² | Tr, MMTr, |
| | | | | | | | or DMTr |
| 287a | Mannose | C5-AEEA | C5-AEEA-GLY | C(O) | 2 | H | |
| 288a,b,c | PA | AEEA | AEEA-GABA | N(H) | 4 | CH₂OZ² | Tr, MMTr, |
| | | | | | | | or DMTr |
| 289a | PA | AEEA | AEEA-GABA | N(H) | 4 | H | |

or a salt thereof.

18. The compound of claim 1, comprising the formula: 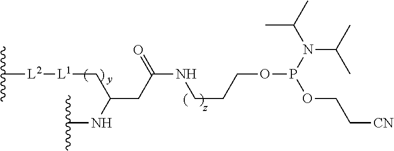
wherein:
y is 0, 1, 2, 3, 4, 5, or 6;
z is 0, 1, 2, 3, 4, 5, or 6; and
L¹ is N(H) and L² is C(O), or L¹ is C(O) and L² is N(H).

19. The compound of claim 21, wherein y is 2 or 4, and z is 4.

20. A compound, comprising a formula: 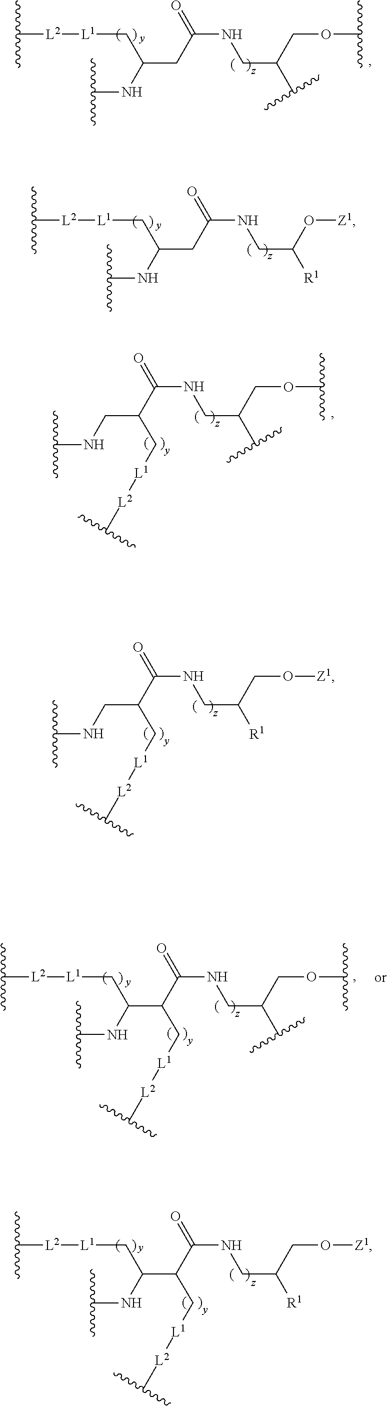
wherein:
y is 0, 1, 2, 3, 4, 5, or 6;
z is 0, 1, 2, 3, 4, 5, or 6;
L¹ is N(H) and L² is C(O), or L¹ is C(O) and L² is N(H);
R¹ is H or CH₂OH; and
Z¹ comprises a solid support or an oligomer; or
R¹ is H and Z¹ is a phosphoramidite

21. A compound, comprising a formula: 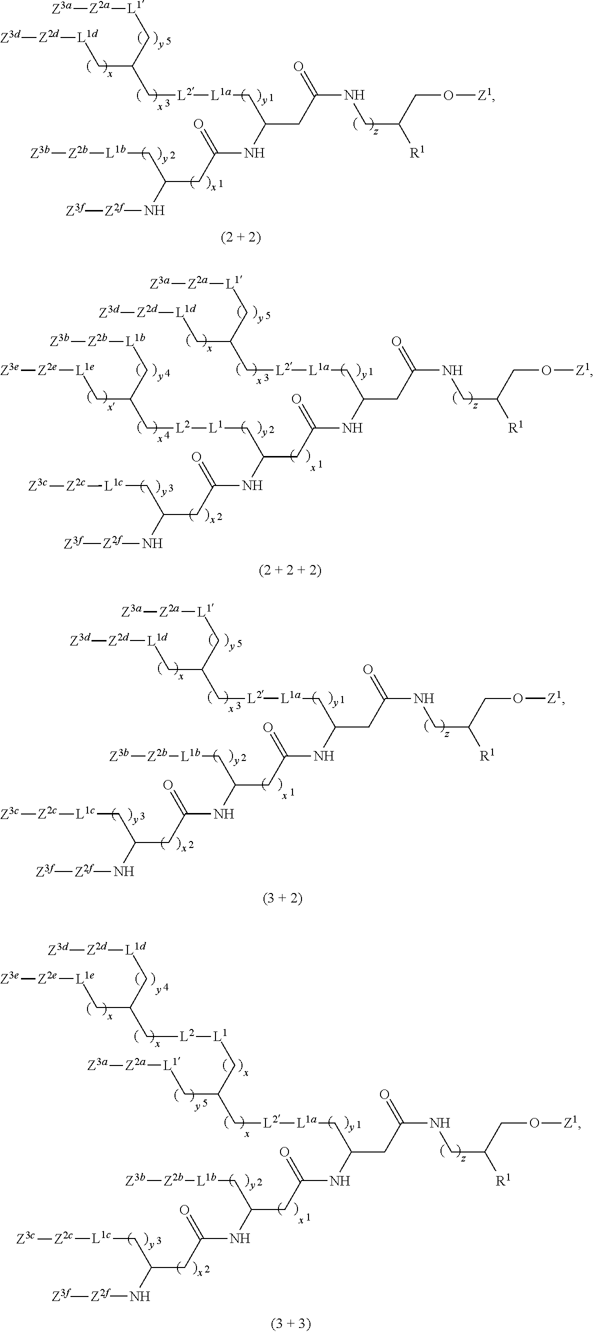 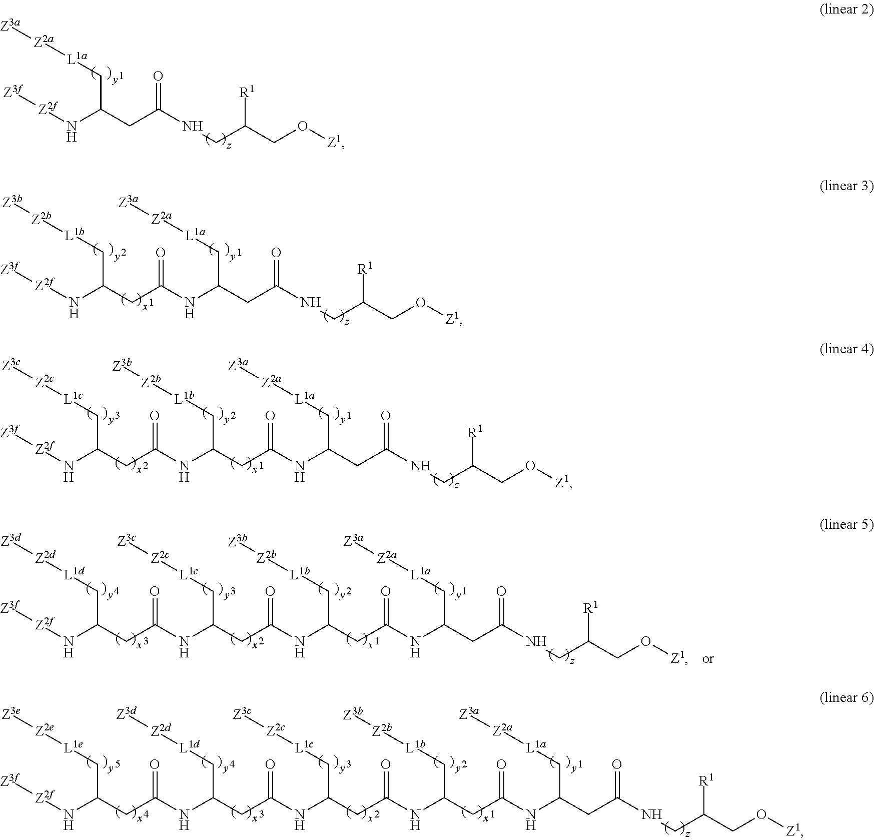
or a salt thereof,
wherein
Z¹ is H, a phosphoramidite, a solid support, or a macromolecule;
R¹ is H, CH₂OH, or CH₂OZ²;
Z² is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
z is 0, 1, 2, 3, 4, 5, or 6;
x, x′, x¹, x², x³, and x⁴ are each, independently, 0, 1, 2, 3, 4, 5, or 6;
y, y′, y¹, y², y³, y⁴, and y⁵, are each, independently, 0, 1, 2, 3, 4, 5, or 6;
Z^{2a}, Z^{2b}, Z^{2c}, Z^{2d}, Z^{2e}, and Z^{2f} are each, independently, —(C₁₋₂₀ alkyl)-, —(C₂₋₂₀ alkenyl)-, —(C₂₋₂₀ alkynyl)-, —(C₃₋₂₀ cycloalkyl)-, —(C₄₋₂₀ cycloalkenyl)-, —(C₅₋₂₀ cycloalkynyl)-, —(C₁₋₂₀ heterocycloalkyl)-, —(C₂₋₂₀ heterocycloalkenyl)-, —(C₂₋₂₀ heterocycloalkynyl)-, and poly glycol such as —(CH₂CH₂O)ₙ—, —(CH₂CH₂CH₂O)ₙ—, —(CH₂CH₂CH₂CH₂O)ₙ—, where n is 1 to about 6;
Z^{3a}, Z^{3b}, Z^{3c}, Z^{3d}, Z^{3e}, and Z^{3f} are each, independently, selected from carbohydrate receptor ligands, such as N-acetylgalactosamine, N-acetylglucosamine, and mannose, lipids such as cholesterol, bile acid derivatives, and fatty acids, cell penetrating peptides (CPPs), specific small molecules showing cell-targeting effects, polymers such as poly glycols, aptamers and antibodies; and
L¹, L^{1′}, L^{1a}, L^{1b}, L^{1c}, L^{1d}, L^{1e}, L², and L^{2′}, are each, independently, N(H) or C(O).

22. The compound of claim 21, wherein:
R¹ is H or CH₂OZ², and Z¹ is H, a solid support, an oligomer, or 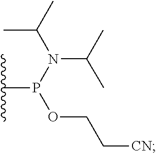 [SMILES: *P(OCCC#N)N(C(C)C)C(C)C]
or R¹ is H, CH₂OH, or CH₂OZ², and Z¹ is H, a solid support, or an oligomer;
Z² is triphenylmethyl, monomethoxytriphenylmethyl, dimethoxytriphenylmethyl, trimethoxytriphenylmethyl, monomethyltriphenylmethyl, dimethyltriphenylmethyl, trimethyltriphenylmethyl, monochlorotriphenylmethyl, dichlorotriphenylmethyl, trichlorotriphenylmethyl, methylsulfonyltriphenylmethyl, monomethoxymethylsulfonyltriphenylmethyl, dimethoxymethylsulfonyltriphenylmethyl, monomethoxydimethylsulfonyltriphenylmethyl, or trimethylsulfonyltriphenylmethyl;
z is 4;
x, x′, x¹, x², x³, and x⁴ are each, independently, 0 or 1;
y, y′, y¹, y², y³, y⁴, and y⁵, are each, independently, 0, 1, 2, 3, 4, 5, or 6;
Z^{2a}, Z^{2b}, Z^{2c}, Z^{2d}, Z^{2e}, and Z^{2f} are each, independently, selected from AEA-GABA, AEEA-GABA, AEEP-GABA, C5, C5-AEA-GABA, C5-AEEA-GABA, C5-AEEA-GLY, C5-AEEP-GABA, C5-GABA, C5-Gly, or GABA; and
Z^{3a}, Z^{3b}, Z^{3c}, Z^{3d}, Z^{3e}, and Z^{3f} are 3p-hydroxy 5-cholenoic acid, ACA, ALA, ARA, BA, CA, Chenocholic acid, Cholesterol, Cholic acid, DDA, DDA 12-OH, DHA, EA, EPA, GalNAc, GLA, GluNAc, HDA, LA, Lithocholic acid, MA, Mannose, OA, PA, each independently selected from PA or CA, each independently selected from PA or DDA, each independently selected from PA or MA, each independently selected from PA or PDA, each independently selected from PA or PGA, each independently selected from PA or TDA, each independently selected from PA or UDA, PDA, PGA, RA, SA, SA 12-OH, SA 18-OH, SA 2-OH, TDA, UDA, or Ursodeoxycholic acid.

23. A composition, comprising the compound of claim 1 and a carrier, optionally wherein the composition is a pharmaceutical composition comprising a pharmaceutically acceptable carrier.

24. A method, comprising administering the compound claim 1, to a subject in need thereof.

25. An article of manufacture, comprising the compound of claim 1, and instructions for pharmaceutical use thereof.
